SEQUENCE LISTING (389 sequences)

SEQ ID NO: 1 - protein, 9 aa, Homo sapiens
LLMWITQCF

SEQ ID NO: 2 - protein, 9 aa, Homo sapiens
GSPFPAAVI

SEQ ID NO: 3 - protein, 9 aa, Homo sapiens
RGPESRLLE

SEQ ID NO: 4 - protein, 9 aa, Homo sapiens
AFLTVKKQM

SEQ ID NO: 5 - protein, 6 aa, Homo sapiens
  MISC_FEATURE (1)..(6): X represents a hydrophobic amino acid, Ala, Phe, Gly, Ile, Leu, Pro, Val.
  MISC_FEATURE (1)..(6): X represents a hydrophobic amino acid, for example,  Ala, Phe, Gly, Ile, Leu, Pro, Val.
XRSKRX

SEQ ID NO: 6 - protein, 3 aa, Homo sapiens
  MISC_FEATURE (1)..(8): X can be Arg or Lys.  N can be an integer from 0 to 6.
  MISC_FEATURE (1)..(8): X can be Arg or Lys.  N can be an integer from 0 to 6 amino acids.
NXN

SEQ ID NO: 7 - protein, 13 aa, Homo sapiens
AKFVAAWTLKAAA

SEQ ID NO: 8 - DNA, 6 bp, Homo sapiens
tctaga

SEQ ID NO: 9 - DNA, 6 bp, Homo sapiens
gggccc

SEQ ID NO: 10 - DNA, 216 bp, Homo sapiens
tctagagccaccatgctggtgacagccatgtgtctgctgggcaatgtcagcttcgtccggagcaagcggctgcggggaccagagtctcggctcctggaggtgcggagcaagcggctgtccccatcttacgcctaccaccagttcgtccggagcaagcggctgggctgtgccttcctgaccgtgaagcagatgcggagcaagcggctgtgagggccc

SEQ ID NO: 11 - protein, 65 aa, Homo sapiens
MLVTAMCLLGNVSFVRSKRLRGPESRLLEVRSKRLSPSYAYHQFVRSKRLGCAFLTVKQMRSKRL

SEQ ID NO: 12 - protein, 9 aa, Homo sapiens
FLGYLILGV

SEQ ID NO: 13 - protein, 15 aa, Homo sapiens
SVSESDTIRSISIAS

SEQ ID NO: 14 - protein, 15 aa, Homo sapiens
LLANGRMPTVLQCVN

SEQ ID NO: 15 - protein, 15 aa, Homo sapiens
RMPTVLQCVNVSVVS

SEQ ID NO: 16 - protein, 12 aa, Homo sapiens
CTACRWKKACQR

SEQ ID NO: 17 - protein, 9 aa, Homo sapiens
VLDGLDVLL

SEQ ID NO: 18 - protein, 10 aa, Homo sapiens
SLYSFPEPEA

SEQ ID NO: 19 - protein, 10 aa, Homo sapiens
ALYVDSLFFL

SEQ ID NO: 20 - protein, 9 aa, Homo sapiens
SLLQHLIGL

SEQ ID NO: 21 - protein, 9 aa, Homo sapiens
LYVDSLFFL

SEQ ID NO: 22 - protein, 11 aa, Homo sapiens
TSEKRPFMCAY

SEQ ID NO: 23 - protein, 9 aa, Homo sapiens
CMTWNQMNL

SEQ ID NO: 24 - protein, 11 aa, Homo sapiens
LSHLQMHSRKH

SEQ ID NO: 25 - protein, 16 aa, Homo sapiens
KRYFKLSHLQMHSRKH

SEQ ID NO: 26 - protein, 8 aa, Homo sapiens
CYMEAVAL

SEQ ID NO: 27 - protein, 9 aa, Homo sapiens
SLLFLLFSL

SEQ ID NO: 28 - protein, 9 aa, Homo sapiens
VLPLTVAEV

SEQ ID NO: 29 - protein, 9 aa, Homo sapiens
ALQGGGPPY

SEQ ID NO: 30 - protein, 9 aa, Homo sapiens
LYPKARLAF

SEQ ID NO: 31 - protein, 9 aa, Homo sapiens
AFLPWHRLF

SEQ ID NO: 32 - protein, 9 aa, Homo sapiens
SLLMWITQC

SEQ ID NO: 33 - protein, 9 aa, Homo sapiens
LAMPFATPM

SEQ ID NO: 34 - protein, 9 aa, Homo sapiens
ARGPESRLL

SEQ ID NO: 35 - protein, 11 aa, Homo sapiens
MLMAQEALAFL

SEQ ID NO: 36 - protein, 11 aa, Homo sapiens
YLAMPFATPME

SEQ ID NO: 37 - protein, 10 aa, Homo sapiens
ASGPGGGAPR

SEQ ID NO: 38 - protein, 10 aa, Homo sapiens
LAAQERRVPR

SEQ ID NO: 39 - protein, 10 aa, Homo sapiens
TVSGNILTIR

SEQ ID NO: 40 - protein, 13 aa, Homo sapiens
APRGPHGGAASGL

SEQ ID NO: 41 - protein, 11 aa, Homo sapiens
MPFATPMEAEL

SEQ ID NO: 42 - protein, 12 aa, Homo sapiens
KEFTVSGNILTI

SEQ ID NO: 43 - protein, 9 aa, Homo sapiens
MPFATPMEA

SEQ ID NO: 44 - protein, 9 aa, Homo sapiens
FATPMEAEL

SEQ ID NO: 45 - protein, 11 aa, Homo sapiens
FATPMEAELAR

SEQ ID NO: 46 - protein, 9 aa, Homo sapiens
LAMPFATPM

SEQ ID NO: 47 - protein, 14 aa, Homo sapiens
SLLMWITQCFLPVF

SEQ ID NO: 48 - protein, 11 aa, Homo sapiens
EAELARRSLAQ

SEQ ID NO: 49 - protein, 12 aa, Homo sapiens
EFYLAMPFATPM

SEQ ID NO: 50 - protein, 25 aa, Homo sapiens
PGVLLKEFTVSGNILTIRLTAADHR

SEQ ID NO: 51 - protein, 12 aa, Homo sapiens
RLLEFYLAMPFA

SEQ ID NO: 52 - protein, 20 aa, Homo sapiens
QGAMLAAQERRVPRAAEVPR

SEQ ID NO: 53 - protein, 13 aa, Homo sapiens
PFATPMEAELARR

SEQ ID NO: 54 - protein, 20 aa, Homo sapiens
PGVLLKEFTVSGNILTIRLT

SEQ ID NO: 55 - protein, 10 aa, Homo sapiens
VLLKEFTVSG

SEQ ID NO: 56 - protein, 18 aa, Homo sapiens
AADHRQLQLSISSCLQQL

SEQ ID NO: 57 - protein, 15 aa, Homo sapiens
LKEFTVSGNILTIRL

SEQ ID NO: 58 - protein, 11 aa, Homo sapiens
KEFTVSGNILT

SEQ ID NO: 59 - protein, 14 aa, Homo sapiens
LLEFYLAMPFATPM

SEQ ID NO: 60 - protein, 14 aa, Homo sapiens
AGATGGRGPRGAGA

SEQ ID NO: 61 - protein, 13 aa, Homo sapiens
TYYRPGVNLSLSC

SEQ ID NO: 62 - protein, 13 aa, Homo sapiens
EIIYPNASLLIQN

SEQ ID NO: 63 - protein, 15 aa, Homo sapiens
YACFVSNLATGRNNS

SEQ ID NO: 64 - protein, 13 aa, Homo sapiens
LWWVNNQSLPVSP

SEQ ID NO: 65 - protein, 13 aa, Homo sapiens
NSIVKSITVSASG

SEQ ID NO: 66 - protein, 9 aa, Homo sapiens
KTWGQYWQV

SEQ ID NO: 67 - protein, 10 aa, Homo sapiens
AMLGTHTMEV

SEQ ID NO: 68 - protein, 9 aa, Homo sapiens
ITDQVPFSV

SEQ ID NO: 69 - protein, 9 aa, Homo sapiens
YLEPGPVTA

SEQ ID NO: 70 - protein, 10 aa, Homo sapiens
LLDGTATLRL

SEQ ID NO: 71 - protein, 10 aa, Homo sapiens
VLYRYGSFSV

SEQ ID NO: 72 - protein, 10 aa, Homo sapiens
SLADTNSLAV

SEQ ID NO: 73 - protein, 9 aa, Homo sapiens
RLMKQDFSV

SEQ ID NO: 74 - protein, 9 aa, Homo sapiens
RLPRIFCSC

SEQ ID NO: 75 - protein, 9 aa, Homo sapiens
LIYRRRLMK

SEQ ID NO: 76 - protein, 9 aa, Homo sapiens
ALLAVGATK

SEQ ID NO: 77 - protein, 10 aa, Homo sapiens
IALNFPGSQK

SEQ ID NO: 78 - protein, 9 aa, Homo sapiens
RSYVPLAHR

SEQ ID NO: 79 - protein, 9 aa, Homo sapiens
VVPCEPPEV

SEQ ID NO: 80 - protein, 9 aa, Homo sapiens
HLYQGCQVV

SEQ ID NO: 81 - protein, 10 aa, Homo sapiens
YLVPQQGFFC

SEQ ID NO: 82 - protein, 9 aa, Homo sapiens
PLQPEQLQV

SEQ ID NO: 83 - protein, 9 aa, Homo sapiens
TLEEITGYL

SEQ ID NO: 84 - protein, 9 aa, Homo sapiens
ALIHHNTHL

SEQ ID NO: 85 - protein, 9 aa, Homo sapiens
PLTSIISAV

SEQ ID NO: 86 - protein, 9 aa, Homo sapiens
VLRENTSPK

SEQ ID NO: 87 - protein, 9 aa, Homo sapiens
TYLPTNASL

SEQ ID NO: 88 - protein, 9 aa, Homo sapiens
RYQLDPKFI

SEQ ID NO: 89 - protein, 9 aa, Homo sapiens
ILFTINFTI

SEQ ID NO: 90 - protein, 9 aa, Homo sapiens
VLFTINFTI

SEQ ID NO: 91 - protein, 9 aa, Homo sapiens
TLNFTITNL

SEQ ID NO: 92 - protein, 9 aa, Homo sapiens
VLQGLLKPL

SEQ ID NO: 93 - protein, 9 aa, Homo sapiens
VLQGLLRPV

SEQ ID NO: 94 - protein, 9 aa, Homo sapiens
RLDPKSPGV

SEQ ID NO: 95 - protein, 9 aa, Homo sapiens
QLYWELSKL

SEQ ID NO: 96 - protein, 9 aa, Homo sapiens
KLTRGIVEL

SEQ ID NO: 97 - protein, 9 aa, Homo sapiens
QLTNGITEL

SEQ ID NO: 98 - protein, 9 aa, Homo sapiens
QLTHNITEL

SEQ ID NO: 99 - protein, 9 aa, Homo sapiens
TLDRNSLYV

SEQ ID NO: 100 - protein, 9 aa, Homo sapiens
FLLSLALML

SEQ ID NO: 101 - protein, 9 aa, Homo sapiens
NLGPWIQQV

SEQ ID NO: 102 - protein, 9 aa, Homo sapiens
ILDSSEEDK

SEQ ID NO: 103 - protein, 10 aa, Homo sapiens
YLPKSWTIQV

SEQ ID NO: 104 - protein, 12 aa, Homo sapiens
WIHEQMERDLKT

SEQ ID NO: 105 - protein, 9 aa, Homo sapiens
LLANAYIYV

SEQ ID NO: 106 - protein, 9 aa, Homo sapiens
YLLCKAFGA

SEQ ID NO: 107 - protein, 9 aa, Homo sapiens
KLSPYVHYT

SEQ ID NO: 108 - protein, 20 aa, Homo sapiens
PDTRPAPGSTAPPAHGVTSA

SEQ ID NO: 109 - protein, 9 aa, Homo sapiens
STAPPVHNV

SEQ ID NO: 110 - protein, 9 aa, Homo sapiens
LLLLTVLTV

SEQ ID NO: 111 - protein, 12 aa, Homo sapiens
PGSTAPPAHGVT

SEQ ID NO: 112 - protein, 9 aa, Homo sapiens
MLAVISCAV

SEQ ID NO: 113 - protein, 9 aa, Homo sapiens
RQKRILVNL

SEQ ID NO: 114 - protein, 9 aa, Homo sapiens
NYNNFYRFL

SEQ ID NO: 115 - protein, 10 aa, Homo sapiens
EYSKECLKEF

SEQ ID NO: 116 - protein, 9 aa, Homo sapiens
EYLSLSDKI

SEQ ID NO: 117 - protein, 9 aa, Homo sapiens
ELVRRILSR

SEQ ID NO: 118 - protein, 9 aa, Homo sapiens
APRGVRMAV

SEQ ID NO: 119 - protein, 20 aa, Homo sapiens
QGAMLAAQERRVPRAAEVPR

SEQ ID NO: 120 - protein, 22 aa, Homo sapiens
CLSRRPWKRSWSAGSCPGMPHL

SEQ ID NO: 121 - protein, 13 aa, Homo sapiens
ILSRDAAPLPRPG

SEQ ID NO: 122 - protein, 9 aa, Homo sapiens
EVDPASNTY

SEQ ID NO: 123 - protein, 10 aa, Homo sapiens
GVYDGREHTV

SEQ ID NO: 124 - protein, 9 aa, Homo sapiens
NYKRCFPVI

SEQ ID NO: 125 - protein, 8 aa, Homo sapiens
SESLKMIF

SEQ ID NO: 126 - protein, 20 aa, Homo sapiens
INKTSGPKRGKHAWTHRLRE

SEQ ID NO: 127 - protein, 20 aa, Homo sapiens
YFSKKEWEKMKSSEKIVYVY

SEQ ID NO: 128 - protein, 20 aa, Homo sapiens
MKLNYEVMTKLGFKVTLPPF

SEQ ID NO: 129 - protein, 20 aa, Homo sapiens
KHAWTHRLRERKQLVVYEEI

SEQ ID NO: 130 - protein, 20 aa, Homo sapiens
LGFKVTLPPFMRSKRAADFH

SEQ ID NO: 131 - protein, 20 aa, Homo sapiens
KSSEKIVYVYMKLNYEVMTK

SEQ ID NO: 132 - protein, 9 aa, Homo sapiens
SLGWLFLLL

SEQ ID NO: 133 - protein, 9 aa, Homo sapiens
LSRLSNRLL

SEQ ID NO: 134 - protein, 9 aa, Homo sapiens
TMNGSKSPV

SEQ ID NO: 135 - protein, 9 aa, Homo sapiens
PLLENVISK

SEQ ID NO: 136 - protein, 9 aa, Homo sapiens
SLSKILDTV

SEQ ID NO: 137 - protein, 9 aa, Homo sapiens
AARAVFLAL

SEQ ID NO: 138 - protein, 9 aa, Homo sapiens
EADPTGHSY

SEQ ID NO: 139 - protein, 9 aa, Homo sapiens
KVLEYVIKV

SEQ ID NO: 140 - protein, 9 aa, Homo sapiens
SLFRAVITK

SEQ ID NO: 141 - protein, 10 aa, Homo sapiens
EVYDGREHSA

SEQ ID NO: 142 - protein, 9 aa, Homo sapiens
RVRFFFPSL

SEQ ID NO: 143 - protein, 10 aa, Homo sapiens
REPVTKAEML

SEQ ID NO: 144 - protein, 10 aa, Homo sapiens
KEADPTGHSY

SEQ ID NO: 145 - protein, 9 aa, Homo sapiens
DPARYEFLW

SEQ ID NO: 146 - protein, 11 aa, Homo sapiens
ITKKVADLVGF

SEQ ID NO: 147 - protein, 9 aa, Homo sapiens
SAFPTTINF

SEQ ID NO: 148 - protein, 9 aa, Homo sapiens
SAYGEPRKL

SEQ ID NO: 149 - protein, 15 aa, Homo sapiens
TSCILESLFRAVITK

SEQ ID NO: 150 - protein, 15 aa, Homo sapiens
PRALAETSYVKVLEY

SEQ ID NO: 151 - protein, 16 aa, Homo sapiens
FLLLKYRAREPVTKAE

SEQ ID NO: 152 - protein, 12 aa, Homo sapiens
EYVIKVSARVRF

SEQ ID NO: 153 - protein, 10 aa, Homo sapiens
YLQLVFGIEV

SEQ ID NO: 154 - protein, 9 aa, Homo sapiens
EYLQLVFGI

SEQ ID NO: 155 - protein, 9 aa, Homo sapiens
EGDCAPEEK

SEQ ID NO: 156 - protein, 14 aa, Homo sapiens
LLKYRAREPVTKAE

SEQ ID NO: 157 - protein, 9 aa, Homo sapiens
KASEKIFYV

SEQ ID NO: 158 - protein, 16 aa, Homo sapiens
EKIQKAFDDIAKYFSK

SEQ ID NO: 159 - protein, 11 aa, Homo sapiens
FGRLQGISPKI

SEQ ID NO: 160 - protein, 18 aa, Homo sapiens
WEKMKASEKIFYVYMKRK

SEQ ID NO: 161 - protein, 15 aa, Homo sapiens
KIFYVYMKRKYEAMT

SEQ ID NO: 162 - protein, 14 aa, Homo sapiens
KIFYVYMKRKYEAM

SEQ ID NO: 163 - protein, 15 aa, Homo sapiens
CEFHACWPAFTVLGE

SEQ ID NO: 164 - protein, 10 aa, Homo sapiens
SSPQGSPEPL

SEQ ID NO: 165 - protein, 9 aa, Homo sapiens
ILIDWLVQV

SEQ ID NO: 166 - protein, 9 aa, Homo sapiens
FLKNVGESV

SEQ ID NO: 167 - protein, 10 aa, Homo sapiens
ELTLGEFLKL

SEQ ID NO: 168 - protein, 9 aa, Homo sapiens
KFLDALISL

SEQ ID NO: 169 - protein, 10 aa, Homo sapiens
ALGGHPLLGV

SEQ ID NO: 170 - protein, 9 aa, Homo sapiens
LPRWPPPQL

SEQ ID NO: 171 - protein, 9 aa, Homo sapiens
ILAKFLHWL

SEQ ID NO: 172 - protein, 9 aa, Homo sapiens
RLVDDFLLV

SEQ ID NO: 173 - protein, 15 aa, Homo sapiens
RPGLLGASVLGLDDI

SEQ ID NO: 174 - protein, 15 aa, Homo sapiens
LTDLQPYMRQFVAHL

SEQ ID NO: 175 - protein, 9 aa, Homo sapiens
VVVGAVGVG

SEQ ID NO: 176 - protein, 9 aa, Homo sapiens
HLSTAFARV

SEQ ID NO: 177 - protein, 9 aa, Homo sapiens
KIFGSLAFL

SEQ ID NO: 178 - protein, 9 aa, Homo sapiens
IISAVVGIL

SEQ ID NO: 179 - protein, 9 aa, Homo sapiens
ALCRWGLLL

SEQ ID NO: 180 - protein, 9 aa, Homo sapiens
ILHNGAYSL

SEQ ID NO: 181 - protein, 9 aa, Homo sapiens
RLLQETELV

SEQ ID NO: 182 - protein, 9 aa, Homo sapiens
VVKGVVFGI

SEQ ID NO: 183 - protein, 10 aa, Homo sapiens
YMIMVKCWMI

SEQ ID NO: 184 - protein, 9 aa, Homo sapiens
SLLSGDWVL

SEQ ID NO: 185 - protein, 9 aa, Homo sapiens
GLQLGVQAV

SEQ ID NO: 186 - protein, 9 aa, Homo sapiens
PLTEYIQPV

SEQ ID NO: 187 - protein, 9 aa, Homo sapiens
SPRWWPTCL

SEQ ID NO: 188 - protein, 9 aa, Homo sapiens
GVALQTMKQ

SEQ ID NO: 189 - protein, 9 aa, Homo sapiens
FMNKFIYEI

SEQ ID NO: 190 - protein, 10 aa, Homo sapiens
QLAVSVILRV

SEQ ID NO: 191 - protein, 14 aa, Homo sapiens
LPAVVGLSPGEQEY

SEQ ID NO: 192 - protein, 9 aa, Homo sapiens
NYARTEDFF

SEQ ID NO: 193 - protein, 9 aa, Homo sapiens
FLIIWQNTM

SEQ ID NO: 194 - protein, 14 aa, Homo sapiens
TLYQDDTLTLQAAG

SEQ ID NO: 195 - protein, 10 aa, Homo sapiens
SLYKFSPFPL

SEQ ID NO: 196 - protein, 9 aa, Homo sapiens
KELEGILLL

SEQ ID NO: 197 - protein, 9 aa, Homo sapiens
RLSSCVPVA

SEQ ID NO: 198 - protein, 10 aa, Homo sapiens
VLLQAGSLHA

SEQ ID NO: 199 - protein, 9 aa, Homo sapiens
LLNAFTVTV

SEQ ID NO: 200 - protein, 8 aa, Homo sapiens
VLFYLGQY

SEQ ID NO: 201 - protein, 10 aa, Homo sapiens
FIASNGVKLV

SEQ ID NO: 202 - protein, 9 aa, Homo sapiens
ETVSEQSNV

SEQ ID NO: 203 - protein, 9 aa, Homo sapiens
FLDEFMEGV

SEQ ID NO: 204 - protein, 8 aa, Homo sapiens
QQITKTEV

SEQ ID NO: 205 - protein, 8 aa, Homo sapiens
YRPRPRRY

SEQ ID NO: 206 - protein, 9 aa, Homo sapiens
MVKISGGPR

SEQ ID NO: 207 - protein, 9 aa, Homo sapiens
EVDPIGHVY

SEQ ID NO: 208 - protein, 9 aa, Homo sapiens
ISGGPRISY

SEQ ID NO: 209 - protein, 9 aa, Homo sapiens
RQKKIRIQL

SEQ ID NO: 210 - protein, 16 aa, Homo sapiens
HLGSRQKKIRIQLRSQ

SEQ ID NO: 211 - protein, 17 aa, Homo sapiens
CATWKVICKSCISQTPG

SEQ ID NO: 212 - protein, 9 aa, Homo sapiens
MIAVFLPIV

SEQ ID NO: 213 - protein, 15 aa, Homo sapiens
HQQYFYKIPILVINK

SEQ ID NO: 214 - protein, 9 aa, Homo sapiens
FLFLLFFWL

SEQ ID NO: 215 - protein, 9 aa, Homo sapiens
TLMSAMTNL

SEQ ID NO: 216 - protein, 9 aa, Homo sapiens
ALDVYNGLL

SEQ ID NO: 217 - protein, 10 aa, Homo sapiens
FLTPKKLQCV

SEQ ID NO: 218 - protein, 10 aa, Homo sapiens
VISNDVCAQV

SEQ ID NO: 219 - protein, 9 aa, Homo sapiens
YYWPRPRRY

SEQ ID NO: 220 - protein, 10 aa, Homo sapiens
VLPDVFIRCV

SEQ ID NO: 221 - protein, 10 aa, Homo sapiens
RYCNLEGPPI

SEQ ID NO: 222 - protein, 24 aa, Homo sapiens
KWTEPYCVIAAVKIFPRFFMVAKQ

SEQ ID NO: 223 - protein, 20 aa, Homo sapiens
KCCKIRYCNLEGPPINSSVF

SEQ ID NO: 224 - protein, 9 aa, Homo sapiens
GLYDGMEHL

SEQ ID NO: 225 - protein, 9 aa, Homo sapiens
FLWGPRALV

SEQ ID NO: 226 - protein, 9 aa, Homo sapiens
VRIGHLYIL

SEQ ID NO: 227 - protein, 15 aa, Homo sapiens
REPFTKAEMLGSVIR

SEQ ID NO: 228 - protein, 14 aa, Homo sapiens
AELVHFLLLKYRAR

SEQ ID NO: 229 - protein, 10 aa, Homo sapiens
LLFGLALIEV

SEQ ID NO: 230 - protein, 9 aa, Homo sapiens
ALKDVEERV

SEQ ID NO: 231 - protein, 9 aa, Homo sapiens
SESIKKKVL

SEQ ID NO: 232 - protein, 9 aa, Homo sapiens
ASSTLYLVF

SEQ ID NO: 233 - protein, 15 aa, Homo sapiens
SSTLYLVFSPSSFST

SEQ ID NO: 234 - protein, 9 aa, Homo sapiens
QGQHFLQKV

SEQ ID NO: 235 - protein, 10 aa, Homo sapiens
EVISCKLIKR

SEQ ID NO: 236 - protein, 9 aa, Homo sapiens
LYATVIHDI

SEQ ID NO: 237 - protein, 9 aa, Homo sapiens
SSKALQRPV

SEQ ID NO: 238 - protein, 9 aa, Homo sapiens
GFKQSSKAL

SEQ ID NO: 239 - protein, 17 aa, Homo sapiens
ATGFKQSSKALQRPVAS

SEQ ID NO: 240 - protein, 16 aa, Homo sapiens
TMKQICKKEIRRLHQY

SEQ ID NO: 241 - protein, 10 aa, Homo sapiens
KILDAVVAQK

SEQ ID NO: 242 - protein, 9 aa, Homo sapiens
RIAECILGM

SEQ ID NO: 243 - protein, 15 aa, Homo sapiens
IGRIAECILGMNPSR

SEQ ID NO: 244 - protein, 10 aa, Homo sapiens
YVDFREYEYY

SEQ ID NO: 245 - protein, 9 aa, Homo sapiens
FLDRFLSCM

SEQ ID NO: 246 - protein, 11 aa, Homo sapiens
SLIAAAAFCLA

SEQ ID NO: 247 - protein, 9 aa, Homo sapiens
VVMSWAPPV

SEQ ID NO: 248 - protein, 25 aa, Homo sapiens
NSNHVASGAGEAAIETQSSSSEEIV

SEQ ID NO: 249 - protein, 10 aa, Homo sapiens
ILFGISLREV

SEQ ID NO: 250 - protein, 9 aa, Homo sapiens
KVVEFLAML

SEQ ID NO: 251 - protein, 13 aa, Homo sapiens
SSALLSIFQSSPE

SEQ ID NO: 252 - protein, 9 aa, Homo sapiens
SFSYTLLSL

SEQ ID NO: 253 - protein, 9 aa, Homo sapiens
VSSFFSYTL

SEQ ID NO: 254 - protein, 15 aa, Homo sapiens
KPLFRRMSSLELVIA

SEQ ID NO: 255 - protein, 9 aa, Homo sapiens
EVDPIGHLY

SEQ ID NO: 256 - protein, 9 aa, Homo sapiens
KVAELVHFL

SEQ ID NO: 257 - protein, 9 aa, Homo sapiens
TFPDLESEF

SEQ ID NO: 258 - protein, 9 aa, Homo sapiens
VAELVHFLL

SEQ ID NO: 259 - protein, 10 aa, Homo sapiens
MEVDPIGHLY

SEQ ID NO: 260 - protein, 9 aa, Homo sapiens
AELVHFLLL

SEQ ID NO: 261 - protein, 9 aa, Homo sapiens
WQYFFPVIF

SEQ ID NO: 262 - protein, 16 aa, Homo sapiens
KKLLTQHFVQENYLEY

SEQ ID NO: 263 - protein, 15 aa, Homo sapiens
RKVAELVHFLLLKYR

SEQ ID NO: 264 - protein, 16 aa, Homo sapiens
ACYEFLWGPRALVETS

SEQ ID NO: 265 - protein, 12 aa, Homo sapiens
VIFSKASSSLQL

SEQ ID NO: 266 - protein, 15 aa, Homo sapiens
VFGIELMEVDPIGHL

SEQ ID NO: 267 - protein, 15 aa, Homo sapiens
GDNQIMPKAGLLIIV

SEQ ID NO: 268 - protein, 15 aa, Homo sapiens
TSYVKVLHHMVKISG

SEQ ID NO: 269 - protein, 16 aa, Homo sapiens
RKVAELVHFLLLKYRA

SEQ ID NO: 270 - protein, 5 aa, Homo sapiens
PMGAP

SEQ ID NO: 271 - protein, 5 aa, Homo sapiens
PMGLP

SEQ ID NO: 272 - protein, 17 aa, Homo sapiens
MRYMILGLLALAAVCSA

SEQ ID NO: 273 - protein, 23 aa, Homo sapiens
MDAMLRGLCCVLLLCGAVFVSPS

SEQ ID NO: 274 - protein, 124 aa, Homo sapiens
MNKVKFYVLFTALLSSLCAHGAPQSITELCSEYHNTQIYTINDKILSYTESMAGKREMVIITFKSGATFQVEVPGSQHIDSQKKAIERMKDTLRITYLTETKIDKLCVWNNKTPNSIAAISMEN

SEQ ID NO: 275 - protein, 12 aa, Homo sapiens
KGILGFVFTLLV

SEQ ID NO: 276 - protein, 20 aa, Homo sapiens
IDKISDVSTIVPYIGPALNI

SEQ ID NO: 277 - protein, 25 aa, Homo sapiens
MLIPIAVGGALAGLVLIVLIAYLVG

SEQ ID NO: 278 - protein, 14 aa, Homo sapiens
TKDNNLLGRFELSG

SEQ ID NO: 279 - protein, 9 aa, Homo sapiens
FPSDSWCYF

SEQ ID NO: 280 - protein, 9 aa, Homo sapiens
SPSYAYHQF

SEQ ID NO: 281 - protein, 9 aa, Homo sapiens
SPSYVYHQF

SEQ ID NO: 282 - protein, 14 aa, Homo sapiens
RSKRLSPSYVYHQF

SEQ ID NO: 283 - DNA, 84 bp, Homo sapiens
aggagcaaaagagtgagccccagctacgtgtaccaccagttctcctcgttttctcactcggggtcgatgcacatggtggtcaag

SEQ ID NO: 284 - protein, 13 aa, Homo sapiens
RSKRLLMWITQCF

SEQ ID NO: 285 - DNA, 78 bp, Homo sapiens
aggagcaaaagactgctgatgtggatcacccagtgcttctcctcgttttctgacgactacacctagtgggtcacgaag

SEQ ID NO: 286 - protein, 12 aa, Homo sapiens
RSKRGSPFPAAV

SEQ ID NO: 287 - DNA, 78 bp, Homo sapiens
aggagcaaaagaggcagccccttccccgccgctgtgacctcctcgttttctccgtcggggaaggggcggcgacactgg

SEQ ID NO: 288 - DNA, 24 bp, Homo sapiens
aggagcaaaagacacagccccagc

SEQ ID NO: 289 - DNA, 24 bp, Homo sapiens
tcttttgctcctgaactggtggta

SEQ ID NO: 290 - DNA, 24 bp, Homo sapiens
taccaccagttcaggagcaaaaga

SEQ ID NO: 291 - DNA, 24 bp, Homo sapiens
tcttttgctcctgaagcactgggt

SEQ ID NO: 292 - DNA, 24 bp, Homo sapiens
acccagtgcttcaggagcaaaaga

SEQ ID NO: 293 - DNA, 18 bp, Homo sapiens
ggtcacagcggcggggaa

SEQ ID NO: 294 - DNA, 31 bp, Homo sapiens
tttttgaaatgttaaaaacaaaattttgttg

SEQ ID NO: 295 - DNA, 20 bp, Homo sapiens
tgatccgaccagcaaaactc

SEQ ID NO: 296 - protein, 180 aa, Homo sapiens
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 297 - protein, 9 aa, Homo sapiens
LMWITQCFL

SEQ ID NO: 298 - protein, 9 aa, Homo sapiens
RLLEFYLAM

SEQ ID NO: 299 - protein, 9 aa, Homo sapiens
GVLLKEFTV

SEQ ID NO: 300 - protein, 9 aa, Homo sapiens
WITQCFLPV

SEQ ID NO: 301 - protein, 9 aa, Homo sapiens
QLSLLMWIT

SEQ ID NO: 302 - protein, 9 aa, Homo sapiens
QQLSLLMWI

SEQ ID NO: 303 - protein, 9 aa, Homo sapiens
SLAQDAPPL

SEQ ID NO: 304 - protein, 9 aa, Homo sapiens
ILTIRLTAA

SEQ ID NO: 305 - protein, 9 aa, Homo sapiens
LWLSISSCL

SEQ ID NO: 306 - protein, 9 aa, Homo sapiens
AQDAPPLPV

SEQ ID NO: 307 - protein, 9 aa, Homo sapiens
LQLSISSCL

SEQ ID NO: 308 - protein, 9 aa, Homo sapiens
SISSCLQQL

SEQ ID NO: 309 - protein, 9 aa, Homo sapiens
CLQQLSLLM

SEQ ID NO: 310 - protein, 9 aa, Homo sapiens
FTVSGNILT

SEQ ID NO: 311 - protein, 9 aa, Homo sapiens
FYLAMPFAT

SEQ ID NO: 312 - skipped

SEQ ID NO: 313 - protein, 9 aa, Homo sapiens
CLQQLSLLM

SEQ ID NO: 314 - protein, 9 aa, Homo sapiens
SCLQQLSLL

SEQ ID NO: 315 - protein, 9 aa, Homo sapiens
QLQLSISSC

SEQ ID NO: 316 - protein, 9 aa, Homo sapiens
NTYASPRFK

SEQ ID NO: 317 - protein, 10 aa, Homo sapiens
SLFEGIDIYT

SEQ ID NO: 318 - protein, 9 aa, Homo sapiens
ALSVMGVYV

SEQ ID NO: 319 - protein, 15 aa, Homo sapiens
YSVYFNLPADTIYTN

SEQ ID NO: 320 - protein, 29 aa, Homo sapiens
EDLTVKIGDFGLATEKSRWSGSHQFEQLS

SEQ ID NO: 321 - protein, 9 aa, Homo sapiens
SYLDSGIHF

SEQ ID NO: 322 - protein, 12 aa, Homo sapiens
FSWAMDLDPKGA

SEQ ID NO: 323 - protein, 10 aa, Homo sapiens
ACDPHSGHFV

SEQ ID NO: 324 - protein, 9 aa, Homo sapiens
CILGKLFTK

SEQ ID NO: 325 - protein, 9 aa, Homo sapiens
AVCPWTWLR

SEQ ID NO: 326 - protein, 9 aa, Homo sapiens
ILDKVLVHL

SEQ ID NO: 327 - protein, 9 aa, Homo sapiens
GLFGDIYLA

SEQ ID NO: 328 - protein, 14 aa, Homo sapiens
MIFEKHGFRRTTPP

SEQ ID NO: 329 - protein, 10 aa, Homo sapiens
SLADEAEVYL

SEQ ID NO: 330 - protein, 10 aa, Homo sapiens
TLDWLLQTPK

SEQ ID NO: 331 - protein, 9 aa, Homo sapiens
ILNAMIAKI

SEQ ID NO: 332 - protein, 9 aa, Homo sapiens
WRRAPAPGA

SEQ ID NO: 333 - protein, 9 aa, Homo sapiens
PVTWRRAPA

SEQ ID NO: 334 - protein, 11 aa, Homo sapiens
FLEGNEVGKTY

SEQ ID NO: 335 - protein, 9 aa, Homo sapiens
KTLTSVFQK

SEQ ID NO: 336 - protein, 9 aa, Homo sapiens
EEKLIVVLF

SEQ ID NO: 337 - protein, 11 aa, Homo sapiens
SELFRSGLDSY

SEQ ID NO: 338 - protein, 9 aa, Homo sapiens
FRSGLDSYV

SEQ ID NO: 339 - protein, 9 aa, Homo sapiens
EAFIQPITR

SEQ ID NO: 340 - protein, 11 aa, Homo sapiens
RVIKNSIRLTL

SEQ ID NO: 341 - protein, 9 aa, Homo sapiens
KINKNPKYK

SEQ ID NO: 342 - protein, 9 aa, Homo sapiens
YTDFHCQYV

SEQ ID NO: 343 - protein, 10 aa, Homo sapiens
LLLDDLLVSI

SEQ ID NO: 344 - protein, 16 aa, Homo sapiens
PYYFAAELPPRNLPEP

SEQ ID NO: 345 - protein, 10 aa, Homo sapiens
ILDTAGREEY

SEQ ID NO: 346 - protein, 9 aa, Homo sapiens
RPHVPESAF

SEQ ID NO: 347 - protein, 9 aa, Homo sapiens
KIFSEVTLK

SEQ ID NO: 348 - protein, 9 aa, Homo sapiens
SHETVIIEL

SEQ ID NO: 349 - protein, 15 aa, Homo sapiens
GELIGILNAAKVPAD

SEQ ID NO: 350 - protein, 9 aa, Homo sapiens
LYSACFWWL

SEQ ID NO: 351 - protein, 9 aa, Homo sapiens
VLHWDPETV

SEQ ID NO: 352 - protein, 9 aa, Homo sapiens
MSLQRQFLR

SEQ ID NO: 353 - protein, 20 aa, Homo sapiens
ISPNSVFSQWRVVCDSLEDY

SEQ ID NO: 354 - protein, 10 aa, Homo sapiens
SLPYWNFATG

SEQ ID NO: 355 - protein, 15 aa, Homo sapiens
SQWRVVCDSLEDYDT

SEQ ID NO: 356 - protein, 9 aa, Homo sapiens
SVYDFFVWL

SEQ ID NO: 357 - protein, 9 aa, Homo sapiens
TLDSQVMSL

SEQ ID NO: 358 - protein, 9 aa, Homo sapiens
LLGPGRPYR

SEQ ID NO: 359 - protein, 9 aa, Homo sapiens
ANDPIFVVL

SEQ ID NO: 360 - protein, 15 aa, Homo sapiens
QCTEVRADTRPWSGP

SEQ ID NO: 361 - protein, 10 aa, Homo sapiens
ALPYWNFATG

SEQ ID NO: 362 - protein, 9 aa, Homo sapiens
KCDICTDEY

SEQ ID NO: 363 - protein, 11 aa, Homo sapiens
SSDYVIPIGTY

SEQ ID NO: 364 - protein, 9 aa, Homo sapiens
MLLAVLYCL

SEQ ID NO: 365 - protein, 10 aa, Homo sapiens
CLLWSFQTSA

SEQ ID NO: 366 - protein, 9 aa, Homo sapiens
YMDGTMSQV

SEQ ID NO: 367 - protein, 11 aa, Homo sapiens
IYMDGTADFSF

SEQ ID NO: 368 - protein, 9 aa, Homo sapiens
QCSGNFMGF

SEQ ID NO: 369 - protein, 12 aa, Homo sapiens
TPRLPSSADVEF

SEQ ID NO: 370 - protein, 9 aa, Homo sapiens
LPSSADVEF

SEQ ID NO: 371 - protein, 10 aa, Homo sapiens
LHHAFVDSIF

SEQ ID NO: 372 - protein, 9 aa, Homo sapiens
SEIWRDIDF

SEQ ID NO: 373 - protein, 15 aa, Homo sapiens
QNILLSNAPLGPQFP

SEQ ID NO: 374 - protein, 13 aa, Homo sapiens
SYLQDSDPDSFQD

SEQ ID NO: 375 - protein, 21 aa, Homo sapiens [sequence omitted]

SEQ ID NO: 376 - protein, 30 aa, Homo sapiens
YTTAEEAAGIGILTVILGVLLLIGCWYCRR

SEQ ID NO: 377 - protein, 9 aa, Homo sapiens
ALNFPGSQK

SEQ ID NO: 378 - protein, 9 aa, Homo sapiens
VYFFLPDHL

SEQ ID NO: 379 - protein, 9 aa, Homo sapiens
RTKQLYPEW

SEQ ID NO: 380 - protein, 10 aa, Homo sapiens
HTMEVTVYHR

SEQ ID NO: 381 - protein, 9 aa, Homo sapiens
SSPGCQPPA

SEQ ID NO: 382 - protein, 10 aa, Homo sapiens
VPLDCVLYRY

SEQ ID NO: 383 - protein, 9 aa, Homo sapiens
LPHSSSHWL

SEQ ID NO: 384 - protein, 8 aa, Homo sapiens
SNDGPTLI

SEQ ID NO: 385 - protein, 15 aa, Homo sapiens
GRAMLGTHTMEVTVY

SEQ ID NO: 386 - protein, 16 aa, Homo sapiens
WNRQLYPEWTEAQRLD

SEQ ID NO: 387 - protein, 18 aa, Homo sapiens
TTEWVETTARELPIPEPE

SEQ ID NO: 388 - protein, 17 aa, Homo sapiens
TGRAMLGTHTMEVTVYH

SEQ ID NO: 389 - protein, 11 aa, Homo sapiens
EAELARRSLAQ

(19) United States
(12) Patent Application Publication
(10) Pub. No.: US 2018/0000912 A1
(43) Pub. Date: Jan. 4, 2018

(54) Virus Vectors Expressing Multiple Epitopes of Tumor Associated Antigens For Inducing Antitumor Immunity

(71) Applicant: New York University, New York, NY (US)

(72) Inventors: Daniel Meruelo, Scarborough, NY (US); Christine Pampeno, New York, NY (US); Alicia Hurtado Martinez, New York, NY (US)

(21) Appl. No.: 15/449,641

(22) Filed: Mar. 3, 2017

Related U.S. Application Data
(60) Provisional application No. 62/303,923, filed on Mar. 4, 2016.

(51) Int. Cl. A61K 39/00; C12N 9/12; C07K 14/47; C12N 7/00; C07K 14/005

(52) U.S. Cl. CPC A61K 39/0011; C12N 7/00; C07K 14/005; C07K 14/4747; C07K 14/4748; C12N 9/12; C12Y 207/12002; A61K 2039/645; C07K 2319/40; A61K 2039/5256; C12N 2770/36122; C12N 2770/36143; C07K 2319/50

(57) ABSTRACT

Provided are polynucleotides and viral vectors, particularly, alphavirus vectors such as Sindbis viral vectors, which encode multiple, e.g., two or more, epitopes of at least one tumor associated antigen in which each epitope is separated by a processing or enzyme cleavage site. The multiple epitopes of the two or more tumor associated antigens encoded by the described polynucleotides and viral vectors may be the same or different. Methods of treating mammalian subjects having a cancer or tumor expressing the tumor associated antigen epitopes are provided, in which the viral vectors encoding the multiple epitopes, as well as other immunostimulatory or immunomodulatory components, generate an anti-cancer or anti-tumor immune response in which high levels of effector T cells increase the survivability of tumored mammalian subjects and result in epitope spreading, thus providing a further enhancement of the immune response.

### CROSS-REFERENCE TO RELATED APPLICATION

This application claims the benefit of U.S. Provisional Application No. 62/303,923, filed Mar. 4, 2016, the entire contents of which are incorporated herein by reference.

### BACKGROUND OF THE INVENTION

Despite available cancer treatments, which may include aggressive surgical approaches and combination chemotherapeutic regimens, implemented over the past two decades, a variety of cancers routinely evade detection and destruction by cells of the immune system and offer a grim prognosis for patients afflicted with such cancers.

Anti-cancer immunity, including protective immunity, is thought to be based both on the magnitude of the immune response and on the phenotype of the memory immune responses, including T central memory cells (Tcm) and T effector memory cells (Tem). Tcm are characterized by a CD62L+ CD127+ phenotype, whereas Tem are defined by a CD62L-CD127+ phenotype. Tem traffic through non-lymphoid tissues and exert immediate effector functions in the periphery, while Tcm localize to the secondary lymphoid organs, where they constitute a secondary line of defense by massively expanding upon encounter with antigens presented by dendritic cells. Induction of T cell memory immune responses is dependent on a variety of factors, such as cytokine milieu, length of antigen stimulation, and dose of antigen. CD8+ T cell memory inflation is characterized by the accumulation of high-frequency, functional Ag-specific CD8+ T cell pools with an effector-memory phenotype and enrichment in peripheral organs. This type of response is more vigorous and desirable, for an effective immune response against cancer growth and recurrence.

Sindbis virus (SV) is an oncolytic alphavirus with a positive-stranded RNA genome that is capable of killing tumor cells through apoptosis. To date, cancer treatment approaches using oncolytic viruses have not generally led to complete cancer or tumor remission. Moreover, some tumor cells may not be efficiently targeted by viruses used in cancer treatments to date, thus underscoring the need to develop new therapies and additional ways to enhance anticancer treatment. Given the many hurdles that currently exist in the treatment and prevention of many types of cancers, there exists a profound need for new and improved anti-cancer therapeutic agents, especially those that elicit an immune response directed against tumor and cancer cells, as well as methods for administering such agents to augment the immune response in the treatment and eradication of tumors and cancers in mammals.

### SUMMARY OF THE INVENTION

The present invention features a polynucleotide that encodes an alphavirus protein or a fragment thereof, and multiple (e.g., two or more) epitopes of one or more tumor associated antigens (TAAs), wherein each epitope is separated by an enzyme cleavage site, as well as viral vectors, viral particles and pharmaceutical compositions comprising the polynucleotide to augment the stimulation of effector T cell responses against a variety of tumor-associated antigens, tumor escape variants, and antigens presented by different HLA haplotypes, thereby inducing anti-tumor (anti-cancer) immunity. In a particular embodiment, the alphavirus protein or a fragment thereof is a Sindbis virus protein or a fragment thereof. In embodiments, the polynucleotide may also encode one or more cytokines, immunostimulatory molecules, or cell signaling molecules, or epitopes thereof.

The invention further features a viral vector or a virus particle, which comprises a polynucleotide that encodes multiple (e.g., two or more) epitopes of one or more tumor associated antigens (TAA), wherein each epitope is separated by an enzyme cleavage site. In an embodiment, the viral vector is an alphavirus vector or a pseudotyped alphavirus vector. In a particular embodiment, the viral vector is a Sindbis viral vector. In other embodiments, the viral vector is a retrovirus or lentivirus pseudotyped with one or more alphavirus envelope proteins, e.g., E1, E2, or E3. In other embodiments, the viral vector is a retrovirus or lentivirus pseudotyped with Sindbis virus envelope proteins, such as E1-E3 or ZZ E2. In an embodiment, the epitopes of the tumor associated antigen comprise 5-50 amino acids. In other embodiments, the epitopes of the tumor associated antigen comprise 5-30 amino acids, 5-25 amino acids, 5-20 amino acids, 7-25 amino acids, 7-20, or 7-14 amino acids. In an embodiment, the enzyme cleavage sites comprise sequences that are recognized by an enzyme as described infra.

In an aspect, the invention provides a polynucleotide which encodes two or more epitopes of one or more tumor associated antigens (TAAs), wherein each epitope is separated by an enzyme cleavage site. In embodiments, the polynucleotide comprises DNA or RNA, which can be single stranded (ss) RNA. In an embodiment, the polynucleotide is carried in a viral vector or viral particle as described infra. In an embodiment, the polynucleotide comprises two or more epitopes which comprise 5-50 amino acids. In an embodiment, the polynucleotide comprises two or more epitopes which comprise 5-30 amino acids. In an embodiment, the one or more tumor associated antigens are expressed on the surface of a cancer or tumor cell (e.g., extracellularly) or are expressed intracellularly inside a cancer or tumor cell. In an embodiment, the two or more epitopes encoded by the polynucleotide comprise an amino acid sequence of a tumor associated antigen listed in any one of Tables 1-28.

In embodiments, two or more epitopes of the one or more of the following tumor associated antigens may be encoded by the polynucleotides, viral vectors, or viral particles described herein: kallikrein 4, papillomavirus binding factor (PBF), preferentially expressed antigen of melanoma (PRAME), Wilms' tumor-1 (WT1), Hydroxysteroid Dehydrogenase Like 1 (HSDL1), mesothelin, cancer testis antigen (NY-ESO-1), carcinoembryonic antigen (CEA), p53, human epidermal growth factor receptor 2/neuro receptor tyrosine kinase (Her2/Neu), carcinoma-associated epithelial cell adhesion molecule (EpCAM), ovarian and uterine carcinoma antigen (CA125), folate receptor a, sperm protein 17, tumor-associated differentially expressed gene-12 (TADG-12), mucin-16 (MUC-16), L1 cell adhesion molecule (L1CAM), mannan-MUC-1, Human endogenous retrovirus K (HERV-K-MEL), Kita-kyushu lung cancer antigen-1 (KK-LC-1), human cancer/testis antigen (KM-HN-1), cancer testis antigen (LAGE-1), melanoma antigen-A1 (MAGE-A1), Sperm surface zona pellucida binding protein (Sp17), Synovial Sarcoma, X Breakpoint 4 (SSX-4), Transient axonal glycoprotein-1 (TAG-1), Transient axonal glycoprotein-2 (TAG-2), Enabled Homolog (ENAH), mammoglobin-A, NY-BR-1, Breast Cancer Antigen, (BAGE-1), B melanoma antigen, melanoma antigen-Al (MAGE-A1), melanoma antigen-A2 (MAGE-A2), mucin k, synovial sarcoma, X breakpoint 2 (SSX-2), Taxol-resistance-associated gene-3 (TRAG-3), Avian Myelocytomatosis Viral Oncogene (c-myc), cyclin B1, mucin 1 (MUC1), p62, survivin, lymphocyte common antigen (CD45), Dickkopf WNT Signaling Pathway Inhibitor 1 (DKK1), telomerase, Kirsten rat sarcoma viral oncogene homolog (K-ras), G250, intestinal carboxyl esterase, alpha-fetoprotein, Macrophage Colony-Stimulating Factor (M-CSF), Prostate-specific membrane antigen (PSMA), caspase 5 (CASP-5), Cytochrome C Oxidase Assembly Factor 1 Homolog (COA-1), O-linked β-N-acetylglucosamine transferase (OGT), Osteosarcoma Amplified 9, Endoplasmic Reticulum Lectin (OS-9), Transforming Growth Factor Beta Receptor 2 (TGF-betaRII), murine leukemia glycoprotein 70 (gp70), Calcitonin Related Polypeptide Alpha (CALCA), Programmed cell death 1 ligand 1 (CD274), Mouse Double Minute 2Homolog (mdm-2), alpha-actinin-4, elongation factor 2, Malic Enzyme 1 (ME1), Nuclear Transcription Factor Y Subunit C (NFYC), G Antigen 1,3 (GAGE-1,3), melanoma antigen-A6 (MAGE-A6), cancer testis antigen XAGE-lb, six transmembrane epithelial antigen of the prostate 1 (STEAP1), PAP, prostate specific antigen (PSA), Fibroblast Growth Factor 5 (FGF5), heat shock protein hsp70-2, melanoma antigen-A9 (MAGE-A9), Arg-specific ADP-ribosyltransferase family C (ARTC1), B-Raf Proto-Oncogene (B-RAF), Serine/Threonine Kinase, beta-catenin, Cell Division Cycle 27 homolog (Cdc27), cyclin dependent kinase 4 (CDK4), cyclin dependent kinase 12 (CDK12), Cyclin Dependent Kinase Inhibitor 2A (CDKN2A), Casein Kinase 1 Alpha 1 (CSNK1A1), Fibronectin 1 (FN1), Growth Arrest Specific 7 (GAS7), Glycoprotein nonmetastatic melanoma protein B (GPNMB), HAUS Augmin Like Complex Subunit 3 (HAUS3), LDLR-fucosyltransferase, Melanoma Antigen Recognized By T-Cells 2 (MART2), myostatin (MSTN), Melanoma Associated Antigen (Mutated) 1 (MUM-1-2-3), Poly(A) polymerase gamma (neo-PAP), myosin class I, Protein phosphatase 1 regulatory subunit 3B (PPP1R3B), Peroxiredoxin-5 (PRDXS), Receptor-type tyrosine-protein phosphatase kappa (PTPRK), Transforming protein N-Ras (N-ras), retinoblastoma-associated factor 600 (RBAF600), sirtuin-2 (SIRT2), SNRPD1, triosephosphate isomerase, Ocular Albinism Type 1 Protein (OA1), member RAS oncogene family (RAB38), Tyrosinase related protein 1-2 (TRP-1-2), Melanoma Antigen Gp75 (gp75), tyrosinase, Melan-A (MART-1), Glycoprotein 100 melanoma antigen (gp100), N-acetylglucosaminyltransferase V gene (GnTVf), Lymphocyte Antigen 6 Complex Locus K (LY6K), melanoma antigen-A10 (MAGE-A10), melanoma antigen-A12 (MAGE-A12), melanoma antigen-C2 (MAGE-C2), melanoma antigen NA88-A, Taxol-resistant-associated protein 3 (TRAG-3), PDZ binding kinase (pbk), caspase 8 (CASP-8), sarcoma antigen 1 (SAGE), Breakpoint Cluster Region-Abelson oncogene (BCR-ABL), fusion protein in leukemia, dek-can, Elongation Factor Tu GTP Binding Domain Containing 2 (EFTUD2), ETS Variant gene 6/acute myeloid leukemia fusion protein (ETV6-AML1), FMS-like tyrosine kinase-3 internal tandem duplications (FLT3-ITD), cyclin-A1, Fibronectin Type III Domain Containing 3B (FDNC3B,) promyelocytic leukemia/retinoic acid receptor alpha fusion protein (pml-RARalpha), melanoma antigen-C1 (MAGE-C1), membrane protein alternative spliced isoform (D393-CD20), melanoma antigen-A4 (MAGE-A4), or melanoma antigen-A3 (MAGE-A3).

In some embodiments, at least one of the two or more epitopes encoded by the polynucleotide is from the tumor associated antigen NY-ESO-1, the tumor associated antigen MAGE-A3 and/or the tumor associated antigen pbk. In a particular embodiment, the polynucleotide encodes an epitope from the tumor associated antigen NY-ESO-1 comprising the amino acid sequence LLMWITQCF (SEQ ID NO: 1) and an epitope from the tumor associated antigen pbk comprising the amino acid sequence GSPFPAAVI (SEQ ID NO: 2). In an embodiment, one of the two or more epitopes encoded by the polynucleotide is from the tumor associated antigen NY-ESO-1 and one of the two or more epitopes is from the tumor associated antigen survivin. In a particular embodiment, the polynucleotide encodes an epitope from the tumor associated antigen NY-ESO-1 comprising the amino acid sequence RGPESRLLE (SEQ ID NO: 3) and an epitope from the tumor associated antigen survivin comprising the amino acid sequence AFLTVKKQM (SEQ ID NO: 4). In an embodiment, the polynucleotide encodes three or more epitopes of one or more tumor associated antigens. In certain embodiments, the three or more epitopes are of the same tumor associated antigen. In other embodiments, the three or more epitopes are from at least one different tumor associated antigen. In certain embodiments, the polynucleotide encodes eight or more epitopes of one or more tumor associated antigens. In embodiments, the polypeptide as described encodes epitopes, particularly, two or more epitopes, of tumor associated antigens expressed on the surface of a cancer or tumor cell or in the cytosol of a cancer or tumor cell of a/an ovarian cancer, breast cancer, testicular cancer, pancreatic cancer, liver cancer, colon cancer, colorectal cancer, thyroid cancer, lung cancer, prostate cancer, kidney cancer, melanoma, squamous cell carcinoma, chronic myeloid leukemia, acute lymphoblastic leukemia, acute myelogenous leukemia, chronic lymphocytic leukemia, promyelocytic leukemia, multiple myeloma, B-cell lymphoma, bladder carcinoma, head and neck cancer, esophageal cancer, brain cancer, pharynx cancer, tongue cancer, synovial cell carcinoma, neuroblastoma, uterine cancer, fibrosarcoma, myxosarcoma, liposarcoma, chondrosarcoma, osteogenic sarcoma, chordoma, angiosarcoma, endotheliosarcoma. lymphangiosarcoma, synovioma, mesothelioma, Ewing's tumor, leiomyosarcoma, rhabdomyosarcoma, basal cell carcinoma, epidermoid carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms'·tumor, cervical cancer, small cell lung carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroglioma, or retinoblastoma.

In some embodiment, the polynucleotide further encodes a processing site or an enzyme cleavage site which is a protease cleavage site. In an embodiment, the enzyme cleavage site is a serine protease cleavage site. In a particular embodiment, the serine protease cleavage site is cleaved by a protein selected from furin, PC1, PC2, PC4, PC5, PACE4, PC7 or a combination thereof. In another particular embodiment, the serine protease cleavage site is cleaved by furin. In an embodiment, the enzyme cleavage site encoded by the polynucleotide comprises the amino acid sequence XRSKRX, (SEQ ID NO: 5), wherein X represents a hydrophobic amino acid. In another embodiment, the enzyme cleavage site encoded by the polynucleotide comprises the amino acid sequence (R/K)Xn(R/K), (SEQ ID NO: 6), wherein X represents an amino acid and n is an integer from 0 to 6. In an embodiment, the polynucleotide comprises a 5′ endoplasmic reticulum signal sequence. In certain embodiments, the polynucleotide comprises a 5′ endoplasmic reticulum signal sequence derived from alphavirus, influenza virus matrix protein-derived peptide M57-68 or tissue plasminogen activator peptide. In an embodiment, the polynucleotide comprises a 3′ sequence encoding an immunogenic protein selected from heat shock protein 70, IgG1 Fc domain, lysosome-associated membrane protein (LAMP), tetanus toxin universal helper T (Th) epitope, or E. coli heat-labile enterotoxin B subunit. In another embodiment, the polynucleotide encodes one or more immunostimulatory proteins. By way of example, such proteins include, without limitation, one or more of IL-1, IL-2, IL-3, IL-4, IL-5, IL-6 IL-7, IL-8, IL-9, IL-10, IL-11, IL-12, IL-13, IL-14, IL-15, IL-16, IL-17, IL-18, IL-19, IL-20 through IL-36, chemokine CCL1 through CCL27, CC chemokine CXCL1 through CXCL13, a CXC chemokine, a C chemokine, a CX3C chemokine, a cytokine or chemokine receptor, a soluble receptor, Transforming Growth Factor-beta (TGF-β), or Tumor Necrosis Factor-alpha (TNFα). In a particular embodiment, the polynucleotide encodes the immunostimulatory protein IL-12. In another embodiment, the polynucleotide further comprises one or more suicide genes, which are capable of converting an inert prodrug, such as, without limitation, ganciclovir, acyclovir, 1-(2-deoxy-2-fluoro-β-D-arabinofuranosyl)-5-iodouracil (FIAU), 6-methoxypurine arabinoside, or 5-fluorocytosine, into a cytotoxic metabolite. In an embodiment, the one or more suicide genes encode cytosine deaminase or thymidine kinase which can be derived from Herpes Simplex Virus (HSVtk) or Varicella Zoster Virus (VZV-tk). As will be appreciated by one skilled in the art, derived from refers to obtaining from, originating from, or producing from, all or a portion of, (typically a functional or active portion of), a polynucleotide, a polypeptide, or a peptide from a source, e.g., a virus, bacterium, microorganism, or biological source.

In another of its aspects, the present invention is directed to a viral vector comprising the polynucleotide as described supra and infra. In embodiments, the viral vector is selected from an alphavirus, a lentivirus, or a retrovirus. In an embodiment, the viral vector is pseudotyped with one or more alphavirus virus envelope proteins. In an embodiment, the viral vector is pseudotyped with alphavirus E1 protein, E2 protein, both the E1 and the E2 proteins, or a fragment thereof. In a particular embodiment, the viral vector is a Sindbis viral vector or is derived from Sindbis virus. In an embodiment, the viral vector is pseudotyped with one or more Sindbis virus envelope proteins. In an embodiment, the viral vector is pseudotyped with Sindbis-ZZ E2 protein or a fragment thereof. In a particular embodiment, the viral vector is a lentivirus pseudotyped with one or more Sindbis virus envelope proteins, which may include the Sindbis-ZZ E2 protein. In a particular embodiment, the viral vector is a retrovirus pseudotyped with one or more Sindbis virus envelope proteins, which may include the Sindbis-ZZ E2 protein. In an embodiment, the viral vector is a replication-defective viral vector. In an embodiment, the viral vector is a replication-competent viral vector. In an embodiment, the viral vector is a non-integrating viral vector. In an embodiment, the viral vector is capable of eliciting an immune response against a tumor or cancer expressing the two or more epitopes of one or more tumor associated antigens following administration to a subject, preferably a human subject or patient who has a cancer or tumor. In an embodiment, the immune response generates cytotoxic T cells that specifically kill the cancer or tumor cells expressing the tumor associated antigen epitopes. In all of the above embodiments, the viral vector contains the polynucleotide described supra and infra (also called a minigene) whose encoded products are expressed in cells following contact of the viral vector with cells in vitro and in vivo.

In a particular aspect, a Sindbis viral vector is provided which comprises a polynucleotide encoding two or more epitopes comprising 5-30 amino acids of a tumor associated antigen, wherein each epitope is separated by a furin enzyme cleavage site. In another particular aspect, a viral vector pseudotyped with one or more Sindbis virus envelope proteins is provided, wherein the viral vector comprises a polynucleotide encoding two or more epitopes comprising 5-30 amino acids of a tumor associated antigen, wherein each epitope is separated by a furin enzyme cleavage site. In embodiments, the two or more epitopes of the above viral vectors comprise an amino acid sequence of a tumor associated antigen listed in any one of Tables 1-28. In an embodiment, the two or more epitopes are of one or more tumor associated antigens selected from the group consisting of kallikrein 4, PBF, PRAME, WT1, HSDL1, mesothelin, NY-ESO-1, CEA, p53, Her2/Neu, EpCAM, CA125, folate receptor a, sperm protein 17, TADG-12, MUC-16, L1CAM, mannan-MUC-1, HERV-K-MEL, KK-LC-1, KM-HN-1, LAGE-1, MAGE-A4, Sp17, SSX-4, TAG-1, TAG-2, ENAH, mammoglobin-A, NY-BR-1, BAGE-1, MAGE-A1, MAGE-A2, mucink, SSX-2, TRAG-3, c-myc, cyclin B1, MUC1, p62, survivin, CD45, DKK1, RU2AS, telomerase, K-ras, G250, hepsin, intestinal carboxyl esterase, alpha-foetoprotein, M-CSF, PSMA, CASP-5, COA-1, OGT, OS-9, TGF-betaRII, gp70, CALCA, CD274, mdm-2, alpha-actinin-4, elongation factor 2, ME1, NFYC, GAGE-1, MAGE-A6, XAGE-1b, PSMA, STEAP1, PAP, PSA, GAGE3, FGFS, hepsin, hsp70-2, MAGE-A9, ARTC1, B-RAF, beta-catenin, Cdc27, CDK4, CDK12, CDKN2A, CLLP, CSNK1A1, FN1, GAS7, GPNMB, HAUS3, LDLR-fucosyltransferase, MART2, MATN, MUM-1, MUM-2, MUM-3, neo-PAP, myosin class I, PPP1R3B, PRDXS, PTPRK, N-ras, RBAF600, SIRT2, SNRPD1, triosephosphate isomerase, OA1, RAB38, TRP-1, gp75, TRP2, tyrosinase, MART-1, gp100, GnTVf, LY6K, MAGE-A10, MAGE-A12, MAGE-C2, NA88-A, TRAG-3, TRP2-INT2g, pbk, CASP-8, SAGE, BCR-ABL, dek-can, EFTUD2, ETV6-AML1, FLT3-ITD, cyclin-A1, FDNC3B, pml-RARalpha, MAGE-C1, D393-CD20, MAGE-A4, and MAGE-A3. In a particular embodiment, at least one of the two or more epitopes is from the tumor associated antigen NY-ESO-1 and at least one of the two or more epitopes is from the tumor associated antigen survivin or pbk. In a particular embodiment, the epitope from the tumor associated antigen NY-ESO-1 comprises the amino acid sequence LLMWITQCF (SEQ ID NO: 1) or the amino acid sequence RGPESRLLE (SEQ ID NO: 3), the epitope from the tumor associated antigen survivin comprises the amino acid sequence AFLTVKKQM (SEQ ID NO: 4), and the epitope from the tumor associated antigen pbk comprises the amino acid sequence GSPFPAAVI (SEQ ID NO: 2). In another embodiment, one of the two or more epitopes is from the tumor associated antigens NY-ESO-1 and one of the two or more epitopes encoded by the viral vector is from the tumor associated antigen survivin. In another embodiment, the epitope from the tumor associated antigen NY-ESO-1 comprises the amino acid sequence RGPESRLLE (SEQ ID NO: 3) and the epitope from the tumor associated antigen survivin comprises the amino acid sequence AFLTVKKQM (SEQ ID NO: 4). In an embodiment, the polynucleotide contained in the viral vector encodes three or more epitopes or eight or more epitopes of one or more tumor associated antigens. In embodiment, the viral vector encodes epitopes, particularly, two or more epitopes, of tumor associated antigens expressed on the surface of a cancer or tumor cell or in the cytosol of a cancer or tumor cell of a/an ovarian cancer, breast cancer, testicular cancer, pancreatic cancer, liver cancer, colorectal cancer, thyroid cancer, lung cancer, prostate cancer, kidney cancer, melanoma, squamous cell carcinoma, chronic myeloid leukemia, acute lymphoblastic leukemia, acute myelogenous leukemia, chronic lymphocytic leukemia, promyelocytic leukemia, multiple myeloma, B-cell lymphoma, bladder carcinoma, head and neck cancer, esophageal cancer, brain cancer, pharynx cancer, tongue cancer, synovial cell carcinoma, neuroblastoma, uterine cancer, fibrosarcoma, myxosarcoma, liposarcoma, chondrosarcoma, osteogenic sarcoma, chordoma, angiosarcoma, endotheliosarcoma. lymphangiosarcoma, synovioma, mesothelioma, Ewing's tumor, leiomyosarcoma, rhabdomyosarcoma, basal cell carcinoma, epidermoid carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms'·tumor, cervical cancer, small cell lung carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroglioma, or retinoblastoma. In an embodiment, the above Sindbis or pseudotyped viral vector comprises a 5′ endoplasmic reticulum signal sequence, which sequence is optionally derived from an alphavirus, influenza virus matrix protein-derived peptide M57-68 or tissue plasminogen activator peptide. In an embodiment, the viral vector comprises a 3′ sequence encoding an immunogenic protein selected from heat shock protein 70, IgG1 Fc domain, lysosome-associated membrane protein (LAMP), tetanus toxin universal helper T (Th) epitope, or E. coli heat-labile enterotoxin B subunit. In embodiments, the polynucleotide contained in the viral vector encodes one or more immunostimulatory proteins selected from IL-1, IL-2, IL-3, IL-4, IL-5, IL-6 IL-7, IL-8, IL-9, IL-10, IL-11, IL-12, IL-13, IL-14, IL-15, IL-16, IL-17, IL-18, IL-19, IL-20 through IL-36, chemokine CCL1 through CCL27, CC chemokine CXCL1 through CXCL13, a CXC chemokine, a C chemokine, a CX3C chemokine, a cytokine or chemokine receptor, a soluble receptor, Transforming Growth Factor-beta (TGF-β), or Tumor Necrosis Factor-alpha (TNFα). In an embodiment, the viral vector comprises one or more suicide genes, which is capable of converting an inert prodrug into a cytotoxic metabolite. By way of example, the inert prodrug may be ganciclovir, acyclovir, 1-(2-deoxy-2-fluoro-β-D-arabinofuranosyl)-5-iodouracil (FIAU), 6-methoxypurine arabinoside, or 5-fluorocytosine. In an embodiment, the one or more suicide genes encode cytosine deaminase or thymidine kinase, which is optionally derived from Herpes Simplex Virus (HSVtk) or Varicella Zoster Virus (VZV-tk). In an embodiment, the viral vector is capable of eliciting an immune response against a tumor or cancer expressing the two or more epitopes of the one or more tumor associated antigens following administration to a subject, preferably a human subject or patient who has a cancer or tumor. In an embodiment, the immune response generates cytotoxic T cells that specifically kill the cancer or tumor cells expressing the tumor associated antigen epitopes. In all of the above embodiments, the Sindbis viral vector or the pseudotyped viral vector contains the polynucleotide described supra and infra (also called a minigene) whose encoded products are expressed in cells following contact of the viral vector with cells in vitro and in vivo.

Provided as another aspect of the invention is a lentiviral vector pseudotyped with one or more genetically engineered Sindbis virus envelope proteins, in which the lentiviral vector comprises the polynucleotide as described supra and infra. Also provided by the invention is a lentiviral vector pseudotyped with one or more genetically engineered Sindbis virus envelope proteins, said lentiviral vector comprising the polynucleotide as described supra and infra, wherein the polynucleotide encodes an epitope of one or more tumor associated antigen selected from NY-ESO-1, MAGE-A3, pbk, survivin, or a combination thereof.

In another aspect, the invention provides a viral particle comprising the viral vector, such as the Sindbis viral vector or the pseudotyped viral vector as described supra and infra. In another aspect, the invention provides a viral particle comprising an alphaviral vector, a lentiviral vector, a retroviral vector, or a pseudotyped vector thereof as described supra and infra.

In another aspect, the invention provides a cell comprising a polynucleotide as described supra and infra. In other aspects, the invention further provides a cell comprising a viral vector or a lentiviral vector as described supra and infra. In an aspect, the invention provides a cell comprising a viral particle as described supra and infra.

In yet another aspect, pharmaceutical compositions are provided which comprise a polynucleotide, viral particle, and/or viral vector as described supra and infra, and a pharmaceutically acceptable vehicle, carrier, or diluent. In an embodiment, the pharmaceutical composition is in liquid dosage form.

In another aspect, a method of inducing an immune response against a cancer or tumor cell expressing one or more epitopes of two or more tumor associated antigens is provided in which the method involves contacting the cancer or tumor cell with an effective amount of a polynucleotide, viral particle, viral vector, and/or pharmaceutical composition as described supra and infra to induce the immune response against the cancer or tumor cell. In an embodiment, the immune response generates cytotoxic T cells that specifically kill the cancer or tumor cells expressing the tumor associated antigen epitopes. In another aspect, a method of treating cancer in a subject who has, or is at risk or having, cancer or tumorigenesis is provided, in which the method involves administering to the subject a therapeutically effective amount of a polynucleotide, viral particle, viral vector, and/or pharmaceutical composition as described supra and infra to treat cancer in the subject. In an embodiment of the method, the subject is preferably a human patient having or at risk of having a cancer or tumor selected from one or more of a/an ovarian cancer, cervical cancer, uterine cancer, breast cancer, testicular cancer, pancreatic cancer, liver cancer, colorectal cancer, thyroid cancer, lung cancer, prostate cancer, kidney cancer, melanoma, squamous cell carcinoma, chronic myeloid leukemia, acute lymphoblastic leukemia, acute myelogenous leukemia, chronic lymphocytic leukemia, promyelocytic leukemia, multiple myeloma, B-cell lymphoma, bladder carcinoma, head and neck cancer, esophageal cancer, brain cancer, pharynx cancer, tongue cancer, synovial cell carcinoma, neuroblastoma, fibrosarcoma, myxosarcoma, liposarcoma, chondrosarcoma, osteogenic sarcoma, chordoma, angiosarcoma, endotheliosarcoma. lymphangiosarcoma, synovioma, mesothelioma, Ewing's tumor, leiomyosarcoma, rhabdomyosarcoma, basal cell carcinoma, epidermoid carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms'·tumor, small cell lung carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroglioma, or retinoblastoma. In a particular embodiment of the methods, the subject's cancer is one or more of ovarian cancer, cervical cancer, breast cancer, or colon cancer. In embodiments of the methods, the polynucleotide, viral particle, viral vector, or pharmaceutical composition encodes two or more epitopes of one or more of the tumor associated antigens NY-ESO-1, p53, sp17, survivin, pbk, CEA, CA125, or WT1. In an embodiment of the methods, the polynucleotide, viral particle, viral vector, or pharmaceutical composition is administered parenterally or as a prophylactic. In embodiments of the methods, the subject is further treated with chemotherapy or radiation. In an embodiment of the methods, a booster is administered to the subject following a decline in the subject's immune response as assessed by determining levels of the subject's effector T-cells. In an embodiment, the booster is a heterologous booster comprising a replication-defective adenoviral vector, such as adenovirus or adeno-associated virus. In an embodiment, the adenoviral booster vector comprises a polynucleotide encoding one or more epitopes of two or more tumor associated antigens, wherein each epitope is separated by a processing site, such as an enzyme cleavage site. In an embodiment, the epitopes comprise an amino acid sequence of a tumor associated antigen listed in any one of Tables 1-28, illustratively, kallikrein 4, PBF, PRAME, WT1, HSDL1, mesothelin, NY-ESO-1, CEA, p53, Her2/Neu, EpCAM, CA125, folate receptor a, sperm protein 17, TADG-12, MUC-16, L1CAM, mannan-MUC-1, HERV-K-MEL, KK-LC-1, KM-HN-1, LAGE-1, MAGE-A4, Sp17, SSX-4, TAG-1, TAG-2, ENAH, mammoglobin-A, NY-BR-1, BAGE-1, MAGE-A1, MAGE-A2, mucink, SSX-2, TRAG-3, c-myc, cyclin B1, MUC1, p62, survivin, CD45, DKK1, RU2AS, telomerase, K-ras, G250, hepsin, intestinal carboxyl esterase, alpha-fetoprotein, M-CSF, PSMA, CASP-5, COA-1, OGT, OS-9, TGF-betaRII, gp70, CALCA, CD274, mdm-2, alpha-actinin-4, elongation factor 2, ME1, NFYC, GAGE-1, MAGE-A6, XAGE-1b, PSMA, STEAP1, PAP, PSA, GAGE3, FGFS, hepsin, hsp70-2, MAGE-A9, ARTC1, B-RAF, beta-catenin, Cdc27, CDK4, CDK12, CDKN2A, CLLP, CSNK1A1, FN1, GAS7, GPNMB, HAUS3, LDLR-fucosyltransferase, MART2, MATN, MUM-1, MUM-2, MUM-3, neo-PAP, myosin class I, PPP1R3B, PRDX5, PTPRK, N-ras, RBAF600, SIRT2, SNRPD1, triosephosphate isomerase, OA1, RAB38, TRP-1, gp75, TRP2, tyrosinase, MART-1, gp100, GnTVf, LY6K, MAGE-A10, MAGE-A12, MAGE-C2, NA88-A, TRAG-3, TRP2-INT2g, pbk, CASP-8, SAGE, BCR-ABL, dek-can, EFTUD2, ETV6-AML1, FLT3-ITD, cyclin-AL FDNC3B, pml-RARalpha, MAGE-C1, D393-CD20, MAGE-A4, or MAGE-A3. In an embodiment, the booster is administered to the subject at least one day to at least two weeks after administration of the polynucleotide, viral particle, viral vector, or pharmaceutical composition. In an embodiment of the methods, the administering of the polynucleotide, viral particle, viral vector, or pharmaceutical composition as described supra and infra, or the boosting, if utilized, causes epitope spreading in the subject. In embodiments, the polynucleotide the viral particle, or the viral vector as described supra and infra further comprise a nucleic acid sequence encoding the amino acid sequence AKFVAAWTLKAAA (SEQ ID NO: 7) for inducing a CD4+ T cell response.

Provided by the invention are therapeutic, prophylactic, or combined therapeutic and prophylactic treatments of mammalian cancers or tumors using the polynucleotides, viral vectors viral particles and pharmaceutical compositions as described supra and infra.

A particular aspect of the invention provides a non-integrating alphavirus vector (e.g., a Sindbis viral vector) molecularly engineered to contain a polynucleotide which encodes two or more epitopes comprising, for example, 5-50 amino acids or 5-30 amino acids, of one or more tumor associated antigens, in which each epitope sequence is separated by processing site, such as an enzyme cleavage site, e.g., a furin enzyme cleavage site, for reproducibility in intracellular processing of the tumor associated antigen epitope polypeptide and peptide products. In some embodiments, the viral vector also contains one or more nucleic acid sequences which encode one or more neo-antigens, cytokines, chemokines, antibodies, mutated oncogenes, or overexpressed oncogenes, for enhancing and improving the immune response against the tumor associated antigen epitopes that is elicited by the viral vectors and viral particles described herein, as well as the therapeutic and/or prophylactic uses thereof. In an aspect, the Sindbis viral vectors as described herein elicit strong T cell responses, including CD8+ T cell responses, against multiple epitopes of tumor associated antigens.

In another aspect, the alphavirus protein or a fragment thereof of the polynucleotides, viral vectors, or viral particles as described herein is derived from one or more of Barmah Forest virus, Barmah Forest virus complex, Eastern equine encephalitis virus (EEEV), Eastern equine encephalitis virus complex, Middelburg virus, Middelburg virus complex, Ndumu virus, Ndumu virus complex, Semliki Forest virus, Semliki Forest virus complex, Bebaru virus, Chikungunya virus, Mayaro virus, Subtype Una virus, O′Nyong Nyong virus, Subtype Igbo-Ora virus, Ross River virus, Subtype Getah virus, Subtype Bebaru virus, Subtype Sagiyama virus, Subtype Me Tri virus, Venezuelan equine encephalitis virus (VEEV), VEEV complex, Cabassou virus, Everglades virus, Mosso das Pedras virus, Mucambo virus, Paramana virus, Pixuna virus, Western equine encephalitis virus (WEEV), Rio Negro virus, Trocara virus, Subtype Bijou Bridge virus, Western equine encephalitis virus complex, Aura virus, Babanki virus, Kyzylagach virus, Sindbis virus, Ockelbo virus, Whataroa virus, Buggy Creek virus, Fort Morgan virus, Highlands J virus, Eilat virus, Salmon pancreatic disease virus (SPDV), Southern elephant seal virus (SESV), Tai Forest virus, or Tonate virus.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “agent” is meant a peptide, polypeptide, nucleic acid molecule, or small molecule chemical compound, antibody, or a fragment thereof.

By “alteration” is meant a change (increase or decrease) in the expression levels or activity of a gene or polypeptide as detected by standard art known methods such as those described herein. As used herein, an alteration includes a 10% change in expression levels, a 25% change, a 40% change, or a 50% or greater change in expression levels.”

By “ameliorate” and “amelioration” is meant decrease, suppress, attenuate, diminish, arrest, or stabilize the development or progression of a disease.

By “analog” or “derivative” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

As used herein, the term “antigen” refers to a substance capable of eliciting a humoral or cell-mediated immune response. An antigen may be capable, e.g., of inducing the generation of antibodies or stimulating T-cell activity through activation of a T-cell receptor. Antigens are typically proteins or polysaccharides, and may be components of bacteria, viruses, and other microorganisms (e.g., coats, capsules, cell walls, capsids, flagella, and toxins). The term as used herein encompasses all substances that can be recognized by the adaptive and innate immune system and by an antibody or antibody fragment in vitro or in vivo.

As used herein, the term “at risk” as it applies to a cell proliferation disease, such as cancer (e.g., a cancer described herein), refers to patients who have undergone tumor debulking surgery or individuals who have a family history of cancer and/or have been diagnosed as having genetic risk factor genes.

As used herein, the term “carrier” refers to a diluent, adjuvant, excipient, or vehicle with which a composition or pharmaceutical composition, e.g., comprising a polynucleotide, viral vector, or viral particle) can be administered. Pharmaceutical and pharmaceutically acceptable carriers include sterile liquids, such as water and oils, including those of petroleum, animal, vegetable or synthetic origin, such as peanut oil, soybean oil, mineral oil, sesame oil, and the like. Water or aqueous saline solutions and aqueous dextrose and glycerol solutions may be employed as carriers, particularly for injectable solutions. Carriers may also include solid dosage forms, including, but not limited to, one or more of a binder (for compressed pills), a glidant, an encapsulating agent, a flavorant, and a colorant. Suitable pharmaceutical carriers are described in “Remington's Pharmaceutical Sciences” by E. W. Martin.

In this disclosure, “comprises,” “comprising,” “containing” and “having” and the like can have the meaning ascribed to them in U.S. Patent law and can mean “ includes,” “including,” and the like; “consisting essentially of” or “consists essentially” likewise has the meaning ascribed in U.S. Patent law and the term is open-ended, allowing for the presence of more than that which is recited so long as basic or novel characteristics of that which is recited is not changed by the presence of more than that which is recited, but excludes prior art embodiments.

“Detect” refers to identifying the presence, absence or amount of a molecule, compound, or agent to be detected.

By “detectable label” is meant a composition that when linked to a molecule of interest renders the latter detectable, via spectroscopic, photochemical, biochemical, immunochemical, or chemical means. For example, useful labels include radioactive isotopes, magnetic beads, metallic beads, colloidal particles, fluorescent dyes, electron-dense reagents, enzymes (for example, as commonly used in an ELISA), biotin, digoxigenin, or haptens.

By “disease” is meant any condition or disorder that adversely affects, damages or interferes with the normal function of a cell, tissue, organ, or part of the body, such as cancer or tumorigenesis.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount. In one embodiment, an effective amount is the amount of an agent of the invention required to reduce or stabilize the rate of proliferation of a cancer cell. In another embodiment, an effective amount is the amount of an agent of the invention required to reduce the survival of a cancer cell. In another embodiment, an effective amount is the amount of an agent of the invention required to induce the death of a cancer cell.

As used herein, the term “endogenous” describes a molecule (e.g., a polypeptide, peptide, nucleic acid, or cofactor) that is found naturally in a particular organism (e.g., a human) or in a particular location within an organism (e.g., an organ, a tissue, or a cell, such as a human cell).

As used herein, the term “epitope” or “antigenic determinant” refers to a site, e.g., an amino acid sequence, on an antigen (e.g., a tumor-associated antigen) to which a ligand, an antibody, or T-cell receptor is capable of binding (e.g., during the induction of an immune response) that can be formed from either contiguous amino acids or discontinuous amino acids that are rendered spatially proximal by the tertiary folding of a protein. Other epitopes are formed by quaternary structures, e.g., by the assembly of several polypeptides. Epitopes formed from contiguous amino acids are typically retained on exposure to denaturing solvents, while epitopes formed by tertiary or quaternary folding are typically lost on treatment with denaturing solvents. An epitope may include, e.g., from 3-30 amino acid residues, or from 5 to 30 or from 5 to 25 amino acid residues, such as 1, 2, 3, 4, 5, 6, 7, 8, 9, 10, 11, 12, 13, 14, 15, 16, 17, 18, 19, 20, 21, 22, 23, 24, 25, 26, 27, 28, 29, or 30 amino acid residues, which may be in a distinct spatial conformation. Methods of determining spatial conformation of epitopes are known in the art and include, e.g., x-ray crystallography and 2-dimensional nuclear magnetic resonance (NMR). Such methods are described in detail, e.g., in Morris, Epitope Mapping Protocols in Methods in Molecular Biology, Vol. 66, (1996).

As used herein, the term “epitope spreading” (also called “antigen spreading”) refers to the diversification of epitope specificity from an initial focused, epitope-specific immune response (e.g., by cytotoxic T cells) directed against a self or foreign antigen or protein, to subdominant and/or cryptic, or mutated epitopes on the protein (intramolecular spreading) or on other proteins (intermolecular spreading). Epitope spreading may enable a patient's immune system to mount an immune response against additional epitopes not initially recognized by cells (e.g., cytotoxic T cells) of the immune system while reducing the possibility of escape variants in the tumor population, and may thus attenuate progression of disease (cancer). In one embodiment, after vaccination with a vector described herein, T cells are generated that respond to tumor associated antigens that were not in the original vaccine formulation, indicating that a secondary round of T cell priming has occurred with antigens derived from tumor cells.

As used herein, the term “exogenous” refers to a molecule (e.g., a polypeptide, peptide nucleic acid, or cofactor) that is not found naturally or endogenously in a particular organism (e.g., a human) or in a particular location within an organism (e.g., an organ, a tissue, or a cell, such as a human cell). Exogenous materials include those that are provided from an external source to an organism or to cultured matter extracted therefrom.

By “fragment” is meant a portion of a polypeptide or nucleic acid molecule. This portion contains at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

As used herein, the term “immune response” refers to a subject's immune system response or reaction to one or more antigens, (e.g., an immunogenic protein or peptide), and/or the epitopes of the antigens, recognized by the immune system as foreign or heterologous. Immune responses include both cell-mediated immune responses (i.e., responses mediated by effector T cells, such as antigen-specific or non-specific T-cells, such as CD8+ T-cells, Th1 cells, Th2 cells, and Th17 cells) as well as humoral immune responses (i.e., responses characterized by B-cell activation and the production of antigen-specific antibodies). The term “immune response” encompasses both the innate immune responses to an antigen or immunogen (e.g., a tumor-associated antigen and/or its associated epitopes) as well as memory responses that are a result of acquired immunity and can involve either B cells or T cells, or both.

The terms “isolated,” “purified,” or “biologically pure” refer to material that is free to varying degrees from components which normally accompany or are associated with it as found in its native state. “Isolate” denotes a degree of separation from original source or surroundings. “Purify” denotes a degree of separation that is higher than isolation. A “purified” or “biologically pure” protein is sufficiently free of other materials such that any impurities do not materially affect the biological properties of the protein or cause other adverse consequences. That is, a nucleic acid or peptide is purified if it is substantially free of cellular material, viral material, or culture medium when produced by recombinant DNA techniques, or chemical precursors or other chemicals when chemically synthesized. Purity and homogeneity are typically determined using analytical chemistry techniques, for example, polyacrylamide gel electrophoresis or high performance liquid chromatography. The term “purified” can denote that a nucleic acid, protein, or peptide gives rise to essentially one band in an electrophoretic gel. For a protein that can be subjected to modifications, for example, phosphorylation or glycosylation, different modifications may give rise to different isolated proteins, which can be separately purified.

By “isolated polynucleotide” is meant a nucleic acid (e.g., a DNA) that is free of the genes which, in the naturally-occurring genome of the organism from which the nucleic acid molecule of the invention is derived, flank the gene. The term therefore includes, for example, a recombinant DNA that is incorporated into a vector; into an autonomously replicating plasmid or virus; or into the genomic DNA of a prokaryote or eukaryote; or that exists as a separate molecule (for example, a cDNA or a genomic or cDNA fragment produced by PCR or restriction endonuclease digestion) independent of other sequences. In addition, the term includes an RNA molecule that is transcribed from a DNA molecule, as well as a recombinant DNA that is part of a hybrid gene encoding additional polypeptide sequence.

By an “isolated polypeptide” is meant a polypeptide that has been separated from components that naturally accompany it. Typically, a polypeptide is isolated when it is at least 60%, by weight, free from the proteins and naturally-occurring organic molecules with which it is naturally associated. Preferably, the preparation is at least 75%, or at least 85%, or at least 90%, or at least 99%, by weight, a desired polypeptide. An isolated polypeptide may be obtained, for example, by extraction from a natural source, by expression of a recombinant nucleic acid encoding such a polypeptide; or by chemically synthesizing the protein. Purity can be measured by any appropriate method, for example, column chromatography, polyacrylamide gel electrophoresis, or by HPLC analysis.

By “marker” is meant any protein or polynucleotide having an alteration in expression level or activity that is associated with a disease or disorder.

A “neo-epitope” as referred to herein is a newly formed (or neo) epitope (e.g., antigenic determinant) that has not been previously recognized by the immune system. Neo-epitopes encompass epitopes on a neoantigen, which is a newly formed antigen. Neoantigens, which are often associated with tumor antigens, are found in oncogenic cells. Within the described viral vectors, large quantities of proteins with the mutated neo-epitope can be generated and secreted into the cytoplasm of antigen-presenting cells of the immune system, where they are processed and used to activate tumor-specific cells, which can then target the cancer cells and destroy them.

As used herein, “obtaining” as in “obtaining an agent” includes synthesizing, purchasing, or otherwise acquiring the agent.

By “polynucleotide” is meant a nucleic acid molecule, e.g., a double-stranded (ds) DNA polynucleotide, a single-stranded (ss) DNA polynucleotide, a dsRNA polynucleotide, or a ssRNA polynucleotide, that encodes one or more polypeptides. The term encompasses positive-sense (i.e., protein-coding) DNA polynucleotides, which are capable of being transcribed to form an RNA transcript, which can be subsequently translated to produce a polypeptide following one or more optional RNA processing events (e.g., intron excision by RNA splicing, or ligation of a 5′ cap or a 3′ polyadenyl tail). The term additionally encompasses positive-sense RNA polynucleotides, capable of being directly translated to produce a polypeptide following one or more optional RNA processing events. As used herein, a polynucleotide may be contained within a viral vector, such as a Sindbis viral vector. A “minigene” as used herein refers to a molecularly engineered polynucleotide, e.g., a multigene construct containing sequences encoding different components, which is designed to encode at least one, preferably, two or more, epitopes of an antigen, such as a tumor associated antigen (TAA), or one or more, preferably, two or more, epitopes of two or more tumor associated antigens. The two or more epitopes may be from the same tumor associated antigen or from different tumor associated antigens. A minigene polynucleotide may further comprise nucleic acid sequences in addition to the epitope-encoding sequences, including, without limitation, framework or motif sequences (e.g., one or more enzyme cleavage sites) and processing sequences, such as a ribosome binding site, a signal sequence (e.g., an endoplasmic reticulum signal sequence), a 5′ flanking region and a 3′ stop codon sequence. The polynucleotide may also contain nucleic acid sequences that encode other antigens (e.g., tumor associated antigens), cell receptors and immunostimulatory or immunomodulatory molecules, such as cytokines, chemokines, cell signaling molecules, and the like. Some or all of the foregoing sequences may be included in the polynucleotide. A minigene may be a polynucleotide, such as a negative-sense DNA or RNA polynucleotide, which serves as a template for the production of a positive-sense polynucleotide.

As used herein, the phrase “pharmaceutically acceptable” refers to molecular entities, biological products and compositions that are physiologically tolerable and do not typically produce an allergic or other adverse reactions, such as gastric upset, dizziness and the like, when administered to a patient (e.g., a human patient).

As used herein, the terms “prevent,” “preventing,” “prevention,” “prophylactic treatment” and the like refer to reducing the probability of developing a disorder or condition in a subject, who does not have, but who is at risk of or susceptible to developing a disorder or condition.

As used herein, the term “pseudotyped” refers to a viral vector that contains one or more foreign viral structural proteins, e.g., envelope glycoproteins. A pseudotyped virus may be one in which the envelope glycoproteins of an enveloped virus or the capsid proteins of a non-enveloped virus originate from a virus that differs from the source of the original virus genome and the genome replication apparatus. (D. A. Sanders, 2002, Curr. Opin. Biotechnol., 13:437-442). The foreign viral envelope proteins of a pseudotyped virus can be utilized to alter host tropism or to increase or decrease the stability of the virus particles. Examples of pseudotyped viral vectors include a retrovirus or lentivirus that contains one or more envelope glycoproteins that do not naturally occur on the exterior of the wild-type retrovirus or lentivirus, such as one or more proteins derived from an alphavirus (e.g., Sindbis virus, such as Sindbis-ZZ E2 protein (Morizono, K. et al., 2010, J. Virol., 84(14):6923-6934), or Sindbis E1, E2 and/or E3 proteins). Pseudotyped viral vectors can infect cells and express and produce proteins encoded by polynucleotides, e.g., “minigenes”, contained within the viral vectors.

By “reduces” is meant a negative alteration of at least 5%, 10%, 25%, 50%, 75%, or 100%.

By “reference” is meant a standard or control condition.

By “specifically binds” is meant a compound or antibody that recognizes and binds a polypeptide of the invention, but which does not substantially recognize and bind other molecules in a sample, for example, a biological sample, which naturally includes a polypeptide of the invention.

By “subject” is meant a mammal, including, but not limited to, a human or non-human mammal, such as a non-human primate, bovine, equine, canine, ovine, or feline mammal. A subject is typically a patient, such as a human patient, who receives treatment for a particular disease or condition as described herein (e.g., a cell proliferation disease, such as cancer or tumor). Examples of subjects and patients include mammals, such as humans, receiving treatment for such diseases or conditions or who are at risk of having such diseases or conditions.

As used herein, the term “suicide gene” refers to a gene encoding a polypeptide capable of inducing cell death, e.g., by apoptosis. Suicide genes may function by encoding a protein or peptide capable of converting a prodrug into a cytotoxic molecule. Exemplary suicide genes include, without limitation, Herpes simplex virus thymidine kinase (HSV-TK), cytosine deaminase, nitroreductase, carboxylesterase, cytochrome P450, and purine nucleoside phosphorylase (PNP), among others.

Ranges provided herein are understood to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

As used herein, the term “therapeutically effective amount” refers to a quantity of a therapeutic agent that is sufficient to treat, diagnose, prevent, and/or delay the onset of one or more symptoms of a disease, disorder, and/or condition upon administration to a patient in need of treatment. In some cases, a therapeutically effective amount may also refer to a quantity of a therapeutic agent that is administered prophylactically (e.g., in advance of the development of full-blown disease) to a subject who is at risk of developing a disease or the symptoms thereof, such as cancer or a tumor.

As used herein, the terms “treat,” treating,” “treatment,” and the like refer to reducing or ameliorating a disorder and/or symptoms associated therewith. It will be appreciated that, although not precluded, treating a disorder or condition does not require that the disorder, condition or symptoms associated therewith be completely eliminated. “Treat” or “treatment” may refer to therapeutic treatment, in which the object is to prevent or slow down (lessen or reduce) an undesired physiological change or disorder, such as the progression of a cell proliferation disorder, such as cancer. Beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. Those in need of treatment include those already with the condition or disorder, as well as those prone to have the condition or disorder or those in whom the condition or disorder is to be prevented.

As used herein, the term “tumor-associated antigen” or “TAA” refers to a protein, polypeptide, or peptide that is expressed by cancer cell, such as a cell within a solid tumor. Tumor-associated antigens include protein or peptide antigens that are expressed on the surface of a cancer cell or that are overexpressed relative to a non-cancerous cell, as well as proteins that arise from mutations of wild-type proteins. Proteins that arise from mutations of wild-type cellular proteins embrace neo-epitopes and neo-antigens that occur in cancer or tumor cells, e.g., mutated k-Ras proteins. Tumor associated antigens thus embrace cell surface receptor proteins, e.g., membrane bound proteins, that are expressed on the surface of a cancer or tumor cell. Tumor associated antigens also embrace intracellular, e.g., cytoplasmic, nuclear, or membrane-bound proteins that are expressed within a cancer or tumor cell. A tumor-associated antigen may be tumor-specific, in which case the expression of the antigen is restricted to a particular type of cancer cell. Alternatively, a tumor-associated antigen may be common to several cancers and thus expressed on the surface of a variety of cancer cell types.

As used herein, the term “vector” refers to a nucleic acid (e.g., a DNA vector, such as a plasmid), a RNA vector, virus or other suitable replicon (e.g., viral vector). A variety of vectors have been developed for the delivery of polynucleotides encoding exogenous proteins into a prokaryotic or eukaryotic cell. A vector may contain a polynucleotide sequence that includes gene of interest (e.g., a gene encoding a tumor-associated antigen and/or an epitope thereof) as well as, for example, additional sequence elements capable of regulating transcription, translation, and/or the integration of these polynucleotide sequences into the genome of a cell. A vector may contain regulatory sequences, such as a promoter, e.g., a subgenomic promoter, region and an enhancer region, which direct gene transcription. A vector may contain polynucleotide sequences that enhance the rate of translation of these genes or improve the stability or nuclear export of the mRNA that results from gene transcription. These sequence elements may include, e.g., 5′ and 3′ untranslated regions, an internal ribosomal entry site (IRES), and/or a polyadenylation signal site in order to direct efficient transcription of a gene carried on the expression vector.

As used herein, the term “vehicle” refers to a solvent, diluent, or carrier component of a pharmaceutical composition.

By “substantially identical” is meant a polypeptide or nucleic acid molecule exhibiting at least 50% identity to a reference amino acid sequence (for example, any one of the amino acid sequences described herein) or nucleic acid sequence (for example, any one of the nucleic acid sequences described herein). Preferably, such a sequence is at least 60%, preferably at least 70%, more preferably 80% or 85%, and most preferably 90%, 95% or even 99% identical at the amino acid level or nucleic acid to the sequence used for comparison, for example, over a specified comparison window. Optimal alignment may be conducted using the homology alignment algorithm of Needleman and Wunsch, 1970, J. Mol. Biol., 48:443. An indication that two peptide or polypeptide sequences are substantially identical is that one peptide or polypeptide is immunologically reactive with specific antibodies raised against the second peptide or polypeptide, although such cross-reactivity is not required for two polypeptides to be deemed substantially identical. Thus, a peptide or polypeptide is substantially identical to a second peptide or polypeptide, for example, where the two differ only by a conservative substitution. Peptides or polypeptides that are “substantially similar” share sequences as noted above except that residue positions which are not identical may differ by conservative amino acid changes. Conservative substitutions typically include, but are not limited to, substitutions within the following groups: glycine and alanine; valine, isoleucine, and leucine; aspartic acid and glutamic acid; asparagine and glutamine; serine and threonine; lysine and arginine; and phenylalanine and tyrosine, and others as known to the skilled person in the art.

Sequence identity is typically measured using sequence analysis software (for example, Sequence Analysis Software Package of the Genetics Computer Group, University of Wisconsin Biotechnology Center, 1710 University Avenue, Madison, Wis. 53705, BLAST, BESTFIT, GAP, or PILEUP/PRETTYBOX programs). Such software matches identical or similar sequences by assigning degrees of homology to various substitutions, deletions, and/or other modifications. Conservative substitutions typically include substitutions within the following groups: glycine, alanine; valine, isoleucine, leucine; aspartic acid, glutamic acid, asparagine, glutamine; serine, threonine; lysine, arginine; and phenylalanine, tyrosine. In an exemplary approach to determining the degree of identity, a BLAST program may be used, with a probability score between e⁻³ and e⁻¹⁰⁰ indicating a closely related sequence.

Polynucleotides and viral nucleic acid molecules useful in the methods of the invention include any nucleic acid molecule that encodes the components of viral vectors described herein and the polypeptide products encoded by the viral vectors, such as alphavirus vectors, Sindbis viral vectors and the like, as well as peptides or fragements thereof. Such nucleic acid molecules need not be 100% identical with the viral vector nucleic acid sequence, but will typically exhibit substantial identity. Polynucleotides having substantial identity to the viral vector sequences are typically capable of hybridizing with at least one strand of the viral vector nucleic acid molecule. Nucleic acid molecules useful in the methods of the invention include any nucleic acid molecule that encodes a polypeptide of the invention or a fragment thereof. Polynucleotides having “substantial identity” to an endogenous sequence are typically capable of hybridizing with at least one strand of a double-stranded nucleic acid molecule. By “hybridize” is meant the pair of nucleic acid molecules to form a double-stranded molecule between complementary polynucleotide sequences (e.g., a gene or nucleic acid sequence described herein), or portions thereof, under various conditions of stringency. (See, e.g., Wahl, G. M. and S. L. Berger (1987) Methods Enzymol. 152:399; Kimmel, A. R. (1987) Methods Enzymol. 152:507).

For example, stringent salt concentration will ordinarily be less than about 750 mM NaCl and 75 mM trisodium citrate, preferably less than about 500 mM NaCl and 50 mM trisodium citrate, and more preferably less than about 250 mM NaCl and 25 mM trisodium citrate. Low stringency hybridization can be obtained in the absence of organic solvent, e.g., formamide, while high stringency hybridization can be obtained in the presence of at least about 35% formamide, and more preferably at least about 50% formamide. Stringent temperature conditions will ordinarily include temperatures of at least about 30° C., more preferably of at least about 37° C., and most preferably of at least about 42° C. Varying additional parameters, such as hybridization time, the concentration of detergent, e.g., sodium dodecyl sulfate (SDS), and the inclusion or exclusion of carrier DNA, are well known to those skilled in the art. Various levels of stringency are accomplished by combining these various conditions as needed. In a preferred: embodiment, hybridization will occur at 30° C. in 750 mM NaCl, 75 mM trisodium citrate, and 1% SDS. In a more preferred embodiment, hybridization will occur at 37° C. in 500 mM NaCl, 50 mM trisodium citrate, 1% SDS, 35% formamide, and 100 .mu.g/ml denatured salmon sperm DNA (ssDNA). In a most preferred embodiment, hybridization will occur at 42° C. in 250 mM NaCl, 25 mM trisodium citrate, 1% SDS, 50% formamide, and 200 μg/ml ssDNA. Useful variations on these conditions will be readily apparent to those skilled in the art.

For most applications, washing steps that follow hybridization will also vary in stringency. Wash stringency conditions can be defined by salt concentration and by temperature. As above, wash stringency can be increased by decreasing salt concentration or by increasing temperature. For example, stringent salt concentration for the wash steps will preferably be less than about 30 mM NaCl and 3 mM trisodium citrate, and most preferably less than about 15 mM NaCl and 1.5 mM trisodium citrate. Stringent temperature conditions for the wash steps will ordinarily include a temperature of at least about 25° C., more preferably of at least about 42° C., and even more preferably of at least about 68° C. In a preferred embodiment, wash steps will occur at 25° C. in 30 mM NaCl, 3 mM trisodium citrate, and 0.1% SDS. In a more preferred embodiment, wash steps will occur at 42 C in 15 mM NaCl, 1.5 mM trisodium citrate, and 0.1% SDS. In a more preferred embodiment, wash steps will occur at 68° C. in 15 mM NaCl, 1.5 mM trisodium citrate, and 0.1% SDS. Additional variations on these conditions will be readily apparent to those skilled in the art. Hybridization techniques are well known to those skilled in the art and are described, for example, in Benton and Davis (Science 196:180, 1977); Grunstein and Hogness (Proc. Natl. Acad. Sci., USA 72:3961, 1975); Ausubel et al. (Current Protocols in Molecular Biology, Wiley Interscience, New York, 2001); Berger and Kimmel (Guide to Molecular Cloning Techniques, 1987, Academic Press, New York); and Sambrook et al., Molecular Cloning: A Laboratory Manual, Cold Spring Harbor Laboratory Press, New York.

Nucleic acids that do not hybridize to each other under stringent conditions are still substantially identical if the polypeptides that they encode are substantially identical. This occurs, for example, when a copy of a nucleic acid is created using the maximum codon degeneracy permitted by the genetic code. In such cases, the nucleic acids typically hybridize under moderately stringent hybridization conditions. Nonlimiting examples of “moderately stringent hybridization conditions” include a hybridization in a buffer of 40% formamide, 1 M NaCl, 1% SDS at 37 C, and a wash in 1×SSC at 45 C. A positive hybridization is at least twice background. Those of ordinary skill will readily recognize that alternative hybridization and wash conditions can be utilized to provide conditions of similar stringency.

By “ortholog” is meant any polypeptide or nucleic acid molecule of an organism that is highly related to a reference protein or nucleic acid sequence from another organism. The degree of relatedness may be expressed as the probability that a reference protein would identify a sequence, for example, in a blast search. The probability that a reference sequence would identify a random sequence as an ortholog is extremely low, less than e⁻¹⁰, e⁻²⁰, e⁻³⁰, e⁻⁴⁰, e⁻⁵⁰, e⁻⁷⁵, e⁻¹⁰⁰. The skilled artisan understands that an ortholog is likely to be functionally related to the reference protein or nucleic acid sequence. In other words, the ortholog and its reference molecule would be expected to fulfill similar, if not equivalent, functional roles in their respective organisms, e.g., mouse and human orthologs.

It is not required that an ortholog, when aligned with a reference sequence, have a particular degree of amino acid sequence identity to the reference sequence. A protein ortholog might share significant amino acid sequence identity over the entire length of the protein, for example, or, alternatively, might share significant amino acid sequence identity over only a single functionally important domain of the protein. Such functionally important domains may be defined by genetic mutations or by structure-function assays. Orthologs may be identified using methods practiced in the art. The functional role of an ortholog may be assayed using methods well known to the skilled artisan. For example, function might be assayed in vivo or in vitro using a biochemical, immunological, or enzymatic assay; or transformation rescue. Alternatively, bioassays may be carried out in tissue culture; function may also be assayed by gene inactivation (e.g., by RNAi, siRNA, or gene knockout), or gene over-expression, as well as by other methods.

Unless specifically stated or obvious from context, as used herein, the term “or” is understood to be inclusive. Unless specifically stated or obvious from context, as used herein, the terms “a”, “an”, and “the” are understood to be singular or plural.

As used herein, the term “about” or “approximately” means within an acceptable error range for the type of value described and the method used to measure the value. For example, these terms can signify within 20%, more preferably within 10%, and most preferably still within 5% of a given value or range. More specifically, “about” can be understood as within 20%, 10%, 9%, 8%, 7%, 6%, 5%, 4%, 3%, 2%, 1%, 0.5%, 0.1%, 0.05%, or 0.01% of the stated value or range. Alternatively, especially in biological systems, the term “about” means within one log unit (i.e., one order of magnitude), preferably within a factor of two of a given value. Unless specifically stated or obvious from context, as used herein, the term “about” is understood as within a range of normal tolerance in the art, for example within 2 standard deviations of the mean. Unless otherwise clear from context, all numerical values provided herein are modified by the term about.

Any compositions or methods provided herein can be combined with one or more of any of the other compositions and methods provided herein.

### BRIEF DESCRIPTION OF THE DRAWINGS

FIGS. 1A and 1B depict schematic representations of the design and sequence of a polynucleotide (minigene) encoding various components, including two or more, e.g., 3, epitopes, of one or more, e.g., 3, tumor associated antigens separated by enzyme cleavage sites (e.g., furin enzyme) as described herein. FIG. 1A shows a schematic representation of the polynucleotide for constructing a Sindbis viral vector encoding multiple (3) epitopes of 3 tumor associated antigens. The polynucleotide construct, named “SV/MG” in FIG. 1A, contains an Xba1 restriction enzyme site (TCTAGA, SEQ ID NO: 8) at its 5′ end and an Apa1 restriction enzyme site (GGGCCC, SEQ ID NO: 9) at its 3′ end for insertion of the polynucleotide into a Sindbis virus vector ‘backbone.’ From 5′ to 3′, the polynucleotide contains a ribosome binding site start codon, an endoplasmic reticulum signal sequence, an epitope of the NY-ESO-1 tumor associated antigen, an epitope of the gp70 glycoprotein tumor associated antigen, an epitope of survivin tumor associated antigen, a furin cleavage site separating each of the tumor associated antigen epitopes and a stop codon. FIG. 1B sets forth the polynucleotide sequence of the polynucleotide (minigene) (SEQ ID NO: 10) described in FIG. 1A and the corresponding amino acid sequences of the polypeptide and peptide components (SEQ ID NO: 11) encoded by the polynucleotide. The component genes and encoded polypeptides/peptides of the polynucleotide are identified below the sequences in FIG. 1B.

FIGS. 2A and 2B present a treatment protocol and a plot of tumor growth following treatment of mice bearing CT26-derived tumors with a Sindbis viral vector encoding multiple epitopes of tumor associated antigens. FIG. 2A depicts the therapeutic treatment protocol for administering the Sindbis viral vector containing the polynucleotide of FIGS. 1A and 1B to mice harboring growing tumors in the CT26 tumor mouse model. FIG. 2B presents a graph showing tumor growth as a function of days after treatment of tumored animals with the Sindbis viral vector encoding multiple epitopes, i.e., SV/MA of FIG. 1A (in which the multiple TAAs include NY-ESO-1, survivin and gp70), versus controls, as described in Example 2, infra. Compared with the controls (Control: mice not receiving any Sindbis viral vector; SV/LacZ: Sindbis viral vector encoding β-galactosidase, an irrelevant bacterial enzyme; and SV/NY-ESO-1, a positive control encoding the NY-ESO-1 tumor associated antigen), the SV/MG viral vector encoding multiple tumor associated antigen epitopes of NY-ESO-1, survivin and gp70 were very effective in inhibiting the growth of CT26 tumor cells following injection into tumored animals (FIG. 2B). Shown below the graph in FIG. 2B are the relative light unit (RLU) values indicating tumor growth in the control and experimental groups of mice treated as described above.

FIG. 3 shows a UV image of a stained agarose gel containing DNA samples following qPCR as described in Example 3, infra. The qPCR was performed with oligonucleotide primers specific for the SV RNA genome. In the gel, Lane (−) contained cDNA from uninfected BHK (control); Lane (+) contained a pSV/MG-CT.26 DNA plasmid (control); Lane M contained a 100 base pair ladder marker (control). The Lanes marked -4, -3, -2, -1 and 0 reflect the dilutions 10⁻⁴, 10⁻³, 10⁻², 10⁻¹ and 10⁰, respectively, of SV/MG-CT.26 virus used to infect BHK cells. The size of the qPCR fragment (˜200 bp) agrees with that obtained with the plasmid DNA control. Because 100 μl of virus was added to the cells, the appearance of viral RNA in a 10⁻⁴ dilution indicated a titer of 10⁵ virus particles/ml. This titer coincided with the titer determined by qPCR CT (threshold cycle) values.

FIGS. 4A-4C show that treatment of tumored (LacZ+ CT26 tumors) mice with a Sindbis viral vector encoding LacZ, a representative tumor associated antigen (“SV/TAA” herein), substantially prolongs survival relative to controls, induces epitope spreading, and circumvents TAA loss. FIG. 4A shows that LacZ+ CT26 tumor-bearing mice were treated with either the SV/LacZ Sindbis viral vector, a control SV vector encoding the GFP protein (SV/GFP), or medium/PBS (Mock) and that only the SV/LacZ Sindbis viral vector induced complete tumor remission (100% animal survival) for at least 60 days. The data are presented as Kaplan-Meier survival plots. Significant values between curves are shown *P<0.05; **P<0.01. FIG. 4B demonstrated using tetramers (Altman, J. D. et al., 1996, Science, 274(5284):94-96) that splenocytes from SV/LacZ-treated mice contained CD8+ T cells specific for both LacZ (not shown) and gp70, an endogenous tumor associated antigen expressed by CT26 cells, thus indicating that epitope spreading had occurred. FIG. 4C presents photographs of a control mouse (“Naïve”) and a mouse that survived its tumors following injection with the SV/LacZ viral vector as described in FIG. 4A (“SV/LacZ survivor”) demonstrating that LacZ (−) CT26 tumors grew in naïve mice, but not in mice treated with the SV/LacZ viral vector encoding LacZ (SV/LacZ survivor mice). These results support the finding that SV/LacZ-induced epitope spreading successfully countered the loss of tumor associated antigen (i.e., LacZ) expression.

FIGS. 5A and 5B show a combination of imaging and flow cytometry to evaluate the effects of treatment/immunotherapy of animals with a Sindbis viral vector encoding at least one tumor associated antigen (SV/luciferase as “SV/TAA”). FIG. 5A shows the results of in vivo imaging used to non-invasively and longitudinally determine the sites of expression of a representative tumor associated antigen, firefly luciferase, after the injection of animals with a Sindbis viral vector encoding luciferase as the tumor associated antigen. As demonstrated by T-cell activation marker CD69 expression levels assessed in the animals, the mediastinal lymph node (MLN), identified as a site of luciferase (as TAA) delivery, was also found to be a site of potent CD8+ T cell activation. ILN=control inguinal lymph nodes (FIG. 5B). The use of encoded luciferase allows the measurement of tumor growth in animal models in which tumor cells are molecularly engineered (e.g., transfected) to express the luciferase gene, which permits imaging of tumor cells and assessing the growth of the tumors comprising these cells.

FIGS. 6A-6D show graphs of tumor growth versus time (days) following injection of mice having LacZ+ CT26 tumors with PBS (control, FIG. 6A) or with the Sindbis viral vector encoding LacZ as tumor associated antigen (SV/LacZ), (FIGS. 6B-6D). The therapeutic effects of SV/LacZ on subcutaneous tumors (i.e., reduced tumor growth as measured by calipers) was not observed in mice depleted of CD4+ T cells (FIG. 6B), CD8+ T cells (FIG. 6C), or both (FIG. 6D), when compared with the results seen for control mice (FIG. 6A).

### DETAILED DESCRIPTION OF THE INVENTION

Provided by the present invention are polynucleotides and viral vectors, particularly, alphavirus vectors, that encode multiple epitopes of one or more tumor associated antigens (TAAs) to induce a potent immune response in a subject against the multiple tumor associated antigens expressed by the subject's cancer or tumor, optimally in the context of HLA/MHC antigens. The polynucleotides and viral vectors as described also result in epitope spreading following administration, which serves to enhance the immune response against the multiple TAAs.

As reported in more detail below, the invention is based, at least in part, on the discovery that a Sindbis vector encoding multiple tumor associated antigens (e.g., NY-1 ESO, survivin, gp70) resulted in the long-term survival of tumor-bearing mice and to the generation of long-lasting CD8+ T cells against multiple tumor antigens. Significantly, therapy with a Sindbis vector encoding multiple tumor associated antigens led to epitope spreading, providing a promising solution to the problem of tumor escape by tumor associated antigen loss or modification. As the gp70 is a murine retroviral glycoprotein, it is particularly useful for preclinical studies. Examples of glycoproteins for similar use but derived from a human virus (lentivirus) include, without limitation, the gp120 and gp41 envelope proteins of the human immunodeficiency virus (HIV), or fragments thereof.

The molecularly engineered viral vectors described herein provide an efficient and effective delivery system designed to harbor the genetic information of one or more tumor antigens (also called tumor associated antigens) as multiple selected epitopes of the tumor associated antigen, including neo-epitopes, and to initiate and perpetuate a specific immune response, which ultimately generates cytotoxic T cells (e.g., effector CD8+ T cells) that are activated to specifically kill the cancer or tumor.

The invention generally features viral vector-based compositions and methods that are useful for treating cancer and tumorigenesis and/or the symptoms thereof in a subject in need thereof, such as a patient having cancer. Methods utilizing viral vectors, which are designed to harbor polynucleotides encoding multiple, e.g., two or more, epitopes of one or more tumor associated antigens (TAAs) as described herein, involve administering a therapeutically effective amount of the viral vector, a viral particle, or a pharmaceutical composition comprising the viral vector or particle to a subject (e.g., a mammal such as a human), in particular, to elicit a T-cell-mediated immune response to the subject's cancer or tumor that expresses the tumor associated antigens and epitopes thereof.

The viral vectors described herein are designed to encode and express multiple epitopes, e.g., amino acid sequences, of tumor associated antigens that are recognized by T cell receptors, i.e., “T cell epitopes.” The expression of multi-epitopes by the viral vectors of the invention can increase the likelihood of triggering an immune response to a variety of tumor antigens and also embraces treatment of subjects having different HLA haplotypes. Such viral vector products may also be designed to contain and express epitopes of tumor associated antigens that have optimal affinity for T cell receptors. Because the polynucleotides, viral vectors and viral particles described herein are designed to carry multiple epitopes of one or more than one tumor associated antigen(s), as well as immunostimulatory and immunomodulatory molecules, these products are capable of targeting multiple cancer and tumor types.

Thus, viral vector products that encode and express multiple epitopes of tumor associated antigens according to the invention provide an approach for treating cancer and tumors that may mimic or augment whole-organism-induced immunity and prevent potential immunopathogenic or suppressive responses, in which the multiple epitopes of one or more tumor associated antigens are recognized by effector T cells to generate a potent immune response in a subject undergoing treatment. The viral vectors as described herein contain multiple epitopes of tumor associated antigens that are designed to be recognized by effector T cells, e.g., CD4⁻ T cells, CD8⁺ T cells, or both. The viral vectors can simultaneously induce responses against different cytotoxic lymphocyte (CTL) determinants, thereby optimizing and maximizing immunogenicity in vivo by inducing a CD8⁺ CTL response of the breadth and strength needed to attack and kill cancer and tumor cells and protect against cancer growth and recurrence.

In accordance with the present invention, the design of polynucleotides, viral vectors, viral particles and cells and pharmaceutical compositions containing these products, which encode and express multiple epitopes, e.g., two or more epitopes, of one or more tumor associated antigens, provides biological products that can be used to expand the activated T cell repertoire. Such activated T cells are thus capable of reacting against (e.g., killing) cancer and tumor cells that express the tumor associated antigens and their associated epitopes, and thus broaden the therapeutic applicability and efficacy of the viral vectors described herein, e.g., alphavirus (e.g., Sindbis virus (SV)), lentivirus, retrovirus, or pseudotyped vectors, constructed to contain a polynucleotide encoding two or more epitopes of one or more tumor associated antigens. In an embodiment, each of the tumor associated antigen epitopes is separated by a processing site, such as an enzyme cleavage site, e.g., a furin cleavage site, for reproducible processing of the expressed epitopes.

According to the present invention, after administration to a subject having a cancer or tumor, the viral vectors and viral particles that encode multiple, e.g., two or more, epitopes derived from one or more tumor-associated antigens (TAAs), or pharmaceutical compositions thereof, deliver the multiple epitopes to cells in the form of RNA. The RNA is processed intracellularly into protein and protein fragments, e.g., epitope peptides, which are optimally presented by cells of the immune system, e.g., macrophages and dendritic cells, in the context of HLA/MHC antigens, to precursors of CD8⁺ T cells. Such antigen presentation by the accessory cells of the immune system activates the CD8⁺ T cells, which proliferate so as to produce large numbers of cytotoxic T cells that kill cancer and tumor cells that express the specific epitopes of the tumor associated antigens, including neo-antigens. Thus, the epitopes encoded by the polynucleotides and viral vectors described herein are optimally provided to elicit a heightened immune response, particularly a T-cell mediated immune response, specifically directed against a cancer cell or a solid tumor expressing one or more of the corresponding tumor associated antigens. In some embodiments, the polynucleotide contained in a viral vector of the invention is termed a minigene or a polynucleotide construct. In some embodiments, the polynucleotide, viral vector, or pharmaceutical composition of the invention may include one or more, preferably two or more, (e.g., 2, 3, 4, 5, 6, 7, 8, 9, 10, 15, 20, 25, 30, 35, 40, 45, 50, 55, 60, 65, 70, 75, 80, 85, 90, or more) epitopes derived from the same tumor associated antigen. For instance, a polynucleotide, viral vector, or pharmaceutical composition of the invention may include one or more copies of the same epitope. In some embodiments, the polynucleotide, viral vector, or pharmaceutical composition of the invention may include one or more, preferably two or more (e.g., 2, 3, 4, 5, 6, 7, 8, 9, 10, 15, 20, 25, 30, 35, 40, 45, 50, 55, 60, 65, 70, 75, 80, 85, 90, or more) epitopes derived from different tumor associated antigens.

### Tumor Associated Antigens (TAAs)

The tumor associated antigens from which the epitopes expressed by polynucleotides and viral vectors of the invention are derived may be associated with, or expressed by, e.g., either extracellularly or intracellularly, a cancer or tumor, such as, without limitation, a/an ovarian cancer, breast cancer, testicular cancer, pancreatic cancer, liver cancer, colorectal cancer, thyroid cancer, lung cancer, prostate cancer, kidney cancer, melanoma, squamous cell carcinoma, chronic myeloid leukemia, acute lymphoblastic leukemia, acute myelogenous leukemia, chronic lymphocytic leukemia, promyelocytic leukemia, multiple myeloma, B-cell lymphoma, bladder carcinoma, head and neck cancer, esophageal cancer, brain cancer, pharynx cancer, tongue cancer, synovial cell carcinoma, neuroblastoma, uterine cancer, fibrosarcoma, myxosarcoma, liposarcoma, chondrosarcoma, osteogenic sarcoma, chordoma, angiosarcoma, endotheliosarcoma. lymphangiosarcoma, synovioma, mesothelioma, Ewing's tumor, leiomyosarcoma, rhabdomyosarcoma, basal cell carcinoma, epidermoid carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms'·tumor, cervical cancer, small cell lung carcinoma, epithelial carcinoma, glioma, astrocytoma, medulloblastoma, craniopharyngioma, ependymoma, pinealoma. Hemangioblastoma, acoustic neuroma, oligodendroglioma, meningioma, neuroglioma, and retinoblastoma. Polynucleotides (minigenes), viral vectors and pharmaceutical compositions of the invention may thus be used to treat a subject, such as a human patient, suffering from one or more of the above conditions.

In an embodiment, two or more different epitopes of one or more tumor associated antigens may be associated with the same cancer or tumor type. In another embodiment, two or more epitopes may be associated with tumor associated antigens of different cancer types, e.g., two or more cancer types. For instance, in some embodiments, a polynucleotide, viral vector, or pharmaceutical composition of the invention includes one or more epitopes of a tumor associated antigen expressed by one type of cancer or tumor cell, e.g., an ovarian cancer cell, and one or more epitopes derived from a tumor associated antigen expressed by another type of cancer or tumor cell, e.g., a breast cancer cell. In some embodiments, a polynucleotide, viral vector, or pharmaceutical composition of the invention includes one or more epitopes, or two or more epitopes, of a tumor associated antigen expressed on the surface of one or more cancer types (e.g., 1, 2, 3, 4, 5, 6, 7, 8, 9, 10, 11, 12, 13, 14, 15, 16, 19, 18, 19, 20, 30, 40, 50, or more cancer or tumor types). In other embodiments, the one or more epitopes, or two or more epitopes, of a tumor associated antigen are expressed intracellularly in one or more cancer or tumor types.

In some embodiments, a polynucleotide, viral vector, or pharmaceutical composition of the invention includes two or more epitopes of one or more tumor associated antigens associated with the above cancer types. Tables 1-28, below, provide a non-limiting list of various tumor associated antigens and epitopes thereof that may be encoded by a polynucleotide, viral vector, or viral particle as described herein, or incorporated into a composition of the invention. Tumor associated antigens and their epitopes encompass human tumor associated antigens and epitopes thereof and human orthologs of tumor associated antigens and epitopes thereof. For instance, in some embodiments, a polynucleotide, viral vector, or pharmaceutical composition of the invention includes one or more, or two or more (e.g., 2, 3, 4, 5, 6, 7, 8, 9, 10, 15, 20, 25, 30, 35, 40, 45, 50, 55, 60, 65, 70, 75, 80, 85, 90, or more) epitopes of one or more of the tumor associated antigens listed in any one of Tables 1-28. In some embodiments, a polynucleotide, viral vector, or pharmaceutical composition of the invention includes one or more, or two or more, (e.g., 2, 3, 4, 5, 6, 7, 8, 9, 10, 15, 20, 25, 30, 35, 40, 45, 50, 55, 60, 65, 70, 75, 80, 85, 90, or more) of the amino acid sequences listed in any one of Tables 1-28.

In an embodiment, each of the epitopes of the tumor associated antigens encoded by a polynucleotide, viral vector, or viral particle of the invention is separated by an enzyme cleavage (or processing) site, for example, a furin cleavage site, or other enzyme cleavage or processing site as described herein. Non-limiting examples of additional processing enzymes for use in cleaving the epitope peptides encoded by the polynucleotides and viral vectors according to the present invention include serine protease, signalase, furin protease, and furin related endopeptidases, such as PC1/2, PC4/5, PACE4, and PC7. These enzymes recognize the processing signal (R/K)Xₙ(R/K), in which Xₙ designates a spacer of any 0-6 amino acids, (SEQ ID NO: 6), (Seidah and Prat, 2012, Nature Reviews Drug Discovery, 11:367-383). The inclusion of an enzyme cleavage site that separates each of the encoded epitopes in the polynucleotide, viral vector, or viral particle as described herein, advantageously allows for reproducibility in processing the expressed epitopes following administration, which provides a safer product for use in treating subjects. For example, having the polynucleotide according to the invention contain enzyme cleavage sites interspersed between each of the nucleic acid sequences encoding the tumor associated antigen epitopes ensures that the processing and production of the epitopes is uniform, especially in cells in vivo, and that the designed polypeptide operates reproducibly to generate the appropriate immune response (e.g., a T cell response) directed against the encoded target antigens. In an embodiment, the tumor associated antigen epitopes are selected based on their binding to MHC/HLA molecules, e.g., for optimal presentation to effector T cells, thus providing reproducibility that ensures an optimal immune response, as described herein.

In other embodiments, the epitopes of the one or more tumor associated antigens are each separated by one enzyme cleavage site. In some embodiments, the epitopes are not separated by enzyme cleavage sites and the encoded sequences are cleaved intracellularly following delivery to cells by the viral vectors described herein.

TABLE 1
Ovarian cancer

| | Tumor- | | |
|---|---|---|---|
| | associated | | |
| No. | antigen | Immunogenic epitopes | Sources |
| 1 | Kallikrein 4 | FLGYLILGV (SEQ ID NO: 12); | Wilkinson et al. Cancer Immunol. |
| | | SVSESDTIRSISIAS (SEQ ID NO: 13); | Immunother. 61(2): 169-79 |
| | | LLANGRMPTVLQCVN (SEQ ID NO: 14); and | (2012). |
| | | RMPTVLQCVNVSVVS (SEQ ID NO: 15) | Hural et al. J. Immunol. |
| | | | 169(1): 557-65 (2002). |
| 2 | PBF | CTACRWKKACQR (SEQ ID NO: 16) | Tsukahara et al. Cancer Res. |
| | | | 64(15): 5442-8 (2004). |
| 3 | PRAME | VLDGLDVLL (SEQ ID NO: 17); | Kessler et al. J. Exp. Med. |
| | | SLYSFPEPEA (SEQ ID NO: 18); | 193(1): 73-88 (2001). |
| | | ALYVDSLFFL (SEQ ID NO: 19); | Ikeda et al. Immunity 6(2): 199-208 |
| | | SLLQHLIGL (SEQ ID NO: 20); and | (1997). |
| | | LYVDSLFFL (SEQ ID NO: 21) | |
| 4 | WT1 | TSEKRPFMCAY (SEQ ID NO: 22); | Asemissen et al. Clin. Cancer |
| | | CMTWNQMNL (SEQ ID NO: 23); | Res. 12(24): 7476-82 (2006) |
| | | LSHLQMHSRKH (SEQ ID NO: 24); | Ohminami et al. Blood. |
| | | KRYFKLSHLQMHSRKH (SEQ ID NO: 25); and | 95(1): 286-93 (2000). |
| | | KRYFKLSHLQMHSRKH (SEQ ID NO: 25) | Guo et al. Blood. 106(4): 1415-8 |
| | | | (2005). |
| | | | Lin et al. J. Immunother. |
| | | | 36(3): 159-70 (2013). |
| | | | Fujiki et al. J. Immunother. |
| | | | 30(3): 282-93 (2007). |
| 5 | HSDL1 | CYMEAVAL (SEQ ID NO: 26) | Wick et al. Clin. Cancer Res. |
| | | | 20(5): 1125-34 (2014). |
| 6 | Mesothelin | SLLFLLFSL (SEQ ID NO: 27) | Hassan et al. Appl. |
| | | VLPLTVAEV (SEQ ID NO: 28) | Immunohistochem. Mol. |
| | | ALQGGGPPY (SEQ ID NO: 29) | Morphol. 13(3): 243-7 (2005). |
| | | LYPKARLAF (SEQ ID NO: 30) | Thomas et al J Exp Med. 2004 |
| | | AFLPWHRLF (SEQ ID NO: 31) | Aug 2; 200(3): 297-306. |
| 7 | NY-ESO-1 | HLA-A2-restricted peptide p157-165 | Jager et al. Proc. Natl. Acad. |
| | | (SLLMWITQC) (SEQ ID NO: 32), HLA-Cw3- | Scie. U.S.A. 103(39): 14453-8 |
| | | restricted p92-100 (LAMP-FATPM) (SEQ ID | (2006). |
| | | NO: 33) and HLA-Cw6-restricted p80-88 | Gnjatic et al. PNAS |
| | | (ARGPESRLL) (SEQ ID NO: 34) | Sep. 26, 2000 vol. 97 no. |
| | | SLLMWITQC (SEQ ID NO: 32) | 20 p. 10919 |
| | | MLMAQEALAFL (SEQ ID NO: 35) | Jager et al. J Exp Med. |
| | | YLAMPFATPME (SEQ ID NO: 36) | 187(2): 265-70 (1998). |
| | | ASGPGGGAPR (SEQ ID NO: 37) | Chen et al. J Immunol. |
| | | LAAQERRVPR (SEQ ID NO: 38) | 165(2): 948-55 (2000). |
| | | TVSGNILTIR (SEQ ID NO: 39) | Valmori et al. Cancer Res. |
| | | APRGPHGGAASGL (SEQ ID NO: 40) | 60(16): 4499-506 (2000). |
| | | MPFATPMEAEL (SEQ ID NO: 41) | Aarnoudse et al. Int J Cancer. |
| | | KEFTVSGNILTI (SEQ ID NO: 42) | 82(3): 442-8 (1999). |
| | | MPFATPMEA (SEQ ID NO: 43) | Eikawa et al. Int J Cancer. |
| | | FATPMEAEL (SEQ ID NO: 44) | 132(2): 345-54 (2013). |
| | | FATPMEAELAR (SEQ ID NO: 45) | Wang et al. J Immunol. |
| | | LAMPFATPM (SEQ ID NO: 46) | 161(7): 3598-606 (1998). |
| | | ARGPESRLL (SEQ ID NO: 34) | Matsuzaki et al. Cancer Immunol |
| | | SLLMWITQCFLPVF (SEQ ID NO: 47) | Immunother. 57(8)1185-95 |
| | | -EAEL-ARRSLAQ (SEQ ID NO: 389) | (2008). |
| | | EFYLAMPFATPM (SEQ ID NO: 49) | Ebert et al. Cancer Res. |
| | | PGVLLKEFTVSGNILTIRL-TAADHR (SEQ ID | 69(3): 1046-54 (2009). |
| | | NO: 50) | Eikawa et al. Int J Cancer. |
| | | RLLEFYLAMPFA (SEQ ID NO: 51) | 132(2): 345-54 (2013). |
| | | QGAMLAAQERRVPRAAE-VPR (SEQ ID NO: | Knights et al. Cancer Immunol |
| | | 52) | Immunother. 58(3): 325-38 |
| | | PFATPMEAELARR (SEQ ID NO: 53) | (2009). |
| | | PGVLLKEFTVSGNILTIRLT (SEQ ID NO: 54) | Jäger et al. Cancer Immun. 2: 12 |
| | | VLLKEFTVSG (SEQ ID NO: 55) (2002). | |
| | | AADHRQLQLSISSCLQQL (SEQ ID NO: 56) | Zeng et al. Proc Natl Acad Sci USA. |
| | | LKEFTVSGNILTIRL (SEQ ID NO: 57) | 98(7): 3964-9 (2001). |
| | | PGVLLKEFTVSGNILTIRL-TAADHR (SEQ ID | Mandic et al. J Immunol. |
| | | NO: 50) | 174(3): 1751-9 (2005). |
| | | LLEFYLAMPFATPMEAEL-ARRSLAQ (SEQ ID | Chen et al. Proc Natl Acad Sci USA. |
| | | NO: 48) | 101(25): 9363-8 (2004). |
| | | KEFTVSGNILT (SEQ ID NO: 58) | Ayyoub et al. Clin Cancer Res. |
| | | LLEFYLAMPFATPM (SEQ ID NO: 59) | 16(18): 4607-15 (2010). |
| | | AGATGGRGPRGAGA (SEQ ID NO: 60) | Slager et al. J Immunol. |
| | | | 172(8): 5095-102 (2004). |
| | | | Mizote et al. Vaccine. |
| | | | 28(32): 5338-46 (2010). |
| | | | Jager et al. J Exp Med. |
| | | | 191(4): 625-30 (2000). |
| | | | Zarour et al. Cancer Res. |
| | | | 60(17): 4946-52 (2000). |
| | | | Zeng et al. J Immunol. |
| | | | 165(2): 1153-9 (2000). |
| | | | Bioley et al. Clin Cancer Res. |
| | | | 15(13): 4467-74 (2009). |
| | | | Zarour et al. Cancer Res. |
| | | | 62(1): 213-8 (2002). |
| | | | Hasegawa et al. Clin Cancer |
| | | | Res. 12(6): 1921-7 (2006). |
| 8 | CEA | TYYRPGVNLSLSC (SEQ ID NO: 61) | Galanis et al. Cancer Res. |
| | | EIIYPNASLLIQN (SEQ ID NO: 62) | 70(3): 875-82 (2010). |
| | | YACFVSNLATGRNNS (SEQ ID NO: 63) | Bast et al. Am. J. Obstet. |
| | | LWWVNNQSLPVSP (SEQ ID NO: 64) | Gynecol. 149(5): 553-9 (1984). |
| | | LWWVNNQSLPVSP (SEQ ID NO: 64) | Crosti et al. J Immunol. |
| | | LWWVNNQSLPVSP (SEQ ID NO: 64) | 176(8): 5093-9 (2006). |
| | | EIIYPNASLLIQN (SEQ ID NO: 62) | Kobayashi et al. Clin Cancer |
| | | NSIVKSITVSASG (SEQ ID NO: 65) | Res. 8(10): 3219-25 (2002). |
| | | KTWGQYWQV (SEQ ID NO: 66) | Campi et al. Cancer Res. |
| | | (A)MLGTHTMEV (SEQ ID NO: 67) | 63(23): 8481-6 (2003). |
| | | ITDQVPFSV (SEQ ID NO: 68) | Bakker et al. Int J Cancer. |
| | | YLEPGPVTA (SEQ ID NO: 69) | 62(1): 97-102 (1995). |
| | | LLDGTATLRL (SEQ ID NO: 70) | Tsai et al. J Immunol. |
| | | VLYRYGSFSV (SEQ ID NO: 71) | 158(4): 1796-802 (1997). |
| | | SLADTNSLAV (SEQ ID NO: 72) | Kawakami et al. J Immunol. |
| | | RLMKQDFSV (SEQ ID NO: 73) | 154(8): 3961-8 (1995). |
| | | RLPRIFCSC (SEQ ID NO: 74) | Cox et al. Science. |
| | | LIYRRRLMK (SEQ ID NO: 75) | 264(5159): 716-9 (1994). |
| | | ALLAVGATK (SEQ ID NO: 76) | Kawakami et al. J Immunol. |
| | | IALNFPGSQK (SEQ ID NO: 77) | 154(8): 3961-8 (1995). |
| | | RSYVPLAHR (SEQ ID NO: 78) | Kawakami et al. J Immunol. |
| | | | 161(12): 6985-92 (1998). |
| | | | Skipper et al. J Immunol. |
| | | | 157(11): 5027-33 (1996). |
| | | | Michaux et al. J Immunol. |
| | | | 192(4): 1962-71 (2014). |
| 9 | p53 | VVPCEPPEV (SEQ ID NO: 79) | Hung et al. Immunol. Rev. |
| | | | 222: 43-69 (2008). |
| 10 | Her2/Neu | HLYQGCQVV (SEQ ID NO: 80) | Nakatsuka et al. Mod. Pathol. |
| | | YLVPQQGFFC (SEQ ID NO: 81) | 19(6): 804-814 (2006). |
| | | PLQPEQLQV (SEQ ID NO: 82) | Pils et al. Br. J. Cancer |
| | | TLEEITGYL (SEQ ID NO: 83) | 96(3): 485-91 (2007). |
| | | ALIHHNTHL (SEQ ID NO: 84) | Scardino et al. Eur J Immunol. |
| | | PLTSIISAV (SEQ ID NO: 85) | 31(11): 3261-70 (2001). |
| | | VLRENTSPK (SEQ ID NO: 86) | Scardino et al. J Immunol. |
| | | TYLPTNASL (SEQ ID NO: 87) | 168(11): 5900-6 (2002). |
| | | | Kawashima et al. Cancer Res. |
| | | | 59(2): 431-5 (1999). |
| | | | Okugawa et al. Eur J Immunol. |
| | | | 30(11): 3338-46 (2000). |
| 11 | EpCAM | RYQLDPKFI (SEQ ID NO: 88) | Spizzo et al. Gynecol. Oncol. |
| | | | 103(2): 483-8 (2006). |
| | | | Tajima et al. Tissue Antigens. |
| | | | 64(6): 650-9 (2004). |
| 12 | CA125 | ILFTINFTI (SEQ ID NO: 89) | Bast et al. Cancer 116(12): 2850-2853 |
| | | VLFTINFTI (SEQ ID NO: 90) | (2010). |
| | | TLNFTITNL (SEQ ID NO: 91) | |
| | | VLQGLLKPL (SEQ ID NO: 92) | |
| | | VLQGLLRPV (SEQ ID NO: 93) | |
| | | RLDPKSPGV (SEQ ID NO: 94) | |
| | | QLYWELSKL (SEQ ID NO: 95) | |
| | | KLTRGIVEL (SEQ ID NO: 96) | |
| | | QLTNGITEL (SEQ ID NO: 97) | |
| | | QLTHNITEL (SEQ ID NO: 98) | |
| | | TLDRNSLYV (SEQ ID NO: 99) | |
| 13 | Folate | FLLSLALML (SEQ ID NO: 100) | Bagnoli et al. Gynecol. Oncol. |
| | receptor α | NLGPWIQQV (SEQ ID NO: 101) | 88: S140-4 (2003). |
| | | | Pampeno et al. (2016) High- |
| | | | ranking In Silico epitopes |
| | | | [determined by 3 |
| | | | algorithms: BISMAS, IEDB, |
| | | | RANKPEP] unpublished |
| 14 | Sperm | ILDSSEEDK (SEQ ID NO: 102) | Chiriva-Inernati et al. J. |
| | protein 17 | | Immunother. 31(8): 693-703 |
| | | | (2008). |
| 15 | TADG-12 | YLPKSWTIQV (SEQ ID NO: 103) | Bellone et al. Cancer 115(4): 800-11 |
| | | WIHEQMERDLKT (SEQ ID NO: 104) | (2009). |
| | | | Underwood et al. BBA Mol. Basis |
| | | | of Disease. 1502(3): 337-350 |
| | | | (2000). |
| 16 | MUC-16 | ILFTINFTI (SEQ ID NO: 89) | Chekmasova et al. Clin. Cancer |
| | | VLFTINFTI (SEQ ID NO: 90) | Res. 16(14): 3594-606 (2010). |
| | | TLNFTITNL (SEQ ID NO: 91) | |
| | | VLQGLLKPL (SEQ ID NO: 92) | |
| | | VLQGLLRPV (SEQ ID NO: 93) | |
| | | RLDPKSPGV (SEQ ID NO: 94) | |
| | | QLYWELSKL (SEQ ID NO: 95) | |
| | | KLTRGIVEL (SEQ ID NO: 96) | |
| | | QLTNGITEL (SEQ ID NO: 97) | |
| | | QLTHNITEL (SEQ ID NO: 98) | |
| | | TLDRNSLYV (SEQ ID NO: 99) | |
| 17 | L1CAM | LLANAYIYV (SEQ ID NO: 105) | Hong et al. J. Immunother. |
| | | YLLCKAFGA (SEQ ID NO: 106) | 37(2): 93-104 (2014). |
| | | KLSPYVHYT (SEQ ID NO: 107) | Pampeno et al. (2016) High- |
| | | | ranking In Silico epitopes |
| | | | [determined by 3 |
| | | | algorithms: BISMAS, IEDB, |
| | | | RANKPEP] |
| | | | unpublished |
| 18 | Mannan- | PDTRPAPGSTAPPAHGVTSA (SEQ ID NO: 108) | Loveland et al. Clin. Cancer Res. |
| | MUC-1 | STAPPVHNV (SEQ ID NO: 109) | 12(3 Pt 1): 869-77 (2006). |
| | | LLLLTVLTV (SEQ ID NO: 110) | Godelaine et al. Cancer Immunol |
| | | PGSTAPPAHGVT (SEQ ID NO: 111) | Immunother. 56(6): 753-9 (2007). |
| | | | Ma et al. Int J Cancer. |
| | | | 129(10): 2427-34 (2011). |
| | | | Wen et al. Cancer Sci. |
| | | | 102(8): 1455-61 (2011). |
| | | | Jerome et al. J Immunol. |
| | | | 151(3): 1654-62 (1993). |
| | | | Brossart et al. Blood. |
| | | | 93(12): 4309-17 (1999). |
| | | | Hiltbold et al. Cancer Res. |
| | | | 58(22): 5066-70 (1998). |
| 19 | HERV-K- | MLAVISCAV (SEQ ID NO: 112) | Schiavetti et al. Cancer Res. |
| | MEL | | 62(19): 5510-6 (2002). |
| 20 | KK-LC-1 | RQKRILVNL (SEQ ID NO: 113) | Fukuyama et al. Cancer Res. |
| | | | 66(9): 4922-8 (2006). |
| 21 | KM-HN-1 | NYNNFYRFL (SEQ ID NO: 114) | Fukuyama et al. Cancer Res. |
| | | EYSKECLKEF (SEQ ID NO: 115) | 66(9): 4922-8 (2006). |
| | | EYLSLSDKI (SEQ ID NO: 116) | Monji et al. Clin Cancer Res. |
| | | | 10(19 Pt 1): 6047-57 (2004). |
| 22 | LAGE-1 | MLMAQEALAFL (SEQ ID NO: 35) | Aarnoudse et al. Int J Cancer. |
| | | SLLMWITQC (SEQ ID NO: 32) | 82(3): 442-8 (1999). |
| | | LAAQERRVPR (SEQ ID NO: 38) | Rimoldi et al. J Immunol. |
| | | ELVRRILSR (SEQ ID NO: 117) | 165(12): 7253-61 (2000). |
| | | APRGVRMAV (SEQ ID NO: 118) | Wang et al. J Immunol. |
| | | SLLMWITQCFLPVF (SEQ ID NO: 47) | 161(7): 3598-606 (1998). |
| | | QGAMLAAQERRVPRAAEVP-R (SEQ ID NO: | Sun et al. Cancer Immunol |
| | | 119) | Immunother. 55(6): 644-52 |
| | | AADHRQLQLSISSCLQQL (SEQ ID NO: 56) | (2006). |
| | | CLSRRPWKRSWSAGSCPG-MPHL (SEQ ID | Slager et al. Cancer Gene Ther. |
| | | NO: 120) | 11(3): 227-36 (2004). |
| | | ILSRDAAPLPRPG (SEQ ID NO: 121) | Zeng et al. Proc Natl Acad Sci USA. |
| | | AGATGGRGPRGAGA (SEQ ID NO: 60) | 98(7): 3964-9 (2001). |
| | | | Slager et al. J Immunol. |
| | | | 172(8): 5095-102 (2004). |
| | | | Jager et al. J Exp Med. |
| | | | 191(4): 625-30 (2000). |
| | | | Slager et al. J Immunol. |
| | | | 170(3): 1490-7 (2003). |
| | | | Wang et al. Immunity. 20(1): 107-18 |
| | | | (2004). |
| | | | Hasegawa et al. Clin Cancer |
| | | | Res. 12(6): 1921-7 (2006). |
| 23 | MAGE-A4 | EVDPASNTY (SEQ ID NO: 122) | Kobayashi et al. Tissue |
| | | GVYDGREHTV (SEQ ID NO: 123) | Antigens. 62(5): 426-32 (2003). |
| | | NYKRCFPVI (SEQ ID NO: 124) | Duffour et al. Eur J Immunol. |
| | | SESLKMIF (SEQ ID NO: 125) | 29(10): 3329-37 (1999). |
| | | | Miyahara et al. Clin Cancer Res. |
| | | | 11(15): 5581-9 (2005). |
| | | | Ottaviani et al. Cancer Immunol |
| | | | Immunother. 55(7): 867-72 |
| | | | (2006) |
| | | | Zhang et al. Tissue Antigens. |
| | | | 60(5): 365-71 (2002). |
| 24 | Sp17 | ILDSSEEDK (SEQ ID NO: 102) | Chiriva-Internati et al. Int J |
| | | | Cancer. 107(5): 863-5 (2003). |
| 25 | SSX-4 | INKTSGPKRGKHAVVTHRLRE (SEQ ID NO: 126) | Ayyoub et al. Clin Immunol. |
| | | YFSKKEWEKMKSSEKIVYVY (SEQ ID NO: 127) | 114(1): 70-8 (2005). |
| | | MKLNYEVMTKLGFKVTLPPF (SEQ ID NO: 128) | Valmori et al. Clin Cancer Res. |
| | | KHAWTHRLRERKQLVVYEEI (SEQ ID NO: 129) | 12(2):398-404 (2006). |
| | | LGFKVTLPPFMRSKRAADFH (SEQ ID NO: 130) | |
| | | KSSEKIVYVYMKLNYEVMTK | (SEQ ID NO: 131) |
| | | KHAWTHRLRERKQLVVYEEI | (SEQ ID NO: 129) |
| 26 | TAG-1 | SLGWLFLLL (SEQ ID NO: 132) | Adair et al. J Immunother. |
| | | LSRLSNRLL (SEQ ID NO: 133) | 31(1):7-17 (2008). |
| 27 | TAG-2 | LSRLSNRLL (SEQ ID NO: 133) | Adair et al. J Immunother. |
| | | | 31(1):7-17 (2008). |

TABLE 2
Breast cancer

| | Tumor- | | |
|---|---|---|---|
| | associated | | |
| No. | antigen | Immunogenic epitopes | Sources |
| 1 | ENAH (hMena) | TMNGSKSPV (SEQ ID NO: 134) | Di Modugno et al. Int. J. |
| | | | Cancer. 109(6): 909-18 (2004). |
| 2 | mammaglobin-A | PLLENVISK (SEQ ID NO: 135) | Jaramillo et al. Int. J. Cancer. |
| | | | 102(5): 499-506 (2002). |
| 3 | NY-BR-1 | SLSKILDTV (SEQ ID NO: 136) | Wang et al. Cancer Res. |
| | | | 66(13): 6826-33 (2006). |
| 4 | EpCAM | RYQLDPKFI (SEQ ID NO: 88) | Gastl et al. Lancet |
| | | | 356(9246): 1981-2 (2000). |
| | | | Tajima, 2004 |
| 5 | NY-ESO-1 | HLA-A2-restricted peptide p157-165 | Jager et al. Proc. Natl. Acad. |
| | | (SLLMWITQC) (SEQ ID NO: 32), HLA-Cw3- | Scie. U.S.A. 103(39): 14453-8 |
| | | restricted p92-100 (LAMP-FATPM) (SEQ ID | (2006). |
| | | NO: 33) and HLA-Cw6-restricted p80-88 | Gnjatic et al. PNAS |
| | | (ARGPESRLL) (SEQ ID NO: 34) | Sep. 26, 2000 vol. 97 |
| | | SLLMWITQC (SEQ ID NO: 32) | no. 20 p. 10919 |
| | | MLMAQEALAFL (SEQ ID NO: 35) | Jager et al. J Exp Med. |
| | | YLAMPFATPME (SEQ ID NO: 36) | 187(2): 265-70 (1998). |
| | | ASGPGGGAPR (SEQ ID NO: 37) | Chen et al. J Immunol. |
| | | LAAQERRVPR (SEQ ID NO: 38) | 165(2): 948-55 (2000). |
| | | TVSGNILTIR (SEQ ID NO: 39) | Valmori et al. Cancer Res. |
| | | APRGPHGGAASGL (SEQ ID NO: 40) | 60(16): 4499-506 (2000). |
| | | MPFATPMEAEL (SEQ ID NO: 41) | Aarnoudse et al. Int J Cancer. |
| | | KEFTVSGNILTI (SEQ ID NO: 42) | 82(3): 442-8 (1999). |
| | | MPFATPMEA (SEQ ID NO: 43) | Eikawa et al. Int J Cancer. |
| | | FATPMEAEL (SEQ ID NO: 44) | 132(2): 345-54 (2013). |
| | | FATPMEAELAR (SEQ ID NO: 45) | Wang et al. J Immunol. |
| | | LAMPFATPM (SEQ ID NO: 46) | 161(7): 3598-606 (1998). |
| | | ARGPESRLL (SEQ ID NO: 34) | Matsuzaki et al. Cancer |
| | | SLLMWITQCFLPVF (SEQ ID NO: 47) | Immunol Immunother. |
| | | LLEFYLAMPFATPMEAEL-ARRSLAQ (SEQ ID | 57(8)1185-95 (2008). |
| | | NO: 48) | Ebert et al. Cancer Res. |
| | | EFYLAMPFATPM (SEQ ID NO: 49) | 69(3): 1046-54 (2009). |
| | | PGVLLKEFTVSGNILTIRL-TAADHR (SEQ ID | Eikawa et al. Int J Cancer. |
| | | NO: 50) | 132(2): 345-54 (2013). |
| | | RLLEFYLAMPFA (SEQ ID NO: 51) | Knights et al. Cancer Immunol |
| | | QGAMLAAQERRVPRAAE-VPR (SEQ ID NO: | Immunother. 58(3): 325-38 |
| | | 52) | (2009). |
| | | PFATPMEAELARR (SEQ ID NO: 53) | Jäger et al. Cancer Immun. |
| | | PGVLLKEFTVSGNILTIRLT (SEQ ID NO: 54) | 2: 12 (2002). |
| | | VLLKEFTVSG (SEQ ID NO: 55) | Zeng et al. Proc Natl Acad Sci |
| | | AADHRQLQLSISSCLQQL (SEQ ID NO: 56) | USA. 98(7): 3964-9 (2001). |
| | | LKEFTVSGNILTIRL (SEQ ID NO: 57) | Mandic et al. J Immunol. |
| | | PGVLLKEFTVSGNILTIRL-TAADHR (SEQ ID | 174(3): 1751-9 (2005). |
| | | NO: 50) | Chen et al. Proc Natl Acad Sci |
| | | LLEFYLAMPFATPMEAEL-ARRSLAQ (SEQ ID | USA. 101(25): 9363-8 (2004). |
| | | NO: 48) | Ayyoub et al. Clin Cancer Res. |
| | | KEFTVSGNILT (SEQ ID NO: 58) | 16(18): 4607-15 (2010). |
| | | LLEFYLAMPFATPM (SEQ ID NO: 59) | Slager et al. J Immunol. |
| | | AGATGGRGPRGAGA (SEQ ID NO: 60) | 172(8): 5095-102 (2004). |
| | | | Mizote et al. Vaccine. |
| | | | 28(32): 5338-46 (2010). |
| | | | Jager et al. J Exp Med. |
| | | | 191(4): 625-30 (2000). |
| | | | Zarour et al. Cancer Res. |
| | | | 60(17): 4946-52 (2000). |
| | | | Zeng et al. J Immunol. |
| | | | 165(2): 1153-9 (2000). |
| | | | Bioley et al. Clin Cancer Res. |
| | | | 15(13): 4467-74 (2009). |
| | | | Zarour et al. Cancer Res. |
| | | | 62(1): 213-8 (2002). |
| | | | Hasegawa et al. Clin Cancer |
| | | | Res. 12(6): 1921-7 (2006). |
| 6 | BAGE-1 | AARAVFLAL (SEQ ID NO: 137) | Boel et al. Immunity. 2(2): 167-75 |
| | | | (1995). |
| 7 | HERV-K-MEL | MLAVISCAV (SEQ ID NO: 112) | Schiavetti et al. Cancer Res. |
| | | | 62(19): 5510-6 (2002). |
| 8 | KK-LC-1 | RQKRILVNL (SEQ ID NO: 113) | Fukuyama et al. Cancer Res. |
| | | | 66(9): 4922-8 (2006). |
| 9 | KM-HN-1 | NYNNFYRFL (SEQ ID NO: 114) | Fukuyama et al. Cancer Res. |
| | | EYSKECLKEF (SEQ ID NO: 115) | 66(9): 4922-8 (2006). |
| | | EYLSLSDKI (SEQ ID NO: 116) | Monji et al. Clin Cancer Res. |
| | | | 10(18 Pt 1): 6047-57 (2004). |
| 10 | LAGE-1 | MLMAQEALAFL (SEQ ID NO: 35) | Aarnoudse et al. Int J Cancer. |
| | | SLLMWITQC (SEQ ID NO: 32) | 82(3): 442-8 (1999). |
| | | LAAQERRVPR (SEQ ID NO: 38) | Rimoldi et al. J Immunol. |
| | | ELVRRILSR (SEQ ID NO: 117) | 165(12): 7253-61 (2000). |
| | | APRGVRMAV (SEQ ID NO: 118) | Wang et al. J Immunol. |
| | | SLLMWITQCFLPVF (SEQ ID NO: 47) | 161(7): 3598-606 (1998). |
| | | QGAMLAAQERRVPRAAEVP-R (SEQ ID NO: | Sun et al. Cancer Immunol |
| | | 119) | Immunother. 55(6): 644-52 |
| | | AADHRQLQLSISSCLQQL (SEQ ID NO: 56) | (2006). |
| | | CLSRRPWKRSWSAGSCPG-MPHL (SEQ ID | Slager et al. Cancer Gene |
| | | NO: 120) | Ther. 11(3): 227-36 (2004). |
| | | ILSRDAAPLPRPG (SEQ ID NO: 121) | Zeng et al. Proc Natl Acad Sci |
| | | AGATGGRGPRGAGA (SEQ ID NO: 60) | USA. 98(7): 3964-9 (2001). |
| | | | Slager et al. J Immunol. |
| | | | 172(8): 5095-102 (2004). |
| | | | Jager et al. J Exp Med. |
| | | | 191(4): 625-30 (2000). |
| | | | Slager et al. J Immunol. |
| | | | 170(3): 1490-7 (2003). |
| | | | Wang et al. Immunity. |
| | | | 20(1): 107-18 (2004). |
| | | | Hasegawa et al. Clin Cancer |
| | | | Res. 12(6): 1921-7 (2006). |
| 11 | MAGE-A1 | EADPTGHSY (SEQ ID NO: 138) | Traversari et al. J Exp Med. |
| | | KVLEYVIKV (SEQ ID NO: 139) | 176(5): 1453-7 (1992). |
| | | SLFRAVITK (SEQ ID NO: 140) | Ottaviani et al. Cancer |
| | | EVYDGREHSA (SEQ ID NO: 141) | Immunol Immunother. |
| | | RVRFFFPSL (SEQ ID NO: 142) | 54(12): 1214-20 (2005). |
| | | EADPTGHSY (SEQ ID NO: 138) | Pascolo et al. Cancer Res. |
| | | REPVTKAEML (SEQ ID NO: 143) | 61(10): 4072-7 (2001). |
| | | KEADPTGHSY (SEQ ID NO: 144) | Chaux et al. J Immunol. |
| | | DPARYEFLW (SEQ ID NO: 145) | 163(5): 2928-36 (1999). |
| | | ITKKVADLVGF (SEQ ID NO: 146) | Luiten et al. Tissue Anitgens. |
| | | SAFPTTINF (SEQ ID NO: 147) | 55(2): 49-52 (2000). |
| | | SAYGEPRKL (SEQ ID NO: 148) | Luiten et al. Tissue Antigens. |
| | | RVRFFFPSL (SEQ ID NO: 142) | 56(1): 77-81 (2000). |
| | | TSCILESLFRAVITK (SEQ ID NO: 149) | Tanzarella et al. Cancer Res. |
| | | PRALAETSYVKVLEY (SEQ ID NO: 150) | 59(11): 2668-74 (1999). |
| | | FLLLKYRAREPVTKAE (SEQ ID NO: 151) | Stroobant et al. Eur J Immunol. |
| | | EYVIKVSARVRF (SEQ ID NO: 152) | 42(6): 1417-28 (2012). |
| | | | Corbiére et al. Tissue |
| | | | Antigens. 63(5): 453-7 (2004). |
| | | | Goodyear et al. Cancer |
| | | | Immunol Immunother. |
| | | | 60(12): 1751-61 (2011). |
| | | | van der Bruggen et al. Eur J |
| | | | Immunol. 24(9): 2134-40 |
| | | | (1994). |
| | | | Wang et al. Cancer Immunol |
| | | | Immunother. 56(6): 807-18 |
| | | | (2007). |
| | | | Chaux et al. J Exp Med. |
| | | | 189(5): 767-78 (1999). |
| | | | Chaux et al. Eur J Immunol. |
| | | | 31(6): 1910-6 (2001). |
| 12 | MAGE-A2 | YLQLVFGIEV (SEQ ID NO: 153) | Kawashima et al. Hum |
| | | EYLQLVFGI (SEQ ID NO: 154) | Immunol. 59(1): 1-14 (1998). |
| | | REPVTKAEML (SEQ ID NO: 143) | Tahara et al. Clin Cancer Res. |
| | | EGDCAPEEK (SEQ ID NO: 155) | 5(8): 2236-41 (1999). |
| | | LLKYRAREPVTKAE (SEQ ID NO: 156) | Tanzarella et al. Cancer Res. |
| | | | 59(11): 2668-74 (1999). |
| | | | Breckpot et al. J Immunol. |
| | | | 172(4): 2232-7 (2004). |
| | | | Chaux et al. J Exp Med. |
| | | | 89(5): 767-78 (1999). |
| 13 | mucink | PDTRPAPGSTAPPAHGVTSA (SEQ ID NO: | Jerome et al. J Immunol. |
| | | 108) | 151(3): 1654-62 (1993). |
| 14 | Sp17 | ILDSSEEDK (SEQ ID NO: 102) | Chiriva-Intemati et al. Int J |
| | | | Cancer. 107(5): 863-5 (2003). |
| 15 | SSX-2 | KASEKIFYV (SEQ ID NO: 157) | Ayyoub et al. J Immunol. |
| | | EKIQKAFDDIAKYFSK (SEQ ID NO: 158) | 168(4): 1717-22 (2002). |
| | | FGRLQGISPKI (SEQ ID NO: 159) | Ayyoub et al. J Immunol. |
| | | WEKMKASEKIFYVYMKRK (SEQ ID NO: 160) | 172(11): 7206-11 (2004). |
| | | KIFYVYMKRKYEAMT (SEQ ID NO: 161) | Neumann et al. Cancer |
| | | KIFYVYMKRKYEAM (SEQ ID NO: 162) | Immunol Immunother. |
| | | | 60(9): 1333-46 (2011). |
| | | | Ayyoub et al. Clin Immunol. |
| | | | 114(1): 70-8 (2005). |
| | | | Neumann et al. Int J Cancer. |
| | | | 112(4): 661-8 (2004). |
| | | | Ayyoub et al. J Clin Invest. |
| | | | 113(8): 1225-33 (2004). |
| 16 | TAG-1 | SLGWLFLLL (SEQ ID NO: 132) | Adair et al. J Immunother. |
| | | LSRLSNRLL (SEQ ID NO: 133) | 31(1): 7-17 (2008). |
| 17 | TAG-2 | LSRLSNRLL (SEQ ID NO: 133) | Adair et al. J Immunother. |
| | | | 31(1): 7-17 (2008). |
| 18 | TRAG-3 | CEFHACWPAFTVLGE (SEQ ID NO: 163) | Janjic et al. J Immunol. |
| | | | 177(4): 2717-27 (2006). |
| 19 | Her2/Neu | HLYQGCQVV (SEQ ID NO: 80) | Nakatsuka et al. Mod. Pathol. |
| | | YLVPQQGFFC (SEQ ID NO: 81) | 19(6): 804-814 (2006). |
| | | PLQPEQLQV (SEQ ID NO: 82) | Pils et al. Br. J. Cancer |
| | | TLEEITGYL (SEQ ID NO: 83) | 96(3): 485-91 (2007). |
| | | ALIHHNTHL (SEQ ID NO: 84) | Scardino et al. Eur J Immunol. |
| | | PLTSIISAV (SEQ ID NO: 85) | 31(11): 3261-70 (2001). |
| | | VLRENTSPK (SEQ ID NO: 86) | Scardino et al. J Immunol. |
| | | TYLPTNASL (SEQ ID NO: 87) | 168(11): 5900-6 (2002). |
| | | | Kawashima et al. Cancer Res. |
| | | | 59(2): 431-5 (1999). |
| | | | Okugawa et al. Eur J Immunol. |
| | | | 30(11): 3338-46 (2000). |
| 20 | c-myc | SSPQGSPEPL (SEQ ID NO: 164) | Helm et al. PLoS ONE 8(10): |
| | | | e77375 (2013). |
| 21 | cyclin B1 | ILIDWLVQV (SEQ ID NO: 165) | Andersen et al. Cancer |
| | | | Immunol Immunother 60: 227 |
| | | | (2011). |
| 22 | MUC1 | STAPPVHNV (SEQ ID NO: 109) | Brossart et al. Blood, 93(12), |
| | | LLLLTVLTV (SEQ ID NO: 110) | 4309-4317 (1999). |
| 23 | p53 | VVPCEPPEV (SEQ ID NO: 79) | Hung et al. Immunol. Rev. |
| | | | 222: 43-69 (2008). |
| | | | http://cancerimmunity.org/ |
| | | | peptide/mutations/ |
| 24 | p62 | FLKNVGESV (SEQ ID NO: 166) | Pampeno et al. (2016) High- |
| | | | ranking In Silico epitopes |
| | | | [determined by 3 algorithms: |
| | | | BISMAS, IEDB, |
| | | | RANKPEP]unpublished |
| 25 | Survivin | ELTLGEFLKL (SEQ ID NO: 167) | Schmitz M Cancer Res. 60: |
| | | | 4845-9 (2000). |

TABLE 3
Testicular cancer

| | Tumor- | | |
|---|---|---|---|
| | associated | | |
| No. | antigen | Immunogenic epitopes | Sources |
| 1 | CD45 | KFLDALISL (SEQ ID NO: 168) | Tomita et al. Cancer Sci. |
| | | | 102(4): 697-705 (2011). |
| 2 | DKK1 | ALGGHPLLGV (SEQ ID NO: 169) | Qian et al. Blood. (5): 1587-94 |
| | | | (2007). |
| 3 | PRAME | VLDGLDVLL (SEQ ID NO: 17), | Kessler et al. J Exp Med. |
| | | SLYSFPEPEA (SEQ ID NO: 18), | 193(1): 73-88 (2001). |
| | | ALYVDSLFFL (SEQ ID NO: 19), | Ikeda et al. Immunity 6(2): 199-208 |
| | | SLLQHLIGL (SEQ ID NO: 20), | (1997). |
| | | LYVDSLFFL (SEQ ID NO: 21) | |
| 4 | RU2AS | LPRWPPPQL (SEQ ID NO: 170) | Van Den Eynde et al. J. Exp. Med. |
| | | | 190(12): 1793-800 (1999). |
| 5 | Telomerase | ILAKFLHWL (SEQ ID NO: 171); | Vonderheide et al. Immunity |
| | | RLVDDFLLV (SEQ ID NO: 172); | 10(6): 673-9 (1999). |
| | | RPGLLGASVLGLDDI (SEQ ID NO: 173); | Miney et al. Proc. Natl. Acad. Sci. |
| | | and LTDLQPYMRQFVAHL (SEQ ID NO: | U.S.A. 97(9): 4796-801 (2000). |
| | | 174) | Schroers et al. Cancer Res. |
| | | | 62(9): 2600-5 (2002). |
| | | | Schroers et al. Clin. Cancer Res. |
| | | | 9(13): 4743-55 (2003). |

TABLE 4
Pancreatic cancer

| | Tumor- | | |
|---|---|---|---|
| | associated | | |
| No. | antigen | Immunogenic epitopes | Sources |
| 1 | ENAH (hMena) | TMNGSKSPV (SEQ ID NO: 134) | Di Modugno et al. Int. J. Cancer. |
| | | | 109(6): 909-18 (2004). |
| 2 | PBF | CTACRWKKACQR (SEQ ID NO: 16) | Tsukahara et al. Cancer Res. |
| | | | 64(15): 5442-8 (2004). |
| 3 | K-ras | VVVGAVGVG (SEQ ID NO: 175) | Gjertsen et al. Int. J. Cancer. |
| | | | 72(5): 784-90 (1997). |
| 4 | Mesothelin | SLLFLLFSL (SEQ ID NO: 27) | Le et al. Clin. Cancer Res. |
| | | VLPLTVAEV (SEQ ID NO: 28) | 18(3): 858-68 (2012). |
| | | ALQGGGPPY (SEQ ID NO: 29) | Hassan et al. Appl. |
| | | LYPKARLAF (SEQ ID NO: 30) | Immunohistochem. Mol. |
| | | AFLPWHRLF (SEQ ID NO: 31) | Morphol. 13(3): 243-7 (2005). |
| | | | Thomas et al J Exp Med. 2004 |
| | | | Aug 2; 200(3): 297-306. |
| 5 | mucink | PDTRPAPGSTAPPAHGVTSA (SEQ ID NO: | Jerome et al. J Immunol. |
| | | 108) | 151(3): 1654-62 (1993). |

TABLE 5
Liver cancer

| | Tumor- | | |
|---|---|---|---|
| | associated | | |
| No. | antigen | Immunogenic epitopes | Sources |
| 1 | G250/MN/ | HLSTAFARV (SEQ ID NO: 176); | Vissers et al. Cancer Res. |
| | CAIX | KIFGSLAFL (SEQ ID NO: 177); | 59(21): 5554-9 (1999). |
| | | IISAVVGIL (SEQ ID NO: 178); | Fisk et al. J Exp Med. |
| | | ALCRWGLLL (SEQ ID NO: 179); | 181(6): 2109-17 (1995). |
| | | ILHNGAYSL (SEQ ID NO: 180); | Brossart et al. Cancer Res. |
| | | RLLQETELV (SEQ ID NO: 181); | 58(4): 732-6 (1998). |
| | | VVKGVVFGI (SEQ ID NO: 182); and | Kawashima et al. Hum Immunol. |
| | | YMIMVKCWMI (SEQ ID NO: 183) | 59(1): 1-14 (1998). |
| | | | Rongcun et al. J Immunol. |
| | | | 163(2): 1037-44 (1999). |
| 2 | Hepsin | SLLSGDWVL (SEQ ID NO: 184); | Guo et al. Scand J Immunol. |
| | | GLQLGVQAV (SEQ ID NO: 185); and | 78(3): 248-57 (2013). |
| | | PLTEYIQPV (SEQ ID NO: 186) | |
| 3 | Intestinal | SPRWWPTCL (SEQ ID NO: 187) | Ronsin et al. J Immunol. |
| | carboxyl | | 163(1): 483-90 (1999). |
| | esterase | | |
| 4 | alpha- | GVALQTMKQ (SEQ ID NO: 188); | Butterfield et al. Cancer Res. |
| | foetoprotein | FMNKFIYEI (SEQ ID NO: 189); and | 59(13): 3134-42 (1999). |
| | | QLAVSVILRV (SEQ ID NO: 190) | Pichard et al. J Immunother. |
| | | | 31(3): 246-53 (2008) |
| | | | Alisa et al. Clin. Cancer Res. |
| | | | 11(18): 6686-94 (2005). |
| 5 | M-CSF | LPAVVGLSPGEQEY (SEQ ID NO: 191) | Probst-Kepper et al. J Exp Med. |
| | | | 193(10): 1189-98 (2001). |
| 6 | PBF | CTACRWKKACQR (SEQ ID NO: 16) | Tsukahara et al. Cancer Res. |
| | | | 64(15): 5442-8 (2004). |
| 7 | PSMA | NYARTEDFF (SEQ ID NO: 192) | Horiguchi et al. Clin Cancer Res. |
| | | | 8(12): 3885-92 (2002). |
| 8 | NY-ESO-1 | HLA-A2-restricted peptide p157-165 | Jager et al. Proc. Natl. Acad. |
| | | (SLLMWITQC) (SEQ ID NO: 32), HLA-Cw3- | Scie. U.S.A. 103(39): 14453-8 |
| | | restricted p92-100 (LAMP-FATPM) (SEQ ID | (2006). |
| | | NO: 33) and HLA-Cw6-restricted p80-88 | Gnjatic et al. PNAS |
| | | (ARGPESRLL) (SEQ ID NO: 34) | Sep. 26, 2000 vol. 97 no. |
| | | SLLMWITQC (SEQ ID NO: 32) | 20 p. 10919 |
| | | MLMAQEALAFL (SEQ ID NO: 35) | Jager et al. J Exp Med. |
| | | YLAMPFATPME (SEQ ID NO: 36) | 187(2): 265-70 (1998). |
| | | ASGPGGGAPR (SEQ ID NO: 37) | Chen et al. J Immunol. |
| | | LAAQERRVPR (SEQ ID NO: 38) | 165(2): 948-55 (2000). |
| | | TVSGNILTIR (SEQ ID NO: 39) | Valmori et al. Cancer Res. |
| | | APRGPHGGAASGL (SEQ ID NO: 40) | 60(16): 4499-506 (2000). |
| | | MPFATPMEAEL (SEQ ID NO: 41) | Aarnoudse et al. Int J Cancer. |
| | | KEFTVSGNILTI (SEQ ID NO: 42) | 82(3): 442-8 (1999). |
| | | MPFATPMEA (SEQ ID NO: 43) | Eikawa et al. Int J Cancer. |
| | | FATPMEAEL (SEQ ID NO: 44) | 132(2): 345-54 (2013). |
| | | FATPMEAELAR (SEQ ID NO: 45) | Wang et al. J Immunol. |
| | | LAMPFATPM (SEQ ID NO: 46) | 161(7): 3598-606 (1998). |
| | | ARGPESRLL (SEQ ID NO: 34) | Matsuzaki et al. Cancer Immunol |
| | | SLLMWITQCFLPVF (SEQ ID NO: 47) | Immunother. 57(8)1185-95 |
| | | LLEFYLAMPFATPMEAEL-ARRSLAQ (SEQ ID | (2008). |
| | | NO: 48) | Ebert et al. Cancer Res. |
| | | EFYLAMPFATPM (SEQ ID NO: 49) | 69(3): 1046-54 (2009). |
| | | PGVLLKEFTVSGNILTIRL-TAADHR (SEQ ID | Eikawa et al. Int J Cancer. |
| | | NO: 50) | 132(2): 345-54 (2013). |
| | | RLLEFYLAMPFA (SEQ ID NO: 51) | Knights et al. Cancer Immunol |
| | | QGAMLAAQERRVPRAAE-VPR (SEQ ID NO: | Immunother. 58(3): 325-38 |
| | | 52) | (2009). |
| | | PFATPMEAELARR (SEQ ID NO: 53) | Jäger et al. Cancer Immun. 2: 12 |
| | | PGVLLKEFTVSGNILTIRLT (SEQ ID NO: 54) | (2002). |
| | | VLLKEFTVSG (SEQ ID NO: 55) | Zeng et al. Proc Natl Acad Sci USA. |
| | | AADHRQLQLSISSCLQQL (SEQ ID NO: 56) | 98(7): 3964-9 (2001). |
| | | LKEFTVSGNILTIRL (SEQ ID NO: 57) | Mandic et al. J Immunol. |
| | | PGVLLKEFTVSGNILTIRL-TAADHR (SEQ ID | 174(3): 1751-9 (2005). |
| | | NO: 50) | Chen et al. Proc Natl Acad Sci USA. |
| | | LLEFYLAMPFATPMEAEL-ARRSLAQ (SEQ ID | 101(25): 9363-8 (2004). |
| | | NO: 48) | Ayyoub et al. Clin Cancer Res. |
| | | KEFTVSGNILT (SEQ ID NO: 58) | 16(18): 4607-15 (2010). |
| | | LLEFYLAMPFATPM (SEQ ID NO: 59) | Slager et al. J Immunol. |
| | | AGATGGRGPRGAGA (SEQ ID NO: 60) | 172(8): 5095-102 (2004). |
| | | | Mizote et al. Vaccine. |
| | | | 28(32): 5338-46 (2010). |
| | | | Jager et al. J Exp Med. |
| | | | 191(4): 625-30 (2000). |
| | | | Zarour et al. Cancer Res. |
| | | | 60(17): 4946-52 (2000). |
| | | | Zeng et al. J Immunol. |
| | | | 165(2): 1153-9 (2000). |
| | | | Bioley et al. Clin Cancer Res. |
| | | | 15(13): 4467-74 (2009). |
| | | | Zarour et al. Cancer Res. |
| | | | 62(1): 213-8 (2002). |
| | | | Hasegawa et al. Clin Cancer |
| | | | Res. 12(6): 1921-7 (2006). |
| 9 | LAGE-1 | MLMAQEALAFL (SEQ ID NO: 35) | Aarnoudse et al. Int J Cancer. |
| | | SLLMWITQC (SEQ ID NO: 32) | 82(3): 442-8 (1999). |
| | | LAAQERRVPR (SEQ ID NO: 38) | Rimoldi et al. J Immunol. |
| | | ELVRRILSR (SEQ ID NO: 117) | 165(12): 7253-61 (2000). |
| | | APRGVRMAV (SEQ ID NO: 118) | Wang et al. J Immunol. |
| | | SLLMWITQCFLPVF (SEQ ID NO: 47) | 161(7): 3598-606 (1998). |
| | | QGAMLAAQERRVPRAAEVP-R (SEQ ID NO: | Sun et al. Cancer Immunol |
| | | 119) | Immunother. 55(6): 644-52 |
| | | AADHRQLQLSISSCLQQL (SEQ ID NO: 56) | (2006). |
| | | CLSRRPWKRSWSAGSCPG-MPHL (SEQ ID | Slager et al. Cancer Gene Ther. |
| | | NO: 120) | 11(3): 227-36 (2004). |
| | | ILSRDAAPLPRPG (SEQ ID NO: 121) | Zeng et al. Proc Natl Acad Sci USA. |
| | | AGATGGRGPRGAGA (SEQ ID NO: 60) | 98(7): 3964-9 (2001). |
| | | | Slager et al. J Immunol. |
| | | | 172(8): 5095-102 (2004). |
| | | | Jager et al. J Exp Med. |
| | | | 191(4): 625-30 (2000). |
| | | | Slager et al. J Immunol. |
| | | | 170(3): 1490-7 (2003). |
| | | | Wang et al. Immunity. 20(1): 107-18 |
| | | | (2004). |
| | | | Hasegawa et al. Clin Cancer |
| | | | Res. 12(6): 1921-7 (2006). |
| 10 | HERV-K- | MLAVISCAV (SEQ ID NO: 112) | Schiavetti et al. Cancer Res. |
| | MEL | | 62(19): 5510-6 (2002). |
| 11 | KK-LC-1 | RQKRILVNL (SEQ ID NO: 113) | Fukuyama et al. Cancer Res. |
| | | | 66(9): 4922-8 (2006). |
| 12 | KM-HN-1 | NYNNFYRFL (SEQ ID NO: 114) | Fukuyama et al. Cancer Res. |
| | | EYSKECLKEF (SEQ ID NO: 115) | 66(9): 4922-8 (2006). |
| | | EYLSLSDKI (SEQ ID NO: 116) | Monji et al. Clin Cancer Res. |
| | | | 10(18 Pt 1): 6047-57 (2004). |
| 13 | Sp17 | ILDSSEEDK (SEQ ID NO: 102) | Chiriva-Internati et al. Int J |
| | | | Cancer. 107(5): 863-5 (2003). |
| 14 | c-myc | SSPQGSPEPL (SEQ ID NO: 164) | Helm et al. PLoS ONE 8(10): |
| | | | e77375 (2013). |
| 15 | cyclin B1 | ILIDWLVQV (SEQ ID NO: 165) | Andersen et al. Cancer Immunol |
| | | | Immunother 60: 227 (2011). |
| 16 | p53 | VVPCEPPEV (SEQ ID NO: 79) | Hung et al. Immunol. Rev. |
| | | | 222: 43-69 (2008). |
| | | | http://cancerimmunity.org/peptide/mutations/ |
| 17 | p62 | FLKNVGESV (SEQ ID NO: 166) | Pampeno et al. (2016) High- |
| | | | ranking In Silico epitopes |
| | | | [determined by 3 algorithms: |
| | | | BISMAS, IEDB, |
| | | | RANKPEP]unpublished |
| 18 | Survivin | ELTLGEFLKL (SEQ ID NO: 167) | Schmitz M Cancer Res. 60: |
| | | | 4845-9 (2000) |

TABLE 6
Colorectal cancer

| | Tumor- | | |
|---|---|---|---|
| | associated | | |
| No. | antigen | Immunogenic epitopes | Sources |
| 1 | ENAH (hMena) | TMNGSKSPV (SEQ ID NO: 134) | Di Modugno et al. Int. J |
| | | | Cancer. 109(6): 909-18 |
| | | | (2004). |
| 2 | Intestinal | SPRWWPTCL (SEQ ID NO: 187) | Ronsin et al. J Immunol. |
| | carboxyl | | 163(1): 483-90 (1999). |
| | esterase | | |
| 3 | CASP-5 | FLIIWQNTM (SEQ ID NO: 193) | Schwitalle et al. Cancer |
| | | | Immun. 4: 14 (2004). |
| 4 | COA-1 | TLYQDDTLTLQAAG (SEQ ID NO: 194) | Maccalli et al. Cancer Res. |
| | | | 63(20): 6735-43 (2003). |
| 5 | OGT | SLYKFSPFPL (SEQ ID NO: 195) | Ripberger. J Clin Immunol. |
| | | | 23(5): 415-23 (2003). |
| 6 | OS-9 | KELEGILLL (SEQ ID NO: 196) | Vigneron et al. Cancer |
| | | | Immun. 2: 9 (2002). |
| 7 | TGF- | RLSSCVPVA (SEQ ID NO: 197) | Linnebacher et al. Int. J. |
| | betaRII | | Cancer. 93(1): 6-11 (2001). |
| 8 | NY-ESO-1 | HLA-A2-restricted peptide p157-165 | Jager et al. Proc. Natl. Acad. |
| | | (SLLMWITQC) (SEQ ID NO: 32), HLA-Cw3- | Scie. U.S.A. 103(39): 14453-8 |
| | | restricted p92-100 (LAMP-FATPM) (SEQ ID | (2006). |
| | | NO: 33) and HLA-Cw6-restricted p80-88 | Gnjatic et al. PNAS |
| | | (ARGPESRLL) (SEQ ID NO: 34) | Sep. 26, 2000 vol. 97 |
| | | SLLMWITQC (SEQ ID NO: 32) | no. 20 p. 10919 |
| | | MLMAQEALAFL (SEQ ID NO: 35) | Jager et al. J Exp Med. |
| | | YLAMPFATPME (SEQ ID NO: 36) | 187(2): 265-70 (1998). |
| | | ASGPGGGAPR (SEQ ID NO: 37) | Chen et al. J Immunol. |
| | | LAAQERRVPR (SEQ ID NO: 38) | 165(2): 948-55 (2000). |
| | | TVSGNILTIR (SEQ ID NO: 39) | Valmori et al. Cancer Res. |
| | | APRGPHGGAASGL (SEQ ID NO: 40) | 60(16): 4499-506 (2000). |
| | | MPFATPMEAEL (SEQ ID NO: 41) | Aarnoudse et al. Int J Cancer. |
| | | KEFTVSGNILTI (SEQ ID NO: 42) | 82(3): 442-8 (1999). |
| | | MPFATPMEA (SEQ ID NO: 43) | Eikawa et al. Int J Cancer. |
| | | FATPMEAEL (SEQ ID NO: 44) | 132(2): 345-54 (2013). |
| | | FATPMEAELAR (SEQ ID NO: 45) | Wang et al. J Immunol. |
| | | LAMPFATPM (SEQ ID NO: 46) | 161(7): 3598-606 (1998). |
| | | ARGPESRLL (SEQ ID NO: 34) | Matsuzaki et al. Cancer |
| | | SLLMWITQCFLPVF (SEQ ID NO: 47) | Immunol Immunother. |
| | | LLEFYLAMPFATPMEAEL-ARRSLAQ (SEQ ID | 57(8)1185-95 (2008). |
| | | NO: 48) | Ebert et al. Cancer Res. |
| | | EFYLAMPFATPM (SEQ ID NO: 49) | 69(3): 1046-54 (2009). |
| | | PGVLLKEFTVSGNILTIRL-TAADHR (SEQ ID NO: | Eikawa et al. Int J Cancer. |
| | | 50) | 132(2): 345-54 (2013). |
| | | RLLEFYLAMPFA (SEQ ID NO: 51) | Knights et al. Cancer Immunol |
| | | QGAMLAAQERRVPRAAE-VPR (SEQ ID NO: 52) | Immunother. 58(3): 325-38 |
| | | PFATPMEAELARR (SEQ ID NO: 53) | (2009). |
| | | PGVLLKEFTVSGNILTIRLT (SEQ ID NO: 54) | Jäger et al. Cancer Immun. |
| | | VLLKEFTVSG (SEQ ID NO: 55) | 2: 12 (2002). |
| | | AADHRQLQLSISSCLQQL (SEQ ID NO: 56) | Zeng et al. Proc Natl Acad Sci |
| | | LKEFTVSGNILTIRL (SEQ ID NO: 57) | USA. 98(7): 3964-9 (2001). |
| | | PGVLLKEFTVSGNILTIRL-TAADHR (SEQ ID NO: | Mandic et al. J Immunol. |
| | | 50) | 174(3): 1751-9 (2005). |
| | | LLEFYLAMPFATPMEAEL-ARRSLAQ (SEQ ID | Chen et al. Proc Natl Acad |
| | | NO: 48) | Sci USA. 101(25): 9363- |
| | | KEFTVSGNILT (SEQ ID NO: 58) | 8 (2004). |
| | | LLEFYLAMPFATPM (SEQ ID NO: 59) | Ayyoub et al. Clin Cancer |
| | | AGATGGRGPRGAGA (SEQ ID NO: 60) | Res. 16(18): 4607-15 (2010). |
| | | | Slager et al. J Immunol. |
| | | | 172(8): 5095-102 (2004). |
| | | | Mizote et al. Vaccine. |
| | | | 28(32): 5338-46 (2010). |
| | | | Jager et al. J Exp Med. |
| | | | 191(4): 625-30 (2000). |
| | | | Zarour et al. Cancer Res. |
| | | | 60(17): 4946-52 (2000). |
| | | | Zeng et al. J Immunol. |
| | | | 165(2): 1153-9 (2000). |
| | | | Bioley et al. Clin Cancer Res. |
| | | | 15(13): 4467-74 (2009). |
| | | | Zarour et al. Cancer Res. |
| | | | 62(1): 213-8 (2002). |
| | | | Hasegawa et al. Clin Cancer |
| | | | Res. 12(6): 1921-7 (2006). |
| 9 | CEA | TYYRPGVNLSLSC (SEQ ID NO: 61) | Duffy, Clin. Chem. 47(4): 624-30 |
| | | EIIYPNASLLIQN (SEQ ID NO: 62) | (2001). |
| | | YACFVSNLATGRNNS (SEQ ID NO: 63) | Parkhurst et al. Mol. Ther. |
| | | LVWVVNNQSLPVSP (SEQ ID NO: 64) | 19(3): 620-6 (2011). |
| | | LVWVVNNQSLPVSP (SEQ ID NO: 64) | Galanis et al. Cancer Res. |
| | | LVWVVNNQSLPVSP (SEQ ID NO: 64) | 70(3): 875-82 (2010). |
| | | EIIYPNASLLIQN (SEQ ID NO: 62) | Bast et al. Am. J. Obstet. |
| | | NSIVKSITVSASG (SEQ ID NO: 65) | Gynecol. 149(5): 553-9 (1984). |
| | | KTWGQYWQV (SEQ ID NO: 66) | Crosti et al. J Immunol. |
| | | (A)MLGTHTMEV (SEQ ID NO: 67) | 176(8): 5093-9 (2006). |
| | | ITDQVPFSV (SEQ ID NO: 68) | Kobayashi et al. Clin Cancer |
| | | YLEPGPVTA (SEQ ID NO: 69) | Res. 8(10): 3219-25 (2002). |
| | | LLDGTATLRL (SEQ ID NO: 70) | Campi et al. Cancer Res. |
| | | VLYRYGSFSV (SEQ ID NO: 71) | 63(23): 8481-6 (2003). |
| | | SLADTNSLAV (SEQ ID NO: 72) | Bakker et al. Int J Cancer. |
| | | RLMKQDFSV (SEQ ID NO: 73) | 62(1): 97-102 (1995). |
| | | RLPRIFCSC (SEQ ID NO: 74) | Tsai et al. J Immunol. |
| | | LIYRRRLMK (SEQ ID NO: 75) | 158(4): 1796-802 (1997). |
| | | ALLAVGATK (SEQ ID NO: 76) | Kawakami et al. J Immunol. |
| | | IALNFPGSQK (SEQ ID NO: 77) | 154(8): 3961-8 (1995). |
| | | RSYVPLAHR (SEQ ID NO: 78) | Cox et al. Science. |
| | | | 264(5159): 716-9 (1994). |
| | | | Kawakami et al. J Immunol. |
| | | | 154(8): 3961-8 (1995). |
| | | | Kawakami et al. J Immunol. |
| | | | 161(12): 6985-92 (1998). |
| | | | Skipper et al. J Immunol. |
| | | | 157(11): 5027-33 (1996). |
| | | | Michaux et al. J Immunol. |
| | | | 192(4): 1962-71 (2014). |
| 10 | HERV-K- | MLAVISCAV (SEQ ID NO: 112) | Schiavetti et al. Cancer Res. |
| | MEL | | 62(19): 5510-6 (2002). |
| 11 | KK-LC-1 | RQKRILVNL (SEQ ID NO: 113) | Fukuyama et al. Cancer Res. |
| | | | 66(9): 4922-8 (2006). |
| 12 | KM-HN-1 | NYNNFYRFL (SEQ ID NO: 114) | Fukuyama et al. Cancer Res. |
| | | EYSKECLKEF (SEQ ID NO: 115) | 66(9): 4922-8 (2006). |
| | | EYLSLSDKI (SEQ ID NO: 116) | Monji et al. Clin Cancer Res. |
| | | | 10(18 Pt 1): 6047-57 (2004). |
| 13 | LAGE-1 | MLMAQEALAFL (SEQ ID NO: 35) | Aarnoudse et al. Int J Cancer. |
| | | SLLMWITQC (SEQ ID NO: 32) | 82(3): 442-8 (1999). |
| | | LAAQERRVPR (SEQ ID NO: 38) | Rimoldi et al. J Immunol. |
| | | ELVRRILSR (SEQ ID NO: 117) | 165(12): 7253-61 (2000). |
| | | APRGVRMAV (SEQ ID NO: 118) | Wang et al. J Immunol. |
| | | SLLMWITQCFLPVF (SEQ ID NO: 47) | 161(7): 3598-606 (1998). |
| | | QGAMLAAQERRVPRAAEVP-R (SEQ ID NO: 119) | Sun et al. Cancer Immunol |
| | | AADHRQLQLSISSCLQQL (SEQ ID NO: 56) | Immunother. 55(6): 644-52 |
| | | CLSRRPWKRSWSAGSCPG-MPHL (SEQ ID NO: | (2006). |
| | | 120) | Slager et al. Cancer Gene |
| | | ILSRDAAPLPRPG (SEQ ID NO: 121) | Ther. 11(3): 227-36 (2004). |
| | | AGATGGRGPRGAGA (SEQ ID NO: 60) | Zeng et al. Proc Natl Acad Sci |
| | | | USA. 98(7): 3964-9 (2001). |
| | | | Slager et al. J Immunol. |
| | | | 172(8): 5095-102 (2004). |
| | | | Jager et al. J Exp Med. |
| | | | 191(4): 625-30 (2000). |
| | | | Slager et al. J Immunol. |
| | | | 170(3): 1490-7 (2003). |
| | | | Wang et al. Immunity. |
| | | | 20(1): 107-18 (2004). |
| | | | Hasegawa et al. Clin Cancer |
| | | | Res. 12(6): 1921-7 (2006). |
| 14 | MAGE-A2 | YLQLVFGIEV (SEQ ID NO: 153) | Kawashima et al. Hum |
| | | EYLQLVFGI (SEQ ID NO: 154) | Immunol. 59(1): 1-14 (1998). |
| | | REPVTKAEML (SEQ ID NO: 143) | Tahara et al. Clin Cancer |
| | | EGDCAPEEK (SEQ ID NO: 155) | Res. 5(8): 2236-41 (1999). |
| | | LLKYRAREPVTKAE (SEQ ID NO: 156) | Tanzarella et al. Cancer Res. |
| | | | 59(11): 2668-74 (1999). |
| | | | Breckpot et al. J Immunol. |
| | | | 172(4): 2232-7 (2004). |
| | | | Chaux et al. J Exp Med. |
| | | | 89(5): 767-78 (1999). |
| 15 | Sp17 | ILDSSEEDK (SEQ ID NO: 102) | Chiriva-Internati et al. Int J |
| | | | Cancer. 107(5): 863-5 (2003). |
| 16 | TAG-1 | SLGWLFLLL (SEQ ID NO: 132) | Adair et al. J Immunother. |
| | | LSRLSNRLL (SEQ ID NO: 133) | 31(1): 7-17 (2008). |
| 17 | TAG-2 | LSRLSNRLL (SEQ ID NO: 133) | Adair et al. J Immunother. |
| | | | 31(1): 7-17 (2008). |
| 18 | c-myc | SSPQGSPEPL (SEQ ID NO: 164) | Helm et al. PLoS ONE 8(10): |
| | | | e77375 (2013). |
| 19 | cyclin B1 | ILIDWLVQV (SEQ ID NO: 165) | Andersen et al. Cancer |
| | | | Immunol Immunother 60: 227 |
| | | | (2011). |
| 20 | MUC1 | STAPPVHNV (SEQ ID NO: 109) | Brossart et al. Blood, 93(12), |
| | | LLLLTVLTV (SEQ ID NO: 110) | 4309-4317 (1999). |
| 21 | p53 | VVPCEPPEV (SEQ ID NO: 79) | Hung et al. Immunol. Rev. |
| | | | 222: 43-69 (2008). |
| | | | http://cancerimmunity.org/peptide/mutations/ |
| 22 | p62 | FLKNVGESV (SEQ ID NO: 166) | Pampeno et al. (2016) High- |
| | | | ranking In Silico epitopes |
| | | | [determined by 3 algorithms: |
| | | | BISMAS, IEDB, |
| | | | RANKPEP]unpublished |
| 23 | Survivin | ELTLGEFLKL (SEQ ID NO: 167) | Schmitz M Cancer Res. 60: |
| | | | 4845-9 (2000). |
| 24 | gp70 | | Castle et al., BMC Genomics |
| | | | 15: 190 (2014) |

TABLE 7
Thyroid cancer

| | Tumor- | | |
|---|---|---|---|
| | associated | | |
| No. | antigen | Immunogenic epitopes | Sources |
| 1 | CALCA | VLLQAGSLHA (SEQ ID NO: 198) | EI Hage et al. Proc. Natl. |
| | | | Acad. Sci. U.S.A. |
| | | | 105(29): 10119-24 (2008). |
| 2 | NY-ESO-1 | HLA-A2-restricted peptide p157-165 | Jager et al. Proc. Natl. Acad. |
| | | (SLLMWITQC) (SEQ ID NO: 32), HLA-Cw3- | Scie. U.S.A. 103(39): 14453-8 |
| | | restricted p92-100 (LAMP-FATPM) (SEQ ID NO: | (2006). |
| | | 33) and HLA-Cw6-restricted p80-88 | Gnjatic et al. PNAS |
| | | (ARGPESRLL) (SEQ ID NO: 34) | Sep. 26, 2000 vol. 97 |
| | | SLLMWITQC (SEQ ID NO: 32) | no. 20 p. 10919 |
| | | MLMAQEALAFL (SEQ ID NO: 35) | Jager et al. J Exp Med. |
| | | YLAMPFATPME (SEQ ID NO: 36) | 187(2): 265-70 (1998). |
| | | ASGPGGGAPR (SEQ ID NO: 37) | Chen et al. J Immunol. |
| | | LAAQERRVPR (SEQ ID NO: 38) | 165(2): 948-55 (2000). |
| | | TVSGNILTIR (SEQ ID NO: 39) | Valmori et al. Cancer Res. |
| | | APRGPHGGAASGL (SEQ ID NO: 40) | 60(16): 4499-506 (2000). |
| | | MPFATPMEAEL (SEQ ID NO: 41) | Aarnoudse et al. Int J Cancer. |
| | | KEFTVSGNILTI (SEQ ID NO: 42) | 82(3): 442-8 (1999). |
| | | MPFATPMEA (SEQ ID NO: 43) | Eikawa et al. Int J Cancer. |
| | | FATPMEAEL (SEQ ID NO: 44) | 132(2): 345-54 (2013). |
| | | FATPMEAELAR (SEQ ID NO: 45) | Wang et al. J Immunol. |
| | | LAMPFATPM (SEQ ID NO: 46) | 161(7): 3598-606 (1998). |
| | | ARGPESRLL (SEQ ID NO: 34) | Matsuzaki et al. Cancer |
| | | SLLMWITQCFLPVF (SEQ ID NO: 47) | Immunol Immunother. |
| | | LLEFYLAMPFATPMEAEL-ARRSLAQ (SEQ ID | 57(8)1185-95 (2008). |
| | | NO: 48) | Ebert et al. Cancer Res. |
| | | EFYLAMPFATPM (SEQ ID NO: 49) | 69(3): 1046-54 (2009). |
| | | PGVLLKEFTVSGNILTIRL-TAADHR (SEQ ID NO: | Eikawa et al. Int J Cancer. |
| | | 50) | 132(2): 345-54 (2013). |
| | | RLLEFYLAMPFA (SEQ ID NO: 51) | Knights et al. Cancer Immunol |
| | | QGAMLAAQERRVPRAAE-VPR (SEQ ID NO: 52) | Immunother. 58(3): 325-38 |
| | | PFATPMEAELARR (SEQ ID NO: 53) | (2009). |
| | | PGVLLKEFTVSGNILTIRLT (SEQ ID NO: 54) | Jäger et al. Cancer Immun. |
| | | VLLKEFTVSG (SEQ ID NO: 55) | 2: 12 (2002). |
| | | AADHRQLQLSISSCLQQL (SEQ ID NO: 56) | Zeng et al. Proc Natl Acad Sci |
| | | LKEFTVSGNILTIRL (SEQ ID NO: 57) | USA. 98(7): 3964-9 (2001). |
| | | PGVLLKEFTVSGNILTIRL-TAADHR (SEQ ID NO: | Mandic et al. J Immunol. |
| | | 50) | 174(3): 1751-9 (2005). |
| | | LLEFYLAMPFATPMEAEL-ARRSLAQ (SEQ ID | Chen et al. Proc Natl Acad |
| | | NO: 48) | Sci USA. 101(25): 9363-8 |
| | | KEFTVSGNILT (SEQ ID NO: 58) | (2004). |
| | | LLEFYLAMPFATPM (SEQ ID NO: 59) | Ayyoub et al. Clin Cancer |
| | | AGATGGRGPRGAGA (SEQ ID NO: 60) | Res. 16(18): 4607-15 (2010). |
| | | | Slager et al. J Immunol. |
| | | | 172(8): 5095-102 (2004). |
| | | | Mizote et al. Vaccine. |
| | | | 28(32): 5338-46 (2010). |
| | | | Jager et al. J Exp Med. |
| | | | 191(4): 625-30 (2000). |
| | | | Zarour et al. Cancer Res. |
| | | | 60(17): 4946-52 (2000). |
| | | | Zeng et al. J Immunol. |
| | | | 165(2): 1153-9 (2000). |
| | | | Bioley et al. Clin Cancer Res. |
| | | | 15(13): 4467-74 (2009). |
| | | | Zarour et al. Cancer Res. |
| | | | 62(1): 213-8 (2002). |
| | | | Hasegawa et al. Clin Cancer |
| | | | Res. 12(6): 1921-7 (2006). |
| 3 | HERV-K- | MLAVISCAV (SEQ ID NO: 112) | Schiavetti et al. Cancer Res. |
| | MEL | | 62(19): 5510-6 (2002). |
| 4 | KK-LC-1 | RQKRILVNL (SEQ ID NO: 113) | Fukuyama et al. Cancer Res. |
| | | | 66(9): 4922-8 (2006). |
| 5 | KM-HN-1 | NYNNFYRFL (SEQ ID NO: 114) | Fukuyama et al. Cancer Res. |
| | | EYSKECLKEF (SEQ ID NO: 115) | 66(9): 4922-8 (2006). |
| | | EYLSLSDKI (SEQ ID NO: 116) | Monji et al. Clin Cancer Res. |
| | | | 10(18 Pt 1): 6047-57 (2004). |
| 6 | LAGE-1 | MLMAQEALAFL (SEQ ID NO: 35) | Aarnoudse et al. Int J Cancer. |
| | | SLLMWITQC (SEQ ID NO: 32) | 82(3): 442-8 (1999). |
| | | LAAQERRVPR (SEQ ID NO: 38) | Rimoldi et al. J Immunol. |
| | | ELVRRILSR (SEQ ID NO: 117) | 165(12): 7253-61 (2000). |
| | | APRGVRMAV (SEQ ID NO: 118) | Wang et al. J Immunol. |
| | | SLLMWITQCFLPVF (SEQ ID NO: 47) | 161(7): 3598-606 (1998). |
| | | QGAMLAAQERRVPRAAEVP-R (SEQ ID NO: 119) | Sun et al. Cancer Immunol |
| | | AADHRQLQLSISSCLQQL (SEQ ID NO: 56) | Immunother. 55(6): 644-52 |
| | | CLSRRPWKRSWSAGSCPG-MPHL (SEQ ID NO: | (2006). |
| | | 120) | Slager et al. Cancer Gene |
| | | ILSRDAAPLPRPG (SEQ ID NO: 121) | Ther. 11(3): 227-36 (2004). |
| | | AGATGGRGPRGAGA (SEQ ID NO: 60) | Zeng et al. Proc Natl Acad Sci |
| | | | USA. 98(7): 3964-9 (2001). |
| | | | Slager et al. J Immunol. |
| | | | 172(8): 5095-102 (2004). |
| | | | Jager et al. J Exp Med. |
| | | | 191(4): 625-30 (2000). |
| | | | Slager et al. J Immunol. |
| | | | 170(3): 1490-7 (2003). |
| | | | Wang et al. Immunity. |
| | | | 20(1): 107-18 (2004). |
| | | | Hasegawa et al. Clin Cancer |
| | | | Res. 12(6): 1921-7 (2006). |
| 7 | Sp17 | ILDSSEEDK (SEQ ID NO: 102) | Chiriva-Internati et al. Int J |
| | | | Cancer. 107(5): 863-5 (2003). |

TABLE 8
Lung cancer

| | Tumor- | | |
|---|---|---|---|
| | associated | | |
| No. | antigen | Immunogenic epitopes | Sources |
| 1 | CD274 | LLNAFTVTV (SEQ ID NO: 199) | Munir et al. Cancer Res. |
| | | | 73(6): 1764-76 (2013). |
| 2 | mdm-2 | VLFYLGQY (SEQ ID NO: 200) | Asai et al. Cancer Immun. 2: 3 |
| | | | (2002). |
| 3 | alpha- | FIASNGVKLV (SEQ ID NO: 201) | Echchakir et al. Cancer Res. |
| | actinin-4 | | 61(10): 4078-83 (2001). |
| 4 | Elongation | ETVSEQSNV (SEQ ID NO: 202) | Hogan et al. Cancer Res. |
| | factor 2 | | 58(22): 5144-50 (1998). |
| | (squamous | | |
| | cell | | |
| | carcinoma | | |
| | of the lung) | | |
| 5 | ME1 (non- | FLDEFMEGV (SEQ ID NO: 203) | Karanikas et al. Cancer Res. |
| | small cell | | 61(9): 3718-24 (2001). |
| | lung | | |
| | carcinoma) | | |
| 6 | NFYC | QQITKTEV (SEQ ID NO: 204) | Takenoyama et al. Int. J |
| | (squamous | | Cancer. 118(8): 1992-7 (2006). |
| | cell | | |
| | carcinoma | | |
| | of the lung) | | |
| 7 | NY-ESO-1 | HLA-A2-restricted peptide p157-165 | Jager et al. Proc. Natl. Acad. |
| | | (SLLMWITQC) (SEQ ID NO: 32), HLA-Cw3- | Scie. U.S.A. 103(39): 14453-8 |
| | | restricted p92-100 (LAMP-FATPM) (SEQ ID | (2006). |
| | | NO: 33) and HLA-Cw6-restricted p80-88 | Gnjatic et al. PNAS |
| | | (ARGPESRLL) (SEQ ID NO: 34) | Sep. 26, 2000 vol. 97 no. |
| | | SLLMWITQC (SEQ ID NO: 32) | 20 p. 10919 |
| | | MLMAQEALAFL (SEQ ID NO: 35) | Jager et al. J Exp Med. |
| | | YLAMPFATPME (SEQ ID NO: 36) | 187(2): 265-70 (1998). |
| | | ASGPGGGAPR (SEQ ID NO: 37) | Chen et al. J Immunol. |
| | | LAAQERRVPR (SEQ ID NO: 38) | 165(2): 948-55 (2000). |
| | | TVSGNILTIR (SEQ ID NO: 39) | Valmori et al. Cancer Res. |
| | | APRGPHGGAASGL (SEQ ID NO: 40) | 60(16): 4499-506 (2000). |
| | | MPFATPMEAEL (SEQ ID NO: 41) | Aarnoudse et al. Int J Cancer. |
| | | KEFTVSGNILTI (SEQ ID NO: 42) | 82(3): 442-8 (1999). |
| | | MPFATPMEA (SEQ ID NO: 43) | Eikawa et al. Int J Cancer. |
| | | FATPMEAEL (SEQ ID NO: 44) | 132(2): 345-54 (2013). |
| | | FATPMEAELAR (SEQ ID NO: 45) | Wang et al. J Immunol. |
| | | LAMPFATPM (SEQ ID NO: 46) | 161(7): 3598-606 (1998). |
| | | ARGPESRLL (SEQ ID NO: 34) | Matsuzaki et al. Cancer |
| | | SLLMWITQCFLPVF (SEQ ID NO: 47) | Immunol Immunother. |
| | | LLEFYLAMPFATPMEAEL-ARRSLAQ (SEQ ID | 57(8)1185-95 (2008). |
| | | NO: 48) | Ebert et al. Cancer Res. |
| | | EFYLAMPFATPM (SEQ ID NO: 49) | 69(3): 1046-54 (2009). |
| | | PGVLLKEFTVSGNILTIRL-TAADHR (SEQ ID | Eikawa et al. Int J Cancer. |
| | | NO: 50) | 132(2): 345-54 (2013). |
| | | RLLEFYLAMPFA (SEQ ID NO: 51) | Knights et al. Cancer Immunol |
| | | QGAMLAAQERRVPRAAE-VPR (SEQ ID NO: | Immunother. 58(3): 325-38 |
| | | 52) | (2009). |
| | | PFATPMEAELARR (SEQ ID NO: 53) | Jäger et al. Cancer Immun. 2: 12 |
| | | PGVLLKEFTVSGNILTIRLT (SEQ ID NO: 54) | (2002). |
| | | VLLKEFTVSG (SEQ ID NO: 55) | Zeng et al. Proc Natl Acad Sci |
| | | AADHRQLQLSISSCLQQL (SEQ ID NO: 56) | USA. 98(7): 3964-9 (2001). |
| | | LKEFTVSGNILTIRL (SEQ ID NO: 57) | Mandic et al. J Immunol. |
| | | PGVLLKEFTVSGNILTIRL-TAADHR (SEQ ID | 174(3): 1751-9 (2005). |
| | | NO: 50) | Chen et al. Proc Natl Acad Sci |
| | | LLEFYLAMPFATPMEAEL-ARRSLAQ (SEQ ID | USA. 101(25): 9363-8 (2004). |
| | | NO: 48) | Ayyoub et al. Clin Cancer Res. |
| | | KEFTVSGNILT (SEQ ID NO: 58) | 16(18): 4607-15 (2010). |
| | | LLEFYLAMPFATPM (SEQ ID NO: 59) | Slager et al. J Immunol. |
| | | AGATGGRGPRGAGA (SEQ ID NO: 60) | 172(8): 5095-102 (2004). |
| | | | Mizote et al. Vaccine. |
| | | | 28(32): 5338-46 (2010). |
| | | | Jager et al. J Exp Med. |
| | | | 191(4): 625-30 (2000). |
| | | | Zarour et al. Cancer Res. |
| | | | 60(17): 4946-52 (2000). |
| | | | Zeng et al. J Immunol. |
| | | | 165(2): 1153-9 (2000). |
| | | | Bioley et al. Clin Cancer Res. |
| | | | 15(13): 4467-74 (2009). |
| | | | Zarour et al. Cancer Res. |
| | | | 62(1): 213-8 (2002). |
| | | | Hasegawa et al. Clin Cancer |
| | | | Res. 12(6): 1921-7 (2006). |
| 8 | GAGE- | YRPRPRRY (SEQ ID NO: 205) | Van den Eynde et al. J Exp |
| | 1,2,8 | | Med. 182(3): 689-98 (1995). |
| 9 | HERV-K- | MLAVISCAV (SEQ ID NO: 112) | Schiavetti et al. Cancer Res. |
| | MEL | | 62(19): 5510-6 (2002). |
| 10 | KK-LC-1 | RQKRILVNL (SEQ ID NO: 113) | Fukuyama et al. Cancer Res. |
| | | | 66(9): 4922-8 (2006). |
| 11 | KM-HN-1 | NYNNFYRFL (SEQ ID NO: 114) | Fukuyama et al. Cancer Res. |
| | | EYSKECLKEF (SEQ ID NO: 115) | 66(9): 4922-8 (2006). |
| | | EYLSLSDKI (SEQ ID NO: 116) | Monji et al. Clin Cancer Res. |
| | | | 10(18 Pt 1): 6047-57 (2004). |
| 12 | LAGE-1 | MLMAQEALAFL (SEQ ID NO: 35) | Aamoudse et al. Int J Cancer. |
| | | SLLMWITQC (SEQ ID NO: 32) | 82(3): 442-8 (1999). |
| | | LAAQERRVPR (SEQ ID NO: 38) | Rimoldi et al. J Immunol. |
| | | ELVRRILSR (SEQ ID NO: 117) | 165(12): 7253-61 (2000). |
| | | APRGVRMAV (SEQ ID NO: 118) | Wang et al. J Immunol. |
| | | SLLMWITQCFLPVF (SEQ ID NO: 47) | 161(7): 3598-606 (1998). |
| | | QGAMLAAQERRVPRAAEVP-R (SEQ ID NO: | Sun et al. Cancer Immunol |
| | | 119) | Immunother. 55(6): 644-52 |
| | | AADHRQLQLSISSCLQQL (SEQ ID NO: 56) | (2006). |
| | | CLSRRPWKRSWSAGSCPG-MPHL (SEQ ID | Slager et al. Cancer Gene Ther. |
| | | NO: 120) | 11(3): 227-36 (2004). |
| | | ILSRDAAPLPRPG (SEQ ID NO: 121) | Zeng et al. Proc Natl Acad Sci |
| | | AGATGGRGPRGAGA (SEQ ID NO: 60) | USA. 98(7): 3964-9 (2001). |
| | | | Slager et al. J Immunol. |
| | | | 172(8): 5095-102 (2004). |
| | | | Jager et al. J Exp Med. |
| | | | 191(4): 625-30 (2000). |
| | | | Slager et al. J Immunol. |
| | | | 170(3): 1490-7 (2003). |
| | | | Wang et al. Immunity. |
| | | | 20(1): 107-18 (2004). |
| | | | Hasegawa et al. Clin Cancer |
| | | | Res. 12(6): 1921-7 (2006). |
| 13 | MAGE-A2 | YLQLVFGIEV (SEQ ID NO: 153) | Kawashima et al. Hum |
| | | EYLQLVFGI (SEQ ID NO: 154) | Immunol. 59(1): 1-14 (1998). |
| | | REPVTKAEML (SEQ ID NO: 143) | Tahara et al. Clin Cancer Res. |
| | | EGDCAPEEK (SEQ ID NO: 155) | 5(8): 2236-41 (1999). |
| | | LLKYRAREPVTKAE (SEQ ID NO: 156) | Tanzarella et al. Cancer Res. |
| | | | 59(11): 2668-74 (1999). |
| | | | Breckpot et al. J Immunol. |
| | | | 172(4): 2232-7 (2004). |
| | | | Chaux et al. J Exp Med. |
| | | | 89(5): 767-78 (1999). |
| 14 | MAGE-A6 | MVKISGGPR (SEQ ID NO: 206) | Zorn et al. Eur J Immunol. |
| | (squamous | EVDPIGHVY (SEQ ID NO: 207) | 29(2): 602-7 (1999). |
| | cell lung | REPVTKAEML (SEQ ID NO: 143) | Benlalam et al. J Immunol. |
| | carcinoma) | EGDCAPEEK (SEQ ID NO: 155) | 171(11): 6283-9 (2003). |
| | | ISGGPRISY (SEQ ID NO: 208) | Tanzarella et al. Cancer Res. |
| | | LLKYRAREPVTKAE (SEQ ID NO: 156) | 59(11): 2668-74 (1999). |
| | | | Breckpot et al. J Immunol. |
| | | | 172(4): 2232-7 (2004). |
| | | | Vantomme et al. Cancer |
| | | | Immun. 3: 17 (2003). |
| | | | Chaux et al. J Exp Med. |
| | | | 189(5): 767-78 (1999). |
| 15 | Sp17 | ILDSSEEDK (SEQ ID NO: 102) | Chiriva-Internati et al. Int J |
| | | | Cancer. 107(5): 863-5 (2003). |
| 16 | TAG-1 | SLGWLFLLL (SEQ ID NO: 132) | Adair et al. J Immunother. |
| | | LSRLSNRLL (SEQ ID NO: 133) | 31(1): 7-17 (2008). |
| 17 | TAG-2 | LSRLSNRLL (SEQ ID NO: 133) | Adair et al. J Immunother. |
| | | | 31(1): 7-17 (2008). |
| 18 | TRAG-3 | CEFHACWPAFTVLGE (SEQ ID NO: 163) | Janjic et al. J Immunol. |
| | | | 177(4): 2717-27 (2006). |
| 19 | XAGE- | RQKKIRIQL (SEQ ID NO: 209) | Ohue et al. Int J Cancer. |
| | 1b/GAGED | HLGSRQKKIRIQLRSQ (SEQ ID NO: 210) | 131(5): E649-58 (2012). |
| | 2a (non- | CATWKVICKSCISQTPG (SEQ ID NO: 211) | Shimono et al. Int J Oncol. |
| | small cell | | 30(4): 835-40 (2007). |
| | lung | | |
| | cancer) | | |
| 20 | c-myc | SSPQGSPEPL (SEQ ID NO: 164) | Helm et al. PLoS ONE 8(10): |
| | | | e77375 (2013). |
| 21 | cyclin B1 | ILIDWLVQV (SEQ ID NO: 165) | Andersen et al. Cancer |
| | | | Immunol Immunother 60: 227 |
| | | | (2011). |
| 22 | Her2/Neu | HLYQGCQVV (SEQ ID NO: 80) | Nakatsuka et al. Mod. Pathol. |
| | | YLVPQQGFFC (SEQ ID NO: 81) | 19(6): 804-814 (2006). |
| | | PLQPEQLQV (SEQ ID NO: 82) | Pils et al. Br. J. Cancer |
| | | TLEEITGYL (SEQ ID NO: 83) | 96(3): 485-91 (2007). |
| | | ALIHHNTHL (SEQ ID NO: 84) | Scardino et al. Eur J Immunol. |
| | | PLTSIISAV (SEQ ID NO: 85) | 31(11): 3261-70 (2001). |
| | | VLRENTSPK (SEQ ID NO: 86) | Scardino et al. J Immunol. |
| | | TYLPTNASL (SEQ ID NO: 87) | 168(11): 5900-6 (2002). |
| | | | Kawashima et al. Cancer Res. |
| | | | 59(2): 431-5 (1999). |
| | | | Okugawa et al. Eur J Immunol. |
| | | | 30(11): 3338-46 (2000). |
| 23 | MUC1 | STAPPVHNV (SEQ ID NO: 109) | Brossart et al. Blood, 93(12), |
| | | LLLLTVLTV (SEQ ID NO: 110) | 4309-4317 (1999). |
| 24 | p53 | VVPCEPPEV (SEQ ID NO: 79) | Hung et al. Immunol. Rev. |
| | | | 222: 43-69 (2008). |
| | | | http://cancerimmunity.org/peptide/ |
| | | | mutations/ |
| 25 | p62 | FLKNVGESV (SEQ ID NO: 166) | Reuschenbach et al. Cancer |
| | | | Immunol. Immunother. 58: 1535-1544 |
| | | | (2009) |
| 26 | Survivin | ELTLGEFLKL (SEQ ID NO: 167) | Reuschenbach et al. Cancer |
| | | | Immunol. Immunother. 58: 1535-1544 |
| | | | (2009) |

TABLE 9
Prostate cancer

| | Tumor- | | |
|---|---|---|---|
| | associated | | |
| No. | antigen | Immunogenic epitopes | Sources |
| 1 | DKK1 | ALGGHPLLGV (SEQ ID NO: 169) | Qian et al. Blood. 110(5): 1587-94 |
| | | | (2007). |
| 2 | ENAH (hMe | TMNGSKSPV (SEQ ID NO: 134) | Di Modugno et al. Int. J. |
| | na) | | Cancer. 109(6): 909-18 (2004). |
| 3 | Kallikrein 4 | FLGYLILGV (SEQ ID NO: 12); | Wilkinson et al. Cancer |
| | | SVSESDTIRSISIAS (SEQ ID NO: 13); | Immunol Immunother. |
| | | LLANGRMPTVLQCVN (SEQ ID NO: 14); and | 61(2): 169-79 (2012). |
| | | RMPTVLQCVNVSVVS (SEQ ID NO: 15) | Hural et al. J. Immunol. |
| | | | 169(1): 557-65 (2002). |
| 4 | PSMA | NYARTEDFF (SEQ ID NO: 192) | Horiguchi et al. Clin Cancer |
| | | | Res. 8(12): 3885-92 (2002). |
| 5 | STEAP1 | MIAVFLPIV (SEQ ID NO: 212) and | Rodeberg et al. Clin. Cancer |
| | | HQQYFYKIPILVINK (SEQ ID NO: 213) | Res. 11(12): 4545-52 (2005). |
| | | | Kobayashi et al. Cancer Res. |
| | | | 67(11): 5498-504 (2007). |
| 6 | PAP | FLFLLFFWL (SEQ ID NO: 214); | Olson et al. Cancer Immunol |
| | | TLMSAMTNL (SEQ ID NO: 215); and | Immunother. 59(6): 943-53 |
| | | ALDVYNGLL (SEQ ID NO: 216) | (2010). |
| 7 | PSA | FLTPKKLQCV (SEQ ID NO: 217) and | Correale et al. J Natl. Cancer |
| | (prostate | VISNDVCAQV (SEQ ID NO: 218) | Inst. 89(4): 293-300 (1997). |
| | carcinoma) | | |
| 8 | NY-ESO-1 | HLA-A2-restricted peptide p157-165 | Jager et al. Proc. Natl. Acad. |
| | | (SLLMWITQC) (SEQ ID NO: 32), HLA-Cw3- | Scie. U.S.A. 103(39): 14453-8 |
| | | restricted p92-100 (LAMP-FATPM) (SEQ ID | (2006). |
| | | NO: 33) and HLA-Cw6-restricted p80-88 | Gnjatic et al. PNAS |
| | | (ARGPESRLL) (SEQ ID NO: 34) | September 26, 2000 vol. 97 |
| | | SLLMWITQC (SEQ ID NO: 32) | no. 20 p. 10919 |
| | | MLMAQEALAFL (SEQ ID NO: 35) | Jager et al. J Exp Med. |
| | | YLAMPFATPME (SEQ ID NO: 36) | 187(2): 265-70 (1998). |
| | | ASGPGGGAPR (SEQ ID NO: 37) | Chen et al. J Immunol. |
| | | LAAQERRVPR (SEQ ID NO: 38) | 165(2): 948-55 (2000). |
| | | TVSGNILTIR (SEQ ID NO: 39) | Valmori et al. Cancer Res. |
| | | APRGPHGGAASGL (SEQ ID NO: 40) | 60(16): 4499-506 (2000). |
| | | MPFATPMEAEL (SEQ ID NO: 41) | Aarnoudse et al. Int J Cancer. |
| | | KEFTVSGNILTI (SEQ ID NO: 42) | 82(3): 442-8 (1999). |
| | | MPFATPMEA (SEQ ID NO: 43) | Eikawa et al. Int J Cancer. |
| | | FATPMEAEL (SEQ ID NO: 44) | 132(2): 345-54 (2013). |
| | | FATPMEAELAR (SEQ ID NO: 45) | Wang et al. J Immunol. |
| | | LAMPFATPM (SEQ ID NO: 46) | 161(7): 3598-606 (1998). |
| | | ARGPESRLL (SEQ ID NO: 34) | Matsuzaki et al. Cancer |
| | | SLLMWITQCFLPVF (SEQ ID NO: 47) | Immunol Immunother. |
| | | LLEFYLAMPFATPMEAEL-ARRSLAQ (SEQ ID | 57(8)1185-95 (2008). |
| | | NO: 48) | Ebert et al. Cancer Res. |
| | | EFYLAMPFATPM (SEQ ID NO: 49) | 69(3): 1046-54 (2009). |
| | | PGVLLKEFTVSGNILTIRL-TAADHR (SEQ ID | Eikawa et al. Int J Cancer. |
| | | NO: 50) | 132(2): 345-54 (2013). |
| | | RLLEFYLAMPFA (SEQ ID NO: 51) | Knights et al. Cancer Immunol |
| | | QGAMLAAQERRVPRAAE-VPR (SEQ ID NO: | Immunother. 58(3): 325-38 |
| | | 52) | (2009). |
| | | PFATPMEAELARR (SEQ ID NO: 53) | Jäger et al. Cancer Immun. |
| | | PGVLLKEFTVSGNILTIRLT (SEQ ID NO: 54) | 2: 12 (2002). |
| | | VLLKEFTVSG (SEQ ID NO: 55) | Zeng et al. Proc Natl Acad Sci |
| | | AADHRQLQLSISSCLQQL (SEQ ID NO: 56) | USA. 98(7): 3964-9 (2001). |
| | | LKEFTVSGNILTIRL (SEQ ID NO: 57) | Mandic et al. J Immunol. |
| | | PGVLLKEFTVSGNILTIRL-TAADHR (SEQ ID | 174(3): 1751-9 (2005). |
| | | NO: 50) | Chen et al. Proc Natl Acad Sci |
| | | LLEFYLAMPFATPMEAEL-ARRSLAQ (SEQ ID | USA. 101(25): 9363-8 (2004). |
| | | NO: 48) | Ayyoub et al. Clin Cancer Res. |
| | | KEFTVSGNILT (SEQ ID NO: 58) | 16(18): 4607-15 (2010). |
| | | LLEFYLAMPFATPM (SEQ ID NO: 59) | Slager et al. J Immunol. |
| | | AGATGGRGPRGAGA (SEQ ID NO: 60) | 172(8): 5095-102 (2004). |
| | | | Mizote et al. Vaccine. |
| | | | 28(32): 5338-46 (2010). |
| | | | Jager et al. J Exp Med. |
| | | | 191(4): 625-30 (2000). |
| | | | Zarour et al. Cancer Res. |
| | | | 60(17): 4946-52 (2000). |
| | | | Zeng et al. J Immunol. |
| | | | 165(2): 1153-9 (2000). |
| | | | Bioley et al. Clin Cancer Res. |
| | | | 15(13): 4467-74 (2009). |
| | | | Zarour et al. Cancer Res. |
| | | | 62(1): 213-8 (2002). |
| | | | Hasegawa et al. Clin Cancer |
| | | | Res. 12(6): 1921-7 (2006). |
| 9 | BAGE-1 | AARAVFLAL (SEQ ID NO: 137) | Boel et al. Immunity. 2(2): 167-75 |
| | (non-small | | (1995). |
| | cell lung | | |
| | carcinoma) | | |
| 10 | GAGE-1,2,8 | YRPRPRRY (SEQ ID NO: 205) | Van den Eynde et al. J Exp |
| | (non-small | | Med. 182(3): 689-98 (1995). |
| | cell lunch | | |
| | carcinoma) | | |
| 11 | GAGE- | YYWPRPRRY (SEQ ID NO: 219) | De Backer et al. Cancer Res. |
| | 3,4,5,6,7 | | 59(13): 3157-65 (1999). |
| | (lung | | |
| | squamous | | |
| | cell | | |
| | carcinoma | | |
| | and lung | | |
| | adenocarcinoma) | | |
| 12 | HERV-K- | MLAVISCAV (SEQ ID NO: 112) | Schiavetti et al. Cancer Res. |
| | MEL | | 62(19): 5510-6 (2002). |
| 13 | KK-LC-1 | RQKRILVNL (SEQ ID NO: 113) | Fukuyama et al. Cancer Res. |
| | | | 66(9): 4922-8 (2006). |
| 14 | KM-HN-1 | NYNNFYRFL (SEQ ID NO: 114) | Fukuyama et al. Cancer Res. |
| | | EYSKECLKEF (SEQ ID NO: 115) | 66(9): 4922-8 (2006). |
| | | EYLSLSDKI (SEQ ID NO: 116) | Monji et al. Clin Cancer Res. |
| | | | 10(18 Pt 1): 6047-57 (2004). |
| 15 | LAGE-1 | MLMAQEALAFL (SEQ ID NO: 35) | Aarnoudse et al. Int J Cancer. |
| | | SLLMWITQC (SEQ ID NO: 32) | 82(3): 442-8 (1999). |
| | | LAAQERRVPR (SEQ ID NO: 38) | Rimoldi et al. J Immunol. |
| | | ELVRRILSR (SEQ ID NO: 117) | 165(12): 7253-61 (2000). |
| | | APRGVRMAV (SEQ ID NO: 118) | Wang et al. J Immunol. |
| | | SLLMWITQCFLPVF (SEQ ID NO: 47) | 161(7): 3598-606 (1998). |
| | | QGAMLAAQERRVPRAAEVP-R (SEQ ID NO: | Sun et al. Cancer Immunol |
| | | 119) | Immunother. 55(6): 644-52 |
| | | AADHRQLQLSISSCLQQL (SEQ ID NO: 56) | (2006). |
| | | CLSRRPWKRSWSAGSCPG-MPHL (SEQ ID | Slager et al. Cancer Gene |
| | | NO: 120) | Ther. 11(3): 227-36 (2004). |
| | | ILSRDAAPLPRPG (SEQ ID NO: 121) | Zeng et al. Proc Natl Acad Sci |
| | | AGATGGRGPRGAGA (SEQ ID NO: 60) | USA. 98(7): 3964-9 (2001). |
| | | | Slager et al. J Immunol. |
| | | | 172(8): 5095-102 (2004). |
| | | | Jager et al. J Exp Med. |
| | | | 191(4): 625-30 (2000). |
| | | | Slager et al. J Immunol. |
| | | | 170(3): 1490-7 (2003). |
| | | | Wang et al. Immunity. |
| | | | 20(1): 107-18 (2004). |
| | | | Hasegawa et al. Clin Cancer |
| | | | Res. 12(6): 1921-7 (2006). |
| 16 | Sp17 | ILDSSEEDK (SEQ ID NO: 102) | Chiriva-Internati et al. Int J |
| | | | Cancer. 107(5): 863-5 (2003). |

TABLE 10
Kidney cancer

| | Tumor- | | |
|---|---|---|---|
| | associated | | |
| No. | antigen | Immunogenic epitopes | Sources |
| 1 | FGF5 | NTYASPRFK (SEQ ID NO: 316) | Hanada et al. Nature. |
| | | | 427(6971): 252-6 (2004). |
| 2 | Hepsin | SLLSGDWVL (SEQ ID NO: 184); | Guo et al. Scand J Immunol. |
| | | GLQLGVQAV (SEQ ID NO: 185); and | 78(3): 248-57 (2013). |
| | | PLTEYIQPV (SEQ ID NO: 186) | |
| 3 | Intestinal | SPRWWPTCL (SEQ ID NO: 187) | Ronsin et al. J Immunol. |
| | carboxyl | | 163(1): 483-90 (1999). |
| | esterase | | |
| 4 | M-CSF | LPAVVGLSPGEQEY (SEQ ID NO: 191) | Probst-Kepper et al. J Exp Med. |
| | | | 193(10): 1189-98 (2001). |
| 5 | RU2AS | LPRWPPPQL (SEQ ID NO: 170) | Van Den Eynde et al. J. Exp. |
| | | | Med. 190(12): 1793-800 (1999). |
| 6 | hsp70-2 | SLFEGIDIYT (SEQ ID NO: 317) | Gaudin et al. J. Immunol. |
| | (renal cell | | 162(3): 1730-8 (1999). |
| | carcinoma) | | |
| 7 | Mannan- | PDTRPAPGSTAPPAHGVTSA (SEQ ID NO: | Loveland et al. Clin. Cancer Res. |
| | MUC-1 (renal | 108) | 12(3 Pt 1): 869-77 (2006). |
| | cell | STAPPVHNV (SEQ ID NO: 109) | Loveland et al. Clin. Cancer Res. |
| | carcinoma) | LLLLTVLTV (SEQ ID NO: 110) | 12(3 Pt 1): 869-77 (2006). |
| | | PGSTAPPAHGVT (SEQ ID NO: 111) | Godelaine et al. Cancer Immunol |
| | | | Immunother. 56(6): 753-9 (2007). |
| | | | Ma et al. Int J Cancer. |
| | | | 129(10): 2427-34 (2011). |
| | | | Wen et al. Cancer Sci. |
| | | | 102(8): 1455-61 (2011). |
| | | | Jerome et al. J Immunol. |
| | | | 151(3): 1654-62 (1993). |
| | | | Brossart et al. Blood. |
| | | | 93(12): 4309-17 (1999). |
| | | | Hiltbold et al. Cancer Res. |
| | | | 58(22): 5066-70 (1998). |
| 8 | MAGE-A9 | ALSVMGVYV (SEQ ID NO: 318) | Oehlrich et al. Int J Cancer. |
| | (renal cell | | 117(2): 256-64 (2005). |
| | carcinoma) | | |

TABLE 11
Melanoma

| | Tumor- | | |
|---|---|---|---|
| | associated | | |
| No. | antigen | Immunogenic epitopes | Sources |
| 1 | Hepsin | SLLSGDWVL (SEQ ID NO: 184); | Guo et al. Scand J Immunol. |
| | | GLQLGVQA (SEQ ID NO: 185); and | 78(3): 248-57 (2013). |
| | | PLTEYIQPV (SEQ ID NO: 186) | |
| 2 | ARTC1 | YSVYFNLPADTIYTN (SEQ ID NO: 319) | Wang et al J Immunol. |
| | | | 174(5): 2661-70 (2005). |
| 3 | B-RAF | EDLTVKIGDFGLATEKSRWSGSHQFEQLS | Sharkey et al. Cancer Res. |
| | | (SEQ ID NO: 320) | 64(5): 1595-9 (2004). |
| 4 | beta- | SYLDSGIHF (SEQ ID NO: 321) | Robbins et al. J. Exp. Med. |
| | catenin | | 183(3): 1185-92 (1996). |
| 5 | Cdc27 | FSWAMDLDPKGA (SEQ ID NO: 322) | Wang et al. Science. |
| | | | 284(5418): 1351-4 (1999). |
| 6 | CDK4 | ACDPHSGHFV (SEQ ID NO: 323) | Wölfel et al. Science. |
| | | | 269(5228): 1281-4 (1995). |
| 7 | CDK12 | CILGKLFTK (SEQ ID NO: 324) | Robbins et al. Nat Med. |
| | | | 19(6): 747-52. (2013). |
| 8 | CDKN2A | AVCPVVTWLR (SEQ ID NO: 325) | Huang et al. J Immunol. |
| | | | 172(10): 6057-64 (2004). |
| 9 | CLPP | ILDKVLVHL (SEQ ID NO: 326) | Corbière et al. Cancer Res. |
| | | | 71(4): 1253-62 (2011). |
| 10 | CSNK1A1 | GLFGDIYLA (SEQ ID NO: 327) | Robbins et al. Nat Med. |
| | | | 19(6): 747-52 (2013). |
| 11 | FN1 | MIFEKHGFRRTTPP (SEQ ID NO: 328) | Wang et al. J Exp Med. |
| | | | 195(11): 1397-406 (2003). |
| 12 | GAS7 | SLADEAEVYL (SEQ ID NO: 329) | Robbins, et al. Nat Med. |
| | | | 19(6): 747-52 (2013). |
| 13 | GPNMB | TLDWLLQTPK (SEQ ID NO: 330) | Lennerz et al. Proc. Natl. Acad. |
| | | | Sci. U.S.A. 102(44): 16013-8 |
| | | | (2005). |
| 14 | HAUS3 | ILNAMIAKI (SEQ ID NO: 331) | Robbins et al. Nat Med. |
| | | | 19(6): 747-52 (2013). |
| 15 | LDLR- | WRRAPAPGA (SEQ ID NO: 332) and | Wang et al. J Exp Med. |
| | fucosyltransferase | PVTWRRAPA (SEQ ID NO: 333) | 189(10): 1659-68 (1999). |
| 16 | MART2 | FLEGNEVGKTY (SEQ ID NO: 334) | Kawakami et al. J Immunol. |
| | | | 166(4): 2871-7 (2001). |
| 17 | MATN | KTLTSVFQK (SEQ ID NO: 335) | Robbins et al. Nat Med. |
| | | | 19(6): 747-52 (2013). |
| 18 | MUM-1 | EEKLIVVLF (SEQ ID NO: 336) | Coulie et al. Proc. Natl. Acad. |
| | | | Sci. U.S.A. 92(17): 7976-80 |
| | | | (1995). |
| 19 | MUM-2 | SELFRSGLDSY (SEQ ID NO: 337) and | Chiari et al. Cancer Res. |
| | | FRSGLDSYV (SEQ ID NO: 338) | 59(22): 5785-92 (1999). |
| 20 | MUM-3 | EAFIQPITR (SEQ ID NO: 339) | Baurain et al. J. Immunol. |
| | | | 164(11): 6057-66 (2000). |
| 21 | neo-PAP | RVIKNSIRLTL (SEQ ID NO: 340) | Topalian et al. Cancer Res. |
| | | | 62(19): 5505-9 (2002). |
| 22 | Myosin | KINKNPKYK (SEQ ID NO: 341) | Zorn, et al. Eur. J. Immunol. |
| | class I | | 29(2): 592-601 (1999). |
| 23 | PPP1R3B | YTDFHCQYV (SEQ ID NO: 342) | Robbins et al. Nat Med. |
| | | | 19(6): 747-52 (2013). |
| | | | Lu et al. J Immunol. |
| | | | 190(12): 6034-42 (2013). |
| 24 | PRDX5 | LLLDDLLVSI (SEQ ID NO: 343) | Sensi et al. Cancer Res. |
| | | | 65(2): 632-40 (2005). |
| 25 | PTPRK | PYYFAAELPPRNLPEP (SEQ ID NO: 344) | Novellino et al. J. Immunol. |
| | | | 170(12): 6363-70 (2003). |
| 26 | N-ras | ILDTAGREEY (SEQ ID NO: 345) | Linard et al. J. Immunol. |
| | | | 168(9): 4802-8 (2002). |
| 27 | RBAF600 | RPHVPESAF (SEQ ID NO: 346) | Lennerz et al. Proc. Natl. Acad. |
| | | | Sci. U.S.A. 102(44): 16013-8 |
| | | | (2005). |
| 28 | SIRT2 | KIFSEVTLK (SEQ ID NO: 347) | Lennerz et al. Proc. Natl. Acad. |
| | | | Sci. U.S.A. 102(44): 16013-8 |
| | | | (2005). |
| 29 | SNRPD1 | SHETVIIEL (SEQ ID NO: 348) | Lennerz et al. Proc. Natl. Acad. |
| | | | Sci. U.S.A. 102(44): 16013-8 |
| | | | (2005). |
| 30 | Triosephosphate | GELIGILNAAKVPAD (SEQ ID NO: 349) | Pieper et al. J Exp Med. |
| | isomerase | | 189(5): 757-66 (1999). |
| 31 | OA1 | LYSACFWWL (SEQ ID NO: 350) | Touloukian et al. J. Immunol. |
| | | | 170(3): 1579-85 (2003). |
| 32 | RAB38/ | VLHWDPETV (SEQ ID NO: 351) | Walton et al. J Immunol. |
| | NY-MEL-1 | | 177(11): 8212-8 (2006). |
| 33 | TRP-1/ | MSLQRQFLR (SEQ ID NO: 352); | Touloukian et al. Cancer Res. |
| | gp75 | ISPNSVFSQWRVVCDSLEDY (SEQ ID NO: 353); | 62(18): 5144-7 (2002). |
| | | SLPYWNFATG (SEQ ID NO: 354); and | Robbins et al. J. Immunol. |
| | | SQWRVVCDSLEDYDT (SEQ ID NO: 355) | (10): 6036-47 (2002). |
| | | | Osen et al. PLoS One. |
| | | | 5(11): e14137 (2010). |
| 34 | TRP-2 | SVYDFFVWL (SEQ ID NO: 356); | Parkhurst et al. Cancer Res. |
| | | TLDSQVMSL (SEQ ID NO: 357); | 58(21): 4895-901 (1998). |
| | | LLGPGRPYR (SEQ ID NO: 358); | Noppen et al. Int. J. Cancer. |
| | | ANDPIFVVL (SEQ ID NO: 359); | 87(2): 241-6 (2000). |
| | | QCTEVRADTRPWSGP (SEQ ID NO: 360); and | Wang et al. J. Exp. Med. |
| | | ALPYWNFATG (SEQ ID NO: 361) | 1184(6): 2207-16 (1996). |
| | | | Wang et al. J. Immunol. |
| | | | 160(2): 890-7 (1998). |
| | | | Castelli et al. J. Immunol. |
| | | | 162(3): 1739-48 (1999). |
| | | | Paschen et al. Clin. Cancer |
| | | | Res. (14): 5241-7 (2005). |
| | | | Robbins et al. J. Immunol. |
| | | | 169(10): 6036-47 (2002). |
| 35 | tyrosinase | KCDICTDEY (SEQ ID NO: 362); | Kittlesen et al. J. Immunol. |
| | | SSDYVIPIGTY (SEQ ID NO: 363); | 160(5): 2099-106 (1998). |
| | | MLLAVLYCL (SEQ ID NO: 364); | Kawakami et al. J. Immunol. |
| | | CLLWSFQTSA (SEQ ID NO: 365); | (12): 6985-92 (1998). |
| | | YMDGTMSQV (SEQ ID NO: 366); | Wölfel et al. Eur. J. Immunol. |
| | | AFLPWHRLF (SEQ ID NO: 31); | 24(3): 759-64 (1994). |
| | | IYMDGTADFSF (SEQ ID NO: 367); | Riley et al. J. Immunother. |
| | | QCSGNFMGF (SEQ ID NO: 368); | 24(3): 212-20 (2001). |
| | | TPRLPSSADVEF (SEQ ID NO: 369); | Skipper et al. J. Exp. Med. |
| | | LPSSADVEF (SEQ ID NO: 370); | 183(2): 527-34 (1996). |
| | | LHHAFVDSIF (SEQ ID NO: 371); | Kang et al. J. Immunol. |
| | | SEIWRDIDF (SEQ ID NO: 372); | 155(3): 1343-8 (1995). |
| | | QNILLSNAPLGPQFP (SEQ ID NO: 373); | Dalet et al. Proc. Natl. Acad. |
| | | SYLQDSDPDSFQD (SEQ ID NO: 374); and | Sci. U.S.A. 108(29): E323-31 |
| | | FLLHHAFVDSIFEQWLQRHRP (SEQ ID NO: | (2011) |
| | | 375) | Lennerz et al. Proc. Natl. Acad. |
| | | | Sci. U.S.A. 102(44): 16013-8 |
| | | | (2005). |
| | | | Benlalam et al. J. Immunol. |
| | | | 171(11): 6283-9 (2003). |
| | | | Morel et al. Int. J. Cancer. |
| | | | 83(6): 755-9 (1999). |
| | | | Brichard et al. Eur. J. Immunol. |
| | | | 26(1): 224-30 (1996). |
| | | | Topalian et al. J. Exp. Med. |
| | | | (5): 1965-71 (1996). |
| | | | Kobayashi et al. Cancer Res. |
| | | | 58(2): 296-301 (1998). |
| 36 | Melan- | YTTAEEAAGIGILTVILGVLLLIGCWYCRR (SEQ | Meng et al. J. Immunother. |
| | A/MART-1 | ID NO: 376) | 23: 525-534 (2011) |
| 37 | gp100/ | ALNFPGSQK (SEQ ID NO: 377) | El Hage et al. Proc. Natl. Acad. |
| | Pmel17 | ALNFPGSQK (SEQ ID NO: 377) | Sci. U.S.A. 105(29): 10119-24 |
| | | VYFFLPDHL (SEQ ID NO: 378) | (2008). |
| | | RTKQLYPEW (SEQ ID NO: 379) | Kawashima et al. Hum |
| | | HTMEVTVYHR (SEQ ID NO: 380) | Immunol. 59(1): 1-14 (1998). |
| | | SSPGCQPPA (SEQ ID NO: 381) | Robbins et al. J Immunol. |
| | | VPLDCVLYRY (SEQ ID NO: 382) | 159(1): 303-8 (1997). |
| | | LPHSSSHWL (SEQ ID NO: 383) | Sensi et al. Tissue Antigens. |
| | | SNDGPTLI (SEQ ID NO: 384) | 59(4): 273-9 (2002). |
| | | GRAMLGTHTMEVTVY (SEQ ID NO: 385) | Lennerz et al. Proc Natl Acad |
| | | WNRQLYPEWTEAQRLD (SEQ ID NO: 386) | Sci USA. 102(44): 16013-8 |
| | | TTEWVETTARELPIPEPE (SEQ ID NO: 387) | (2005). |
| | | TGRAMLGTHTMEVTVYH (SEQ ID NO: 388) | Benlalam et al. J Immunol. |
| | | GRAMLGTHTMEVTVY (SEQ ID NO: 385) | 171(11): 6283-9 (2003). |
| | | | Vigneron et al. Tissue Antigens. |
| | | | 65(2): 156-62 (2005). |
| | | | Castelli et al. J Immunol. |
| | | | 162(3): 1739-48 (1999). |
| | | | Touloukian et al. J Immunol. |
| | | | 164(7): 3535-42 (2000). |
| | | | Parkhurst et al. J Immunother. |
| | | | 27(2): 79-91 (2004). |
| | | | Lapointe et al. J Immunol. |
| | | | 167(8): 4758-64 (2001). |
| | | | Kobayashi et al. Cancer Res. |
| | | | 61(12): 4773-8 (2001). |
| 38 | NY-ESO-1 | HLA-A2-restricted peptide p157-165 | Jager et al. Proc. Natl. Acad. |
| | | (SLLMWITQC) (SEQ ID NO: 32), HLA-Cw3- | Scie. U.S.A. 103(39): 14453-8 |
| | | restricted p92-100 (LAMP-FATPM) (SEQ ID | (2006). |
| | | NO: 33) and HLA-Cw6-restricted p80-88 | Gnjatic et al. PNAS |
| | | (ARGPESRLL) (SEQ ID NO: 34) | Sep. 26, 2000 vol. 97 no. |
| | | SLLMWITQC (SEQ ID NO: 32) | 20 p. 10919 |
| | | MLMAQEALAFL (SEQ ID NO: 35) | Jager et al. J Exp Med. |
| | | YLAMPFATPME (SEQ ID NO: 36) | 187(2): 265-70 (1998). |
| | | ASGPGGGAPR (SEQ ID NO: 37) | Chen et al. J Immunol. |
| | | LAAQERRVPR (SEQ ID NO: 38) | 165(2): 948-55 (2000). |
| | | TVSGNILTIR (SEQ ID NO: 39) | Valmori et al. Cancer Res. |
| | | APRGPHGGAASGL (SEQ ID NO: 40) | 60(16): 4499-506 (2000). |
| | | MPFATPMEAEL (SEQ ID NO: 41) | Aarnoudse et al. Int J Cancer. |
| | | KEFTVSGNILTI (SEQ ID NO: 42) | 82(3): 442-8 (1999). |
| | | MPFATPMEA (SEQ ID NO: 43) | Eikawa et al. Int J Cancer. |
| | | FATPMEAEL (SEQ ID NO: 44) | 132(2): 345-54 (2013). |
| | | FATPMEAELAR (SEQ ID NO: 45) | Wang et al. J Immunol. |
| | | LAMPFATPM (SEQ ID NO: 46) | 161(7): 3598-606 (1998). |
| | | ARGPESRLL (SEQ ID NO: 34) | Matsuzaki et al. Cancer |
| | | SLLMWITQCFLPVF (SEQ ID NO: 47) | Immunol Immunother. |
| | | LLEFYLAMPFATPMEAEL-ARRSLAQ (SEQ ID | 57(8)1185-95 (2008). |
| | | NO: 48) | Ebert et al. Cancer Res. |
| | | EFYLAMPFATPM (SEQ ID NO: 49) | 69(3): 1046-54 (2009). |
| | | PGVLLKEFTVSGNILTIRL-TAADHR (SEQ ID | Eikawa et al. Int J Cancer. |
| | | NO: 50) | 132(2): 345-54 (2013). |
| | | RLLEFYLAMPFA (SEQ ID NO: 51) | Knights et al. Cancer Immunol |
| | | QGAMLAAQERRVPRAAE-VPR (SEQ ID NO: | Immunother. 58(3): 325-38 |
| | | 52) | (2009). |
| | | PFATPMEAELARR (SEQ ID NO: 53) | Jäger et al. Cancer Immun. 2: 12 |
| | | PGVLLKEFTVSGNILTIRLT (SEQ ID NO: 54) | (2002). |
| | | VLLKEFTVSG (SEQ ID NO: 55) | Zeng et al. Proc Natl Acad Sci |
| | | AADHRQLQLSISSCLQQL (SEQ ID NO: 56) | USA. 98(7): 3964-9 (2001). |
| | | LKEFTVSGNILTIRL (SEQ ID NO: 57) | Mandic et al. J Immunol. |
| | | PGVLLKEFTVSGNILTIRL-TAADHR (SEQ ID | 174(3): 1751-9 (2005). |
| | | NO: 50) | Chen et al. Proc Natl Acad Sci |
| | | LLEFYLAMPFATPMEAEL-ARRSLAQ (SEQ ID | USA. 101(25): 9363-8 (2004). |
| | | NO: 48) | Ayyoub et al. Clin Cancer Res. |
| | | KEFTVSGNILT (SEQ ID NO: 58) | 16(18): 4607-15 (2010). |
| | | LLEFYLAMPFATPM (SEQ ID NO: 59) | Slager et al. J Immunol. |
| | | AGATGGRGPRGAGA (SEQ ID NO: 60) | 172(8): 5095-102 (2004). |
| | | | Mizote et al. Vaccine. |
| | | | 28(32): 5338-46 (2010). |
| | | | Jager et al. J Exp Med. |
| | | | 191(4): 625-30 (2000). |
| | | | Zarour et al. Cancer Res. |
| | | | 60(17): 4946-52 (2000). |
| | | | Zeng et al. J Immunol. |
| | | | 165(2): 1153-9 (2000). |
| | | | Bioley et al. Clin Cancer Res. |
| | | | 15(13): 4467-74 (2009). |
| | | | Zarour et al. Cancer Res. |
| | | | 62(1): 213-8 (2002). |
| | | | Hasegawa et al. Clin Cancer |
| | | | Res. 12(6): 1921-7 (2006). |
| 39 | BAGE-1 | AARAVFLAL (SEQ ID NO: 137) | Boel et al. Immunity. 2(2): 167-75 |
| | | | (1995). |
| 40 | GAGE- | YRPRPRRY (SEQ ID NO: 205) | Van den Eynde et al. J Exp |
| | 1,2,8 | | Med. 182(3): 689-98 (1995). |
| 41 | GAGE- | YYWPRPRRY (SEQ ID NO: 219) | De Backer et al. Cancer Res. |
| | 3,4,5,6,7 | | 59(13): 3157-65 (1999). |
| | (cutaneous | | |
| | melanoma) | | |
| 42 | GnTVf | VLPDVFIRC(V) (SEQ ID NO: 220) | Guilloux et al. J Exp Med. |
| | | | 183(3): 1173-83 (1996). |
| 43 | HERV-K- | MLAVISCAV (SEQ ID NO: 112) | Schiavetti et al. Cancer Res. |
| | MEL | | 62(19): 5510-6 (2002). |
| 44 | KK-LC-1 | RQKRILVNL (SEQ ID NO: 113) | Fukuyama et al. Cancer Res. |
| | | | 66(9): 4922-8 (2006). |
| 45 | KM-HN-1 | NYNNFYRFL (SEQ ID NO: 114) | Fukuyama et al. Cancer Res. |
| | | EYSKECLKEF (SEQ ID NO: 115) | 66(9): 4922-8 (2006). |
| | | EYLSLSDKI (SEQ ID NO: 116) | Monji et al. Clin Cancer Res. |
| | | | 10(18 Pt 1): 6047-57 (2004). |
| 46 | LAGE-1 | MLMAQEALAFL (SEQ ID NO: 35) | Aamoudse et al. Int J Cancer. |
| | | SLLMWITQC (SEQ ID NO: 32) | 82(3): 442-8 (1999). |
| | | LAAQERRVPR (SEQ ID NO: 38) | Rimoldi et al. J Immunol. |
| | | ELVRRILSR (SEQ ID NO: 117) | 165(12): 7253-61 (2000). |
| | | APRGVRMAV (SEQ ID NO: 118) | Wang et al. J Immunol. |
| | | SLLMWITQCFLPVF (SEQ ID NO: 47) | 161(7): 3598-606 (1998). |
| | | QGAMLAAQERRVPRAAEVP-R (SEQ ID NO: | Sun et al. Cancer Immunol |
| | | 119) | Immunother. 55(6): 644-52 |
| | | AADHRQLQLSISSCLQQL (SEQ ID NO: 56) | (2006). |
| | | CLSRRPWKRSWSAGSCPG-MPHL (SEQ ID | Slager et al. Cancer Gene Ther. |
| | | NO: 120) | 11(3): 227-36 (2004). |
| | | ILSRDAAPLPRPG (SEQ ID NO: 121) | Zeng et al. Proc Natl Acad Sci |
| | | AGATGGRGPRGAGA (SEQ ID NO: 60) | USA. 98(7): 3964-9 (2001). |
| | | | Slager et al. J Immunol. |
| | | | 172(8): 5095-102 (2004). |
| | | | Jager et al. J Exp Med. |
| | | | 191(4): 625-30 (2000). |
| | | | Slager et al. J Immunol. |
| | | | 170(3): 1490-7 (2003). |
| | | | Wang et al. Immunity. |
| | | | 20(1): 107-18 (2004). |
| | | | Hasegawa et al. Clin Cancer |
| | | | Res. 12(6): 1921-7 (2006). |
| 47 | LY6K | RYCNLEGPPI (SEQ ID NO: 221) | Suda et al. Cancer Sci. |
| | | KVVTEPYCVIAAVKIFPRFFMV-AKQ (SEQ ID | 98(11): 1803-8 (2007). |
| | | NO: 222) | Tomita et al. Oncoimmunology. |
| | | KCCKIRYCNLEGPPINSSVF (SEQ ID NO: 223) | 3: e28100 (2014). |
| 48 | MAGE-A1 | EADPTGHSY (SEQ ID NO: 138) | Traversari et al. J Exp Med. |
| | | KVLEYVIKV (SEQ ID NO: 139) | 176(5): 1453-7 (1992). |
| | | SLFRAVITK (SEQ ID NO: 140) | Ottaviani et al. Cancer Immunol |
| | | EVYDGREHSA (SEQ ID NO: 141) | Immunother. 54(12): 1214-20 |
| | | RVRFFFPSL (SEQ ID NO: 142) | (2005). |
| | | EADPTGHSY (SEQ ID NO: 138) | Pascolo et al. Cancer Res. |
| | | REPVTKAEML (SEQ ID NO: 143) | 61(10): 4072-7 (2001). |
| | | KEADPTGHSY (SEQ ID NO: 144) | Chaux et al. J Immunol. |
| | | DPARYEFLW (SEQ ID NO: 145) | 163(5): 2928-36 (1999). |
| | | ITKKVADLVGF (SEQ ID NO: 146) | Luiten et al. Tissue Antigens. |
| | | SAFPTTINF (SEQ ID NO: 147) | 55(2): 149-52 (2000). |
| | | SAYGEPRKL (SEQ ID NO: 148) | Luiten et al. Tissue Antigens. |
| | | RVRFFFPSL (SEQ ID NO: 142) | 56(1): 77-81 (2000). |
| | | TSCILESLFRAVITK (SEQ ID NO: 149) | Tanzarella et al. Cancer Res. |
| | | PRALAETSYVKVLEY (SEQ ID NO: 150) | 59(11): 2668-74 (1999). |
| | | FLLLKYRAREPVTKAE (SEQ ID NO: 151) | Stroobant et al. Eur J Immunol. |
| | | EYVIKVSARVRF (SEQ ID NO: 152) | 42(6): 1417-28 (2012). |
| | | | Corbière et al. Tissue Antigens. |
| | | | 63(5): 453-7 (2004). |
| | | | Goodyear et al. Cancer |
| | | | Immunol Immunother. |
| | | | 60(12): 1751-61 (2011). |
| | | | van der Bruggen et al. Eur J |
| | | | Immunol. 24(9): 2134-40 (1994). |
| | | | Wang et al. Cancer Immunol |
| | | | Immunother. 56(6): 807-18 |
| | | | (2007). |
| | | | Chaux et al. J Exp Med. |
| | | | 189(5): 767-78 (1999). |
| | | | Chaux et al. Eur J Immunol. |
| | | | 31(6): 1910-6 (2001). |
| 49 | MAGE-A6 | MVKISGGPR (SEQ ID NO: 206) | Zorn et al. Eur J Immunol. |
| | | EVDPIGHVY (SEQ ID NO: 207) | 29(2): 602-7 (1999). |
| | | REPVTKAEML (SEQ ID NO: 143) | Benlalam et al. J Immunol. |
| | | EGDCAPEEK (SEQ ID NO: 155) | 171(11): 6283-9 (2003). |
| | | ISGGPRISY (SEQ ID NO: 208) | Tanzarella et al. Cancer Res. |
| | | LLKYRAREPVTKAE (SEQ ID NO: 156) | 59(11): 2668-74 (1999). |
| | | | Breckpot et al. J Immunol. |
| | | | 172(4): 2232-7 (2004). |
| | | | Vantomme et al. Cancer |
| | | | Immun. 3: 17 (2003). |
| | | | Chaux et al. J Exp Med. |
| | | | 189(5): 767-78 (1999). |
| 50 | MAGE-A10 | GLYDGMEHL (SEQ ID NO: 224) | Huang et al. J Immunol. |
| | | DPARYEFLW (SEQ ID NO: 145) | 162(11): 6849-54 (1999). |
| | | | Chaux et al. J Immunol. |
| | | | 163(5): 2928-36 (1999). |
| 51 | MAGE-A12 | FLWGPRALV (SEQ ID NO: 225) | van der Bruggen et al. Eur J |
| | | VRIGHLYIL (SEQ ID NO: 226) | Immunol. 24(12): 3038-43 |
| | | EGDCAPEEK (SEQ ID NO: 155) | (1994). |
| | | REPFTKAEMLGSVIR (SEQ ID NO: 227) | Heidecker et al. J Immunol. |
| | | AELVHFLLLKYRAR (SEQ ID NO: 228) | 164(11): 6041-5 (2000). |
| | | | Panelli et al. J Immunol. |
| | | | 164(8): 4382-92 (2000). |
| | | | Breckpot et al. J Immunol. |
| | | | 172(4): 2232-7 (2004). |
| | | | Wang et al. Cancer Immunol |
| | | | Immunother. 56(6): 807-18 |
| | | | (2007). |
| | | | Chaux et al. J Exp Med. |
| | | | 189(5): 767-78 (1999). |
| 52 | MAGE-C2 | LLFGLALIEV (SEQ ID NO: 229) | Ma et al. Int J Cancer. |
| | | ALKDVEERV (SEQ ID NO: 230) | 109(5): 698-702 (2004). |
| | | SESIKKKVL (SEQ ID NO: 231) | Godelaine et al. Cancer |
| | | ASSTLYLVF (SEQ ID NO: 232) | Immunol Immunother. |
| | | SSTLYLVFSPSSFST (SEQ ID NO: 233) | 56(6): 753-9 (2007). |
| | | | Ma et al. Int J Cancer. |
| | | | 129(10): 2427-34 (2011). |
| | | | Wen et al. Cancer Sci. |
| | | | 102(8): 1455-61 (2011). |
| 53 | NA88-A | QGQHFLQKV (SEQ ID NO: 234) | Moreau-Aubry et al. J Exp Med. |
| | | | 191(9): 1617-24 (2000). |
| 54 | Sp17 | ILDSSEEDK (SEQ ID NO: 102) | Chiriva-Internati et al. Int J |
| | | | Cancer. 107(5): 863-5 (2003). |
| 55 | SSX-2 | KASEKIFYV (SEQ ID NO: 157) | Ayyoub et al. J Immunol. |
| | | EKIQKAFDDIAKYFSK (SEQ ID NO: 158) | 168(4): 1717-22 (2002). |
| | | FGRLQGISPKI (SEQ ID NO: 159) | Ayyoub et al. J Immunol. |
| | | WEKMKASEKIFYVYMKRK (SEQ ID NO: 160) | 172(11): 7206-11 (2004). |
| | | KIFYVYMKRKYEAMT (SEQ ID NO: 161) | Neumann et al. Cancer |
| | | KIFYVYMKRKYEAM (SEQ ID NO: 162) | Immunol Immunother. |
| | | | 60(9): 1333-46 (2011). |
| | | | Ayyoub et al. Clin Immunol. |
| | | | 114(1): 70-8 (2005). |
| | | | Neumann et al. Int J Cancer. |
| | | | 112(4): 661-8 (2004). |
| | | | Ayyoub et al. J Clin Invest. |
| | | | 113(8): 1225-33 (2004). |
| 56 | SSX-4 | INKTSGPKRGKHAVVTHRLRE (SEQ ID NO: 126) | Ayyoub et al. J Immunol. |
| | | YFSKKEWEKMKSSEKIVYVY (SEQ ID NO: 127) | 174(8): 5092-9 (2005). |
| | | MKLNYEVMTKLGFKVTLPPF (SEQ ID NO: 128) | Valmori et al. Clin Cancer Res. |
| | | KHAWTHRLRERKQLVVYEEI (SEQ ID NO: 129) | 12(2): 398-404 (2006). |
| | | LGFKVTLPPFMRSKRAADFH (SEQ ID NO: 130) | |
| | | KSSEKIVYVYMKLNYEVMTK (SEQ ID NO: 131) | |
| | | KHAVVTHRLRERKQLVVYEEI (SEQ ID NO: 129) | |
| 57 | TRAG-3 | CEFHACWPAFTVLGE (SEQ ID NO: 163) | Janjic et al. J Immunol. |
| | | | 177(4): 2717-27 (2006). |
| 58 | TRP2- | EVISCKLIKR (SEQ ID NO: 235) | Lupetti et al. J Exp Med. |
| | INT2g | | 188(6): 1005-16 (1998). |
| 59 | pbk | GSPFPAAVI (SEQ ID NO: 2) | Morgan et al., J. Immunol. |
| | | | 171: 3287-3295 (2003) |

TABLE 12
Squamous cell carcinoma

| | Tumor- | | |
|---|---|---|---|
| | associated | | |
| No. | antigen | Immunogenic epitopes | Sources |
| 1 | CASP-8 | FPSDSWCYF (SEQ ID NO: 279) | Mandruzzato et al. J. Exp. Med. |
| | | | 186(5): 785-93 (1997). |
| 2 | p53 | VVPCEPPEV (SEQ ID NO: 79) | Ito et al. Int. J. Cancer. |
| | | | 120(12): 2618-24 (2007). |
| 3 | SAGE | LYATVIHDI (SEQ ID NO: 236) | Miyahara et al. Clin Cancer |
| | | | Res. 11(15): 5581-9 (2005). |

TABLE 13
Chronic myeloid leukemia

| | Tumor- | | |
|---|---|---|---|
| | associated | | |
| No. | antigen | Immunogenic epitopes | Sources |
| 1 | BCR-ABL | SSKALQRPV (SEQ ID NO: 237); | Yotnda et al. J. Clin. Invest. |
| | | GFKQSSKAL (SEQ ID NO: 238); | 101(10): 2290-6 (1998). |
| | | ATGFKQSSKALQRPVAS (SEQ ID NO: 239); and | Bosch et al. Blood. 88(9): 3522-7 |
| | | ATGFKQSSKALQRPVAS (SEQ ID NO: 239) | (1996). |
| | | | Makita et al. Leukemia. |
| | | | 16(12): 2400-7 (2002). |
| 2 | dek-can | TMKQICKKEIRRLHQY (SEQ ID NO: 240) | Makita et al. Leukemia. |
| | | | 16(12): 2400-7 (2002). |
| 3 | EFTUD2 | KILDAVVAQK (SEQ ID NO: 241) | Lennerz et al. Proc. Natl. Acad. |
| | | | Sci. U.S.A. 102(44): 16013-8 |
| | | | (2005). |
| 4 | GAGE- | YYWPRPRRY (SEQ ID NO: 219) | De Backer et al. Cancer Res. |
| | 3,4,5,6,7 | | 59(13): 3157-65 (1999). |

TABLE 14
Acute lymphoblastic leukemia

| | Tumor- | | |
|---|---|---|---|
| | associated | | |
| No. | antigen | Immunogenic epitopes | Sources |
| 1 | ETV6- | RIAECILGM (SEQ ID NO: 242) and | Yotnda et al. J. Clin. Invest. |
| | AML1 | IGRIAECILGMNPSR (SEQ ID NO: 243) | (2): 455-62 (1998). |
| | | | Yun et al. Tissue Antigens. |
| | | | 54(2): 153-61 (1999). |
| 2 | GAGE- | YYWPRPRRY (SEQ ID NO: 219) | De Backer et al. Cancer Res. |
| | 3,4,5,6,7 | | 59(13): 3157-65 (1999). |

TABLE 15
Acute myelogenous leukemia

| | Tumor- | | |
|---|---|---|---|
| | associated | | |
| No. | antigen | Immunogenic epitopes | Sources |
| 1 | FLT3-ITD | YVDFREYEYY (SEQ ID NO: 244) | Graf et al. Blood. 109(7): 2985-8 |
| | | | (2007). |
| 2 | Cyclin-A1 | FLDRFLSCM (SEQ ID NO: 245) and | Ochsenreither et al. Blood. |
| | | SLIAAAAFCLA (SEQ ID NO: 246) | 119(23): 5492-501 (2012). |
| 3 | GAGE- | YYWPRPRRY (SEQ ID NO: 219) | De Backer et al. Cancer Res. |
| | 3,4,5,6,7 | | 59(13): 3157-65 (1999). |

TABLE 16
Chronic lymphocytic leukemia

| | Tumor- | | |
|---|---|---|---|
| | associated | | |
| No. | antigen | Immunogenic epitopes | Sources |
| 1 | FNDC3B | VVMSWAPPV (SEQ ID NO: 247) | Rajasagi et al. Blood. |
| | | | 124(3): 453-62 (2014). |
| 2 | GAGE- | YYWPRPRRY (SEQ ID NO: 219) | De Backer et al. Cancer Res. |
| | 3,4,5,6,7 | | 59(13): 3157-65 (1999). |

TABLE 17
Promyelocytic leukemia

| | Tumor- | | |
|---|---|---|---|
| | associated | | |
| No. | antigen | Immunogenic epitopes | Sources |
| 1 | pml- | NSNHVASGAGEAAIETQSSSSEEIV (SEQ ID | Gambacorti-Passerini et al. |
| | RARalpha | NO: 248) | Blood. 81(5): 1369-75 (1993). |
| 2 | GAGE- | YYWPRPRRY (SEQ ID NO: 219) | De Backer et al. Cancer Res. |
| | 3,4,5,6,7 | | 59(13): 3157-65 (1999). |

TABLE 18
Multiple myeloma

| | Tumor- | | |
|---|---|---|---|
| | associated | | |
| No. | antigen | Immunogenic epitopes | Sources |
| 1 | MAGE-C1 | ILFGISLREV (SEQ ID NO: 249) | Anderson et al. Cancer Immunol |
| | | KVVEFLAML (SEQ ID NO: 250) | Immunother. 60(7): 985-97 (2011). |
| | | SSALLSIFQSSPE (SEQ ID NO: 251) | Nuber et al. Proc Natl Acad Sci USA. |
| | | SFSYTLLSL (SEQ ID NO: 252) | 107(34): 15187-92 (2010). |
| | | VSSFFSYTL (SEQ ID NO: 253) | |
| 2 | NY-ESO-1 | HLA-A2-restricted peptide p157-165 | Jager et al. Proc. Natl. Acad. |
| | | (SLLMWITQC) (SEQ ID NO: 32), HLA-Cw3- | Scie. U.S.A. 103(39): 14453-8 |
| | | restricted p92-100 (LAMP-FATPM) (SEQ ID | (2006). |
| | | NO: 33) and HLA-Cw6-restricted p80-88 | Gnjatic et al. PNAS |
| | | (ARGPESRLL) (SEQ ID NO: 34) | Sep. 26, 2000 vol. 97 no. |
| | | SLLMWITQC (SEQ ID NO: 32) | 20 p. 10919 |
| | | MLMAQEALAFL (SEQ ID NO: 35) | Jager et al. J Exp Med. |
| | | YLAMPFATPME (SEQ ID NO: 36) | 187(2): 265-70 (1998). |
| | | ASGPGGGAPR (SEQ ID NO: 37) | Chen et al. J Immunol. |
| | | LAAQERRVPR (SEQ ID NO: 38) | 165(2): 948-55 (2000). |
| | | TVSGNILTIR (SEQ ID NO: 39) | Valmori et al. Cancer Res. |
| | | APRGPHGGAASGL (SEQ ID NO: 40) | 60(16): 4499-506 (2000). |
| | | MPFATPMEAEL (SEQ ID NO: 41) | Aarnoudse et al. Int J Cancer. |
| | | KEFTVSGNILTI (SEQ ID NO: 42) | 82(3): 442-8 (1999). |
| | | MPFATPMEA (SEQ ID NO: 43) | Eikawa et al. Int J Cancer. |
| | | FATPMEAEL (SEQ ID NO: 44) | 132(2): 345-54 (2013). |
| | | FATPMEAELAR (SEQ ID NO: 45) | Wang et al. J Immunol. |
| | | LAMPFATPM (SEQ ID NO: 46) | 161(7): 3598-606 (1998). |
| | | ARGPESRLL (SEQ ID NO: 34) | Matsuzaki et al. Cancer Immunol |
| | | SLLMWITQCFLPVF (SEQ ID NO: 47) | Immunother. 57(8)1185-95 |
| | | LLEFYLAMPFATPMEAEL-ARRSLAQ (SEQ ID | (2008). |
| | | NO: 48) | Ebert et al. Cancer Res. |
| | | EFYLAMPFATPM (SEQ ID NO: 49) | 69(3): 1046-54 (2009). |
| | | PGVLLKEFTVSGNILTIRL-TAADHR (SEQ ID | Eikawa et al. Int J Cancer. |
| | | NO: 50) | 132(2): 345-54 (2013). |
| | | RLLEFYLAMPFA (SEQ ID NO: 51) | Knights et al. Cancer Immunol |
| | | QGAMLAAQERRVPRAAE-VPR (SEQ ID NO: | Immunother. 58(3): 325-38 (2009). |
| | | 52) | Jäger et al. Cancer Immun. 2: 12 |
| | | PFATPMEAELARR (SEQ ID NO: 53) | (2002). |
| | | PGVLLKEFTVSGNILTIRLT (SEQ ID NO: 54) | Zeng et al. Proc Natl Acad Sci USA. |
| | | VLLKEFTVSG (SEQ ID NO: 55) | 98(7): 3964-9 (2001). |
| | | AADHRQLQLSISSCLQQL (SEQ ID NO: 56) | Mandic et al. J Immunol. |
| | | LKEFTVSGNILTIRL (SEQ ID NO: 57) | 174(3): 1751-9 (2005). |
| | | PGVLLKEFTVSGNILTIRL-TAADHR (SEQ ID | Chen et al. Proc Natl Acad Sci USA. |
| | | NO: 50) | 101(25): 9363-8 (2004). |
| | | LLEFYLAMPFATPMEAEL-ARRSLAQ (SEQ ID | Ayyoub et al. Clin Cancer Res. |
| | | NO: 48) | 16(18): 4607-15 (2010). |
| | | KEFTVSGNILT (SEQ ID NO: 58) | Slager et al. J Immunol. |
| | | LLEFYLAMPFATPM (SEQ ID NO: 59) | 172(8): 5095-102 (2004). |
| | | AGATGGRGPRGAGA (SEQ ID NO: 60) | Mizote et al. Vaccine. |
| | | | 28(32): 5338-46 (2010). |
| | | | Jager et al. J Exp Med. |
| | | | 191(4): 625-30 (2000). |
| | | | Zarour et al. Cancer Res. |
| | | | 60(17): 4946-52 (2000). |
| | | | Zeng et al. J Immunol. |
| | | | 165(2): 1153-9 (2000). |
| | | | Bioley et al. Clin Cancer Res. |
| | | | 15(13): 4467-74 (2009). |
| | | | Zarour et al. Cancer Res. |
| | | | 62(1): 213-8 (2002). |
| | | | Hasegawa et al. Clin Cancer Res. |
| | | | 12(6): 1921-7 (2006). |
| 3 | LAGE-1 | MLMAQEALAFL (SEQ ID NO: 35) | Aarnoudse et al. Int J Cancer. |
| | | SLLMWITQC (SEQ ID NO: 32) | 82(3): 442-8 (1999). |
| | | LAAQERRVPR (SEQ ID NO: 38) | Rimoldi et al. J Immunol. |
| | | ELVRRILSR (SEQ ID NO: 117) | 165(12): 7253-61 (2000). |
| | | APRGVRMAV (SEQ ID NO: 118) | Wang et al. J Immunol. |
| | | SLLMWITQCFLPVF (SEQ ID NO: 47) | 161(7): 3598-606 (1998). |
| | | QGAMLAAQERRVPRAAEVP-R (SEQ ID NO: | Sun et al. Cancer Immunol |
| | | 119) | Immunother. 55(6): 644-52 (2006). |
| | | AADHRQLQLSISSCLQQL (SEQ ID NO: 56) | Slager et al. Cancer Gene Ther. |
| | | CLSRRPWKRSWSAGSCPG-MPHL (SEQ ID | 11(3): 227-36 (2004). |
| | | NO: 120) | Zeng et al. Proc Natl Acad Sci USA. |
| | | ILSRDAAPLPRPG (SEQ ID NO: 121) | 98(7): 3964-9 (2001). |
| | | AGATGGRGPRGAGA (SEQ ID NO: 60) | Slager et al. J Immunol. |
| | | | 172(8): 5095-102 (2004). |
| | | | Jager et al. J Exp Med. |
| | | | 191(4): 625-30 (2000). |
| | | | Slager et al. J Immunol. |
| | | | 170(3): 1490-7 (2003). |
| | | | Wang et al. Immunity. 20(1): 107-18 |
| | | | (2004). |
| | | | Hasegawa et al. Clin Cancer Res. |
| | | | 12(6): 1921-7 (2006). |
| 4 | HERV-K- | MLAVISCAV (SEQ ID NO: 112) | Schiavetti et al. Cancer Res. |
| | MEL | | 62(19): 5510-6 (2002). |
| 5 | KK-LC-1 | RQKRILVNL (SEQ ID NO: 113) | Fukuyama et al. Cancer Res. |
| | | | 66(9): 4922-8 (2006). |
| 6 | KM-HN-1 | NYNNFYRFL (SEQ ID NO: 114) | Fukuyama et al. Cancer Res. |
| | | EYSKECLKEF (SEQ ID NO: 115) | 66(9): 4922-8 (2006). |
| | | EYLSLSDKI (SEQ ID NO: 116) | Monji et al. Clin Cancer Res. |
| | | | 10(18 Pt 1): 6047-57 (2004). |
| 7 | Sp17 | ILDSSEEDK (SEQ ID NO: 102) | Chiriva-Internati et al. Int J |
| | | | Cancer. 107(5): 863-5 (2003). |

TABLE 19
B-cell lymphoma

| | Tumor- | | |
|---|---|---|---|
| | associated | | |
| No. | antigen | Immunogenic epitopes | Source |
| 1 | D393-CD20 | KPLFRRMSSLELVIA (SEQ ID NO: 254) | Vauchy et al. Int J Cancer. |
| | | | 137(1): 116-26 (2015). |

TABLE 20
Bladder carcinoma

| | Tumor- | | |
|---|---|---|---|
| | associated | | |
| No. | antigen | Immunogenic epitopes | Sources |
| 1 | BAGE-1 | AARAVFLAL (SEQ ID NO: 137) | Boel et al. Immunity. 2(2): 167-75 |
| | | | (1995). |
| 2 | GAGE- | YRPRPRRY (SEQ ID NO: 205) | Van den Eynde et al. J Exp Med. |
| | 1,2,8 | | 182(3): 689-98 (1995). |
| 3 | GAGE- | YYWPRPRRY (SEQ ID NO: 219) | De Backer et al. Cancer Res. |
| | 3,4,5,6,7 | | 59(13): 3157-65 (1999). |
| 4 | MAGE-A4 | EVDPASNTY (SEQ ID NO: 122) | Kobayashi et al. Tissue |
| | (transitional | GVYDGREHTV (SEQ ID NO: 123) | Antigens. 62(5): 426-32 (2003). |
| | cell | NYKRCFPVI (SEQ ID NO: 124) | Duffour et al. Eur J Immunol. |
| | carcinoma | SESLKMIF (SEQ ID NO: 125) | 29(10): 3329-37 (1999). |
| | of urinary | | Miyahara et al. Clin Cancer Res. |
| | bladder) | | 11(15): 5581-9 (2005). |
| | | | Ottaviani et al. Cancer Immunol |
| | | | Immunother. 55(7): 867-72 |
| | | | (2006). |
| | | | Zhang et al. Tissue Antigens. |
| | | | 60(5): 365-71 (2002). |
| 5 | MAGE-A6 | MVKISGGPR (SEQ ID NO: 206) | Zorn et al. Eur J Immunol. |
| | | EVDPIGHVY (SEQ ID NO: 207) | 29(2): 602-7 (1999). |
| | | REPVTKAEML (SEQ ID NO: 143) | Benlalam et al. J Immunol. |
| | | EGDCAPEEK (SEQ ID NO: 155) | 171(11): 6283-9 (2003). |
| | | ISGGPRISY (SEQ ID NO: 208) | Tanzarella et al. Cancer Res. |
| | | LLKYRAREPVTKAE (SEQ ID NO: 156) | 59(11): 2668-74 (1999). |
| | | | Breckpot et al. J Immunol. |
| | | | 172(4): 2232-7 (2004). |
| | | | Vantomme et al. Cancer Immun. |
| | | | 3: 17 (2003). |
| | | | Chaux et al. J Exp Med. |
| | | | 189(5): 767-78 (1999). |
| 6 | SAGE | LYATVIHDI (SEQ ID NO: 236) | Miyahara et al. Clin Cancer Res. |
| | | | 11(15): 5581-9 (2005). |
| 7 | NY-ESO-1 | HLA-A2-restricted peptide p157-165 | Jager et al. Proc. Natl. Acad. |
| | | (SLLMWITQC) (SEQ ID NO: 32), HLA-Cw3- | Scie. U.S.A. 103(39): 14453-8 |
| | | restricted p92-100 (LAMP-FATPM) (SEQ ID | (2006). |
| | | NO: 33) and HLA-Cw6-restricted p80-88 | Gnjatic et al. PNAS |
| | | (ARGPESRLL) (SEQ ID NO: 34) | Sep. 26, 2000 vol. 97 no. |
| | | SLLMWITQC (SEQ ID NO: 32) | 20 p. 10919 |
| | | MLMAQEALAFL (SEQ ID NO: 35) | Jager et al. J Exp Med. |
| | | YLAMPFATPME (SEQ ID NO: 36) | 187(2): 265-70 (1998). |
| | | ASGPGGGAPR (SEQ ID NO: 37) | Chen et al. J Immunol. |
| | | LAAQERRVPR (SEQ ID NO: 38) | 165(2): 948-55 (2000). |
| | | TVSGNILTIR (SEQ ID NO: 39) | Valmori et al. Cancer Res. |
| | | APRGPHGGAASGL (SEQ ID NO: 40) | 60(16): 4499-506 (2000). |
| | | MPFATPMEAEL (SEQ ID NO: 41) | Aarnoudse et al. Int J Cancer. |
| | | KEFTVSGNILTI (SEQ ID NO: 42) | 82(3): 442-8 (1999). |
| | | MPFATPMEA (SEQ ID NO: 43) | Eikawa et al. Int J Cancer. |
| | | FATPMEAEL (SEQ ID NO: 44) | 132(2): 345-54 (2013). |
| | | FATPMEAELAR (SEQ ID NO: 45) | Wang et al. J Immunol. |
| | | LAMPFATPM (SEQ ID NO: 46) | 161(7): 3598-606 (1998). |
| | | ARGPESRLL (SEQ ID NO: 34) | Matsuzaki et al. Cancer Immunol |
| | | SLLMWITQCFLPVF (SEQ ID NO: 47) | Immunother. 57(8)1185-95 |
| | | LLEFYLAMPFATPMEAEL-ARRSLAQ (SEQ ID | (2008). |
| | | NO: 48) | Ebert et al. Cancer Res. |
| | | EFYLAMPFATPM (SEQ ID NO: 49) | 69(3): 1046-54 (2009). |
| | | PGVLLKEFTVSGNILTIRL-TAADHR (SEQ ID | Eikawa et al. Int J Cancer. |
| | | NO: 50) | 132(2): 345-54 (2013). |
| | | RLLEFYLAMPFA (SEQ ID NO: 51) | Knights et al. Cancer Immunol |
| | | QGAMLAAQERRVPRAAE-VPR (SEQ ID NO: | Immunother. 58(3): 325-38 |
| | | 52) | (2009). |
| | | PFATPMEAELARR (SEQ ID NO: 53) | Jäger et al. Cancer Immun. 2: 12 |
| | | PGVLLKEFTVSGNILTIRLT (SEQ ID NO: 54) | (2002). |
| | | VLLKEFTVSG (SEQ ID NO: 55) | Zeng et al. Proc Natl Acad Sci USA. |
| | | AADHRQLQLSISSCLQQL (SEQ ID NO: 56) | 98(7): 3964-9 (2001). |
| | | LKEFTVSGNILTIRL (SEQ ID NO: 57) | Mandic et al. J Immunol. |
| | | PGVLLKEFTVSGNILTIRL-TAADHR (SEQ ID | 174(3): 1751-9 (2005). |
| | | NO: 50) | Chen et al. Proc Natl Acad Sci USA. |
| | | LLEFYLAMPFATPMEAEL-ARRSLAQ (SEQ ID | 101(25): 9363-8 (2004). |
| | | NO: 48) | Ayyoub et al. Clin Cancer Res. |
| | | KEFTVSGNILT (SEQ ID NO: 58) | 16(18): 4607-15 (2010). |
| | | LLEFYLAMPFATPM (SEQ ID NO: 59) | Slager et al. J Immunol. |
| | | AGATGGRGPRGAGA (SEQ ID NO: 60) | 172(8): 5095-102 (2004). |
| | | | Mizote et al. Vaccine. |
| | | | 28(32): 5338-46 (2010). |
| | | | Jager et al. J Exp Med. |
| | | | 191(4): 625-30 (2000). |
| | | | Zarour et al. Cancer Res. |
| | | | 60(17): 4946-52 (2000). |
| | | | Zeng et al. J Immunol. |
| | | | 165(2): 1153-9 (2000). |
| | | | Bioley et al. Clin Cancer Res. |
| | | | 15(13): 4467-74 (2009). |
| | | | Zarour et al. Cancer Res. |
| | | | 62(1): 213-8 (2002). |
| | | | Hasegawa et al. Clin Cancer |
| | | | Res. 12(6): 1921-7 (2006). |
| 8 | LAGE-1 | MLMAQEALAFL (SEQ ID NO: 35) | Aarnoudse et al. Int J Cancer. |
| | | SLLMWITQC (SEQ ID NO: 32) | 82(3): 442-8 (1999). |
| | | LAAQERRVPR (SEQ ID NO: 38) | Rimoldi et al. J Immunol. |
| | | ELVRRILSR (SEQ ID NO: 117) | 165(12): 7253-61 (2000). |
| | | APRGVRMAV (SEQ ID NO: 118) | Wang et al. J Immunol. |
| | | SLLMWITQCFLPVF (SEQ ID NO: 47) | 161(7): 3598-606 (1998). |
| | | QGAMLAAQERRVPRAAEVP-R (SEQ ID NO: | Sun et al. Cancer Immunol |
| | | 119) | Immunother. 55(6): 644-52 |
| | | AADHRQLQLSISSCLQQL (SEQ ID NO: 56) | (2006). |
| | | CLSRRPWKRSWSAGSCPG-MPHL (SEQ ID | Slager et al. Cancer Gene Ther. |
| | | NO: 120) | 11(3): 227-36 (2004). |
| | | ILSRDAAPLPRPG (SEQ ID NO: 121) | Zeng et al. Proc Natl Acad Sci USA. |
| | | AGATGGRGPRGAGA (SEQ ID NO: 60) | 98(7): 3964-9 (2001). |
| | | | Slager et al. J Immunol. |
| | | | 172(8): 5095-102 (2004). |
| | | | Jager et al. J Exp Med. |
| | | | 191(4): 625-30 (2000). |
| | | | Slager et al. J Immunol. |
| | | | 170(3): 1490-7 (2003). |
| | | | Wang et al. Immunity. 20(1): 107-18 |
| | | | (2004). |
| | | | Hasegawa et al. Clin Cancer |
| | | | Res. 12(6): 1921-7 (2006). |
| 9 | HERV-K- | MLAVISCAV (SEQ ID NO: 112) | Schiavetti et al. Cancer Res. |
| | MEL | | 62(19): 5510-6 (2002). |
| 10 | KK-LC-1 | RQKRILVNL (SEQ ID NO: 113) | Fukuyama et al. Cancer Res. |
| | | | 66(9): 4922-8 (2006). |
| 11 | KM-HN-1 | NYNNFYRFL (SEQ ID NO: 114) | Fukuyama et al. Cancer Res. |
| | | EYSKECLKEF (SEQ ID NO: 115) | 66(9): 4922-8 (2006). |
| | | EYLSLSDKI (SEQ ID NO: 116) | Monji et al. Clin Cancer Res. |
| | | | 10(18 Pt 1): 6047-57 (2004). |
| 12 | Sp17 | ILDSSEEDK (SEQ ID NO: 102) | Chiriva-Internati et al. Int J |
| | | | Cancer. 107(5): 863-5 (2003). |

TABLE 21
Head and neck cancer

| | Tumor- | | |
|---|---|---|---|
| | associated | | |
| No. | antigen | Immunogenic epitopes | Sources |
| 1 | BAGE-1 | AARAVFLAL (SEQ ID NO: 137) | Boel et al. Immunity. 2(2): 167-75 |
| | (head and | | (1995). |
| | neck | | |
| | squamous | | |
| | cell | | |
| | carcinoma) | | |
| 2 | GAGE- | YRPRPRRY (SEQ ID NO: 205) | Van den Eynde et al. J Exp Med. |
| | 1,2,8 | | 182(3): 689-98 (1995). |
| 3 | GAGE- | YYWPRPRRY (SEQ ID NO: 219) | De Backer et al. Cancer Res. |
| | 3,4,5,6,7 | | 59(13): 3157-65 (1999). |
| 4 | LY6K | RYCNLEGPPI (SEQ ID NO: 221) | Suda et al. Cancer Sci. |
| | | KWTEPYCVIAAVKIFPRFFMV-AKQ (SEQ ID | 98(11): 1803-8 (2007). |
| | | NO: 222) | Tomita et al. Oncoimmunology. |
| | | KCCKIRYCNLEGPPINSSVF (SEQ ID NO: 223) | 3: e28100 (2014). |
| 5 | MAGE-A3 | EVDPIGHLY (SEQ ID NO: 255) | Gaugler et al. J Exp Med. |
| | (head and | FLWGPRALV (SEQ ID NO: 225) | 179(3): 921-30 (1994). |
| | neck | KVAELVHFL (SEQ ID NO: 256) | van der Bruggen et al. Eur J |
| | squamous | TFPDLESEF (SEQ ID NO: 257) | Immunol. 24(12): 3038-43 (1994). |
| | cell | VAELVHFLL (SEQ ID NO: 258) | Kawashima et al. Hum Immunol. |
| | carcinoma) | MEVDPIGHLY (SEQ ID NO: 259) | 59(1): 1-14 (1998). |
| | | EVDPIGHLY (SEQ ID NO: 255) | Oiso et al. Int J Cancer. |
| | | REPVTKAEML (SEQ ID NO: 143) | 81(3): 387-94 (1999). |
| | | AELVHFLLL (SEQ ID NO: 260) | Miyagawa et al. Oncology. |
| | | MEVDPIGHLY (SEQ ID NO: 259) | 70(1): 54-62 (2006). |
| | | WQYFFPVIF (SEQ ID NO: 261) | Bilsborough et al. Tissue |
| | | EGDCAPEEK (SEQ ID NO: 155) | Antigens. 60(1): 16-24 (2002). |
| | | KKLLTQHFVQENYLEY (SEQ ID NO: 262) | Schultz et al. Tissue Antigens. |
| | | RKVAELVHFLLLKYR (SEQ ID NO: 263) | 57(2)103-9 (2001). |
| | | KKLLTQHFVQENYLEY (SEQ ID NO: 262) | Tanzarella et al. Cancer Res. |
| | | ACYEFLWGPRALVETS (SEQ ID NO: 264) | 59(11): 2668-74 (1999). |
| | | RKVAELVHFLLLKYR (SEQ ID NO: 263) | Schultz et al. J Exp Med. |
| | | VIFSKASSSLQL (SEQ ID NO: 265) | 195(4): 391-9 (2002). |
| | | VFGIELMEVDPIGHL (SEQ ID NO: 266) | Herman et al. Immunogenetics. |
| | | GDNQIMPKAGLLIIV (SEQ ID NO: 267) | 43(6): 377-83 (1996). |
| | | TSYVKVLHHMVKISG (SEQ ID NO: 268) | Russo et al. Proc Natl Acad Sci |
| | | RKVAELVHFLLLKYRA (SEQ ID NO: 269) | USA. 97(5): 2185-90 (2000). |
| | | FLLLKYRAREPVTKAE (SEQ ID NO: 151) | Breckpot et al. J Immunol. |
| | | | 172(4): 2232-7 (2004). |
| | | | Schultz et al. Cancer Res. |
| | | | 60(22): 6272-5 (2000). |
| | | | Cesson et al. Cancer Immunol |
| | | | Immunother. 60(1): 23-35 (2011). |
| | | | Schultz et al. J Immunol. |
| | | | 172(2): 1304-10 (2004). |
| | | | Zhang et al. J Immunol. |
| | | | 171(1): 219-25 (2003). |
| | | | Cesson et al. Cancer Immunol |
| | | | Immunother. 60(1): 23-35 (2010). |
| | | | Kobayashi et al. Cancer Res. |
| | | | 61(12): 4773-8 (2001). |
| | | | Cesson et al. Cancer Immunol |
| | | | Immunother. 60(1): 23-35 (2011). |
| | | | Consogno et al. Blood. |
| | | | 101(3): 1038-44 (2003). |
| | | | Manici et al. J Exp Med. |
| | | | 189(5): 871-6 (1999). |
| | | | Chaux et al. J Exp Med. |
| | | | 189(5): 767-78 (1999). |
| 6 | MAGE-A6 | MVKISGGPR (SEQ ID NO: 206) | Zorn et al. Eur J Immunol. |
| | | EVDPIGHVY (SEQ ID NO: 207) | 29(2): 602-7 (1999). |
| | | REPVTKAEML (SEQ ID NO: 143) | Benlalam et al. J Immunol. |
| | | EGDCAPEEK (SEQ ID NO: 155) | 171(11): 6283-9 (2003). |
| | | ISGGPRISY (SEQ ID NO: 208) | Tanzarella et al. Cancer Res. |
| | | LLKYRAREPVTKAE (SEQ ID NO: 156) | 59(11): 2668-74 (1999). |
| | | | Breckpot et al. J Immunol. |
| | | | 172(4): 2232-7 (2004). |
| | | | Vantomme et al. Cancer Immun. |
| | | | 3: 17 (2003). |
| | | | Chaux et al. J Exp Med. |
| | | | 189(5): 767-78 (1999). |
| 7 | SAGE | LYATVIHDI (SEQ ID NO: 236) | Miyahara et al. Clin Cancer Res. |
| | | | 11(15): 5581-9 (2005). |

TABLE 22
Esophageal cancer

| | Tumor- | | |
|---|---|---|---|
| | associated | | |
| No. | antigen | Immunogenic epitopes | Sources |
| 1 | GAGE- | YYWPRPRRY (SEQ ID NO: 219) | De Backer et al. Cancer Res. |
| | 3,4,5,6,7 | | 59(13): 3157-65 (1999). |
| | (Esophageal | | |
| | squamous | | |
| | cell | | |
| | carcinoma | | |
| | and | | |
| | esophageal | | |
| | adenocarcinoma) | | |
| 2 | MAGE-A2 | YLQLVFGIEV (SEQ ID NO: 153) | Kawashima et al. Hum Immunol. |
| | | EYLQLVFGI (SEQ ID NO: 154) | 59(1): 1-14 (1998). |
| | | REPVTKAEML (SEQ ID NO: 143) | Tahara et al. Clin Cancer Res. |
| | | EGDCAPEEK (SEQ ID NO: 155) | 5(8): 2236-41 (1999). |
| | | LLKYRAREPVTKAE (SEQ ID NO: 156) | Tanzarella et al. Cancer Res. |
| | | | 59(11): 2668-74 (1999). |
| | | | Breckpot et al. J Immunol. |
| | | | 172(4): 2232-7 (2004). |
| | | | Chaux et al. J Exp Med. |
| | | | 189(5): 767-78 (1999). |
| 3 | MAGE-A6 | MVKISGGPR (SEQ ID NO: 206) | Zorn et al. Eur J Immunol. |
| | | EVDPIGHVY (SEQ ID NO: 207) | 29(2): 602-7 (1999). |
| | | REPVTKAEML (SEQ ID NO: 143) | Benlalam et al. J Immunol. |
| | | EGDCAPEEK (SEQ ID NO: 155) | 171(11): 6283-9 (2003). |
| | | ISGGPRISY (SEQ ID NO: 208) | Tanzarella et al. Cancer Res. |
| | | LLKYRAREPVTKAE (SEQ ID NO: 156) | 59(11): 2668-74 (1999). |
| | | | Breckpot et al. J Immunol. |
| | | | 172(4): 2232-7 (2004). |
| | | | Vantomme et al. Cancer Immun. |
| | | | 3: 17 (2003). |
| | | | Chaux et al. J Exp Med. |
| | | | 189(5): 767-78 (1999). |
| 4 | NY-ESO-1 | HLA-A2-restricted peptide p157-165 | Jager et al. Proc. Natl. Acad. |
| | | (SLLMWITQC) (SEQ ID NO: 32), HLA-Cw3- | Scie. U.S.A. 103(39): 14453-8 |
| | | restricted p92-100 (LAMP-FATPM) (SEQ ID | (2006). |
| | | NO: 33) and HLA-Cw6-restricted p80-88 | Gnjatic et al. PNAS |
| | | (ARGPESRLL) (SEQ ID NO: 34) | Sep. 26, 2000 vol. 97 no. |
| | | SLLMWITQC (SEQ ID NO: 32) | 20 p. 10919 |
| | | MLMAQEALAFL (SEQ ID NO: 35) | Jager et al. J Exp Med. |
| | | YLAMPFATPME (SEQ ID NO: 36) | 187(2): 265-70 (1998). |
| | | ASGPGGGAPR (SEQ ID NO: 37) | Chen et al. J Immunol. |
| | | LAAQERRVPR (SEQ ID NO: 38) | 165(2): 948-55 (2000). |
| | | TVSGNILTIR (SEQ ID NO: 39) | Valmori et al. Cancer Res. |
| | | APRGPHGGAASGL (SEQ ID NO: 40) | 60(16): 4499-506 (2000). |
| | | MPFATPMEAEL (SEQ ID NO: 41) | Aarnoudse et al. Int J Cancer. |
| | | KEFTVSGNILTI (SEQ ID NO: 42) | 82(3): 442-8 (1999). |
| | | MPFATPMEA (SEQ ID NO: 43) | Eikawa et al. Int J Cancer. |
| | | FATPMEAEL (SEQ ID NO: 44) | 132(2): 345-54 (2013). |
| | | FATPMEAELAR (SEQ ID NO: 45) | Wang et al. J Immunol. |
| | | LAM PFATPM (SEQ ID NO: 46) | 161(7): 3598-606 (1998). |
| | | ARGPESRLL (SEQ ID NO: 34) | Matsuzaki et al. Cancer Immunol |
| | | SLLMWITQCFLPVF (SEQ ID NO: 47) | Immunother. 57(8)1185-95 |
| | | LLEFYLAMPFATPMEAEL-ARRSLAQ (SEQ ID | (2008). |
| | | NO: 48) | Ebert et al. Cancer Res. |
| | | EFYLAMPFATPM (SEQ ID NO: 49) | 69(3): 1046-54 (2009). |
| | | PGVLLKEFTVSGNILTIRL-TAADHR (SEQ ID | Eikawa et al. Int J Cancer. |
| | | NO: 50) | 132(2): 345-54 (2013). |
| | | RLLEFYLAMPFA (SEQ ID NO: 51) | Knights et al. Cancer Immunol |
| | | QGAMLAAQERRVPRAAE-VPR (SEQ ID NO: | Immunother. 58(3): 325-38 |
| | | 52) | (2009). |
| | | PFATPMEAELARR (SEQ ID NO: 53) | Jäger et al. Cancer Immun. 2: 12 |
| | | PGVLLKEFTVSGNILTIRLT (SEQ ID NO: 54) | (2002). |
| | | VLLKEFTVSG (SEQ ID NO: 55) | Zeng et al. Proc Natl Acad Sci USA. |
| | | AADHRQLQLSISSCLQQL (SEQ ID NO: 56) | 98(7): 3964-9 (2001). |
| | | LKEFTVSGNILTIRL (SEQ ID NO: 57) | Mandic et al. J Immunol. |
| | | PGVLLKEFTVSGNILTIRL-TAADHR (SEQ ID | 174(3): 1751-9 (2005). |
| | | NO: 50) | Chen et al. Proc Natl Acad Sci USA. |
| | | LLEFYLAMPFATPMEAEL-ARRSLAQ (SEQ ID | 101(25): 9363-8 (2004). |
| | | NO: 48) | Ayyoub et al. Clin Cancer Res. |
| | | KEFTVSGNILT (SEQ ID NO: 58) | 16(18): 4607-15 (2010). |
| | | LLEFYLAMPFATPM (SEQ ID NO: 59) | Slager et al. J Immunol. |
| | | AGATGGRGPRGAGA (SEQ ID NO: 60) | 172(8): 5095-102 (2004). |
| | | | Mizote et al. Vaccine. |
| | | | 28(32): 5338-46 (2010). |
| | | | Jager et al. J Exp Med. |
| | | | 191(4): 625-30 (2000). |
| | | | Zarour et al. Cancer Res. |
| | | | 60(17): 4946-52 (2000). |
| | | | Zeng et al. J Immunol. |
| | | | 165(2): 1153-9 (2000). |
| | | | Bioley et al. Clin Cancer Res. |
| | | | 15(13): 4467-74 (2009). |
| | | | Zarour et al. Cancer Res. |
| | | | 62(1): 213-8 (2002). |
| | | | Hasegawa et al. Clin Cancer |
| | | | Res. 12(6): 1921-7 (2006). |
| 5 | LAGE-1 | MLMAQEALAFL (SEQ ID NO: 35) | Aarnoudse et al. Int J Cancer. |
| | | SLLMWITQC (SEQ ID NO: 32) | 82(3): 442-8 (1999). |
| | | LAAQERRVPR (SEQ ID NO: 38) | Rimoldi et al. J Immunol. |
| | | ELVRRILSR (SEQ ID NO: 117) | 165(12): 7253-61 (2000). |
| | | APRGVRMAV (SEQ ID NO: 118) | Wang et al. J Immunol. |
| | | SLLMWITQCFLPVF (SEQ ID NO: 47) | 161(7): 3598-606 (1998). |
| | | QGAMLAAQERRVPRAAEVP-R (SEQ ID NO: | Sun et al. Cancer Immunol |
| | | 119) | Immunother. 55(6): 644-52 |
| | | AADHRQLQLSISSCLQQL (SEQ ID NO: 56) | (2006). |
| | | CLSRRPWKRSWSAGSCPG-MPHL (SEQ ID | Slager et al. Cancer Gene Ther. |
| | | NO: 120) | 11(3): 227-36 (2004). |
| | | ILSRDAAPLPRPG (SEQ ID NO: 121) | Zeng et al. Proc Natl Acad Sci USA. |
| | | AGATGGRGPRGAGA (SEQ ID NO: 60) | 98(7): 3964-9 (2001). |
| | | | Slager et al. J Immunol. |
| | | | 172(8): 5095-102 (2004). |
| | | | Jager et al. J Exp Med. |
| | | | 191(4): 625-30 (2000). |
| | | | Slager et al. J Immunol. |
| | | | 170(3): 1490-7 (2003). |
| | | | Wang et al. Immunity. 20(1): 107-18 |
| | | | (2004). |
| | | | Hasegawa et al. Clin Cancer |
| | | | Res. 12(6): 1921-7 (2006). |
| 6 | HERV-K- | MLAVISCAV (SEQ ID NO: 112) | Schiavetti et al. Cancer Res. |
| | MEL | | 62(19): 5510-6 (2002). |
| 7 | KK-LC-1 | RQKRILVNL (SEQ ID NO: 113) | Fukuyama et al. Cancer Res. |
| | | | 66(9): 4922-8 (2006). |
| 8 | KM-HN-1 | NYNNFYRFL (SEQ ID NO: 114) | Fukuyama et al. Cancer Res. |
| | | EYSKECLKEF (SEQ ID NO: 115) | 66(9): 4922-8 (2006). |
| | | EYLSLSDKI (SEQ ID NO: 116) | Monji et al. Clin Cancer Res. |
| | | | 10(18 Pt 1): 6047-57 (2004). |
| 9 | Sp17 | ILDSSEEDK (SEQ ID NO: 102) | Chiriva-Internati et al. Int J |
| | | | Cancer. 107(5): 863-5 (2003). |

TABLE 23
Brain cancer

| | Tumor- | | |
|---|---|---|---|
| | associated | | |
| No. | antigen | Immunogenic epitopes | Sources |
| 1 | TAG-1 | SLGWLFLLL (SEQ ID NO: 132) | Adair et al. J lmmunother. 31(1): 7-17 |
| | | LSRLSNRLL (SEQ ID NO: 133) | (2008). |
| 2 | TAG-2 | LSRLSNRLL (SEQ ID NO: 133) | Adair et al. J lmmunother. 31(1): 7-17 |
| | | | (2008). |

TABLE 24
Pharynx cancer

| | Tumor- | | |
|---|---|---|---|
| | associated | | |
| No. | antigen | Immunogenic epitopes | Sources |
| 1 | TAG-1 | SLGWLFLLL (SEQ ID NO: 132) | Adair et al. J lmmunother. 31(1): 7-17 |
| | | LSRLSNRLL (SEQ ID NO: 133) | (2008). |
| 2 | TAG-2 | LSRLSNRLL (SEQ ID NO: 133) | Adair et al. J lmmunother. 31(1): 7-17 |
| | | | (2008). |

TABLE 25
Tumors of the tongue

| | Tumor- | | |
|---|---|---|---|
| | associated | | |
| No. | antigen | Immunogenic epitopes | Sources |
| 1 | TAG-1 | SLGWLFLLL (SEQ ID NO: 132) | Adair et al. J Immunother. 31(1): 7-17 |
| | | LSRLSNRLL (SEQ ID NO: 133) | (2008). |
| 2 | TAG-2 | LSRLSNRLL (SEQ ID NO: 133) | Adair et al. J Immunother. 31(1): 7-17 |
| | | | (2008). |

TABLE 26
Synovial cell sarcoma

| | Tumor- | | |
|---|---|---|---|
| | associated | | |
| No. | antigen | Immunogenic epitopes | Sources |
| 1 | NY-ESO-1 | HLA-A2-restricted peptide p157-165 | Jager et al. Proc. Natl. Acad. |
| | | (SLLMWITQC) (SEQ ID NO: 32), HLA-Cw3- | Scie. U.S.A. 103(39): 14453-8 |
| | | restricted p92-100 (LAMP-FATPM) (SEQ ID | (2006). |
| | | NO: 33) and HLA-Cw6-restricted p80-88 | Gnjatic et al. PNAS |
| | | (ARGPESRLL) (SEQ ID NO: 34) | Sep. 26, 2000 vol. 97 no. |
| | | SLLMWITQC (SEQ ID NO: 32) | 20 p. 10919 |
| | | MLMAQEALAFL (SEQ ID NO: 35) | Jager et al. J Exp Med. |
| | | YLAMPFATPME (SEQ ID NO: 36) | 187(2): 265-70 (1998). |
| | | ASGPGGGAPR (SEQ ID NO: 37) | Chen et al. J Immunol. |
| | | LAAQERRVPR (SEQ ID NO: 38) | 165(2): 948-55 (2000). |
| | | TVSGNILTIR (SEQ ID NO: 39) | Valmori et al. Cancer Res. |
| | | APRGPHGGAASGL (SEQ ID NO: 40) | 60(16): 4499-506 (2000). |
| | | MPFATPMEAEL (SEQ ID NO: 41) | Aarnoudse et al. Int J Cancer. |
| | | KEFTVSGNILTI (SEQ ID NO: 42) | 82(3): 442-8 (1999). |
| | | MPFATPMEA (SEQ ID NO: 43) | Eikawa et al. Int J Cancer. |
| | | FATPMEAEL (SEQ ID NO: 44) | 132(2): 345-54 (2013). |
| | | FATPMEAELAR (SEQ ID NO: 45) | Wang et al. J Immunol. |
| | | LAMPFATPM (SEQ ID NO: 46) | 161(7): 3598-606 (1998). |
| | | ARGPESRLL (SEQ ID NO: 34) | Matsuzaki et al. Cancer |
| | | SLLMWITQCFLPVF (SEQ ID NO: 47) | Immunol Immunother. |
| | | LLEFYLAMPFATPMEAEL-ARRSLAQ (SEQ ID | 57(8)1185-95 (2008). |
| | | NO: 48) | Ebert et al. Cancer Res. |
| | | EFYLAMPFATPM (SEQ ID NO: 49) | 69(3): 1046-54 (2009). |
| | | PGVLLKEFTVSGNILTIRL-TAADHR (SEQ ID | Eikawa et al. Int J Cancer. |
| | | NO: 50) | 132(2): 345-54 (2013). |
| | | RLLEFYLAMPFA (SEQ ID NO: 51) | Knights et al. Cancer Immunol |
| | | QGAMLAAQERRVPRAAE-VPR (SEQ ID NO: | Immunother. 58(3): 325-38 |
| | | 52) | (2009). |
| | | PFATPMEAELARR (SEQ ID NO: 53) | Jäger et al. Cancer Immun. 2: 12 |
| | | PGVLLKEFTVSGNILTIRLT (SEQ ID NO: 54) | (2002). |
| | | VLLKEFTVSG (SEQ ID NO: 55) | Zeng et al. Proc Natl Acad Sci USA. |
| | | AADHRQLQLSISSCLQQL (SEQ ID NO: 56) | 98(7): 3964-9 (2001). |
| | | LKEFTVSGNILTIRL (SEQ ID NO: 57) | Mandic et al. J Immunol. |
| | | PGVLLKEFTVSGNILTIRL-TAADHR (SEQ ID | 174(3): 1751-9 (2005). |
| | | NO: 50) | Chen et al. Proc Natl Acad Sci USA. |
| | | LLEFYLAMPFATPMEAEL-ARRSLAQ (SEQ ID | 101(25): 9363-8 (2004). |
| | | NO: 48) | Ayyoub et al. Clin Cancer Res. |
| | | KEFTVSGNILT (SEQ ID NO: 58) | 16(18): 4607-15 (2010). |
| | | LLEFYLAMPFATPM (SEQ ID NO: 59) | Slager et al. J Immunol. |
| | | AGATGGRGPRGAGA (SEQ ID NO: 60) | 172(8): 5095-102 (2004). |
| | | | Mizote et al. Vaccine. |
| | | | 28(32): 5338-46 (2010). |
| | | | Jager et al. J Exp Med. |
| | | | 191(4): 625-30 (2000). |
| | | | Zarour et al. Cancer Res. |
| | | | 60(17): 4946-52 (2000). |
| | | | Zeng et al. J Immunol. |
| | | | 165(2): 1153-9 (2000). |
| | | | Bioley et al. Clin Cancer Res. |
| | | | 15(13): 4467-74 (2009). |
| | | | Zarour et al. Cancer Res. |
| | | | 62(1): 213-8 (2002). |
| | | | Hasegawa et al. Clin Cancer |
| | | | Res. 12(6): 1921-7 (2006). |
| 2 | LAGE-1 | MLMAQEALAFL (SEQ ID NO: 35) | Aarnoudse et al. Int J Cancer. |
| | | SLLMWITQC (SEQ ID NO: 32) | 82(3): 442-8 (1999). |
| | | LAAQERRVPR (SEQ ID NO: 38) | Rimoldi et al. J Immunol. |
| | | ELVRRILSR (SEQ ID NO: 117) | 165(12): 7253-61 (2000). |
| | | APRGVRMAV (SEQ ID NO: 118) | Wang et al. J Immunol. |
| | | SLLMWITQCFLPVF (SEQ ID NO: 47) | 161(7): 3598-606 (1998). |
| | | QGAMLAAQERRVPRAAEVP-R (SEQ ID NO: | Sun et al. Cancer Immunol |
| | | 119) | Immunother. 55(6): 644-52 |
| | | AADHRQLQLSISSCLQQL (SEQ ID NO: 56) | (2006). |
| | | CLSRRPWKRSWSAGSCPG-MPHL (SEQ ID | Slager et al. Cancer Gene Ther. |
| | | NO: 120) | 11(3): 227-36 (2004). |
| | | ILSRDAAPLPRPG (SEQ ID NO: 121) | Zeng et al. Proc Natl Acad Sci USA. |
| | | AGATGGRGPRGAGA (SEQ ID NO: 60) | 98(7): 3964-9 (2001). |
| | | | Slager et al. J Immunol. |
| | | | 172(8): 5095-102 (2004). |
| | | | Jager et al. J Exp Med. |
| | | | 191(4): 625-30 (2000). |
| | | | Slager et al. J Immunol. |
| | | | 170(3): 1490-7 (2003). |
| | | | Wang et al. Immunity. |
| | | | 20(1): 107-18 (2004). |
| | | | Hasegawa et al. Clin Cancer |
| | | | Res. 12(6): 1921-7 (2006). |
| 3 | HERV-K- | MLAVISCAV (SEQ ID NO: 112) | Schiavetti et al. Cancer Res. |
| | MEL | | 62(19): 5510-6 (2002). |
| 4 | KK-LC-1 | RQKRILVNL (SEQ ID NO: 113) | Fukuyama et al. Cancer Res. |
| | | | 66(9): 4922-8 (2006). |
| 5 | KM-HN-1 | NYNNFYRFL (SEQ ID NO: 114) | Fukuyama et al. Cancer Res. |
| | | EYSKECLKEF (SEQ ID NO: 115) | 66(9): 4922-8 (2006). |
| | | | EYLSLSDKI (SEQ ID NO: 116) |
| 6 | Sp17 | ILDSSEEDK (SEQ ID NO: 102) | Chiriva-Internati et al. Int J |
| | | | Cancer. 107(5): 863-5 (2003). |

TABLE 27
Neuroblastoma

| | Tumor- | | |
|---|---|---|---|
| | associated | | |
| No. | antigen | Immunogenic epitopes | Sources |
| 1 | NY-ESO-1 | HLA-A2-restricted peptide p157-165 | Jager et al. Proc. Natl. Acad. Scie. |
| | | (SLLMWITQC) (SEQ ID NO: 32), HLA-Cw3- | U.S.A. 103(39): 14453-8 (2006). |
| | | restricted p92-100 (LAMP-FATPM) (SEQ ID | Gnjatic et al. PNAS |
| | | NO: 33) and HLA-Cw6-restricted p80-88 | Sep. 26, 2000 vol. 97 no. 20 |
| | | (ARGPESRLL) (SEQ ID NO: 34) | p. 10919 |
| | | SLLMWITQC (SEQ ID NO: 32) | Jager et al. J Exp Med. |
| | | MLMAQEALAFL (SEQ ID NO: 35) | 187(2): 265-70 (1998). |
| | | YLAMPFATPME (SEQ ID NO: 36) | Chen et al. J Immunol. 165(2): 948-55 |
| | | ASGPGGGAPR (SEQ ID NO: 37) | (2000). |
| | | LAAQERRVPR (SEQ ID NO: 38) | Valmori et al. Cancer Res. |
| | | TVSGNILTIR (SEQ ID NO: 39) | 60(16): 4499-506 (2000). |
| | | APRGPHGGAASGL (SEQ ID NO: 40) | Aarnoudse et al. Int J Cancer. |
| | | MPFATPMEAEL (SEQ ID NO: 41) | 82(3): 442-8 (1999). |
| | | KEFTVSGNILTI (SEQ ID NO: 42) | Eikawa et al. Int J Cancer. |
| | | MPFATPMEA (SEQ ID NO: 43) | 132(2): 345-54 (2013). |
| | | FATPMEAEL (SEQ ID NO: 44) | Wang et al. J Immunol. |
| | | FATPMEAELAR (SEQ ID NO: 45) | 161(7): 3598-606 (1998). |
| | | LAMPFATPM (SEQ ID NO: 46) | Matsuzaki et al. Cancer Immunol |
| | | ARGPESRLL (SEQ ID NO: 34) | Immunother. 57(8)1185-95 (2008). |
| | | SLLMWITQCFLPVF (SEQ ID NO: 47) | Ebert et al. Cancer Res. |
| | | LLEFYLAMPFATPMEAEL-ARRSLAQ (SEQ | 69(3): 1046-54 (2009). |
| | | ID NO: 48) | Eikawa et al. Int J Cancer. |
| | | EFYLAMPFATPM (SEQ ID NO: 49) | 132(2): 345-54 (2013). |
| | | PGVLLKEFTVSGNILTIRL-TAADHR (SEQ ID | Knights et al. Cancer Immunol |
| | | NO: 50) | Immunother. 58(3): 325-38 (2009). |
| | | RLLEFYLAMPFA (SEQ ID NO: 51) | Jäger et al. Cancer Immun. 2: 12 |
| | | QGAMLAAQERRVPRAAE-VPR (SEQ ID NO: | (2002). |
| | | 52) | Zeng et al. Proc Natl Acad Sci USA. |
| | | PFATPMEAELARR (SEQ ID NO: 53) | A. 98(7): 3964-9 (2001). |
| | | PGVLLKEFTVSGNILTIRLT (SEQ ID NO: 54) | Mandic et al. J Immunol. |
| | | VLLKEFTVSG (SEQ ID NO: 55) | 174(3): 1751-9 (2005). |
| | | AADHRQLQLSISSCLQQL (SEQ ID NO: 56) | Chen et al. Proc Natl Acad Sci USA. |
| | | LKEFTVSGNILTIRL (SEQ ID NO: 57) | 101(25): 9363-8 (2004). |
| | | PGVLLKEFTVSGNILTIRL-TAADHR (SEQ ID | Ayyoub et al. Clin Cancer Res. |
| | | NO: 50) | 16(18): 4607-15 (2010). |
| | | LLEFYLAMPFATPMEAEL-ARRSLAQ (SEQ | Slager et al. J Immunol. |
| | | ID NO: 48) | 172(8): 5095-102 (2004). |
| | | KEFTVSGNILT (SEQ ID NO: 58) | Mizote et al. Vaccine. 28(32): 5338-46 |
| | | LLEFYLAMPFATPM (SEQ ID NO: 59) | (2010). |
| | | AGATGGRGPRGAGA (SEQ ID NO: 60) | Jager et al. J Exp Med. |
| | | | 191(4): 625-30 (2000). |
| | | | Zarour et al. Cancer Res. |
| | | | 60(17): 4946-52 (2000). |
| | | | Zeng et al. J Immunol. |
| | | | 165(2): 1153-9 (2000). |
| | | | Bioley et al. Clin Cancer Res. |
| | | | 15(13): 4467-74 (2009). |
| | | | Zarour et al. Cancer Res. |
| | | | 62(1): 213-8 (2002). |
| | | | Hasegawa et al. Clin Cancer Res. |
| | | | 12(6): 1921-7 (2006). |
| 2 | LAGE-1 | MLMAQEALAFL (SEQ ID NO: 35) | Aarnoudse et al. Int J Cancer. |
| | | SLLMWITQC (SEQ ID NO: 32) | 82(3): 442-8 (1999). |
| | | LAAQERRVPR (SEQ ID NO: 38) | Rimoldi et al. J Immunol. |
| | | ELVRRILSR (SEQ ID NO: 117) | 165(12): 7253-61 (2000). |
| | | APRGVRMAV (SEQ ID NO: 118) | Wang et al. J Immunol. |
| | | SLLMWITQCFLPVF (SEQ ID NO: 47) | 161(7): 3598-606 (1998). |
| | | QGAMLAAQERRVPRAAEVP-R (SEQ ID NO: | Sun et al. Cancer Immunol |
| | | 119) | Immunother. 55(6): 644-52 (2006). |
| | | AADHRQLQLSISSCLQQL (SEQ ID NO: 56) | Slager et al. Cancer Gene Ther. |
| | | CLSRRPWKRSWSAGSCPG-MPHL (SEQ ID | 11(3): 227-36 (2004). |
| | | NO: 120) | Zeng et al. Proc Natl Acad Sci USA. |
| | | ILSRDAAPLPRPG (SEQ ID NO: 121) | 98(7): 3964-9 (2001). |
| | | AGATGGRGPRGAGA (SEQ ID NO: 60) | Slager et al. J Immunol. |
| | | | 172(8): 5095-102 (2004). |
| | | | Jager et al. J Exp Med. |
| | | | 191(4): 625-30 (2000). |
| | | | Slager et al. J Immunol. |
| | | | 170(3): 1490-7 (2003). |
| | | | Wang et al. Immunity. 20(1): 107-18 |
| | | | (2004). |
| | | | Hasegawa et al. Clin Cancer Res. |
| | | | 12(6): 1921-7 (2006). |
| 3 | HERV-K- | MLAVISCAV (SEQ ID NO: 112) | Schiavetti et al. Cancer Res. |
| | MEL | | 62(19): 5510-6 (2002). |
| 4 | KK-LC-1 | RQKRILVNL (SEQ ID NO: 113) | Fukuyama et al. Cancer Res. |
| | | | 66(9): 4922-8 (2006). |
| 5 | KM-HN-1 | NYNNFYRFL (SEQ ID NO: 114) | Fukuyama et al. Cancer Res. |
| | | EYSKECLKEF (SEQ ID NO: 115) | 66(9): 4922-8 (2006). |
| | | EYLSLSDKI (SEQ ID NO: 116) | Monji et al. Clin Cancer Res. 10(18 |
| | | | Pt 1): 6047-57 (2004). |
| 6 | Sp17 | ILDSSEEDK (SEQ ID NO: 102) | Chiriva-Internati et al. Int J Cancer. |
| | | | 107(5): 863-5 (2003). |

TABLE 28
Uterine cancer

| | Tumor- | | |
|---|---|---|---|
| | associated | | |
| No. | antigen | Immunogenic epitopes | Sources |
| 1 | NY-ESO-1 | HLA-A2-restricted peptide p157-165 | Jager et al. Proc. Natl. Acad. Scie. |
| | | (SLLMWITQC) (SEQ ID NO: 32), HLA-Cw3- | U.S.A. 103(39): 14453-8 (2006). |
| | | restricted p92-100 (LAMP-FATPM) (SEQ | Gnjatic et al. PNAS |
| | | ID NO: 33) and HLA-Cw6-restricted p80-88 | Sep. 26, 2000 vol. 97 no. 20 |
| | | (ARGPESRLL) (SEQ ID NO: 34) | p. 10919 |
| | | SLLMWITQC (SEQ ID NO: 32) | Jager et al. J Exp Med. 187(2): 265-70 |
| | | MLMAQEALAFL (SEQ ID NO: 35) | (1998). |
| | | YLAMPFATPME (SEQ ID NO: 36) | Chen et al. J Immunol. 165(2): 948-55 |
| | | ASGPGGGAPR (SEQ ID NO: 37) | (2000). |
| | | LAAQERRVPR (SEQ ID NO: 38) | Valmori et al. Cancer Res. |
| | | TVSGNILTIR (SEQ ID NO: 39) | 60(16): 4499-506 (2000). |
| | | APRGPHGGAASGL (SEQ ID NO: 40) | Aarnoudse et al. Int J Cancer. |
| | | MPFATPMEAEL (SEQ ID NO: 41) | 82(3): 442-8 (1999). |
| | | KEFTVSGNILTI (SEQ ID NO: 42) | Eikawa et al. Int J Cancer. |
| | | MPFATPMEA (SEQ ID NO: 43) | 132(2): 345-54 (2013). |
| | | FATPMEAEL (SEQ ID NO: 44) | Wang et al. J Immunol. |
| | | FATPMEAELAR (SEQ ID NO: 45) | 161(7): 3598-606 (1998). |
| | | LAMPFATPM (SEQ ID NO: 46) | Matsuzaki et al. Cancer Immunol |
| | | ARGPESRLL (SEQ ID NO: 34) | Immunother. 57(8)1185-95 (2008). |
| | | SLLMWITQCFLPVF (SEQ ID NO: 47) | Ebert et al. Cancer Res. |
| | | LLEFYLAMPFATPMEAEL-ARRSLAQ (SEQ | 69(3): 1046-54 (2009). |
| | | ID NO: 48) | Eikawa et al. Int J Cancer. |
| | | EFYLAMPFATPM (SEQ ID NO: 49) | 132(2): 345-54 (2013). |
| | | PGVLLKEFTVSGNILTIRL-TAADHR (SEQ | Knights et al. Cancer Immunol |
| | | ID NO: 50) | Immunother. 58(3): 325-38 (2009). |
| | | RLLEFYLAMPFA (SEQ ID NO: 51) | Jäger et al. Cancer Immun. 2: 12 |
| | | QGAMLAAQERRVPRAAE-VPR (SEQ ID | (2002). |
| | | NO: 52) | Zeng et al. Proc Natl Acad Sci USA. |
| | | PFATPMEAELARR (SEQ ID NO: 53) | 98(7): 3964-9 (2001). |
| | | PGVLLKEFTVSGNILTIRLT (SEQ ID NO: | Mandic et al. J Immunol. |
| | | 54) | 174(3): 1751-9 (2005). |
| | | VLLKEFTVSG (SEQ ID NO: 55) | Chen et al. Proc Natl Acad Sci USA. |
| | | AADHRQLQLSISSCLQQL (SEQ ID NO: 56) | 101(25): 9363-8 (2004). |
| | | LKEFTVSGNILTIRL (SEQ ID NO: 57) | Ayyoub et al. Clin Cancer Res. |
| | | PGVLLKEFTVSGNILTIRL-TAADHR (SEQ | 16(18): 4607-15 (2010). |
| | | ID NO: 50) | Slager et al. J Immunol. |
| | | LLEFYLAMPFATPMEAEL-ARRSLAQ (SEQ | 172(8): 5095-102 (2004). |
| | | ID NO: 48) | Mizote et al. Vaccine. 28(32): 5338-46 |
| | | KEFTVSGNILT (SEQ ID NO: 58) | 46 (2010). |
| | | LLEFYLAMPFATPM (SEQ ID NO: 59) | Jager et al. J Exp Med. 191(4): 625-30 |
| | | AGATGGRGPRGAGA (SEQ ID NO: 60) | (2000). |
| | | | Zarour et al. Cancer Res. |
| | | | 60(17): 4946-52 (2000). |
| | | | Zeng et al. J Immunol. 165(2): 1153-9 |
| | | | (2000). |
| | | | Bioley et al. Clin Cancer Res. |
| | | | 15(13): 4467-74 (2009). |
| | | | Zarour et al. Cancer Res. |
| | | | 62(1): 213-8 (2002). |
| | | | Hasegawa et al. Clin Cancer Res. |
| | | | 12(6): 1921-7 (2006). |
| 2 | LAGE-1 | MLMAQEALAFL (SEQ ID NO: 35) | Aarnoudse et al. Int J Cancer. |
| | | SLLMWITQC (SEQ ID NO: 32) | 82(3): 442-8 (1999). |
| | | LAAQERRVPR (SEQ ID NO: 38) | Rimoldi et al. J Immunol. |
| | | ELVRRILSR (SEQ ID NO: 117) | 165(12): 7253-61 (2000). |
| | | APRGVRMAV (SEQ ID NO: 118) | Wang et al. J Immunol. |
| | | SLLMWITQCFLPVF (SEQ ID NO: 47) | 161(7): 3598-606 (1998). |
| | | QGAMLAAQERRVPRAAEVP-R (SEQ ID | Sun et al. Cancer Immunol |
| | | NO: 119) | Immunother. 55(6): 644-52 (2006). |
| | | AADHRQLQLSISSCLQQL (SEQ ID NO: 56) | Slager et al. Cancer Gene Ther. |
| | | CLSRRPWKRSWSAGSCPG-MPHL (SEQ | 11(3): 227-36 (2004). |
| | | ID NO: 120) | Zeng et al. Proc Natl Acad Sci USA. |
| | | ILSRDAAPLPRPG (SEQ ID NO: 121) | 98(7): 3964-9 (2001). |
| | | AGATGGRGPRGAGA (SEQ ID NO: 60) | Slager et al. J Immunol. |
| | | | 172(8): 5095-102 (2004). |
| | | | Jager et al. J Exp Med. 191(4): 625-30 |
| | | | (2000). |
| | | | Slager et al. J Immunol. |
| | | | 170(3): 1490-7 (2003). |
| | | | Wang et al. Immunity. 20(1): 107-18 |
| | | | (2004). |
| | | | Hasegawa et al. Clin Cancer Res. |
| | | | 12(6): 1921-7 (2006). |
| 3 | HERV-K- | MLAVISCAV (SEQ ID NO: 112) | Schiavetti et al. Cancer Res. |
| | MEL | | 62(19): 5510-6 (2002). |
| 4 | KK-LC-1 | RQKRILVNL (SEQ ID NO: 113) | Fukuyama et al. Cancer Res. |
| | | | 66(9): 4922-8 (2006). |
| 5 | KM-HN-1 | NYNNFYRFL (SEQ ID NO: 114) | Fukuyama et al. Cancer Res. |
| | | EYSKECLKEF (SEQ ID NO: 115) | 66(9): 4922-8 (2006). |
| | | EYLSLSDKI (SEQ ID NO: 116) | Monji et al. Clin Cancer Res. 10(18 |
| | | | Pt 1): 6047-57 (2004). |
| 6 | Sp17 | ILDSSEEDK (SEQ ID NO: 102) | Chiriva-Internati et al. Int J Cancer. |
| | | | 107(5): 863-5 (2003). |

Additional examples of TAAs are known in the art and are described, for example, in Reuschenbach et al., Cancer Immunol. Immunother. 58:1535-1544 (2009); Parmiani et al., J. Nat. Cancer Inst. 94:805-818 (2002); Zarour et al., Cancer Medicine. (2003); Bright et al., Hum. Vaccin. Immunother. 10:3297-3305 (2014); Wurz et al., Ther. Adv. Med. Oncol. 8:4-31 (2016); Criscitiello, Breast Care 7:262-266 (2012); Chester et al., J. Immunother. Cancer 3:7 (2015); Li et al., Mol. Med. Report 1:589-594 (2008); Liu et al., J. Hematol. Oncol. 3:7 (2010); Bertino et al., Biomed. Res. Int. 731469 (2015); and Suri et al., World J. Gastrointest. Oncol. 7:492-502 (2015).

The polynucleotides (minigenes), viral vectors and viral particles of the invention encode two or more epitopes (e.g., 2, 3, 4, 5, 6, 7, 8, 9, 10, 11, 12, 13, 14, 15, 16, 17, 18, 19, 20, 30, 40, 50, 60, 70, 80, 90, or more), of one or more tumor associated antigens that are expressed by a tumor or cancer cell present within a patient in need of treatment for a cancer or a tumor. In embodiments, the two or more TAA-derived epitopes and the tumor associated antigens suitable for use in the polynucleotide and virus vector and particle products, and the compositions and methods of the invention are those listed in any one of Tables 1-28. In embodiments, the polynucleotides (minigenes), viral vectors, viral particles, and pharmaceutical compositions of the invention encode multiple, e.g., two or more, epitopes of one or more tumor associated antigens that are sufficiently immunologically cross-reactive with one or more tumor associated antigens or epitopes thereof expressed by a cancer or tumor to elicit an immune response directed against the cancer or tumor expressing the TAA epitopes upon administration to a subject, such as a patient afflicted with a cancer or tumor.

Any tumor associated antigen (TAA) having epitopes and expressed by a cancer cell or solid tumor can be utilized in conjunction with the compositions and methods of the invention. However, it is expected that variability may exist in the efficacy of different TAAs and their associated epitopes to induce or increase an immune response in a subject, because some TAAs and/or their epitopes may potentially induce more robust responses (i.e., immunodominant TAAs). Relevant reports, e.g., preclinical and clinical study reports, can be used to guide the choice of TAAs or epitopes thereof to be incorporated into a polynucleotide (minigene), viral vector, viral particle, or pharmaceutical composition of the invention. In some embodiments, coding sequences of TAAs or the epitopes thereof that are capable of inducing a robust immune response, that bind MHC class I proteins with high affinity, or that bind MHC class II proteins with high affinity are incorporated into the polynucleotide, viral vector, viral particle, or pharmaceutical composition of the invention. By way of example, NY-ESO-1, the cancer-testis antigen, is desirable for use as a tumor associated antigen for cancer immunotherapy, because it is expressed in several different cancer and tumor types, e.g., breast cancer, lung cancer, melanoma, as well as in the testis and placenta; however, it is not expressed in other normal adult tissues.

A variety of resources are available to inform the skilled practitioner about the selection of TAAs or multiple epitopes thereof for use in the viral vector-based, anti-cancer therapeutics described herein. For example, the National Cancer Institute (NCI) formed a committee of experts to evaluate cancer antigen data from clinical trials performed over a 5-year period. The NCI committee formulated criteria and ranked the 75 representative TAAs using a weighted analytical hierarchy process (Cheevers et al., Clin Cancer Res., 15: 5323-5337, 2009). Those having skill in the pertinent art are familiar with the use of databases for the selection of TAAs or multiple epitopes thereof for inclusion in a polynucleotide, viral vector, viral particle, or pharmaceutical composition of the invention. Such references include, without limitation, van der Bruggen P. et al., Peptide database: T cell-defined tumor antigens. Cancer Immun, 2013. URL: http://www.cancerimmunity.org/peptide/; Vigneron et al. Cancer Immun. 2013; 13: 15; TANTIGEN: Tumor T cell Antigen Database, http://cvc.dfci.harvard.edu/tadb/; HPtaa database, http://www.bioinfo.org.cn/hptaa/; Backert, L. and Kohlbacher, O., 2015, Genome Medicine, 7:119; Nielsen, M. et al., 2010, Immunology, 130(3):319-328; Wang, P. et al., 2008, PLoS Comput. Biol., 44(4):e10000048; Wang, P. et al., 2010, BMC Bioinformatics, 11:568; Chang, S. T. et al., 2006, Bioinformatics, 22(22):2761-2767; Guillaume, P. et al., 2009, Cancer Immun. (http://www.cancerimmunity.org/tetramers/); Chen, Y. T. et al., 2000, In: Rosenberg, S. A., Ed., Principles and practice of the biologic therapy of cancer, 3^{rd} ed. Philadelphia, Pa.: Lippincott Williams & Wilkins, pp. 557-570. In addition to available publications, putative epitopes can also be analyzed for binding strength to T cell receptors using the algorithms available at different web-based sources presented in Table 29 below. An example of the use of the algorithms listed in Table 29 for epitope selection is set forth in Example 6, infra.

In a more personalized vaccine approach, the tumor associated antigens, and epitopes thereof, expressed by a patient's tumor can be identified from a biopsy or from a biological sample of the patient when a biopsy is not possible. A biological sample obtained from a subject (patient) may include, without limitation, blood, serum, plasma, urine, feces, sputum, saliva, tears, cerebrospinal fluid, peritoneal fluid, skin, tissue, cells, scrapings of tissue and skin, and processed, e.g., homogenized or reconstituted, forms thereof. Serological analysis of cDNA expression libraries (SEREX) has previously been used to identify human TAAs. A subject's serum sample can also be tested against panels of known TAA proteins by using either ELISA or Western blot assays. Epitopes of TAAs identified from the subject's serum can be further tested for the capacity to stimulate effector activity of the patient's T cells using methods known in the art, such as Elispot assays that measure T cell activation.

TABLE 29
URLs of Algorithms to Rank HLA/MHC Epitope Binding

| Name | URL |
|---|---|
| ANNPRED | http://www.imtech.res.in/raghava/nhlapred/ |
| | neural.html |
| BIMAS | http://www-bimas.cit.nih.gov/molbio/hla_bind/ |
| EPIMHC | http://imed.med.ucm.es/epimhc/ |
| HLABIND | http://atom.research.microsoft.com/hlabinding/ |
| | hlabinding.aspx |
| IEDB | http://tools.immuneepitope.org/analyze/html/ |
| | mhc_binding.html |
| KISS | http://cbio.ensmp.fr/kiss |
| MOTIF_SCAN | http://www.hiv.lanl.gov/content/immunology/ |
| | motif_scan/motif_scan |
| MULTIPRED | http://antigen.i2r.a-star.edu.sg/multipred/ |
| NetMHC | http://www.cbs.dtu.dk/services/NetMHC/ |
| NetMHCpan | http://www.cbs.dtu.dk/services/NetMHCpan/ |
| PEPVAC | http://imed.med.ucm.es/PEPVAC/ |
| POPI | http://iclab.life.nctu.edu.tw/POPI/ |
| PREDEP | http://margalit.huji.ac.il/Teppred/mhc-bind/ |
| | index.html |
| RANKPEP | http://imed.med.ucm.es/Tools/rankpep.html |
| SVMHC | http://www-bs.informatik.uni-tuebingen.de/SVMHC/ |
| SVRMHC | http://SVRMHC.umn.edu/SVRMHCdb |
| SYFFPEITHI | http://www.syfpeithi.de/Scripts/MHCServer.dll/ |
| | EpitopePrediction.htm |

### Epitope Selection

In general, CD8⁺ cytotoxic T cells are programmed to recognize peptides (epitope amino acid sequences) associated with the MHC class I molecules on all nucleated cells. These peptides or epitopes have certain general characteristics. Typically, epitopes that are capable of eliciting a CD8⁺ T cell response are amino acid sequences or peptides that bind to MHC class I molecules and are about 3-50 amino acids in length, or about 3-30 amino acids in length, or about 5-30 amino acids in length, or about 5-25 amino acids in length, or about 7-20 amino acids in length, or about 8-10 amino acids in length. Without wishing to be bound by theory, the epitopic peptide lies in an elongated conformation along the MHC class I peptide-binding groove. However, variations in peptide length appear to be accommodated, in most cases, by a kinking in the peptide backbone. Therefore, some length variation in CD8⁻ T cell activating epitopes is possible.

Epitopes that are capable of eliciting a CD4⁺ T cell response are typically peptides (epitope amino acid sequences) that bind to MHC class II molecules. Peptides that bind to MHC class II molecules are at least 13 amino acids in length and can be much longer. The epitopic peptide lies in an extended conformation along the MHC class II peptide-binding groove. It is held in this groove both by peptide side chains that protrude into shallow and deep pockets lined by polymorphic residues and by interactions between the peptide backbone and the side chains of conserved amino acids that line the peptide-binding cleft in all MHC class II molecules. Because the peptide is bound by its backbone and allowed to emerge from both ends of the binding groove there is, in principle, no upper limit to the length of peptides that could bind to MHC class II molecules. However, longer peptides bound to MHC class II molecules are typically trimmed by peptidases to a length of 13-17 amino acids in most cases.

While selection of epitopes expected to elicit a T cell response can be guided by the literature, databases (Vigneron, N. et al., 2013, Database of T cell-defined tumor antigens. Cancer Immun., Vol. 13; and the Immune Epitope Database) and in silico algorithms (Table 29), such approaches are not intended to be limiting, and any means of detecting TAA epitopes generally consistent with the above description of epitopes found in association with tumor cells can be used. Databases curate data from the literature that indicate whether epitopes have been successful in eliciting immune responses. Many epitope prediction algorithms are available, some of which are listed in Table 29. Computer programs using various criteria are available to analyze amino acid sequences for peptide regions that are most likely to bind MHC receptors and T cells, including structure, physicochemical properties, flexibility, charge and protease processing (Yang and Yu, 2009, Rev. Med. Virol., 19:77-96). Amino acid sequences of tumor associated antigen proteins can be analyzed using several algorithms to find the best consensus epitopes for eliciting anti-cancer/anti-tumor immune responses. An example of the use of epitope prediction algorithms to select epitopes for use in the present invention is set forth in Example 6, infra.

Experimental binding assays, such as the iTopia Epitope Discovery System (Beckman Coulter) further refine the selection of epitopes. The iTopia screening assay allows for prioritization of predicted epitopes based on MHC binding affinity and peptide MHC complex stability. Epitopes restricted to HLA alleles that are present in the population at high frequencies can be chosen to broaden the applicability of the TAA-derived epitopes included in the polynucleotides (minigenes), viral vectors, viral particles, and compositions described herein. Frequencies of HLA I and HLA II alleles are compiled for worldwide populations and are available to the skilled practitioner, e.g., at www.allelefrequencies.net; bioinformatics.bethematchclinical.org. When several epitopes for a given TAA are under consideration, it may be useful to select those TAA epitopes that bind to the most frequent HLA alleles to allow for personalized treatment of an individual patient.

### Polynucleotides Encoding Epitopes of Tumor Associated Antigens and Other Polypeptides

The polynucleotides (minigenes) as described for incorporation into Sindbis viral vectors, for example, may further include sequences encoding molecules that augment peptide epitope-MHC interactions. For example, calreticulin and calnexin represent integral proteins in the production of MHC class I Proteins. Calnexin binds to newly synthesized MHC class I α-chains as they enter the endoplasmic reticulum, thus retaining them in a partly folded state. After β₂-microglobulin binds to the peptide-loading complex (PLC), calreticulin (along with ERp57) takes over the function of chaperoning the MHC class I protein, while tapasin links the complex to the transporter associated with antigen processing (TAP) complex. This association prepares the MHC class I molecule for binding an antigen for presentation on the cell surface. Thus, a Sindbis viral replicon particle can be constructed that encodes calreticulin (CRT) linked to the polynucleotide encoding multiple epitopes of one or more tumor associated antigens.

By way of example, a polynucleotide (minigene) can be constructed via polymerase chain reaction (PCR) using a series of overlapping DNA oligomer primers in a process known as gene ‘Splicing by Overlap Extension’ or gene “SOEing” (Horton, R. M., et al., 2013. BioTechniques, 8(5):528-535; (November 1990); Horton et al., Biotechniques. 2013; 54:129-133). Furin processing of multi-epitope polypeptides efficiently induces T cell activation. As Sindbis virus polypeptides are naturally processed by furin, the polynucleotides (minigenes), viral vectors, viral particles, and pharmaceutical compositions of the invention are designed to include furin cleavage sites to separate the multiple epitope coding sequences. For instance, compositions of the invention may include the Sindbis furin digestion sequence XRSKRX, (SEQ ID NO: 5), in which X designates a hydrophobic residue. Non-limiting examples of additional processing enzymes for use in cleaving the epitope peptides encoded by the polynucleotides and viral vectors according to the present invention include furin related endopeptidases, such as PC1/2, PC4/5, PACE4, and PC7. These enzymes recognize the processing signal (R/K)Xₙ(R/K), in which Xₙ designates a spacer of any 0-6 amino acids, (SEQ ID NO: 6), (Seidah and Prat, 2012, Nature Reviews Drug Discovery, 11:367-383). Nucleic acid sequences encoding contiguous epitopes (Thompson et al., 1998, J. Immunol., 160:1717-23) or epitopes with spacers, such as AAA or GGG, may be included in the polynucleotide (minigene) or viral vectors described herein, thus allowing for cellular processing. In some embodiments, a polynucleotide, viral vector, or pharmaceutical composition of the invention encodes contiguous epitopes without enzyme cleavage sites or spacers.

The cysteine protease cathepsin S (CAT S) is also suitable for use in the proteolytic processing of the peptides and polypeptides encoded by the polynucleotide (minigene) or viral vector of the invention. CAT S is located in the endosomal compartment of antigen presenting cells, such as dendritic cells, macrophages, and B-lymphocytes, and may play a role in antigen processing for presentation, particularly on MHC II. The endolytic cleavage sites for CAT S are PMGAP ((SEQ ID NO: 270) and PMGLP (SEQ ID NO: 271).

A tumor associated antigen-derived epitope peptide encoded by the polynucleotides (minigenes) or viral vectors of the invention may contain, for example, from 5-50 amino acid residues. In embodiments, the epitopes of the tumor associated antigen comprise 5-30 amino acid residues, 5-25 amino acid residues, 5-20 amino acid residues, 7-25 amino acid residues, 7-20 amino acid residues, or 7-14 amino acid residues. By way of nonlimiting example, a polynucleotide of the invention encode from 21 to 42 residues. Since approximately 3700 nucleotides encoding Sindbis structural genes are removed from a replicon vector during the production of a Sindbis virus vector encoding multiple epitopes of one or more tumor associated antigens, it is estimated that from about 60 to 90 epitope-encoding sequences flanked by furin-cleavage sites can be inserted into a viral vector of the invention, e.g., a pT7StuI-R/epitope vector as described herein.

### Polynucleotides and Viral Vectors Encoding Multiple Epitopes of One or More Tumor Associated Antigens

In some embodiments, a viral vector, viral particle, or pharmaceutical composition containing a polynucleotide (minigene) that encodes two or more epitopes of one or more tumor associated antigens, in which the epitopes induce a robust immune response (such as a humoral or cell-mediated immune response) is provided. In an embodiment, the polynucleotide encodes an alphavirus protein, or a fragment thereof as described herein. In an embodiment, the polynucleotide encodes a Sindbis virus protein, or a fragment thereof as described herein. The immune response elicited may be assessed, for example, by determining the antibody titer generated against the tumor associated antigen or the extent of TAA-mediated T-cell activation in a patient in vivo, or in a biological sample obtained from the patient. Methods of selecting tumor associated antigens and epitopes thereof that induce a robust humoral or cell-mediated immune response and that may be incorporated into the polynucleotides, viral vectors, viral particles, or compositions of the invention are described in further detail herein.

In certain embodiments, and without wishing to be limiting, a polynucleotide (minigene), polynucleotide, viral vector, virus particle, or pharmaceutical composition of the invention contains a polynucleotide that encodes two or more epitopes of one or more of the following tumor associated antigens NY-ESO-1, CEA, k-Ras, c-myc, HPV E6, HPV E7, cyclin B1, Her2, MUC1, p53, p62, survivin, WT1, sp17, and Pdz-Binding Kinase (PBK). For example, in some embodiments, the polynucleotide (minigene), viral vector, virus particle, or pharmaceutical composition comprises a polynucleotide that encodes one or more epitopes of the tumor associated antigen NY-ESO-1 (e.g., an epitope of NY-ESO-1 listed in any one of Tables 1-28), and one or more epitopes of the tumor associated antigen CEA (e.g., an epitope of CEA listed in any one of Tables 1-28). In some embodiments, the polynucleotide (minigene), viral vector, virus particle, or pharmaceutical composition comprises a polynucleotide that encodes one or more epitopes of NY-ESO-1 (e.g., an epitope of NY-ESO-1 listed in any one of Tables 1-28), and one or more epitopes of the tumor associated antigen k-Ras (e.g., an epitope of k-Ras listed in any one of Tables 1-28). In some embodiments, the polynucleotide (minigene), viral vector, virus particle, or pharmaceutical composition comprises a polynucleotide that encodes one or more epitopes of NY-ESO-1 (e.g., an epitope of NY-ESO-1 listed in any one of Tables 1-28), and one or more epitopes of the tumor associated antigen c-myc. In some embodiments, the polynucleotide (minigene), viral vector, virus particle, or pharmaceutical composition comprises a polynucleotide that encodes one or more epitopes of NY-ESO-1 (e.g., an epitope of NY-ESO-1 listed in any one of Tables 1-28), and one or more epitopes of cyclin B1. In some embodiments, the polynucleotide (minigene), viral vector, virus particle, or pharmaceutical composition comprises a polynucleotide that encodes one or more epitopes of NY-ESO-1 (e.g., an epitope of NY-ESO-1 listed in any one of Tables 1-28), and one or more epitopes of Her2 (e.g., an epitope of Her2 listed in any one of Tables 1-28). In some embodiments, the polynucleotide (minigene), viral vector, virus particle, or pharmaceutical composition comprises a polynucleotide that encodes one or more epitopes of NY-ESO-1 (e.g., an epitope of NY-ESO-1 listed in any one of Tables 1-28), and one or more epitopes of MUC1. In some embodiments, the polynucleotide (minigene), viral vector, virus particle, or pharmaceutical composition comprises a polynucleotide that encodes one or more epitopes of NY-ESO-1 (e.g., an epitope of NY-ESO-1 listed in any one of Tables 1-28), and one or more epitopes of p53 (e.g., an epitope of p53 listed in any one of Tables 1-28). In some embodiments, the polynucleotide (minigene), viral vector, virus particle, or pharmaceutical composition comprises a polynucleotide that encodes one or more epitopes of NY-ESO-1 (e.g., an epitope of NY-ESO-1 listed in any one of Tables 1-28), and one or more epitopes of p62. In some embodiments, the polynucleotide (minigene), viral vector, virus particle, or pharmaceutical composition comprises a polynucleotide that encodes one or more epitopes of NY-ESO-1 (e.g., an epitope of NY-ESO-1 listed in any one of Tables 1-28), and one or more epitopes of survivin or an epitope thereof. In some embodiments, the polynucleotide (minigene), viral vector, virus particle, or pharmaceutical composition comprises a polynucleotide that encodes one or more epitopes of NY-ESO-1 (e.g., an epitope of NY-ESO-1 listed in any one of Tables 1-28), and one or more epitopes of WT1 (e.g., an epitope of WT1 listed in any one of Tables 1-28). In some embodiments, the polynucleotide (minigene), viral vector, virus particle, or pharmaceutical composition comprises a polynucleotide that encodes one or more epitopes of NY-ESO-1 (e.g., an epitope of NY-ESO-1 listed in any one of Tables 1-28), and one or more epitopes of sp17 (e.g., an epitope of sp17 listed in any one of Tables 1-28). In some embodiments, the polynucleotide (minigene), viral vector, virus particle, or pharmaceutical composition comprises a polynucleotide that encodes one or more epitopes of NY-ESO-1 (e.g., an epitope of NY-ESO-1 listed in any one of Tables 1-28), and one or more epitopes of gp70. In some embodiments, the polynucleotide (minigene), viral vector, virus particle, or pharmaceutical composition comprises a polynucleotide that encodes one or more epitopes of NY-ESO-1 (e.g., an epitope of NY-ESO-1 listed in any one of Tables 1-28) and one or more epitopes of pbk (a PDZ binding kinase that is overexpressed in many tumors). In some embodiments, the polynucleotide (minigene), viral vector, virus particle, or pharmaceutical composition comprises a polynucleotide that encodes one or more epitopes of NY-ESO-1 (e.g., an epitope of NY-ESO-1 listed in any one of Tables 1-28) and one or more epitopes of survivin.

In other embodiments, the polynucleotide (minigene), viral vector, virus particle, or pharmaceutical composition comprises a polynucleotide that encodes one or more epitopes of NY-ESO-1 (e.g., an epitope of NY-ESO-1 listed in any one of Tables 1-28), one or more epitopes of p53 (e.g., an epitope of p53 listed in any one of Tables 1-28), one or more epitopes of sp17 (e.g., an epitope of sp17 listed in any one of Tables 1-28), one or more epitopes of survivin, and one or more epitopes of WT1 (e.g., an epitope of WT1 listed in any one of Tables 1-28). In some embodiments, the polynucleotide (minigene), viral vector, virus particle, or pharmaceutical composition comprises a polynucleotide that encodes one or more epitopes of NY-ESO-1 (e.g., an epitope of NY-ESO-1 listed in any one of Tables 1-28), one or more epitopes of gp70, and one or more epitopes of pbk, e.g., as described in Example 2, infra.

### Viruses and Viral Vectors

### Alphavirus, Sindbis Virus and Sindbis Virus Vectors

Alphaviruses belong to the group IV Togaviridae family of viruses that are small, spherical, enveloped, positive-sense, single-stranded RNA viruses. Most alphaviruses infect and replicate in vertebrate hosts and in hematophagous arthropods, such as mosquitoes. Alphavirus virions are spherical with an iscoahedral nucleocapsid enclosed in a lipid-protein envelope. Alphavirus RNA is a single 42S strand of approximately 4×10⁶ daltons that is capped and polyadenylated. The alphavirus envelope comprises a lipid bilayer derived from the host cell plasma membrane and contains two viral glycoproteins, E1 (48,000 daltons) and E2 (52,000 daltons). A third, small E3 protein (10,000-12,000 daltons) is released from the virus as a soluble protein in alphaviruses other than Semliki Forest virus, where the E3 protein remains virus-associated.

As described herein, polynucleotides encoding an alphavirus protein, or a fragment thereof, and two or more epitopes of one or more tumor associated antigens, wherein each epitope is separated by an enzyme cleavage site are embraced by the invention. In addition, the present invention encompasses viral vectors and particles that are pseudotyped with proteins, e.g., envelope proteins, from other virus types. The polynucleotides, viral vectors and viral particles described herein encompass nucleic acid sequences and polypeptide sequences of members of the Alphavirus genus, including various strains, antigenic complexes, species and subtypes. Encompassed by the invention are alphaviruses, phylogenetically related alphaviruses, alphavirus complexes, and their structural components, such as envelope proteins, e.g., E1, as described, for example, in Powers, A. M. et al., 2011, J. Virol., 75(21):10118-10131. Nonlimiting examples of alphaviruses, and polynucleotides and proteins thereof, as well as fragments of their polynucleotides and proteins, that may be used in the polynucleotides, viral vectors and viral particles as described herein include Barmah Forest virus, Barmah Forest virus complex, Eastern equine encephalitis virus (EEEV), Eastern equine encephalitis virus complex, Middelburg virus, Middelburg virus complex, Ndumu virus, Ndumu virus complex, Semliki Forest virus, Semliki Forest virus complex, Bebaru virus, Chikungunya virus, Mayaro virus, Subtype Una virus, O′Nyong Nyong virus, Subtype Igbo-Ora virus, Ross River virus, Subtype Getah virus, Subtype Bebaru virus, Subtype Sagiyama virus, Subtype Me Tri virus, Venezuelan equine encephalitis virus (VEEV), VEEV complex, Cabassou virus, Everglades virus, Mosso das Pedras virus, Mucambo virus, Paramana virus, Pixuna virus, Western equine encephalitis virus (WEEV), Rio Negro virus, Trocara virus, Subtype Bijou Bridge virus, Western equine encephalitis virus complex, Aura virus, Babanki virus, Kyzylagach virus, Sindbis virus, Ockelbo virus, Whataroa virus, Buggy Creek virus, Fort Morgan virus, Highlands J virus, Eilat virus, Salmon pancreatic disease virus (SPDV), Southern elephant seal virus (SESV), Tai Forest virus and Tonate virus.

As an alphavirus, Sindbis virus is a small, enveloped, positive-sense, single strand RNA virus. Other members of the alphavirus genus include, without limitation, Semliki Forest virus (SFV), Venezuelan equine encephalitis virus (VEEV) and Ross River Virus (RRV). Alphaviruses, including Sindbis virus, form spherical particles of 60-70 nm in diameter; the icosahedral structures of many alphaviruses have been defined to very high resolutions by cryo-electron microscopy (cryo-EM) and crystallographic studies, revealing details of the interactions between the structural proteins (Jose, J. et al., 2009, Future Microbiol., 4:837-856). The genome is composed of a single strand of positive-sense RNA that is approximately 11 to 12 kb in length and encodes four nonstructural proteins (nsP1-nsP4) involved in virus replication and pathogenesis, and five structural proteins that compose the virion particle, i.e., the nucleocapsid protein C and the envelope proteins, P62 (proteolytically cleaved into the mature envelope proteins E2 and E3) and the E1 protein. Alphaviruses exhibit efficient replication and have broad range of susceptible and permissive hosts; therefore, these viruses are highly suitable for heterologous gene expression and as gene therapy delivery vectors. Alphavirus vectors are suitable for use in encoding the polynucleotides (minigenes) for delivering the multi-epitopes of tumor associated antigens as described herein.

Any Sindbis viral vector is suitable for use in conjunction with the polynucleotides, virus vectors, compositions and methods of the present invention, including replication-competent vectors (see, e.g., U.S. Pat. No. 8,282,916) and replication-defective vectors (see, e.g., U.S. Pat. Nos. 7,303,898, 7,306,792, and 8,093,021). Replication-defective vectors are preferred for use in the present invention, as they offer another layer of protection against infection of healthy tissues. Sindbis vectors can also be constructed to contain more than one subgenomic promoter to express more than one gene using methods known in the art.

By way of example, to produce the pT7StuI-R/epitope vector, the replicon plasmid encoding the Sindbis replicase genes (nsP1-nsP4) and a helper plasmid, encoding the viral structural genes (capsid protein C, E1, E2, E3, and 6K), were transcribed in vitro. To limit viral replication in vivo, the replicon genes have been separated from the structural genes, which additionally contain a mutated packaging signal to prevent incorporation into virus particles (Bredenbeek, P. J. et al., 1993, J Virol 67: 6439-6446). Virus particles were produced by transient transfection of baby hamster kidney (BHK) cells with in vitro synthesized Sindbis replicon RNA and helper RNA transcripts. Within the cell, genomic RNA was replicated by the Sindbis replicase and expressed from the capped replicon RNA transcript. Structural proteins were expressed from the helper RNA transcript. Only the replicon RNA was packaged into the capsid to form the nucleocapsid, which then associates with the viral glycoproteins E1 and E2 and buds out of the cell. The resulting virion contained the capped SV single-stranded RNA message for nsP1-nsP4 genes, which encode the viral replicase, a subgenomic promoter (Psg) from which the replicase can transcribe an inserted gene of interest and a poly A tail.

To formulate a Sindbis viral vector encoding multiple TAA epitopes (“SV/TAA”) and exhibiting the potential to stimulate an anti-tumor T cell repertoire, a polynucleotide (e.g., a DNA minigene) encoding multiple T cell recognition epitopes, each separated by enzyme cleavage sites, was inserted into a Sindbis vector (e.g., pT7StuI-R LacZ#202; U.S. Pat. No. 8,093,021). Because SV/TAA virions induce a strong innate immune response and express TAA epitopes that activate CD8+ T cells, the viral vectors of the invention do not require signal and immunogenic peptides, although such peptide may be included, if desired. If desired, vectors can be readily manipulated to include immune-enhancing elements as described below.

### Lentivirus

Lentiviral vectors are particularly useful for long-term expression of genes, as they have the ability to infect both dividing and non-dividing cells. Third generation lentiviral systems are preferred for increased safety (Breckpot, K., et al., 2007, Gene Ther, 14: 847-862). These include, e.g., a transfer plasmid into which nucleic acid sequences encoding two or more epitopes of a tumor associated antigen is inserted, a packaging plasmid for gag and pol genes and another packaging plasmid for the rev gene. For optimal expression, the transfer expression vectors contain a splice donor, a packaging signal (psi), a Rev-responsive element (RRE), splice acceptor, central poly-purine tract (cPPT), and Wood chuck hepatitis virus transcriptional response element (WPRE) (Shaw and Cornetta, 2014, Biomedicines, 2:14-35). Transfer vector constructs may also contain a promoter for expression in mammalian cells. Constitutive promoters, such as the cytomegalovirus (CMV), mammalian beta-actin, or ubiquitin promoters may be incorporated into a composition of the invention. In some embodiments, tissue-specific promoters are utilized, such as CD4+ T cell-specific promoters.

Plasmids for generating lentiviral vectors can be obtained from Addgene (Cambridge, Mass., a non-profit plasmid repository) and modified, as necessary, using standard techniques in the art. Standard 3^{rd} generation packaging plasmids can be used. Suitable transfer vectors include, for example, pLX301, pFUGW, and pWPXL. These vectors contain all of the requisite characteristics mentioned above. To increase safety, the lentivirus transfer vectors can be mutated to decrease integration and increase episomal replication in infected cells. For instance, using standard techniques known in the field, the following modifications can be performed: a deletion within the U3 region of the 3′ LTR to create a self-inactivating LTR (SIN-LTR) is made; LTR att sites within the U3 and U5 LTR regions are deleted or mutated; the 3′ LTR-proximal polypurine tract (PPT) are deleted or modified (Shaw and Cornetta, 2014).

Pseudotyped viral vectors and virions are also suitable for use in connection with the polynucleotides and compositions of the invention. Such virions contain a viral particle and one or more foreign virus envelope proteins. (D.A. Sanders, 2002, Curr. Opin. Biotechnol., 13:437-442). In some embodiments, a viral vector of the invention may be a lentivirus containing an alphavirus protein or a fragment thereof, e.g., an envelope protein or a functional fragment thereof. In some embodiments, a viral vector of the invention may be a lentivirus containing a Sindbis virus envelope glycoprotein, or certain Sindbis virus envelope glycoproteins. By way of example, to produce a construct (e.g., a pseudotyped viral vector) comprising a lentivirus backbone pseudotyped with one or more Sindbis envelope proteins, a Sindbis envelope plasmid, e.g., T7 DM helper #101 (U.S. Pat. No. 8,093,021) is transfected into BHK or 293 cells along with the lentiviral plasmids resulting in pseudotyped virions.

### Retrovirus

Retroviral vectors are also suitable for use according to the invention. In some embodiments, the retroviral vector is Moloney murine leukemia virus (Mo-MuLV) pseudotyped with Sindbis envelope proteins. Pseudotyping can be performed using methods known in the art (see, e.g., Sharkey et al., 2001, J. Virology, 75(6):2653-2659). In some embodiments, the Mo-MuLV-based retrovirus particles are engineered to include and express the glycoproteins of the alphavirus Ross River virus (RRV) using methods known and practiced in the art.

### Sindbis Virus Envelope Pseudotyped Vectors

The Sindbis virus (SV) envelope is advantageous for use as a gene or polynucleotide delivery vector. SV is a blood-borne virus with a relatively long half-life. Stable virus is easily produced and can be concentrated for administration. Modification of the Sindbis E2 envelope protein, which binds to cell surface molecules, does not affect the E1 fusogenic envelope protein that is required for cell entry, thus allowing for engineered targeting of the virus. Sindbis virus specifically targets tumors by interacting with the high-affinity laminin receptor (LAMR) (U.S. Pat. No. 7,306,792), which is over-expressed by many tumors, and does not infect normal tissues. As a blood-borne virus, Sindbis virus is capable of contacting disseminated metastatic tumor cells via the bloodstream.

Sindbis viral envelope structural proteins can pseudotype other viral vectors, such as lentivirus, retrovirus and Vesicular Stomatitis virus (VSV) to improve their targeting capabilities and increase virion stability. In particular, the Sindbis-ZZ protein, designed to contain the Fc binding domain of S. aureus protein A inserted into the E2 envelope protein (U.S. Pat. No. 6,432,699), is useful in conjunction with cell surface specific antibodies for redirecting the targeting of SV and other vectors.

In certain embodiments in which long-term, stable expression of multiple epitopes is desired, retroviral or lentiviral vectors pseudotyped with wild type or engineered Sindbis virus envelope proteins are employed. Lentiviral vectors are advantageous for infection of both dividing and non-dividing cells. Like the Sindbis virus genome, the lentivirus genome can be split into two or three vectors, and genes can be modified or deleted to improve safety. A retrovirus subtype lentivirus naturally integrates into the host genome. However, vectors containing either long terminal repeats (LTR) or integrase enzyme mutations can exist as stable, non-integrating episomes in the cell nucleus (Breckpot, K., et al., 2007, Gene Ther., 14:847-862).

### Enhancement of Immunogenicity of the Described Viral Vectors, e.g., Sindbis Viral Vector

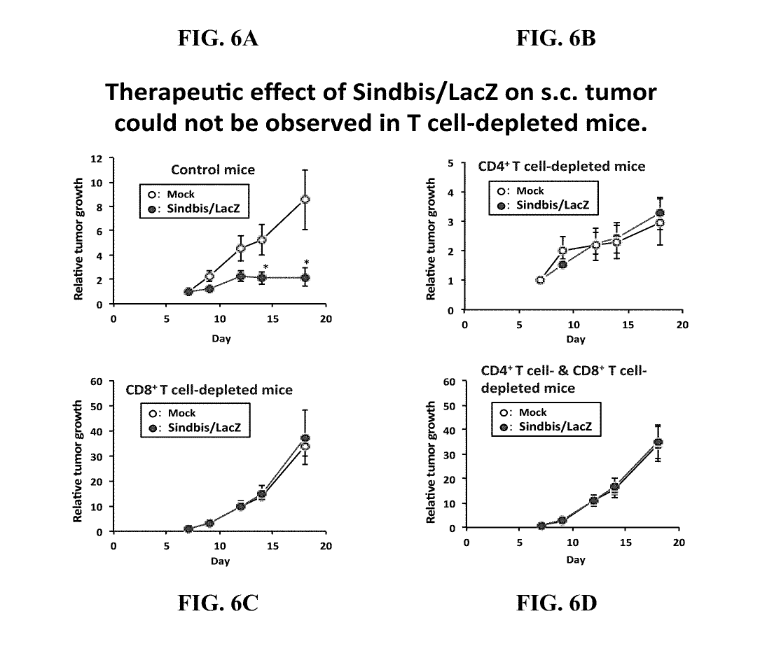

Augmentation of the immune response elicited by the multiple TAA-associated epitopes encoded by the viral vectors described herein, such as the pT7StuI-R/epitope vector, is encompassed by the invention. For example, promoting an increase in CD4⁺ T cells (T cell help) can enhance cross-presentation of tumor antigens and stimulate the production of CD8+ memory T cells. Indeed, an immune response and anti-cancer therapy provided by a Sindbis viral vector encoding multiple epitopes of one or more tumor associated antigens (SV/TAA) was obviated when mice were depleted of CD4 T cells (FIG. 6A-6D).

The Pan HLA-DR reactive epitope, AKFVAAWTLKAAA (PADRE), (SEQ ID NO: 7), is capable of generating antigen-specific CD4+ T cells that bind various HLA class II molecules with high affinity to stimulate T cell help (Alexander, J. et al., 1994, Immunity, 1:751-761). In certain embodiments, the polynucleotide (minigene), viral vector, or viral particle of the invention contains a sequence encoding the PADRE epitope in addition to sequences encoding multiple, e.g., two or more, epitopes of one or more tumor associated antigens in which the epitope sequences are separated by processing sites such as enzyme cleavage sites. In addition, sequences encoding cognate CD4⁺ T cell epitopes and sequences encoding CD8+ T cell epitopes can be included in the polynucleotides and the viral vectors to potentiate efficacy.

Inclusion of an endoplasmic reticulum (ER) signal sequence can facilitate multi-epitope polypeptide translocation into the ER where furin digestion will take place. Potential ER signal peptides include sequences such as, an alphavirus endoplasmic reticulum signal sequence (Garoff, H. et al., 1990, J. Cell. Biol., 111:867-876), influenza virus matrix protein derived peptide, M57-68 (Anderson, K. et al., 1991, J Exp Med, 174: 489-492), or tissue plasminogen activator peptide (Aurisicchio, L. et al., 2014, Oncoimmunology 3:e27529). Signal sequences for use in the present invention are set forth below.

The additional ER signal-encoding nucleic acid sequences that can be incorporated into the polynucleotide (minigene) and viral vectors described herein to enhance intracellular processing of the multi-epitope polypeptide following administration include, without limitation, Adenovirus ER signal: MRYMILGLLALAAVCSA (SEQ ID NO: 272) and Tissue plasminogen activator peptide: MDAMLRGLCCVLLLCGAVFVSPS (SEQ ID NO: 273).

Nucleic acid sequences encoding immunogenic peptides can also be included in the polynucleotide (minigene) and viral vectors as described herein. Such sequences include, without limitation, E. coli heat labile enterotoxin subunit B (LTB): MNKVKFYVLFTALLSSLCAHGAPQSITELCSEYHNTQIYTINDKILSYTESMAGKREMVII TFKSGATFQVEVPGSQHIDSQKKAIERMKDTLRITYLTETKIDKLCVWNNKTPNSIAAIS MEN (SEQ ID NO: 274); Influenza virus matrix protein M57-68 KGILGFVFTLLV (SEQ ID NO: 275); Tetanus toxin fragment c: IDKISDVSTIVPYIGPALNI (SEQ ID NO: 276); Lysosome-associated membrane protein (LAMP): MLIPIAVGGALAGLVLIVLIAYLVG (SEQ ID NO: 277); and Hsp70 peptide: TKDNNLLGRFELSG (SEQ ID NO: 278).

In some embodiments, the inclusion of nucleic acid sequences encoding polypeptide adjuvants at the carboxyl terminus (3′ end) of the polynucleotide (minigene) or viral vector described herein is employed to augment the immune response after administration and expression. Exemplary sequences useful for enhancement of the immune response include heat shock protein 70, lysosome-associated membrane protein (LAMP), the universal helper T cell (Th) epitope from tetanus toxin, and the E. coli heat-labile enterotoxin B subunit (Facciabene, A. et al., 2007, Vaccine, 26: 47-58; and 2006, Hum Gene Ther., 17: 81-92).

In other embodiments, nucleic acid sequences encoding epitopes of mutated or overexpressed oncogenes, cytokines, chemokines, antibodies, and known immunogenic TAAs, separated by processing sites, such as enzyme, e.g., furin, cleavage sites, are included in the polynucleotides (minigenes) and viral vectors described herein. Mutated oncogenes may minimize self-genes that might trigger autoimmunity. By linking all these genes in tandem with only enzyme cleavage sites between them, the expression of all of these genes can be driven from one or more subgenomic promoter(s) in the vector. By way of nonlimiting example, polynucleotide sequences encoding multiple epitopes of one or more oncogenes, or mutated forms thereof, which may be included in the polynucleotides and viral vectors of the invention, include androgen receptor (Olson, B. M. et al., 2013, Cancer Immunol. Immunother., 62(3):585-596), Her-2/neu (Parmiani, G. et al., 2002, J. Natl. Cancer Inst., 94(11):805-818), P53 (Ito, D. et al., 2007, Int. J. Cancer, 120(12):2618-2624), EphA2 (Tandon, M. et al., 2011, Expert Opin. Ther. Targets, 15(1):31-51), K-Ras (Gjertsen, M. K. et al., 1997, Int. J. Cancer, 72(5):784-790) and H-Ras (Fossum, B. et al., 1993, J. Immunol., 23:2687-2691). In other embodiments, nonlimiting examples of polynucleotide sequences encoding multiple epitopes of one or more immunotherapy enhancing genes that may be included in the polynucleotides and viral vectors of the invention include survivin (Siegel, S. A. et al., 2003, Br. J. Haematol., 122:911-914; Yang, Z. et al., 2008, Mol. Immunol., 45:1674-1681), WT1 (Miwa, H. et al., 1992, Leukemia, 6:405; Oji, Y. et al., 1999, Japan. J. Cancer. Res., 90:194; Oka Y. et al., 2000, J. Immunol. 2000, 164(4):1873-80; Li Z. et al., 2008, Microbiol. Immunol., 52:551-558). HTERT (Bright, R. K., et al., 2014, Human Vaccines & Immunotherapeutics, 10(11):3297-3305), tumor protein D52 (Bright, R. K., et al., 2014, Ibid.), IL-12 (Tseng, J. C. et al., 2004, Cancer Res., 64:6684-6692; Tseng, J. C. et al., 2004, Nature Biotechnol., 22:70-77; Granot, T. et al., 2013, Mol. Ther., 22(1):112-122; Granot, T. et al., 2011, PLoS One, 6(6):e20598), interferon-gamma (Granot, T. et al., 2013, Mol. Ther., 22(1):112-122; Granot, T. et al., 2011, PLoS One, 6(6):e20598) and calreticulin (Wang, H. T. et al., 2012, Int. J. Cancer, 130:2892-2902).

### Modulating the Immune Response Elicited by Sindbis Viral Vectors Encoding Multiple (Two or More) Tumor Associated Antigen Epitopes

In addition to activating CD8+ T cells and eliciting their responsiveness to tumor antigens and epitopes thereof, therapy with Sindbis viral vectors encoding multiple epitopes of tumor associated antigens can activate additional immune (or nonimmune) cells, including, but not limited to CD4+ T cells, natural killer (NK) cells, macrophages, monocytes, dendritic cells, neutrophils, and other cells, as well as the humoral immune response. Epitope spreading can occur not only in CD8+ T cells, but also in CD4+ T cells (Granot, T., and D. Meruelo, 2012, Cancer Gene TheR., 19: 588-591; Granot, T. et al., 2011, PLoS One 6: e20598; Granot, T. et al., 2014, Mol Ther, 22:112-122). To create optimal conditions for T cell stimulation in the lymph nodes, an embodiment of the invention encompasses polynucleotides and viral vectors, such as Sindbis virus expression vectors, that contain and deliver nucleic acid sequences encoding multiple (e.g., two or more) epitopes of (one or more) tumor associated antigens in conjunction with nucleic acid sequences (genes) encoding certain immune stimulating cytokines. Such immune stimulating cytokines include, but are not limited to, the interleukins IL-1, IL-2, IL-3, IL-4, IL-5, IL-6 IL-7, IL-8, IL-9, IL-10, IL-11, IL-12, IL-13, IL-14, IL-15, IL-16, and IL-17. Additional cytokines include IL-18 through IL-36.

Nucleic acid sequences encoding chemokines can also be included in the polynucleotide and viral vector nucleic acid sequences, including, but not limited to, CCL1 through CCL27 and other CC chemokines; CXCL1 through CXCL13 and other CXC chemokines; C chemokines; and CX3C chemokines. Nucleic acid sequences encoding cytokine or chemokine receptors and soluble receptors can also be used. Nucleic acid sequences encoding additional immune modulators that can be used and incorporated in the nucleic acid sequences of the polynucleotides and viral vectors, e.g., SV/TAA, of the invention include, without limitation, TGF-β and TNFα. Different combinations of the above-mentioned (or alternative) cytokines can also be used. It will be appreciated that nucleic acid sequences (genes) encoding immune stimulating molecules can be expressed from an additional promoter inserted into, for example, a Sindbis virus vector encoding multiple TAA epitopes as described herein, or may be included in a separate vector that is co-administered.

### Pharmaceutical Compositions

The present invention includes pharmaceutical compositions or formulations for treating subjects who are afflicted with cancer or a tumor, or who are at risk of developing cancer or a tumor. In an embodiment, the pharmaceutical composition includes a polynucleotide (minigene) encoding multiple epitopes, e.g., two or more, of a tumor associated antigen, wherein each epitope is separated by an enzyme cleavage site, e.g., a furin cleavage site, as well as other sequences for processing and expressing the encoded epitopes as described herein, and other coding sequences that may be included in the polynucleotide, e.g., immunostimulatory molecule coding sequence, and a pharmaceutically acceptable carrier, excipient, or diluent. In an embodiment, the pharmaceutical composition includes a viral vector or particle, e.g., a Sindbis viral vector or a pseudotyped viral vector as described herein, containing a polynucleotide (minigene) encoding multiple epitopes, e.g., two or more, of a tumor associated antigen, wherein each epitope is separated by an enzyme cleavage site, e.g., a furin cleavage site, as well as other sequences for processing and expressing the encoded epitopes as described herein, and other coding sequences that may be included in the polynucleotide, e.g., immunostimulatory molecule coding sequence, and a pharmaceutically acceptable carrier, excipient, or diluent. When formulated in a pharmaceutical composition, a therapeutic compound or product of the present invention can be admixed with a pharmaceutically acceptable carrier, diluent, or excipient.

The administration of a composition comprising a combination of agents herein for the treatment of a cancer or tumor may be by any suitable means that results in a concentration of the therapeutic that, combined with other components, is effective in ameliorating, reducing, or stabilizing a cancer in a subject. The composition may be administered systemically, for example, formulated in a pharmaceutically-acceptable buffer such as physiological saline. Routes of administration include, for example, subcutaneous (s.c.), intravenous (i.v.), intraperitoneal (i.p.), intramuscular (i.m.), or intradermal administration, e.g., by injection, that optimally provide continuous, sustained levels of the agent in the patient. The amount of the therapeutic agent to be administered varies depending upon the manner of administration, the age, physical condition and body weight of the patient, and with the clinical symptoms of the cancer or tumor. Generally, amounts will be in the range of those used for other viral vector-based agents employed in the treatment of a cancer or tumor, although in certain instances lower amounts will be needed if the agent exhibits increased specificity. A composition is administered at a dosage that shows a therapeutic effect, such as increasing immune cell (e.g., effector T cell; CD8+ T cell) levels, particular, TAA epitope-specific T cell levels, or that decreases cancer cell proliferation as determined by methods known to one skilled in the art.

The therapeutic agent(s) may be contained in any appropriate amount in any suitable carrier substance, and is/are generally present in an amount of 1-95% by weight of the total weight of the composition. The composition may be provided in a dosage form that is suitable for a parenteral (e.g., subcutaneous, intravenous, intramuscular, or intraperitoneal) administration route, such that the agent, such as a viral vector described herein, is systemically delivered. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Pharmaceutical compositions according to the invention may be formulated to release the active agent substantially immediately upon administration or at any predetermined time or time period after administration. The latter types of compositions are generally known as controlled release formulations, which include (i) formulations that create a substantially constant concentration of the agent within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with a tumor; (v) formulations that allow for convenient dosing, such that doses are administered, for example, once every one or two weeks; and (vi) formulations that target a cancer using carriers or chemical derivatives to deliver the therapeutic agent to a particular cell type (e.g., cancer or tumor cell). For some applications, controlled release formulations obviate the need for frequent dosing during the day to sustain the plasma level of the administered agent at a therapeutic level.

Methods by which to obtain controlled release in which the rate of release outweighs the rate of metabolism of the agent in question are not meant to be limiting. By way of example, controlled release is obtained by appropriate selection of various formulation parameters and ingredients, including, e.g., various types of controlled release compositions and coatings. Thus, the therapeutic agent is formulated with appropriate excipients into a pharmaceutical composition that, upon administration, releases the agent in a controlled manner. Examples include single or multiple unit tablet or capsule compositions, oil solutions, suspensions, emulsions, microcapsules, microspheres, molecular complexes, nanoparticles, patches, and liposomes.

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation, and can be found, for example, in Remington: The Science and Practice of Pharmacy, supra.

Compositions for parenteral delivery and administration may be provided in unit dosage forms (e.g., in single-dose ampules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent (e.g., a polynucleotide, viral vector or particle described herein), the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

In some embodiments, the composition comprising the active therapeutic(s) (i.e., a polynucleotide, viral vector or particle described herein) is formulated for intravenous delivery. As noted above, the pharmaceutical compositions according to the invention may be in the form suitable for sterile injection. To prepare such a composition, the suitable therapeutic(s) are dissolved or suspended in a parenterally acceptable liquid vehicle. Acceptable vehicles and solvents that may be employed include water, water adjusted to a suitable pH by addition of an appropriate amount of hydrochloric acid, sodium hydroxide or a suitable buffer, 1,3-butanediol, Ringer's solution, isotonic sodium chloride solution and dextrose solution. The aqueous formulation may also contain one or more preservatives (e.g., methyl, ethyl or n-propyl p-hydroxybenzoate). In cases where one of the agents is only sparingly or slightly soluble in water, a dissolution enhancing or solubilizing agent can be added, or the solvent may include 10-60% w/w of propylene glycol or the like.

### Methods of Delivery

Administration of a polynucleotide (minigene), viral vector, or pharmaceutical composition of the invention to a subject, e.g., a patient having cancer, to treat one or more of the above cancers, may cause epitope spreading within the patient. One of the disadvantages of prior cancer vaccine strategies has been the heterogeneity and genomic instability of tumor cell populations, which, coupled with the selective pressure induced by treatment, can lead to tumor evasion by loss or modification of a tumor associated antigen used in the vaccine. In this context, an advantageous aspect of the present invention is the potential to induce epitope spreading, i.e., the expansion of an anti-tumor T cell response directed against epitopes of tumor associated antigens that are endogenous to a cancer or tumor cell, but not actively delivered by the vector during therapy with a cancer vaccine. Clinical trials are increasingly incorporating the analysis of epitope spreading, and in some cases a positive correlation between the induction of epitope spreading and therapeutic efficacy has been shown.

In embodiments, the polynucleotide (minigene), viral vector, viral particle, or pharmaceutical composition of the invention, which is useful for eliciting a T cell response against the multiple epitopes of tumor associated antigens that are encoded by these agents, may be delivered, such as to a cell (particularly a cancer or tumor cell) in any manner such that the polynucleotide, viral vector, particle or composition is functional and active to express the encoded sequences. Illustratively, a polynucleotide encoding amino acid sequences of multiple tumor associated antigen epitopes may be delivered to cells for heterologous expression of the epitopes in the cells. Thus, the present invention features polynucleotides, viral vectors, or viral particles delivered to a cell by contacting the cell with a composition comprising the polynucleotides, viral vectors, or viral particles or by heterologously expressing the polynucleotides, viral vectors, or viral particles in the cell.

### Polynucleotide Therapy

One therapeutic approach for treating a cancer or tumorigenesis is polynucleotide therapy using a polynucleotide encoding the tumor associated antigen epitopes, such as two or more epitopes of one or more tumor associated antigens, of the invention. Expression of such polynucleotides or nucleic acid molecules in relevant cells is expected to stimulate an immune response, such as a cytotoxic T cell response, reduce survival of the cell and/or increase cell death. Such nucleic acid molecules can be delivered to cells of a subject having a cancer or tumor. The nucleic acid molecules must be delivered to the cells of a subject in a form in which they can be taken up so that therapeutically effective levels of the encoded products can be produced.

Transducing viral (e.g., retroviral, adenoviral, and adeno-associated viral) vectors can be used for delivering encoded proteins and peptide products to cells, especially because of their high efficiency of infection and stable integration and expression (see, e.g., Cayouette et al., Human Gene Therapy, 8:423-430, 1997; Kido et al., Current Eye Research, 15:833-844, 1996; Bloomer et al., Journal of Virology, 71:6641-6649, 1997; Naldini et al., Science, 272:263-267, 1996; and Miyoshi et al., Proc. Natl. Acad. Sci. U.S.A., 94:10319, 1997). For example, a polynucleotide encoding multiple epitopes of one or more tumor associated antigens can be cloned into a vector, e.g., a Sindbis virus vector or a pseudotyped virus vector, as described herein, and expression can be driven from its endogenous promoter, from a retroviral long terminal repeat, or from a promoter specific for a target cell type of interest. Other viral vectors that can be used include, for example, a vaccinia virus, a bovine papilloma virus, or a herpes virus (see, for example, the vectors of Miller, Human Gene Therapy, 15-14, 1990; Friedman, Science, 244:1275-1281, 1989; Eglitis et al., BioTechniques, 6:608-614, 1988; Tolstoshev et al., Current Opinion in Biotechnology, 1:55-61, 1990; Sharp, The Lancet, 337:1277-1278, 1991; Cornetta et al., Nucleic Acid Research and Molecular Biology, 36:311-322, 1987; Anderson, Science, 226:401-409, 1984; Moen, Blood Cells, 17:407-416, 1991; Miller et al., Biotechnology, 7:980-990, 1989; Le Gal La Salle et al., Science, 259:988-990, 1993; and Johnson, Chest, 107:77S-83S, 1995). Retroviral vectors are well developed and have been used, for example, as described in Rosenberg et al., NEJM, 323:370, 1990; Anderson et al., and U.S. Pat. No. 5,399,346. In some embodiments, the viral vector containing a polynucleotide or minigene encoding multiple tumor associated antigen epitopes is administered systemically.

As will be appreciated by the skilled practitioner, non-viral approaches can also be employed for the introduction of therapeutic polypeptide to a cell of a subject requiring induction of a T cell epitope immune response to inhibit growth of a cancer or tumor or to induce cancer or tumor cell death. For example, a nucleic acid molecule can be introduced into a cell by administering the nucleic acid in the presence of lipofection (Feigner et al., Proc. Natl. Acad. Sci. U.S.A. 84:7413, 1987; Ono et al., Neuroscience Letters, 17:259, 1990; Brigham et al., Am. J. Med. Sci., 298:278, 1989; Staubinger et al., Methods in Enzymology, 101:512, 1983), asialoorosomucoid-polylysine conjugation (Wu et al., Journal of Biological Chemistry, 263:14621, 1988; Wu et al., Journal of Biological Chemistry, 264:16985, 1989), or by micro-injection under surgical conditions (Wolff et al., Science, 247:1465, 1990). In addition, the nucleic acids can be administered in combination with a liposome and protamine.

Gene transfer can also be achieved using in vitro transfection methods. Such methods include the use of calcium phosphate, DEAE dextran, electroporation, and protoplast fusion. Liposomes can also be potentially beneficial for delivery of DNA into a cell.

cDNA expression for use in polynucleotide therapy methods can be directed from any suitable promoter (e.g., the Sindbis virus promoter, the human cytomegalovirus (CMV), simian virus 40 (SV40), or metallothionein promoters), and regulated by any appropriate mammalian regulatory element. For example, if desired, enhancers known to preferentially direct gene expression in specific cell types can be used to direct the expression of a nucleic acid. The enhancers used can include, without limitation, those that are characterized as tissue- or cell-specific enhancers. Alternatively, regulation can be mediated by the cognate regulatory sequences or, if desired, by regulatory sequences derived from a heterologous source, including any of the promoters or regulatory elements described above.

### Methods of Administration and Treatment Protocols

Provided are methods of administering a therapeutic agent to a subject in need, such as a subject having cancer or a tumor, or identified as being in need of such treatment), in which an effective amount of a polynucleotide, viral vector, or viral particle as described herein, or a composition described herein, is administered to a subject to produce a therapeutic effect. According to the present invention, a therapeutic effect includes, without limitation, an epitope-specific immune response against cancer and tumor cells expressing TAA-associated epitopes on their surface, e.g., by effector T cells (e.g., CD8+ T cells) activated by the multiple epitopes encoded by the polynucleotide or viral vector, such as a Sindbis virus vector encoding multiple epitopes of tumor associated antigens, optionally in association with MHC Class I or Class II molecules. Identifying a subject in need of such treatment can be in the judgment of a subject or a health care professional and can be subjective (e.g. opinion) or objective (e.g. measurable by a test or diagnostic method).

The therapeutic methods of the invention (which include prophylactic treatment) in general comprise administration of a therapeutically effective amount of the agents described herein, such as a polynucleotide, a viral vector, a viral particle, or composition containing the aforementioned agents, to a subject (e.g., animal, human) in need thereof, including a mammal, particularly a human. Such treatment will be suitably administered to subjects, particularly humans, suffering from, having, susceptible to, or at risk for cancer or a tumor. Determination of those subjects “at risk” can be made by any objective or subjective determination by a diagnostic test or opinion of a subject or health care provider (e.g., genetic test, enzyme or protein marker or biomarker, family history, and the like). The polynucleotide and viral vector agents described herein may be also used in the treatment of any other diseases or disorders in which multiple epitopes of one or more tumor associated antigens may be implicated.

In preclinical studies using mice, a single intraperitoneal (i.p.) injection of a therapeutically effective amount of the Sindbis viral vector encoding multiple (e.g., two or more) epitopes of one or more tumor associated antigens (SV/TAA), (˜10⁷ virus particles), resulted in rapid immunogenic delivery to lymph nodes and elicited a detectable CD8+ mediated immune response directed against the tumor (Example 5, infra). It will be appreciated by the skilled practitioner that other regimens may be necessary for achieving a maximal response in human subjects. For example, in human patients, therapeutically effective amounts of the vectors of the present invention can broadly range between about 6 and about 12 Logio vector particles/kg per treatment administered in between about 1 and about 8 i.p. injections over a time period of between about 1 week and many weeks, with the possibility of injecting one or more booster injections, week, months, or years, e.g., 1 or more years, later.

Viral vectors, polynucleotides (minigenes) and pharmaceutical compositions of the present invention can be used therapeutically to treat patients suffering from cancer or tumors, or prophylactically to vaccinate patients at risk for certain cancers or tumors, such as a prophylactic vaccine for cancer in the general population. A prophylactically effective amount of the vectors of the present invention may range between about 10² TU (transducing units) per kilogram body weight of the recipient and about 10⁶ TU kilogram body weight of the recipient. Mouse models of relevant cancers can be used to optimize dosages and regimens. To promote an effective, persistent immune response that includes both effector and memory CD8+ T cells, optimal dosage and immunization intervals are established. A CD8+ T cell response to an initial alphavirus vaccine quickly contracts, allowing development of memory T cells. Prior to this contraction, additional administration of the viral vector does not increase the immune response (Knudsen, M. L. et al., 2014, J Virol., 88:12438-12451). The strong type I interferon (IFN) response to alphavirus RNA amplification stimulates the generation of memory T cells by activating dendritic cells to promote cross-priming (Fuertes, M. B. et al., J Exp Med, 208: 2005-2016).

A typical treatment regime using a composition of the invention may include SV/multi-TAA epitope viral vector administration followed by monitoring lymphocytes, several times per week, using flow cytometry to determine the peak and decline of effector CD8+ T cells (CD62L⁻ CD127⁻). At this point, a boost of vector can be administered allowing an increase in effector memory T cells (CD62L⁻ CD127⁺), central memory T cells (CD62L⁺ CD127⁺) and T cells with persistent high recall capacity (CD27⁺ CD43⁻). Efficacy is determined by positive immune response and low tumor recurrence.

The present invention is not limited with respect to the vectors used for immunization and boost(s). The distribution of T cell subpopulations induced by a DNA-launched alphavirus replicon can be altered by heterologous boost (Knudsen, M. L. et al., 2-14, J. Virology, 88:12438-12451). For example, boosting with a poxvirus vector (Modified Vaccinia Ankara or MVA) can boost the expansion of T cell compartments that can greatly augment efficacy. In this embodiment, the viral vector employed in the booster administration encodes multiple (e.g., two or more) epitopes of one or more tumor associated antigens. Any antigen delivery system can be used to boost the immune response induced by the vectors of the present invention. Non-limiting examples include replication-defective adenoviruses, fowl pox viruses, vaccinia virus, influenza virus, Sendai virus, naked DNA, plasmids and peptides (Woodland, D. L., 2004, TRENDS in Immunology, Vol. 25(2):98-104).

Exemplary routes of vector administration include, without limitation, parenteral administration, such as by intraperitoneal, intravenous, subcutaneous, stereotactic, intramuscular, intranasal, intradermal, intraorbital, intranodular and intratumoral injection. Other modes of administration may include oral, intracranial, ocular, intraorbital, intra-aural, rectal, intravaginal, suppositories, intrathecal, inhalation, aerosol, and the like.

In a certain embodiment, the vector used for treatment is a defective Sindbis viral vector, the tumor is a cancer or tumor, such as ovarian cancer, and the two or more encoded epitopes of the tumor associated antigens include p53, SP17, survivin, WT1, and NY-ESO-1. In another embodiment the TAAs are NY-ESO-1, gp70, and pbk. In another embodiment the TAAs include NY-ESO-1 and survivin.

Patients to whom the viral vectors of the present invention are administered may also benefit from adjunct or additional treatments, such as chemotherapy and or radiation treatments, as well known to those having skill in the art. In particular, the SV/TAA Sindbis viral vector can be combined with chemotherapy treatment. In certain cases, SV and chemotherapy synergize (e.g., US Patent Application Publication No. 2016/0008431), thus providing the potential for an improved treatment effect and/or outcome. Suitable chemotherapy includes, without limitation, chemotherapy treatment that stimulates the immune system, or that inhibits suppressor elements in the immune system, or that affects tumor cells and makes them more susceptible to T cell (or other immune cell) cytotoxicity. For example, there are certain chemotherapies that can facilitate treatment and therapy with the SV/TAA viral vector described herein because they attenuate the activity of immunosuppressive cells, thereby enhancing immunostimulation by the SV/TAA viral vector. In addition, chemotherapy may enhance tumor cell susceptibility to T cell mediated cytotoxicity.

### Kits

The invention provides kits for the treatment or prevention of cancer or tumors, particularly those expressing multiple epitopes of one or more tumor associated antigens. In one embodiment, the kit includes a therapeutic or prophylactic composition containing an effective amount of a polynucleotide, viral vector, or viral particle as described herein, which comprises a polynucleotide that encodes two or more epitopes of one or more tumor associated antigens separated by enzymes cleavage sites. In an embodiment, the polynucleotide encodes an alphavirus protein or a fragment thereof. In an embodiment, the alphavirus protein or a fragment thereof is a Sindbis virus protein or a fragment thereof. In embodiments, the epitopes and tumor associated antigens are those presented in Tables 1-28 supra. In some embodiments, the kit comprises a sterile container which contains the therapeutic or prophylactic composition; such containers can be boxes, ampoules, bottles, vials, tubes, bags, pouches, blister-packs, or other suitable container forms known in the art. The containers can be made of plastic, glass, laminated paper, metal foil, or other materials suitable for holding medicaments.

If desired, a composition comprising one or more TAA multiple epitope-encoding viral vector agents of the invention is provided together with instructions for administering the agent to a subject having or at risk of developing cancer or a tumor. The instructions will generally include information about the use of the composition for the treatment or prevention of the cancer or tumor. In other embodiments, the instructions include at least one of the following: description of the therapeutic agent; dosage schedule and administration for treatment or prevention of ischemia or symptoms thereof; precautions; warnings; indications; counter-indications; overdosage information; adverse reactions; animal pharmacology; clinical studies; and/or references. The instructions may be printed directly on the container (when present), or as a label applied to the container, or as a separate sheet, pamphlet, card, or folder supplied in or with the container.

The practice of the present invention employs, unless otherwise indicated, conventional techniques of molecular biology (including recombinant techniques), microbiology, cell biology, biochemistry and immunology, which are well within the purview of the skilled artisan. Such techniques are explained fully in the literature, such as, “Molecular Cloning: A Laboratory Manual”, second edition (Sambrook, 1989); “Oligonucleotide Synthesis” (Gait, 1984); “Animal Cell Culture” (Freshney, 1987); “Methods in Enzymology” “Handbook of Experimental Immunology” (Weir, 1996); “Gene Transfer Vectors for Mammalian Cells” (Miller and Calos, 1987); “Current Protocols in Molecular Biology” (Ausubel, 1987); “PCR: The Polymerase Chain Reaction”, (Mullis, 1994); “Current Protocols in Immunology” (Coligan, 1991). These techniques are applicable to the production of the polynucleotides, viral vectors and viral particles of the invention, and, as such, may be considered in making and practicing the invention. Particularly useful techniques for particular embodiments will be discussed in the sections that follow.

The following examples are put forth so as to provide those of ordinary skill in the art with a complete disclosure and description of how to make and use the assay, screening, and therapeutic methods of the invention, and are not intended to limit the scope of what the inventors regard as their invention.

### EXAMPLES

### Example 1

### Methods

Vector preparation: Construction of recombinant viral vectors was performed using standard techniques well known to those of ordinary skill in the field of molecular biology, including, but not limited to, plasmid purification, restriction endonuclease digestion, ligation, transformation, polymerase chain reaction and DNA sequencing (e.g., Current Protocols in Molecular Biology, EM. Ausubel et al. (Eds), John Wiley and Sons, Inc., NY, USA. (1998) and Molecular Cloning: A Laboratory Manual (2nd Ed.), J. Sambrook, E. F. Fritsch and T. Maniatis (Eds), Cold Spring Harbor Laboratory Press, NY, USA. (1989)).

For the experiments using Sindbis viral vector encoding LacZ (SV/LacZ) as an immunogenic SV/TAA agent, and SV/Fluc and SV/GFP as control vectors, the vectors were produced as previously described. (Tseng J. C. et al,., 2004, Nat. Biotechnol., 22:70-77). Briefly, plasmids carrying the replicon (SinRep5-LacZ, SinRep5-GFP, or SinRep5-Fluc) or DHBB helper RNAs (SinRep5-tBB) were linearized with Xhol (for SinRep5-LacZ, SinRep5-GFP, and SinRep5-tBB) or PacI (for SinRep5-Fluc). In vitro transcription was performed using the mMessage mMachine RNA transcription kit (Ambion, Austin, Tex.). Helper and replicon RNAs were then electroporated into BHK cells and incubated at 37° C. in α-MEM supplemented with 10% FBS. After 12 hours, the medium was replaced with OPTI-MEM I (Invitrogen, Carlsbad, Calif.), supplemented with CaCl₂ (100 μg/ml), and cells were incubated at 37° C. After 24 hours, the supernatant was collected, centrifuged to remove cellular debris, and frozen at −80° C. Vector titers were determined as known in the art (Tseng J. C., et al., 2002, J Natl Cancer Inst., 94:1790-1802) and were similar in all three vectors (SV/LacZ, SV/Fluc, and SV/GFP).
Cell lines and Cell Culture: Baby hamster kidney (BHK), CT26.WT, and LacZ-expressing CT26.CL25 cells were obtained from the American Type Culture Collection (ATCC), (Manassas, Va.). BHK cells were maintained in minimum essential α-modified media (α-MEM) (Mediatech, Va.) with 10% fetal bovine serum (FBS) (Atlanta Biologicals, Norcross, Ga.). CT26.WT, CT26.CL25 cells were maintained in Dulbecco modified essential media (DMEM) containing 4.5 g/L glucose (Mediatech) supplemented with 10% FBS. All basal media was supplemented with 100 mg/mL of penicillin-streptomycin (Mediatech) and 0.5 mg/mL of amphotericin B (Mediatech).
Virion Production: Sindbis virus vectors were produced as described in U.S. Pat. Nos. 7,303,898, 7,306,792, and 8,093,021. Briefly, plasmids carrying the replicon pT7StuI-R or DHBB helper RNAs (SinRep5-tBB) were linearized with appropriate restriction enzymes. In vitro transcription was performed using the mMessage RNA transcription kit (Ambion, Tex.) according to the manufacturer's instructions. Helper and replicon RNAs were then electroporated into BHK cells and incubated at 37° C. in MEM supplemented with 10% FBS. After 12 hours, the medium was replaced with OPTIMEM I (Life Sciences, CA) supplemented with CaCl₂ (100 g/mL) and cells were incubated at 37° C. After 24 hours, the supernatant was collected, centrifuged to remove cellular debris, and frozen at −80° C. Titers of the vectors were determined using RT-qPCR as practiced in the art.
Mice, Tumor Inoculation and Therapeutic Efficacy: 4-8-week-old female BALB/c mice were purchased from Taconic (Germantown, N.Y.). For an i.p. tumor model, 2.5×10⁴ or 5×10⁴ CT26.CL25 cells in 0.2 mL PBS were injected i.p. into each mouse. For the lung tumor model, 0.3×10⁶ CT26.WT.Fluc or CT26.CL25.Fluc cells in 0.2 ml PBS were injected intravenously into each mouse. Therapeutic efficacy was monitored in three ways: tumor volume (for subcutaneous tumors, measured with mechanical calipers), tumor luminescence and survival. Noninvasive bioluminescent imaging was performed using the IVIS Spectrum imaging system (Caliper Life Sciences, Inc., MA), and tumor growth was quantified using the Living Image 3.0 software (Caliper Life Sciences). Survival of the animals was monitored and recorded daily.
Flow cytometry: Flow cytometry was used to analyze lymphocytes extracted from organs, peritoneum or peripheral blood. Cells were treated with 1× RBC lysis buffer (eBioscience) to eliminate red blood cells. Peritoneal cells were collected and stained with various Abs, washed twice with HBSS buffer (Mediatech), and analyzed using an LSR II machine (BD Biosciences, San Jose, Calif.). Data were analyzed using FlowJo (Tree Star, San Carlos, Calif.).
Bioluminescent imaging of SV/Fluc: Tumor-bearing and tumor-free mice were injected with SV/Fluc (˜10⁷ plaque-forming units in 0.5 ml of OPTI-MEM I 0.5 ml) i.p. After the treatment, bioluminescence signal was detected by IVIS at the indicated time points (Tseng, J. C. et al., 2004).

### Example 2

### Construction of a Sindbis Viral Vector Expressing Multiple Epitopes for Inducing Anti-Tumor Immunity

A polynucleotide (DNA sequence; minigene) encoding multiple T cell recognition epitopes separated by furin enzyme cleavage sites was synthesized by GeneArt® (Life Technologies Corp., Waltham Mass.) using standard molecular biology methods. The synthetic polynucleotide contained a ribosome binding site, a translation start codon, an endoplasmic reticulum signal sequence, followed by furin cleavage sites interspersed with the epitope-encoding sequences, a stop codon and restriction enzyme sites that allowed the polynucleotide sequence to be inserted into XbaI/ApaI restriction endonuclease sites of the Sindbis replicon pT7StuI-RLacZ #202 (WO 2015/035213 A2) to replace the LacZ gene. The Sindbis replicon contained a viral sub-genomic promoter sequence upstream from the Xbal site and a mRNA poly A sequence located downstream of the Apal site. This synthesized DNA sequence and its encoded amino acid sequences are as follows:

### DNA Sequence

(SEQ ID NO: 10)
TCTAGAGCCACCATGCTGGTGACAGCCATGTGTCTGCTGGGCAATGTCAG
CTTCGTCCGGAGCAAGCGGCTGCGGGGACCAGAGTCTCGGCTCCTGGAGG
TGCGGAGCAAGCGGCTGTCCCCATCTTACGCCTACCACCAGTTCGTCCGG
AGCAAGCGGCTGGGCTGTGCCTTCCTGACCGTGAAGCAGATGCGGAGCAA
GCGGCTGTGAGGGCCC

### Amino Acid Sequence

(SEQ ID NO: 11)
MLVTAMCLLGNVSFVRSKRLRGPESRLLEVRSKRLSPSYAYHQFVRSKRL
GCAFLTVKQMRSKRL*

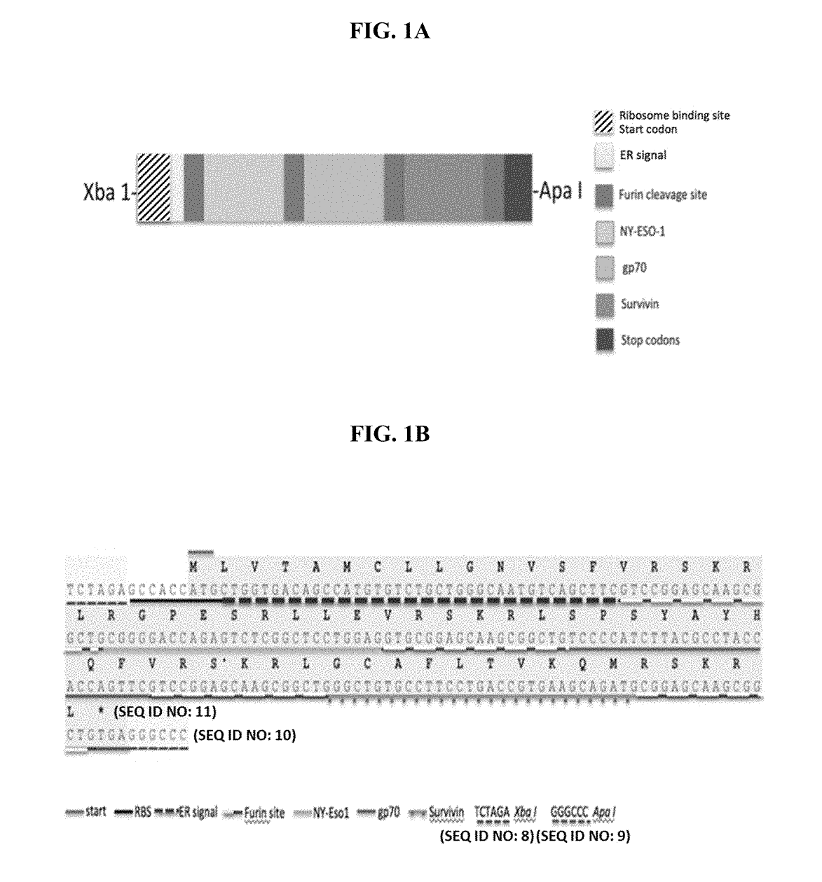

The synthesized polynucleotide sequence was inserted into the GeneArt pMX plasmid and provided as a DNA plasmid. The plasmid was transformed into NEB 5-alpha competent E. coli cells (New England BioLabs). Clones were grown and plasmid DNA was purified. The clones were verified by DNA sequencing (Macrogen USA). The restriction enzymes Xbal and Apal were used to excise the DNA polynucleotide (minigene) from the pMX plasmid vector. Following extraction, the polynucleotide (minigene) was cloned into the pT7StuI-RLacZ #202 vector. Schematically, the minigene as described is illustrated in FIG. 1A and the exact sequence arrangement is shown in FIG. 1B.

Because Sindbis virus polypeptides are naturally processed by furin, a nucleic acid sequence encoding the Sindbis furin digestion motif, XRSKRX (SEQ ID NO: 5), where X is a hydrophobic residue, was incorporated into the polynucleotide to allow proper processing of the encoded epitopes of the tumor associated antigens. A ribosomal binding site, start codon and an alphavirus endoplasmic reticulum (ER) signal sequence were also encoded at the 5′ flanking region of the furin-epitope-furin sequences. The ER signal sequence was included to facilitate multi-epitope polypeptide translocation into the ER where furin digestion occurs. A stop codon was included at the 3′ end of the polynucleotide (minigene). The restriction enzyme sites, Xbal and Apal, were molecularly engineered into the 5′ and 3′ ends, respectively, of the polynucleotide in order to clone the synthesized polynucleotide sequence into the Sindbis virus vector nucleic acid directly downstream of the viral subgenomic promoter that drives high levels of transcription.

In this Example, two or more epitopes, i.e., 3 different epitopes, of different tumor associated antigens were incorporated into the Sindbis viral vector, namely, an epitope of human NY-ESO-1, as described herein, which is a tumor associated antigen expressed in human ovarian cancers and other human cancers; an epitope of gp70, an endogenous murine leukemia virus antigen; and an epitope of survivin, an anti-apoptotic protein that is highly expressed in many tumors. The three epitopes are presented in Table 30 and are highly expressed in CT26 tumors, but have low expression in normal mouse tissues.

TABLE 30
Epitopes included in SV/MG

| | | Amino | | |
|---|---|---|---|---|
| Antigen | Epitope | acids | MHC I | Reference |
| GP70 | SPSYAYHQF | 423-431 | H-2L^{d} | Slansky, J. E. et al., 2000, Immunity, 13: 529-538 |
| | (SEQ ID | | | |
| | NO: 280) | | | |
| NY-ESO-1 | RGPESRLLE | 81-89 | H-2D^{d} | Muraoka, D., et al., 2013, Vaccine, 31: 2110-2118 |
| | (SEQ ID NO: 3) | | | |
| Survivin | AFLTVKKQM | 83-91 | H-2K^{d} | Siegel, S. et al., 2003, Br. J. Haematol., 122: 911-914; |
| | (SEQ ID NO: 4) | | | Yang, Z. et al., 2008, Mol. Immunol., 45: 1674-1681. |

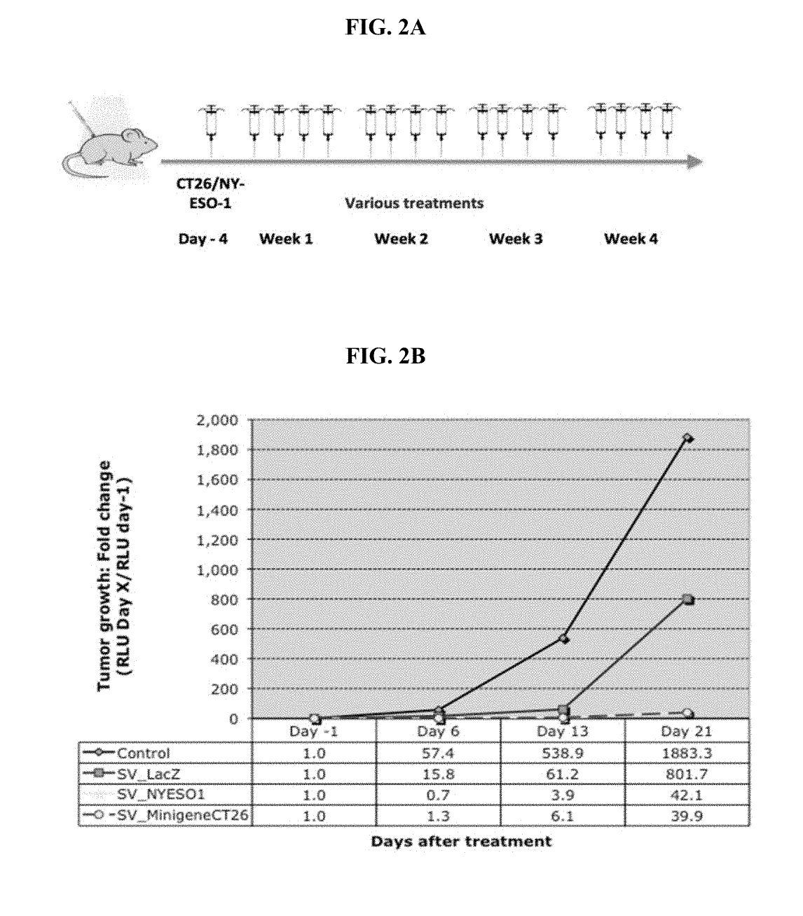

Determining in vivo anti-tumor efficacy: To test the anti-tumor efficacy of the Sindbis viral vector encoding multiple (3) epitopes of different tumor associated antigens (TAAs), as described above, (denoted “SV/MG” or “SV/MG-CT26” herein), a Balb/c CT26 colon carcinoma tumor model was used in which CT26/NY-ESO-1 cells were injected intraperitoneally into BALB/c mice. CT26 is a murine colon cancer cell line that was transfected with human NY-ESO-1 cDNA and stably expresses human NY-ESO-1 and its epitopes, and is available from the American Type Culture Collection (ATCC, Manassas, Va.). When injected into susceptible mice, the cells form solid tumors in the animals. CT26 cells can also be transfected with proteins, e.g., LacZ, luciferase, GFP, to aid in detecting tumors in animal studies. An exemplary administration regimen is shown FIG. 2A.
Imaging of tumors: Bioluminescence signals were periodically monitored using the IVIS system. Living Image software (Xenogen Corp., Alameda, Calif.) was used to grid the imaging data and integrate the total bioluminescence signals (RLU) in each boxed region to obtain the data shown in FIG. 2B. Wild-type CT26 cells and LacZ-expressing CT26 cells (CT26.CL25 (LacZ) cells) were obtained from the American Type Culture Collection (Manassas, Va.). The CT26.CL.25 (LacZ) cells express several tumor associated antigens. (Castle, J. C. et al., 2014, BMC Genomics, 15:190). CT26.CL25 cells expressing the NY-ESO-1 epitope are as described in Gnjatic, S. et al., 2006, Adv Cancer Res, 95:1-30. Firefly luciferase (Fluc)-expressing CT26 cells (CT26.WT.Fluc and CT26.CL25.Fluc) for noninvasive bioluminescent imaging were generated by stable transfection of a Fluc-expressing plasmid into the CT26.WT and CT26.CL25 cells. The Fluc-expressing plasmid was constructed by introducing an SV40 promoter sequence into the multi-cloning site of the pGL4.20 vector (Promega, Wis.) (Granot, T. et al., 2014, Mol. Ther., 22:112-122).

As shown in FIG. 2B, the growth of CT26/NY-ESO-1 tumor cells in animals treated with the multi-TAA epitope Sindbis virus vector (SV/MG) was strikingly lower compared to that in animals treated with the negative control and irrelevant control Sindbis viral vectors for an extended time period, e.g., to Day 27 post administration. Controls shown in FIG. 2B were mice that had not received Sindbis viral vectors (control), mice that had received SV/LacZ, a Sindbis viral vector that encodes the bacterial enzyme beta-galactosidase (LacZ), an irrelevant tumor associated antigen; and a positive control Sindbis viral vector, SV/NY-ESO-1, which encodes the NY-ESO-1 tumor associated antigen and which effectively reduced the growth of CT26/NY-ESO-1 tumor cells in animals harboring the tumors.

In a related example, another Sindbis viral vector encoding multiple epitopes of tumor associated antigens (e.g., called the SV/MG vector) can be prepared using the same techniques described above for testing in the CT26 tumor mouse model. The Sindbis viral vector created to treat tumors in the CT26 mouse model encodes an epitope of the tumor associated antigen NY-ESO-1, an epitope of the viral antigen gp70, and an epitope from the tumor associated antigen Pbk, also termed TOPK for T-cell-originated protein kinase. Advantageously, these epitopes are highly expressed in CT26.CL25 tumor cells, but have low expression in mouse tissues. The epitope sequences included in the SV/MG vector are shown in the below Table 31. In an embodiment, epitope sequences of HIV gp120 or gp41 and an epitope sequence from human pbk or a human pbk ortholog may be included in the SV vector.

TABLE 31
Epitopes used in the Sindbis
virus multi-TAA epitope vector

| Antigen | MHC 1 | Epitope |
|---|---|---|
| NY-ESO-1 | H2D^{d} | LLMWITQCF (SEQ ID NO: 1) |
| MuLV gp70 | H2L^{d} | SPSYVYHQF (SEQ ID NO: 281) |
| Pbk | H2D^{d} | GSPFPAAVI (SEQ ID NO: 2) |

The polynucleotide comprising multiple epitope sequences of tumor associated antigens NY-ESO-1, gp70 and pbk for Sindbis viral vector expression was prepared by synthesizing double-stranded oligomers and DNA primers (GeneLink Inc.) as set forth below. Routine PCR technology was used to generate two fragments which have their ends modified by mis-priming so that they shared a region of homology. When these two fragments were mixed, denatured and reannealed, the 3′-end of the top strand of fragment annealed onto the 3′-end of the bottom strand of fragment, and this overlap was extended to form the recombinant product. This process was reiterated until all epitope fragments were incorporated.

### A. Oligomers

Gp70
(SEQ ID NO: 282)
R S K R L S P S Y V Y H Q F
(SEQ ID NO: 283)
AGG AGC AAA AGA GTG AGC CCC AGC TAC GTG TAC CAC
CAG TTC TCC TCG TTT TCT CAC TCG GGG TCG ATG CAC
ATG GTG GTC AAG
NY-ESO-1
(SEQ ID NO: 284)
R S K R L L M W I T Q C F
(SEQ ID NO: 285)
AGG AGC AAA AGA CTG CTG ATG TGG ATC ACC CAG TGC
TTC TCC TCG TTT TCT GAC GAC TAC ACC TAG TGG GTC
ACG AAG
Pbk
(SEQ ID NO: 286)
R S K R G S P F P A A V
(SEQ ID NO: 287)
AGG AGC AAA AGA GGC AGC CCC TTC CCC GCC GCT GTG
ACC TCC TCG TTT TCT CCG TCG GGG AAG GGG CGG CGA
CAC TGG
RSKR = Furin sequence

### B. Primers

Primer 1:

(SEQ ID NO: 288)

5′ agg agc aaa aga cac agc ccc agc 3′
Primer 2:

(SEQ ID NO: 289)

5′ tct ttt gct cct gaa ctg gtg gta 3′
Primer 3:

(SEQ ID NO: 290)

5′ tac cac cag ttc agg agc aaa aga 3′
Primer 4:

(SEQ ID NO: 291)

5′ tct ttt gct cct gaa gca ctg ggt 3′
Primer 5:

(SEQ ID NO: 292)

5′ acc cag tgc ttc agg agc aaa aga 3′
Primer 6:

(SEQ ID NO: 293)

5′ ggt cac age ggc ggg gaa 3′

PCR and splicing by overhang extension (SOE) PCR reactions were carried out in a thermocycler for 25 cycles, each consisting of 1 min at 94° C., 2 min at 50° C., and 3 min at 72° C. Taq-PCR reactions were performed with reaction buffer containing dNTP's (200 μM), forward and reverse primers (0.5 μM/each) and 1μ Taq-DNA polymerase in a final volume of 20 μl. PCR products were analyzed by electrophoresis in agarose gels and DNA bands were excised from the gel and purified with a gel extraction kit (Zymo Research). The completed multi-epitope fragment was blunt-end ligated into the Nael site of the pT7StuI-R ΔLacZ #202 plasmid vector, transformed into E. coli, purified and sequenced.

### Example 3

### Sindbis Viral Vector Encoding Multiple Epitopes of Tumor Associated Antigens Produces Polyepitope mRNA

An experiment was conducted to determine whether the Sindbis viral vector (SV/MG-CT26) encoding multiple epitopes of tumor associated antigens, namely, NY-ESO-1, gp70 and survivin as described in Example 2 supra, produced the correct multiple epitope mRNA. For the experiment, ten-fold serial dilutions of the Sindbis virus vector encoding multiple epitopes, called “SV/MG-CT.26” herein (10⁰-10¹¹) were used to infect 2×10⁴ baby hamster kidney cells. After an overnight incubation, the cells were collected by centrifugation, and RNA was isolated using a Qiagen kit (Qiagen, Hilden, Germany) according to the manufacturer's instructions. RNA was quantified using a nanodrop spectrophotometer.

One microgram (1 μg) of each sample was reversed transcribed using ThermoScript (Life Technologies, CA) according to the manufacturer's instructions. The cDNA5_R reverse primer 5′ TTTTTGAAATGTTAAAAACAAAATTTTGTTG (SEQ ID NO: 294) was used at a concentration of 50 μM to transcribe the RNA into cDNA. Quantitative PCR (qPCR) was performed using 5 μl out of the 30 μl total cDNA reaction. Syber green master reaction mix was used according to the manufacturer's instructions (BioRad, CA). A standard curve was generated using 10-fold dilutions of pT7StuI-R-MG_CT26 plasmid DNA from which the viral vector was made. For performing qPCR, the Forward primer was: Sindbis position 7692: TGATCCGACCAGCAAAACTC (SEQ ID NO: 295), and the Reverse primer was cDNA5_R pos. 7990: TTTTTGAAATGTTAAAAACAAAATTTTGTTG (SEQ ID NO: 294). The primer concentration used was 10 μM. qPCR was performed using a MyiQ cycler (BioRad, CA). The dilution factors and picograms (pg) of transcript produced are presented in the table below.

TABLE 32

| Dilution Factor | Transcript (in pg) |
|---|---|
| 10⁰ | 1122 |
| 10⁻² | 26.5 |
| 10⁻⁴ | 1.39 |

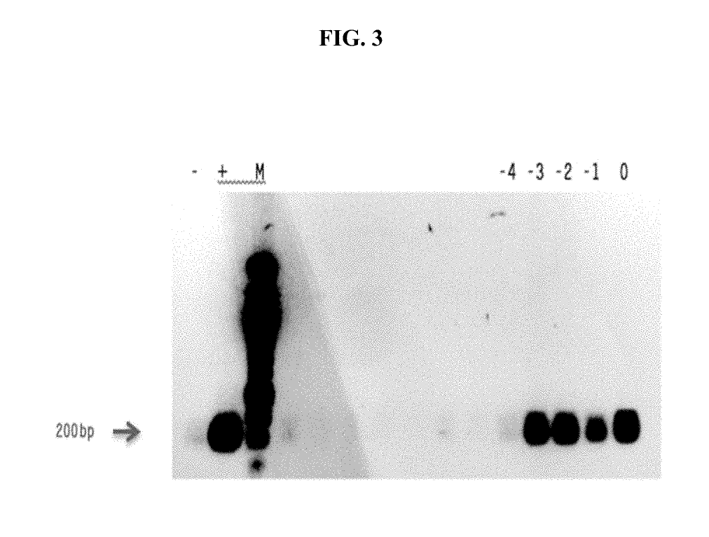

FIG. 3 presents a UV image of stained qPCR DNA products subjected to agarose gel electrophoresis. In the UV image of stained DNA samples from qPCR, the Lanes are identified as follows: Lane (−): cDNA from uninfected BHK; Lane (+): RNA/cDNA from pSV/MG plasmid; Lane M, 100 base pair ladder marker; Lane (−4), Lane (−3), Lane (−2), Lane (−1) and Lane (0) show qPCR products at the 10⁻⁴, 10⁻³, 10⁻², 10⁻¹, and 10⁰, respectively, dilutions, respectively, of RNA from baby hamster kidney (BHK) cells infected with SV/MG-CT.26 at the stated dilutions. The results from the qPCR experiment as presented in Table 32 indicate that the polynucleotide encoding multiple epitopes of the NY-ESO-1, gp70 and survivin tumor associated antigens was transcribed in BHK cells. Agarose gel electrophoresis indicates that the qPCR transcript is the expected size of 204 base pairs (bp). (FIG. 3).

### Example 4

### Preclinical Prophylactic and Therapeutic Treatment with SV/Multi-Epitope Vector

The treatment protocol presented in Table 33 was used in testing the prophylactic, therapeutic and combined treatment of CT26/NY-ESO-1 tumor cells by administering a Sindbis viral vector encoding multiple epitopes of tumor associated antigens, in particular, the NY-ESO-1 cancer antigen, as described in Example 2, supra, and shown in FIG. 2A. The protocol was designed to determine the effects of prophylactic treatment of animals with a Sindbis viral vector expressing epitopes from multiple tumor associated antigens, i.e., SV/NY-ESO-1-gp70-survivin prior to inoculation of tumor cells (e.g., a Sindbis viral vector vaccine). The protocol was also designed to determine the effects of additional boosting inoculations administered at two time intervals; the effects of vector therapy only after tumor inoculation; and the effects of combined vaccine and therapeutic vector treatment.

TABLE 33
Treatment protocols using a SV/multi-epitope vector

| | | | | Treat with | Treat with |
|---|---|---|---|---|---|
| | | | | SV/multi- | SV/multi- |
| | | | Inject tumor | epitope | epitope |
| | Immunization | Boost | cells | vector | vector |
| Day | 1 | 7 | 17 | None | None |
| | 1 | 7 | 17 | 24 | 31 |
| | None | None | 17 | 24 | 31 |
| | None | None | 17 | None | None |
| | 1 | 21 | 31 | None | None |
| | 1 | 21 | 31 | 38 | 45 |
| | None | None | 31 | 38 | 45 |
| | None | None | 31 | None | None |

### Example 5

### Clinical Treatment with a Sindbis Virus-Multi Epitope Vector

A Sindbis virus vector encoding multiple epitopes of ovarian cancer tumor associated antigens (SV/Multi-epitope vector), including, for example, two or more of NY-ESO-1, CEA or CA-125 (Schwab, C. L. et al., 2014, Immunotherapy, 6:1279-1293) would be advantageous for use in treating ovarian cancer. Screening of tumors from patients who have undergone tumor debulking surgery can be used to determine whether treatment with a Sindbis viral vector encoding multiple epitopes of tumor associated antigens will be beneficial based on the presence of TAAs on cancer or tumor cells of the patients and on the patient's specific antigen presenting HLA haplotypes, e.g., as described in Example 6, infra. Following administration of the Sindbis viral vector encoding multiple epitopes of one or more tumor associated antigens, a body fluid sample, e.g., blood, serum, or plasma, of selected patients can be obtained to monitor blood lymphocytes in order to examine the patient's immune response and guide the treatment regimen. For example, a patient's blood can be analyzed over time for the presence of effector CD8⁺ T cells, and to determine if the effector cells decline and memory (CD27⁺CD43⁻CD8⁺) T cells appear. Routine techniques in the art are suitable for analyzing the patient's blood sample for the presence of the appropriate T cells, e.g., flow cytometry, immunohistochemistry, staining (e.g., immunofluorescent staining). When a memory cell response is detected, a second administration of the Sindbis viral vector encoding tumor associated antigen epitopes can be administered to boost the patient's immune response.

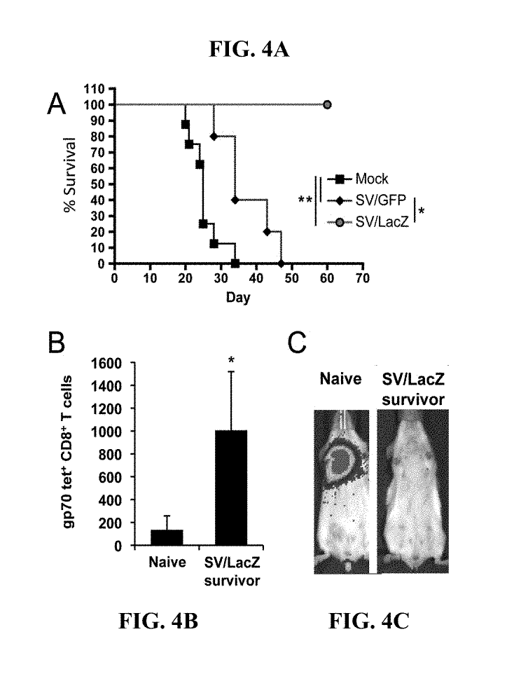

That a SV vector expressing the exemplary tumor associated antigen (TAA) LacZ was effective in the CT26 tumor mouse model and maintained the survival of mice having LacZ-expressing CT26 tumors, (FIG. 4A), as well as induced the diversification of the CD8⁻ T cell response to a tumor model (FIG. 4B), has been demonstrated by the inventors' studies using a Sindbis viral vector encoding the bacterial β-galactosidase (LacZ) enzyme, (SV/LacZ), and a comparator control Sindbis viral vector encoding green fluorescence protein (GFP), (SV/GFP). FIG. 4A shows a survival plot of mice treated with the different Sindbis viral vectors described above. For these studies, CT26 tumor-bearing mice were treated, 4 days after tumor inoculation, with either the SV/LacZ vector, the control SV/GFP vector, or media (Mock). Intraperitoneal inoculations of 10⁷ virus particles in 0.5 ml Optimem (Mediatech, Va.) were administered to the mice. Only the SV/LacZ vector was found to induce complete tumor remission for at least 60 days. FIG. 4B involved the use of tetramers, labeled tetrameric MHC molecules, (Altman, J. D. et al., 1996, Science, 274(5284):94-96) as a sensitive means for identifying specific T cells in mice treated with the Sindbis vector SV/LacZ. Following treatment with the Sindbis vector encoding LacZ, splenocytes from the SV/LacZ-treated mice were found to contain CD8⁺ T cells specific for both LacZ and gp70, an endogenous CT26 tumor associated antigen. The production of effector T cells directed against an antigen different from that produced by the SV/LacZ vector administered to the mice thus indicated that epitope spreading had occurred in the SV/LacZ treated animals. FIG. 4C presents photographs of representative mice imaged 14 days post-treatment with the SV/LacZ vector or naïve controls, in which tumors (CT26 colon tumors) were found to grow in naive mice (i.e., those not treated with SV/LacZ), but not in mice treated with the SV/LacZ vector expressing LacZ antigen (SV/LacZ survivor mice).

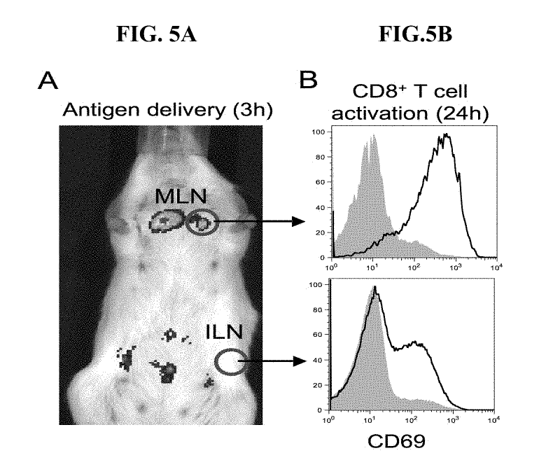

The results presented in FIG. 5B demonstrate that SV/LacZ-induced epitope spreading was successful in countering the loss of LacZ expression. Such SV/LacZ-dependent epitope spreading generated by administering the SV/LacZ vector to mice in the CT26 tumor mouse model contributed significantly to the complete suppression of growth of tumors in the mice treated with the SV/LacZ Sindbis viral vector, and their survival, as evidenced by the negative tumor cell growth in the SV/LacZ-treated mouse (FIG. 4C). These results evidence that SV vectors carrying β galactosidase (LacZ) had a remarkable therapeutic effect in mice bearing LacZ-expressing CT26 tumors.

FIGS. 5A and 5B show the combination of imaging and flow cytometry used to assess the results of in vivo treatment (immunotherapy) using a Sindbis viral vector expressing at least one epitope derived from a tumor associated antigen (SV/TAA), i.e., LacZ polypeptide, and firefly luciferase for imaging of virus delivery. FIG. 5A shows representative results of in vivo imaging that was used to non-invasively and longitudinally determine in mice the sites of expression of the luciferase tumor associated antigen encoded by a Sindbis viral vector, as described herein, after injection of the mice with the SV/TAA vector. At 3 hours after SV/TAA vector inoculation the mice were imaged. At 24 hours, the mediastinal and inguinal lymph nodes were extracted and Tcells were isolated and assessed for the presence of the T-cell activation marker CD69. Compared with the expression levels of the control T-cell activation marker CD69 in inguinal lymph nodes (ILN) (FIGS. 5A and 5B), the mediastinal lymph node (MLN) was identified as a site of delivery of the luciferase antigen (FIG. 5A) and was also found to be a site of potent CD8+ T cell activation after 24 hours (FIG. 5B).

FIGS. 6A-6D present graphs of relative tumor growth in mice having subcutaneous LacZ+ CT26 tumors versus the number of days following treatment with a Sindbis viral vector encoding the LacZ polypeptide (e.g., SV/LacZ) as described above. The results presented in the graphs were obtained from experiments in which control or vector-treated tumored mice were depleted of CD8+ and CD4+ T cells using an anti-CD8 antibody and an anti-CD4 antibody, as follows: 0.4 mg of each type of antibody in 0.2 ml PBS were injected into each mouse, starting 1 day before the first treatment with the SV/LacZ viral vector or mock control, and the antibodies were then injected every 2-3 days for 2 weeks thereafter. Mock control mice were injected with PBS. LacZ+ CT26 tumor-bearing mice were treated with either the SV/LacZ viral vector (Sindbis/LacZ) or with PBS (Mock). Tumor growth was determined by caliper measurement. FIG. 6A shows the results using control tumored mice, either mock-treated or treated with the SV/LacZ vector. FIG. 6B shows the results using tumored mice depleted of CD4⁺ T cells, either mock-treated or treated with the SV/LacZ vector. FIG. 6C shows the results using tumored mice depleted of CD8+ T cells, either mock-treated or treated with the SV/LacZ vector. FIG. 6D shows the results using tumored mice depleted of both CD4⁺ T cells and CD8⁺ T cells, either mock-treated or treated with the SV/LacZ vector.

The results depicted in FIGS. 6B-6D demonstrate that a therapeutic effect of the SV/LacZ vector on decreasing the growth of subcutaneous tumors was observed in the control mice having a normal complement of T cells, while a therapeutic effect was not observed in T cell-depleted mice. In accordance with the present invention, the therapeutic benefit obtained from treatment with a Sindbis viral vector encoding at least one, preferably two or more, epitopes of one or more tumor associated antigens, i.e., a SV/TAA viral vector, does not necessarily require the direct targeting of tumor cells. As supported by the Examples herein, SV/TAA therapy involved transient early delivery of the tumor associated antigen to lymph nodes draining the injection site, in particular, the mediastinal lymph nodes (MLN) in the case of intraperitoneal injection of the SV/TAA viral vector as demonstrated in FIG. 5A. Treatment with a SV/TAA viral vector also induced a potent TAA-specific CD8⁺ T cell response that was subsequently redirected against tumor cells expressing the cognate TAA. Further, SV/TAA therapy led to epitope spreading, providing a possible solution to the problem of tumor escape by TAA loss or modification, and SV/TAA therapy ultimately led to long-term survival of tumor-bearing mice, and to the generation of long-lasting memory CD8⁺ T cells against multiple TAAs. FIGS. 6A-6D provide evidence that the in vivo therapeutic effect of treatment with a Sindbis viral vector encoding at least one, preferably two or more, tumor associated antigen epitopes is T-cell-dependent, as tumor reduction following administration of the SV/LacZ viral vector was not observed in T-cell-depleted mice (FIGS. 6B-6D).

The results from the in vivo experiments utilizing the SV vector encoding multiple tumor associated epitopes evidence that SV provides an effective therapeutic platform for the immunogenic delivery of multiple TAA epitopes. Moreover, the therapeutic benefit obtained from SV/TAA generated an anti-tumor immune response that results in tumor cell killing, even if the tumor cells themselves are not directly targeted by the vector. SV/TAA therapy involves transient early delivery of the TAA epitopes to lymph nodes draining the injection site, in particular, the MLN in the case of i.p. SV injection. In addition, SV/TAA therapy induced a potent TAA-specific CD8⁺ T-cell response that is subsequently redirected against tumor cells expressing the cognate TAA and leads to epitope spreading, thus providing a possible solution to the problem of tumor escape by TAA loss or modification. As shown by the experimental results herein, SV/TAA therapy ultimately leads to long-term survival of tumor-bearing mice and to the generation of long-lasting memory CD8⁺ T cells against multiple TAAs.

### Example 6

### Prediction of Tumor Associated Antigen Epitopes for Use in Sindbis Viral Vectors

Multiple epitopic amino acid sequences of one or more tumor associated antigens for incorporation into the Sindbis viral vector according to the invention can be analyzed using the Immune Epitope Database, (www.IEDB.org), e.g., to rank epitope binding to BALB/c H2^{d} class I MHC.

This Example provides different epitope prediction algorithms for use in the selection of multiple epitopes encoded and expressed by the polynucleotides and viral vectors described herein. The amino acid sequence of the tumor associated antigen NY-ESO-1 was analyzed by the three predictions programs, namely, BIMAS: Biolnformatics and Molecular Analysis Section, ranks peptides by predicted dissociation constants from HLA alleles; IEDB: Immune Epitope Database (IEDB.org); and Rankpep for the prediction of peptide binding to MHC molecules as described below.

The NY-ESO-1 sequence analyzed for determining epitopes to generate an optimal T cell response is presented below.

NY-ESO-1 sequence>gi|4503119|ref|NP_001318.1|
cancer/testis antigen
1 [Homo sapiens]
(SEQ ID NO: 296)
MQAEGRGTGGSTGDADGPGGPGIPDGPGGNAGGPGEAGATGGRGPRGAGA
ARASGPGGGAPRGPHGGAASGLNGCCRCGARGPESRLLEFYLAMPFATPM
EAELARRSLAQDAPPLPVPGVLLKEFTVSGNILTIRLTAADHRQLQLSIS
SCLQQLSLLMWITQCFLPVFLAQPPSGQRR

HLA-A0201, a common human allele, was used for screening epitopes.
BIMAS: BioInformatics and Molecular Analysis Section (BIMAS), Center for Information Technology, National Institutes of Health, (http://www-bimas.cit.nih.gov). This web site allows users to locate and rank 8-mer, 9-mer, or 10-mer peptides that contain peptide-binding motifs for HLA class I molecules. The rankings employ amino acid/position coefficient tables deduced from the literature by Dr. Kenneth Parker, of Boston's Children's Hospital, Harvard Medical School, and of the National Institute of Allergy and Infectious Diseases (NIAID) at the National Institutes of Health (NIH) in Bethesda, Maryland. The Web site was created by Ronald Taylor of BIMAS, Computational Bioscience and Engineering Laboratory (CBEL), Division of Computer Research & Technology (CIT), National Institutes of Health, in collaboration with Dr. Parker. Information and Background on the HLA peptide motif searches that can be conducted via BISMAS is available via (https://www-bimas.cit.nih.gov/molbio/hla_bind/hla_motif_search_info.html). BISMAS provides HLA Peptide Binding Predictions and (an) algorithm(s) that ranks peptides by predicted dissociation constants from HLA alleles. HLA Peptide Binding Predictions ranks potential 8- to 10-mer peptides based on a predicted half-time of dissociation to HLA class 1 molecules. References for analysis of peptide/MHC Class I peptide binding motifs and ranking HLA-binding peptides include, e.g., Maier, R. et al., 1994, Immunogenetics, 40:306-308; Raghavan, et al., 1996, Protein Science, 5:2080-2088; Parker, K. C. et al., 1994, J. Immunol., 152:163-175; and Rammensee, H. G. et al., 1999, Immunogenetics, 50:213-219. Another database and computer software source (H.G. Rammensee) for obtaining information on epitope sequences based on analysis of peptide sequences and MHC specificity is SYFPEITHI (BMI Biomedical Informatics, SYFPEITHI@BMI-Heidelberg.com).

Table 34 shows HLA peptide motif search results, and associated user parameters and scoring information obtainable via BIMAS. The amino acid sequences set forth in the “Subsequence Residue Listing” in Table 34 are as follows: LMWITQCFL (SEQ ID NO: 297), RLLEFYLAM (SEQ ID NO: 298), GVLLKEFTV (SEQ ID NO: 299), WITQCFLPV (SEQ ID NO: 300), QLSLLMWIT (SEQ ID NO: 301), QQLSLLMWI (SEQ ID NO: 302), SLLMWITQC (SEQ ID NO: 32), SLAQDAPPL (SEQ ID NO: 303), ILTIRLTAA (SEQ ID NO: 304), and LWLSISSCL (SEQ ID NO: 305).

TABLE 34
HLA peptide motif search results
User Parameters and Scoring Information

| method selected to limit number of results | explicit number |
|---|---|
| number of results requested | 20 |
| HLA molecule type selected | A_0201 |
| length selected for subsequences to be scored | 9 |
| echoing mode selected for input squence | Y |
| echoing format | numbered lines |
| length of user's input peptide sequence | 180 |
| number of subsequence scores calculated | 172 |
| number of top-scoring subsequences reported back in scoring output table | 20 |

Scoring Results

| | | | Score (Estimate of Half Time |
|---|---|---|---|
| | | | of Disassociation of a Molecule |
| Rank | Start Position | Subsequence Residue Listing | Containing This Subsequence) |
| 1 | 159 | LMWITQCFL | 1197.321 |
| 2 | 86 | RLLEFYLAM | 429.578 |
| 3 | 120 | GVLLKEPTV | 130.601 |
| 4 | 161 | WITQCFLPV | 83.584 |
| 5 | 155 | QLSLLMWIT | 52.704 |
| 6 | 154 | QQLSLLMWI | 49.509 |
| 7 | 157 | GLLMWOTQC | 42.278 |
| 8 | 108 | SLAQDAPPL | 21.362 |
| 9 | 132 | ILTIRLTAA | 19.425 |
| 10 | 145 | LQLSISSCL | 13.624 |

IEDB: Immune Epitope Database (IEDB.org). The IEDB prediction tool uses a consensus of different algorithms that predict epitope binding to HLA alleles. The epitopes are then ranked—lower percentiles predict higher binding. The results of the prediction of MHC-1 binding are shown in the below Table 35. The peptides shown in Table 35 are identified as follows: SLAQDAPPL (SEQ ID NO: 303), LMWITQCFL (SEQ ID NO: 297), RLLEFYLAM (SEQ ID NO: 298), WITQCFLPV (SEQ ID NO: 300), GVLLKEFTV (SEQ ID NO: 299), AQDAPPLPV (SEQ ID NO: 306), QQLSLLMWI (SEQ ID NO: 302), LLMWITQCF (SEQ ID NO: 1), LQLSISSCL (SEQ ID NO: 307), SLLMWITQC (SEQ ID NO: 32), ILTIRLTAA (SEQ ID NO: 304), SISSCLQQL (SEQ ID NO: 308), LAMPFATPM (SEQ ID NO: 46), FATPMEAEL (SEQ ID NO: 44), CLQQLSLLM (SEQ ID NO: 309), FTVSGNILT (SEQ ID NO: 310), FYLAMPFAT (SEQ ID NO: 311).

TABLE 35

| | | Start | End | Length | | | |
|---|---|---|---|---|---|---|---|
| Allele [symbol] | # [symbol] | [symbol] | [symbol] | [symbol] | Peptide [symbol] | Method used [symbol] | Percentile_rank [symbol] |
| HLA-A*02:01 | 1 | 108 | 116 | 9 | SLAQDAPPL | Consensus (ann/smm/comblib_sidney2008) | 0.8 |
| HLA-A*02:01 | 1 | 159 | 167 | 9 | SMWITQCFL | Consensus (ann/smm/comblib_sidney2008) | 1.1 |
| HLA-A*02:01 | 1 | 86 | 94 | 9 | RLLEFYLAM | Consensus (ann/smm/comblib_sidney2008) | 1.4 |
| HLA-A*02:01 | 1 | 161 | 169 | 9 | WITQCFLPV | Consensus (ann/smm/comblib_sidney2008) | 2 |
| HLA-A*02:01 | 1 | 120 | 128 | 9 | GVLLKEFTV | Consensus (ann/smm/comblib_sidney2008) | 3 |
| HLA-A*02:01 | 1 | 110 | 118 | 9 | AQDAPPLPV | Consensus (ann/smm/comblib_sidney2008) | 3.2 |
| HLA-A*02:01 | 1 | 154 | 162 | 9 | QQLSLLMWI | Consensus (ann/smm/comblib_sidney2008) | 3.6 |
| HLA-A*02:01 | 1 | 158 | 166 | 9 | LLMWITQCF | Consensus (ann/smm/comblib_sidney2008) | 4 |
| HLA-A*02:01 | 1 | 145 | 153 | 9 | LQLSISSCL | Consensus (ann/smm/comblib_sidney2008) | 4.9 |
| HLA-A*02:01 | 1 | 157 | 165 | 9 | SLLMWITQC | Consensus (ann/smm/comblib_sidney2008) | 5.3 |
| HLA-A*02:01 | 1 | 132 | 140 | 9 | ILTIRLTAA | Consensus (ann/smm/comblib_sidney2008) | 5.6 |
| HLA-A*02:01 | 1 | 148 | 156 | 9 | SISSCLQQL | Consensus (ann/smm/comblib_sidney2008) | 5.6 |
| HLA-A*02:01 | 1 | 92 | 100 | 9 | LAMPFATPM | Consensus (ann/smm/comblib_sidney2008) | 6.5 |
| HLA-A*02:01 | 1 | 96 | 104 | 9 | FATPMEAEL | Consensus (ann/smm/comblib_sidney2008) | 6.7 |
| HLA-A*02:01 | 1 | 152 | 180 | 9 | CLQQLSLLM | Consensus (ann/smm/comblib_sidney2008) | 6.9 |
| HLA-A*02:01 | 1 | 126 | 134 | 9 | FTVSGNILT | Consensus (ann/smm/comblib_sidney2008) | 7.5 |
| HLA-A*02:01 | 1 | 90 | 98 | 9 | FYLAMPFAT | Consensus (ann/smm/comblib_sidney2008) | 7.7 |

Rankpep: This epitope experimental tool uses experimental data from known peptides that bind MHC/HLA and then compares sequences using a position specific scoring matrix. Rankpep uses Position Specific Scoring Matrices (PSSMs) or profiles from a set of aligned peptides (e.g., peptides aligned by structural or sequence similarity) known to bind to a given MHC molecule as the predictor of MHC-peptide binding. (http://imed.med.ucm.es/Tools/rankpep_help.html). It also takes into account which peptides are likely to be processed by proteases. (Reche P. A. et al., 2002, Prediction of MHC Class I Binding Peptides Using Profile Motifs, Human Immunology, 63: 701-709; Reche P. A. et al., 2004, Enhancement to the RANKPEP resource for the prediction of peptide binding to MHC molecules using profiles, Immunogenetics, 56:405-419; Reche P. A. and Reinherz E. L., 2007, Prediction of peptide-MHC binding using profiles. Methods Mol Biol., 409:185-200. Nonlimiting examples of MHC databases, including gene sequence, polymorphisms, etc., include IMGT (ImMunoGene Tics database); IMGT/HLA database, dbMHC (database at NCBI), Allele Frequencies database; HLA Informatics group; IHWG (International Histocompatibility Working Group); Genetics and Molecular Genetics of the MHC; and the Tumor Gene Database. Nonlimiting examples of peptide databases include MHCPEP, SYFPEITHI, HIV Molecular Immunology Database, MHCPEP HLA Ligand/Motif Database; MHCBN Database (comprehensive database of MHC binding and nonbinding peptides); HLA Ligand/Motif Database; JenPep Database (MHC and TAP ligands, T and B cell epitopes); FIMM Database (T and B cell epitopes); and MPID (MHC-peptide interaction database).

The results of the prediction of peptides binding to MHC molecules based on Rankpep output is shown in the below Table 36. The peptide sequences shown in Table 36 are identified as follows: TVSGNILTI (SEQ ID NO: 312), SISSCLQQL (SEQ ID NO: 308), RLLEFYLAM (SEQ ID NO: 298), CLQQLSLLM (SEQ ID NO: 313), SLAQDAPPL (SEQ ID NO: 303), SLLMWITQC (SEQ ID NO: 32), SCLQQLSLL (SEQ ID NO: 314), ILTIRLTAA (SEQ ID NO: 304), QLQLSISSC (SEQ ID NO: 315), and WITQCFLPV (SEQ ID NO: 300).

TABLE 36

| RANK | POS | N | SEQUENCE | C | MW (Da) | SCORE | % OPT |
|---|---|---|---|---|---|---|---|
| 1 | 127 | KEF | TVSGNILTI | RLT | 899.04 | 85.0 | 66.41% |
| 2 | 148 | LQL | SISSCLQQL | SLL | 960.12 | 84.0 | 65.62% |
| 3 | 86 | PES | RLLEFYLAM | PFA | 1137.42 | 78.0 | 60.94% |
| 4 | 152 | ISS | CLQQLSLLM | WIT | 1030.31 | 74.0 | 57.81% |
| 5 | 108 | ARR | SLAQDAPPL | PVP | 893.02 | 64.0 | 50.00% |
| 6 | 157 | QQL | SLLMWITQC | FLP | 1053.33 | 62.0 | 48.44% |
| 7 | 151 | SIS | SCLQQLSLL | MWI | 986.2 | 60.0 | 46.88% |
| 8 | 132 | SGN | ILTIRLTAA | DHR | 953.19 | 59.0 | 46.09% |
| 9 | 144 | DHR | QLQLSISSC | LQQ | 960.12 | 56.0 | 43.75% |
| 10 | 161 | LLM | WITQCFLPV | FLA | 1065.33 | 55.0 | 42.97% |

In the above Table 36, the light-gray highlighted rows 1-5 represent predicted binders. Rows 2, 5 and 7 of the table provide information about peptides with C-termini predicted by cleavage models.

### Analysis of Results

The results of the epitope analysis of the NY-ESO-1 tumor associated antigen showed the ranking of several different epitopes in the protein using the above-described algorithms. A NY-ESO-1 epitope frequently used for cancer immunotherapy is SLLMWITQC (SEQ ID NO: 32). The rank of this epitope as determined by the use of the three algorithms was as follows: BIMAS: 7; IEDB: 10; Rankpep: 7 (10+7+7=24), as shown in Table 37 below.

The results indicate that peptide RLLEFYLAM (SEQ ID NO: 298) may also be a good epitope as it is ranked highly by all three algorithms.

TABLE 37
Ranking of Epitopes Based on BIMAS,
IEDB and RANKPEP algorithms

| EPITOPE | BIMAS | IEDB | RANKPEP |
|---|---|---|---|
| SLAQDAPPL (SEQ ID NO: 303) | 8 | 1 | 5 |
| LMWITQCFL (SEQ ID NO: 297) | 1 | 2 | — |
| RLLEFYLAM (SEQ ID NO: 298) | 2 | 3 | 3 |
| WITQCFLPV (SEQ ID NO: 300) | 4 | 4 | 10 |
| GVLLKEFTV (SEQ ID NO: 299) | 3 | 5 | — |
| SLLMWITQC (SEQ ID NO: 32) | 7 | 10 | 7 |

### Other Embodiments

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

All patents and publications mentioned in this specification are herein incorporated by reference to the same extent as if each independent patent and publication was specifically and individually indicated to be incorporated by reference.

What is claimed is:

1. A polynucleotide encoding an alphavirus protein, or a fragment thereof, and two or more epitopes of one or more tumor associated antigens, wherein each epitope is separated by an enzyme cleavage site.

2. The polynucleotide of claim 1, wherein the alphavirus protein, or a fragment thereof, is a Sindbis virus protein, or a fragment thereof.

3. The polynucleotide of claim 1, wherein the two or more epitopes comprise an amino acid sequence of a tumor associated antigen listed in any one of Tables 1-28.

4. The polynucleotide of claim 3, wherein the two or more epitopes are of one or more tumor associated antigens selected from kallikrein 4, papillomavirus binding factor (PBF), preferentially expressed antigen of melanoma (PRAME), Wilms' tumor-1 (WT1), Hydroxysteroid Dehydrogenase Like 1 (HSDL1), mesothelin, cancer testis antigen (NY-ESO-1), carcinoembryonic antigen (CEA), p53, human epidermal growth factor receptor 2/neuro receptor tyrosine kinase (Her2/Neu), carcinoma-associated epithelial cell adhesion molecule EpCAM), ovarian and uterine carcinoma antigen (CA125), folate receptor a, sperm protein 17, tumor-associated differentially expressed gene-12 (TADG-12), mucin-16 (MUC-16), L1 cell adhesion molecule (L1CAM), mannan-MUC-1, Human endogenous retrovirus K (HERV-K-MEL), Kita-kyushu lung cancer antigen-1 (KK-LC-1), human cancer/testis antigen (KM-HN-1), cancer testis antigen (LAGE-1), melanoma antigen-A1 (MAGE-A1), Sperm surface zona pellucida binding protein (Sp17), Synovial Sarcoma, X Breakpoint 4 (SSX-4), Transient axonal glycoprotein-1 (TAG-1), Transient axonal glycoprotein-2 (TAG-2), Enabled Homolog (ENAH), mammoglobin-A, NY-BR-1, Breast Cancer Antigen, (BAGE-1), B melanoma antigen, melanoma antigen-A1 (MAGE-A1), melanoma antigen-A2 (MAGE-A2), mucin k, synovial sarcoma, X breakpoint 2 (SSX-2), Taxol-resistance-associated gene-3 (TRAG-3), Avian Myelocytomatosis Viral Oncogene (c-myc), cyclin B1, mucin 1 (MUC1), p62, survivin, lymphocyte common antigen (CD45), Dickkopf WNT Signaling Pathway Inhibitor 1 (DKK1), telomerase, Kirsten rat sarcoma viral oncogene homolog (K-ras), G250, intestinal carboxyl esterase, alpha-fetoprotein, Macrophage Colony-Stimulating Factor (M-CSF), Prostate-specific membrane antigen (PSMA), caspase 5 (CASP-5), Cytochrome C Oxidase Assembly Factor 1 Homolog (COA-1), O-linked (3-N-acetylglucosamine transferase (OGT), Osteosarcoma Amplified 9, Endoplasmic Reticulum Lectin (OS-9), Transforming Growth Factor Beta Receptor 2 (TGF-betaRII), murine leukemia glycoprotein 70 (gp70), Calcitonin Related Polypeptide Alpha (CALCA), Programmed cell death 1 ligand 1 (CD274), Mouse Double Minute 2Homolog (mdm-2), alpha-actinin-4, elongation factor 2, Malic Enzyme 1 (ME1), Nuclear Transcription Factor Y Subunit C (NFYC), G Antigen 1,3 (GAGE-1,3), melanoma antigen-A6 (MAGE-A6), cancer testis antigen XAGE-1b, six transmembrane epithelial antigen of the prostate 1 (STEAP1), PAP, prostate specific antigen (PSA), Fibroblast Growth Factor 5 (FGF5), heat shock protein hsp70-2, melanoma antigen-A9 (MAGE-A9), Arg-specific ADP-ribosyltransferase family C (ARTC1), B-Raf Proto-Oncogene (B-RAF), Serine/Threonine Kinase, beta-catenin, Cell Division Cycle 27 homolog (Cdc27), cyclin dependent kinase 4 (CDK4), cyclin dependent kinase 12 (CDK12), Cyclin Dependent Kinase Inhibitor 2A (CDKN2A), Casein Kinase 1 Alpha 1 (CSNK1A1), Fibronectin 1 (FN1), Growth Arrest Specific 7 (GAS7), Glycoprotein nonmetastatic melanoma protein B (GPNMB), HAUS Augmin Like Complex Subunit 3 (HAUS3), LDLR-fucosyltransferase, Melanoma Antigen Recognized By T-Cells 2 (MART2), myostatin (MSTN), Melanoma Associated Antigen (Mutated) 1 (MUM-1-2-3), Poly(A) polymerase gamma (neo-PAP), myosin class I, Protein phosphatase 1 regulatory subunit 3B (PPP1R3B), Peroxiredoxin-5 (PRDXS), Receptor-type tyrosine-protein phosphatase kappa (PTPRK), Transforming protein N-Ras (N-ras), retinoblastoma-associated factor 600 (RBAF600), sirtuin-2 (SIRT2), SNRPD1, triosephosphate isomerase, Ocular Albinism Type 1 Protein (OA1), member RAS oncogene family (RAB38), Tyrosinase related protein 1-2 (TRP-1-2), Melanoma Antigen Gp75 (gp75), tyrosinase, Melan-A (MART-1), Glycoprotein 100 melanoma antigen (gp100), N-acetylglucosaminyltransferase V gene (GnTVf), Lymphocyte Antigen 6 Complex Locus K (LY6K), melanoma antigen-A10 (MAGE-A10), melanoma antigen-A12 (MAGE-A12), melanoma antigen-C2 (MAGE-C2), melanoma antigen NA88-A, Taxol-resistant-associated protein 3 (TRAG-3), BDZ binding kinase (pbk), caspase 8 (CASP-8), sarcoma antigen 1 (SAGE), Breakpoint Cluster Region-Abelson oncogene (BCR-ABL), fusion protein in leukemia, dek-can, Elongation Factor Tu GTP Binding Domain Containing 2 (EFTUD2), ETS Variant gene 6/acute myeloid leukemia fusion protein (ETV6-AML1), FMS-like tyrosine kinase-3 internal tandem duplications (FLT3-ITD), cyclin-A1, Fibronectin Type III Domain Containing 3B (FDNC3B,) promyelocytic leukemia/retinoic acid receptor alpha fusion protein (pml-RARalpha), melanoma antigen-C1 (MAGE-C1), membrane protein alternative spliced isoform (D393-CD20), melanoma antigen-A4 (MAGE-A4), or melanoma antigen-A3 (MAGE-A3).

5. The polynucleotide of claim 4, wherein the epitope from the tumor associated antigen NY-ESO-1 comprises the amino acid sequence LLMWITQCF (SEQ ID NO: 1), the epitope from the tumor associated antigen pbk comprises the amino acid sequence GSPFPAAVI (SEQ ID NO: 2), the epitope from the tumor associated antigen NY-ESO-1 comprises the amino acid sequence RGPESRLLE (SEQ ID NO: 3), and the epitope from the tumor associated antigen survivin comprises the amino acid sequence AFLTVKKQM (SEQ ID NO: 4).

6. The polynucleotide of claim 1, wherein the epitopes are of tumor associated antigens expressed on the surface of a cancer cell of a/an ovarian cancer, breast cancer, testicular cancer, pancreatic cancer, liver cancer, colon cancer, colorectal cancer, thyroid cancer, lung cancer, prostate cancer, kidney cancer, melanoma, squamous cell carcinoma, chronic myeloid leukemia, acute lymphoblastic leukemia, acute myelogenous leukemia, chronic lymphocytic leukemia, promyelocytic leukemia, multiple myeloma, B-cell lymphoma, bladder carcinoma, head and neck cancer, esophageal cancer, brain cancer, pharynx cancer, tongue cancer, synovial cell carcinoma, neuroblastoma, uterine cancer, fibrosarcoma, myxosarcoma, liposarcoma, chondrosarcoma, osteogenic sarcoma, chordoma, angiosarcoma, endotheliosarcoma. lymphangiosarcoma, synovioma, mesothelioma, Ewing's tumor, leiomyosarcoma, rhabdomyosarcoma, basal cell carcinoma, epidermoid carcinoma, adenocarcinoma, sweat gland carcinoma, sebaceous gland carcinoma, papillary carcinoma, papillary adenocarcinomas, cystadenocarcinoma, medullary carcinoma, bronchogenic carcinoma, renal cell carcinoma, hepatoma, bile duct carcinoma, choriocarcinoma, seminoma, embryonal carcinoma, Wilms'·tumor, cervical cancer, small cell lung carcinoma, epithelial carcinoma, glioma, astrocytoma, medulloblastoma, craniopharyngioma, ependymoma, pinealoma, hemangioblastoma, acoustic neuroma, oligodendroglioma, meningioma, neuroglioma, or retinoblastoma.

7. The polynucleotide of claim 1, wherein the protease cleavage site is cleaved by furin.

8. The polynucleotide of claim 1, further comprising one or more suicide genes.

9. The polynucleotide of 8, wherein the one or more suicide genes is capable of converting an inert prodrug into a cytotoxic metabolite, and the inert prodrug is selected from the group consisting of ganciclovir, acyclovir, 1-(2-deoxy-2-fluoro-β-D-arabinofuranosyl)-5-iodouracil (FIAU), 6-methoxypurine arabinoside, and 5-fluorocytosine.

10. The polynucleotide of claim 9, wherein the one or more suicide genes encodes thymidine kinase or cytosine deaminase.

11. A viral particle or viral vector comprising the polynucleotide of claim 1.

12. The viral vector of claim 11, wherein the viral vector is derived from a Sindbis virus or pseudotyped with one or more Sindbis virus envelope proteins.

13. A Sindbis viral vector comprising a polynucleotide encoding two or more epitopes comprising 5-30 amino acids of a tumor associated antigen, wherein each epitope is separated by a furin enzyme cleavage site.

14. The viral vector of claim 13, wherein the viral vector is capable of eliciting an immune response against a tumor or cancer expressing the two or more epitopes of the one or more tumor associated antigens following administration to a subject.

15. A lentiviral vector pseudotyped with one or more genetically engineered Sindbis virus envelope proteins, said lentiviral vector comprising the polynucleotide of claim 1.

16. A lentiviral vector pseudotyped with one or more genetically engineered Sindbis virus envelope proteins, said lentiviral vector comprising the polynucleotide of claim 1, wherein the polynucleotide encodes an epitope of one or more tumor associated antigens selected from NY-ESO-1, MAGE-A3, pbk, survivin, or a combination thereof.

17. A cell comprising the polynucleotide of claim 1.

18. A pharmaceutical composition comprising the polynucleotide of claim 1 or a viral vector comprising said polynucleotide and a pharmaceutically acceptable vehicle, carrier, or diluent.

19. A method of inducing an immune response against a cancer or tumor cell or treating cancer in a subject, the method comprising contacting the cancer or tumor cell with an effective amount of the polynucleotide of claim 1, or a viral particle or viral vector comprising said polynucleotide, thereby inducing an immune response against the cancer or tumor cell or treating the cancer.

20. The method of claim 19, wherein the cancer is one or more of ovarian cancer, cervical cancer, breast cancer, or colon cancer.

21. The method if claim 19, wherein the polynucleotide encodes two or more epitopes of one or more of the tumor associated antigens NY-ESO-1, p53, sp17, survivin, pbk, CEA, CA125, or WT1.

22. The method of claim 19, wherein the administering causes epitope spreading in the subject.

23. A viral vector pseudotyped with one or more alphavirus envelope proteins, wherein the viral vector comprises a polynucleotide encoding two or more epitopes comprising 5-30 amino acids of a tumor associated antigen, wherein each epitope is separated by an enzyme cleavage site.

24. The viral vector of claim 23, wherein the enzyme cleavage site is a furin enzyme cleavage site.
